(19) United States
(12) Patent Application Publication
(10) Pub. No.: US 2023/0293647 A1
(43) Pub. Date: Sep. 21, 2023

(54) Polypeptide

(71) Applicant: AUTOLUS LIMITED, London (GB)

(72) Inventors: Mathieu Ferrari, London (GB); Shimobi Onuoha, London (GB); Martin Pulé, London (GB); Alexander Kinna, London (GB); Leila Mekkaoui, London (GB); Preeta Datta, London (GB)

(21) Appl. No.: 17/917,678

(22) PCT Filed: Apr. 9, 2021
(86) PCT No.: PCT/GB2021/050876

§ 371 Date: Oct. 7, 2022

(30) Foreign Application Priority Data
Apr. 9, 2020 (GB) 2005333.6
Aug. 26, 2020 (GB) 2013372.4
Mar. 3, 2021 (GB) 2103001.0

(51) Int. Cl. A61K 38/48; A61P 31/14; C12N 9/48; C12N 15/85

(52) U.S. Cl. CPC A61K 38/4813; A61P 31/14; C12N 9/485; C12Y 304/17023; C12N 15/85; C12N 2800/107

(57) ABSTRACT

The present invention provides polypeptides with coronavirus neutralising capacity. It further provides nucleic acids, vectors, cells, pharmaceutical compositions and medical uses that exploit the polypeptides of the invention

### FIELD OF THE INVENTION

The present invention relates to molecules with therapeutic activity against a coronavirus infection.

### BACKGROUND TO THE INVENTION

In December 2019, a novel coronavirus (SARS-CoV-2, COVID-19 or 2019-nCoV) crossed species barriers to infect humans and was effectively transmitted from person to person, leading to a pneumonia outbreak first reported in Wuhan, China. This virus causes coronavirus disease-19 (COVID-19) with influenza like symptoms ranging from mild disease to severe lung injury and multi-organ failure, eventually leading to death, especially in older patients with other co-morbidities. The WHO has declared that COVID-19 is a public health emergency of pandemic proportions. The SARS-CoV-2 pandemic is not only an enormous burden to public health but has already markedly affected civil societies and the global economy.

SARS-CoV-2 is currently considered a worldwide pandemic outbreak, with more than 100 million confirmed cases and more than 2 million confirmed deaths as of January 2021. Trials are currently ongoing for the antiviral reagents Remdesivir (Gilead), Chloroquine and hydroxychloroquine, and Ritonavir/Lopinavir (Kaletra, AbbVie). Other companies such as EliLilly/AbCeller, Takeda, and Regeneron are also testing neutralising antibodies, while a number of vaccine strategies have been approved (notably those from Astra Zeneca, Pfizer/BioNTech and Moderna) or are currently being investigated.

Recent structural data has elucidated the mechanism by which SARS-CoV-2 viral particles enter cells. SARS-CoV-2 has been shown to bind to angiotensin-converting enzyme 2 (ACE2) via the spike protein (S protein) on its surface (FIG. 1). In a recent publication, Lei at al. described a recombinant ACE2-Fc fusion protein able to neutralize SARS-CoV-2 (Lei et al., 2020, bioRxiv 2020.02.01.929976; https://doi.org/10.1101/2020.02.01.929976). A similar construct was also effective against SARS-CoV in 2003 (Moore et al., 2004, J Virol 78:10628-35). A pilot clinical trial from GSK (NCT01597635) using a recombinant version of ACE2 (GSK2586881) proved to be well tolerated in patients with acute respiratory distress syndrome (Khan et al., 2017, Crit Care 21:234). However, it is not clear whether the catalytic activity of ACE2 will have an adverse effect on the renin-angiotensin system and provoke detrimental and possibly long-term effects on patients.

A recent study reported that CD147 may bind S protein of SARS-CoV-2 and possibly be involved in host cell invasion (Wang et al., bioRxiv 2020.03.14.988345; https://doi.org/10.1101/2020.03.14.988345). Consequently, meplazumab, a humanised anti-CD147 antibody is being tested in patients with SARS-CoV-2 pneumonia.

Although SARS-CoV and SARS-CoV-2 S proteins share a high amino acid homology (76.5%), neutralising antibodies against SARS-CoV have been shown to decrease SARS-CoV-2 infectivity but did not block it entirely (Hoffmann et al., 2020, Cell, in press; Walls et al., 2020, Cell, in press). Moreover, due to the nature of SARS-CoV-2, a high mutation rate is expected as a result of selective pressure. Several strain variants have already been described in the literature, which in some cases presented mutations within the receptor binding domain (RDB) of the S protein (Ou et al., 2020, bioRxiv 2020.03.15.991844; doi:10.1101/2020.03.15.991844; Wang et al., 2020, J Med Virol, early view; doi:10.1002/jmv.25762). Some of the RDB mutations resulted in a higher binding affinity for ACE2 (Ou et al., 2020, bioRxiv 2020.03.15.991844; doi:10.1101/2020.03.15.991844). This occurrence may prevent successful application of neutralising antibodies in clinical therapy.

Paradoxically, non-neutralizing antibodies to variable S domains may enable an alternative infection pathway via Fc receptor-mediated uptake. These antibodies can act to enhance viral infection by aiding viral entry into target and non-target cells. This mechanism of improved virus uptake, termed antibody-dependent enhancement (ADE) of infection. Prior studies involving anti-spike protein antibodies (Yip et al., 2014, Virol J 11:82) and vaccine candidates for SARS-CoV (Wang et al., 2014, Biochem Biophys Res Commun 451:208-14) and Middle East Respiratory Syndrome coronavirus (MERS-CoV) (Agrawal et al., 2016, Hum Vaccine Immunother 12:2351-6) demonstrate vaccination-induced ADE of disease, including infection of phagocytic antigen presenting cells (APC)—so-called extrinsic ADE.

Once internalised, these immunocomplexes may modulate innate antiviral cells responses to increase virus production substantially in each cell, a process termed intrinsic ADE. Together, extrinsic and intrinsic ADE are thought to prompt the massive release of inflammatory and vasoactive mediators that ultimately contribute to disease severity. Sequence and structural conservation of S motifs suggests that SARS and MERS vaccine ADE risks may foreshadow the risks of SARS-CoV-2 S-based vaccine or antibody mediated approaches.

The rapid mutation rate of coronaviruses, and particularly of SARS-CoV-2, has already revealed weaknesses in the antibody and vaccine approaches. Therefore, there is a need in the art to provide effective therapeutics for the treatment of human-infecting coronaviruses and, especially, SARS-CoV-2.

### SUMMARY OF ASPECTS OF THE INVENTION

The present inventors have generated a series of molecular clamps with ability to neutralise coronaviruses, and SARS-CoV-2 in particular. These molecular clamps are based on angiotensin converting enzyme type 2 (ACE2), CD147 and/or antibodies that are specific for the coronavirus S protein. The avidity of these molecules for coronavirus virions has been increased following different genetic engineering approaches, ranging from oligomerisation to combining these molecules into fusion proteins. Through stronger interactions with the coronavirus S protein and/or the virions, the inefficient neutralisation capacity of previous targeting approaches is improved. Moreover, the use of two or more binding events minimises the risk of viral escape mechanisms through viral mutation.

Thus, in a first aspect, the present invention provides a polypeptide comprising:
a) a domain A which comprises a variant of the ectodomain of human angiotensin converting enzyme type 2 (hACE2) which maintains the capacity of hACE2 to interact with the S protein of coronavirus, and
b) a domain O which comprises an oligomerisation domain, wherein the oligomerisation domain comprises an IgG Fc region variant which does not interact with FcγRI, FcγRIIa and FcγRIII.

The variant of the ectodomain of hACE2 thereof which maintains the capacity of hACE2 to interact with the S protein of coronavirus may comprise the sequence shown as SEQ ID NO: 104.

The IgG Fc region variant which does not interact with FcγRI, FcγRIIa and FcγRIII may comprise the sequence shown as SEQ ID NO: 121.

The polypeptide according to the first aspect may comprise or consist of the amino acid sequence shown as SEQ ID NO: 125.

In a second aspect, the present invention provides a nucleic acid encoding the polypeptide according to the first aspect of the invention.

In a third aspect, the present invention provides an expression cassette comprising the nucleic acid according to the second aspect of the invention.

In a fourth aspect, the present invention provides a vector comprising the nucleic acid according to the second aspect of the invention or the expression cassette according to the third aspect of the invention.

In an fifth aspect, the present invention provides a cell comprising the nucleic acid according to the second aspect of the invention or the expression cassette according to the third aspect of the invention, or the vector according to the fourth aspect of the invention.

In a sixth aspect, the present invention provides a pharmaceutical composition comprising the polypeptide according to the first aspect of the invention, or the nucleic acid according to the second aspect of the invention, or the expression cassette according to the third aspect of the invention, or the vector according to the fourth aspect of the invention, or the cell according to the fifth aspect of the invention and a pharmaceutically acceptable carrier, diluent, excipient or adjuvant.

In a seventh aspect, the present invention provides a polypeptide according to the first aspect of the invention, or a pharmaceutical composition according to the sixth aspect of the invention, for use in medicine.

In an eighth aspect, the present invention provides a polypeptide according to the first aspect of the invention, or a pharmaceutical composition according to the sixth aspect of the invention, for use in the treatment of a coronavirus infection or a condition or disorder resulting from this infection.

In a ninth aspect, the present invention provides a use of a polypeptide according to the first aspect of the invention, or a pharmaceutical composition according to the sixth aspect of the invention, in the manufacturing of a medicament for the treatment of a coronavirus infection or a condition or disorder resulting from this infection.

In an tenth aspect, the present invention provides a method for treating a coronavirus infection or a condition or disorder resulting from this infection in a subject in need thereof comprising a step of administering a polypeptide according to the first aspect of the invention, or a pharmaceutical composition according to the sixth aspect of the invention, to the subject.

In a eleventh aspect, the present invention provides a method of neutralising a coronavirus infection, comprising a step of contacting a polypeptide according to the first aspect of the invention, or a pharmaceutical composition according to the sixth aspect of the invention, with a cell infected with said coronavirus.

In a twelfth aspect, the present invention provides a method for treating a subject having COVID-19 of unknown SARS-CoV-2 strain, comprising a step of administering a polypeptide according the first aspect of the invention, or a pharmaceutical composition according to the sixth aspect of the invention to the subject.

In a thirteenth aspect, the present invention provides a method for treating a subject previously immunised with a vaccine based on S protein depicted under Uniprot accession number P0DTC2, comprising a step of administering a polypeptide according the first aspect of the invention, or a pharmaceutical composition according to the sixth aspect of the invention to the subject.

In a fourteenth aspect, the present invention provides a method for treating a subject previously treated with antibodies specific to S protein depicted under Uniprot accession number P0DTC2, comprising a step of administering a polypeptide according the first aspect of the invention, or a pharmaceutical composition according to the sixth aspect of the invention to the subject.

In a fifteenth aspect, the present invention provides a method for treating a subject previously infected with a first SARS-CoV-2 strain who is currently infected with a second SARS-CoV-2 strain, wherein the first and second SARS-CoV-2 strains are different, comprising a step of administering a polypeptide according the first aspect of the invention, or a pharmaceutical composition according to the sixth aspect of the invention to the subject.

In a sixteenth aspect, the present invention provides a method for treating a coronavirus infection of one SARS-CoV-2 strain selected from wild-type, variant D614G, variant A222V, variant S477N, variant B.1.1.7, variant B.1.351, and variant B.1.1.28, comprising a step of administering a polypeptide according the first aspect of the invention, or a pharmaceutical composition according to the sixth aspect of the invention to the subject.

In a seventeenth aspect, the present invention provides a method of neutralising a coronavirus infection, comprising a step of contacting a polypeptide according the first aspect of the invention, or a pharmaceutical composition according to the sixth aspect of the invention with a cell infected with said coronavirus.

The coronavirus in the eighth, ninth, tenth, and seventeenth aspects of the invention may be SARS-CoV-2.

### Further Aspects of the Invention

The present invention also relates to the aspects listed in the following numbered paragraphs:

1. A polypeptide comprising:
a) a domain A which comprises the ectodomain of human angiotensin converting enzyme type 2 (hACE2), a fragment thereof comprising the amino acid sequence shown as SEQ ID NO: 3, or a variant thereof which maintains the capacity of hACE2 to interact with the S protein of coronavirus, and
b) a domain O which comprises an oligomerisation domain,

wherein the oligomerisation domain is selected from an IgG Fc region or a variant thereof which does not interact with FcγRI, FcγRIIa and FcγRIII, an IgM Fc region, an IgA Fc region, a collagen XVIII trimerizing structural element, a collagen XV trimerizing structural element, a foldon domain, a TenC domain and a coiled coil domain,

with the proviso that if the domain A consists of the ectodomain of hACE2 then the domain O is not the IgG1 Fc region.

2. A polypeptide comprising:
a) a domain A which comprises the ectodomain of human angiotensin converting enzyme type 2 (hACE2), a fragment thereof comprising the amino acid sequence shown as SEQ ID NO: 3, or a variant thereof which maintains the capacity of hACE2 to interact with the S protein of coronavirus,
b) a domain O which comprises an oligomerisation domain, and
c) a domain C which comprises the ectodomain of CD147 or a fragment thereof comprising the amino acid sequence shown as SEQ ID NO: 8 or SEQ ID NO: 9.

3. The polypeptide according to paragraph 2, wherein the oligomerisation domain is selected from an IgG Fc region or a variant thereof which does not interact with FcγRI, FcγRIIa and FcγRIII, an IgM Fc region, an IgA Fc region, a collagen XVIII trimerizing structural element, a collagen XV trimerizing structural element, a foldon domain, a TenC domain and a coiled coil domain.

4. The polypeptide according to any of paragraphs 1 to 3, wherein the variant thereof which maintains the capacity of hACE2 to interact with the S protein of coronavirus comprises the sequence shown as SEQ ID NO: 104.

5. The polypeptide according to any of paragraphs 1 to 4, further comprising a domain ABD, wherein the domain ABD comprises an antigen-binding domain that binds specifically to a coronavirus spike protein (S protein).

6. The polypeptide according to paragraph 5, wherein the ectodomain of hACE2, or a fragment thereof comprising the amino acid sequence shown as SEQ ID NO: 3, or a variant thereof which maintains the capacity of hACE2 to interact with the S protein of coronavirus, and the antigen-binding domain bind to different epitopes on the S protein of coronavirus.

7. The polypeptide according to paragraph 4, wherein the ectodomain of CD147, or a fragment thereof comprising the amino acid sequence shown as SEQ ID NO: 8 or SEQ ID NO: 9, and the antigen-binding domain bind to different epitopes on the coronavirus S protein.

8. The polypeptide according to any of paragraphs 1 to 7, further comprising a domain ALB which comprises an antigen-binding domain that binds specifically to albumin, or an albumin domain, or an antigen-binding domain that binds specifically to albumin, or an albumin-binding-peptide, or an albumin-binding domain of a Streptococcus protein.

9. The polypeptide according to any of paragraphs 1 to 8, wherein each of domain A, domain O, and, where present, domain C, domain ABD and/or domain ALB, are joined by a linker.

10. The polypeptide according to paragraph 4, which comprises the amino acid sequence shown as SEQ ID NO: 125.

11. A polypeptide comprising:
a) a domain C which comprises the ectodomain of CD147 or a fragment thereof comprising the amino acid sequence shown as SEQ ID NO: 8 or a fragment thereof comprising the amino acid sequence shown as SEQ ID NO: 9, and
b) a domain O which comprises an oligomerisation domain.

12. The polypeptide according paragraph 11, wherein the oligomerisation domain is selected from an IgG Fc region or a variant thereof which does not interact with FcγRI, FcγRIIa and FcγRIII, an IgM Fc region, an IgA Fc region, a collagen XVIII trimerizing structural element, a collagen XV trimerizing structural element, a foldon domain, a TenC domain and a coiled coil domain.

13. The polypeptide according to any of paragraphs 11 or 12, further comprising a domain ABD, wherein the domain ABD comprises an antigen-binding domain that binds specifically to a coronavirus S protein.

14. The polypeptide according to any of c paragraphs laims 11 to 13, further comprising a domain ALB which comprises an antigen-binding domain that binds specifically to albumin, or an albumin domain, or an antigen-binding domain that binds specifically to albumin, or an albumin-binding-peptide, or an albumin-binding domain of a Streptococcus protein.

15. The polypeptide according to any of paragraphs 11 to 14, wherein each of domain C, domain O, and, where present, domain ABD and/or domain ALB, are joined by a linker.

16. The polypeptide according to paragraph 15, wherein the linker is (Gly4Ser)3 (SEQ ID NO: 6).

17. A polypeptide comprising:
a) a domain ABD which comprises an antigen-binding domain that binds specifically to a coronavirus S protein, and
b) a domain O which comprises an oligomerisation domain.

18. The polypeptide according paragraph 17, wherein the oligomerisation domain is selected from an IgG Fc region or a variant thereof which does not interact with FcγRI, FcγRIIa and FcγRIII, an IgM Fc region, an IgA Fc region, a collagen XVIII trimerizing structural element, a collagen XV trimerizing structural element, a foldon domain, a TenC domain and a coiled coil domain.

19. The polypeptide according to any of paragraphs 17 or 18, further comprising a domain ALB which comprises an antigen-binding domain that binds specifically to albumin, or an albumin domain, or an antigen-binding domain that binds specifically to albumin, or an albumin-binding-peptide, or an albumin-binding domain of a Streptococcus protein.

20. The polypeptide according to any of paragraphs 17 to 19, wherein domain ABD and domain O, and, where present, domain ALB, are joined by a linker.

21. The polypeptide according to paragraph 20, wherein the linker is (Gly4Ser)3 (SEQ ID NO: 6).

22. The polypeptide according to any of paragraphs 4 to 10 and 13 to 21, wherein the antigen-binding domain binds to the S1 subunit or the S2 subunit of the coronavirus S protein.

23. The polypeptide according to any of paragraphs 4 to 10 and 13 to 22, wherein the antigen-binding domain is selected from a scFv or a domain antibody (dAb or VH).

24. The polypeptide according to any of paragraphs 4 to 10 and 13 to 23, wherein the antigen-binding domain comprises the CDR1, CDR2 and CDR3 from one of the following sequences:
a VH sequence of SEQ ID NO: 10 and a VL sequence of SEQ ID NO: 11;
a VH sequence of SEQ ID NO: 12 and a VL sequence of SEQ ID NO: 13;
a VH sequence of SEQ ID NO: 14 and a VL sequence of SEQ ID NO: 15;
a VH sequence of SEQ ID NO: 16 and a VL sequence of SEQ ID NO: 17;
a VH sequence of SEQ ID NO: 18 and a VL sequence of SEQ ID NO: 19;
a VH sequence of SEQ ID NO: 20 and a VL sequence of SEQ ID NO: 21;
a VH sequence of SEQ ID NO: 22 and a VL sequence of SEQ ID NO: 23;
a VH sequence of SEQ ID NO: 24 and a VL sequence of SEQ ID NO: 25;
a VH sequence of SEQ ID NO: 26 and a VL sequence of SEQ ID NO: 27;
a VH sequence of SEQ ID NO: 28 and a VL sequence of SEQ ID NO: 29;
a VH sequence of SEQ ID NO: 30 and a VL sequence of SEQ ID NO: 31;
a VH sequence of SEQ ID NO: 32 and a VL sequence of SEQ ID NO: 33;
a VH sequence of SEQ ID NO: 34 and a VL sequence of SEQ ID NO: 35;
a VH sequence of SEQ ID NO: 36 and a VL sequence of SEQ ID NO: 37;
a VH sequence of SEQ ID NO: 38 and a VL sequence of SEQ ID NO: 39;
a VH sequence of SEQ ID NO: 40 and a VL sequence of SEQ ID NO: 41;
a VH sequence of SEQ ID NO: 42 and a VL sequence of SEQ ID NO: 43;
a VH sequence of SEQ ID NO: 44 and a VL sequence of SEQ ID NO: 45;
a VH sequence of SEQ ID NO: 46 and a VL sequence of SEQ ID NO: 47;
a VH sequence of SEQ ID NO: 48 and a VL sequence of SEQ ID NO: 49;
a VH sequence of SEQ ID NO: 50 and a VL sequence of SEQ ID NO: 51;
a VH sequence of SEQ ID NO: 52 and a VL sequence of SEQ ID NO: 53;
a VH sequence of SEQ ID NO: 58;
a VH sequence of SEQ ID NO: 59; and
a VH sequence of SEQ ID NO: 60.

25. The polypeptide according to paragraph 24, wherein the antigen-binding domain comprises
a VH sequence of SEQ ID NO: 10 and a VL sequence of SEQ ID NO: 11;
a VH sequence of SEQ ID NO: 12 and a VL sequence of SEQ ID NO: 13;
a VH sequence of SEQ ID NO: 14 and a VL sequence of SEQ ID NO: 15;
a VH sequence of SEQ ID NO: 16 and a VL sequence of SEQ ID NO: 17;
a VH sequence of SEQ ID NO: 18 and a VL sequence of SEQ ID NO: 19;
a VH sequence of SEQ ID NO: 20 and a VL sequence of SEQ ID NO: 21;
a VH sequence of SEQ ID NO: 22 and a VL sequence of SEQ ID NO: 23;
a VH sequence of SEQ ID NO: 24 and a VL sequence of SEQ ID NO: 25;
a VH sequence of SEQ ID NO: 26 and a VL sequence of SEQ ID NO: 27;
a VH sequence of SEQ ID NO: 28 and a VL sequence of SEQ ID NO: 29;
a VH sequence of SEQ ID NO: 30 and a VL sequence of SEQ ID NO: 31;
a VH sequence of SEQ ID NO: 32 and a VL sequence of SEQ ID NO: 33;
a VH sequence of SEQ ID NO: 34 and a VL sequence of SEQ ID NO: 35;
a VH sequence of SEQ ID NO: 36 and a VL sequence of SEQ ID NO: 37;
a VH sequence of SEQ ID NO: 38 and a VL sequence of SEQ ID NO: 39;
a VH sequence of SEQ ID NO: 40 and a VL sequence of SEQ ID NO: 41;
a VH sequence of SEQ ID NO: 42 and a VL sequence of SEQ ID NO: 43;
a VH sequence of SEQ ID NO: 44 and a VL sequence of SEQ ID NO: 45;
a VH sequence of SEQ ID NO: 46 and a VL sequence of SEQ ID NO: 47;
a VH sequence of SEQ ID NO: 48 and a VL sequence of SEQ ID NO: 49;
a VH sequence of SEQ ID NO: 50 and a VL sequence of SEQ ID NO: 51;
a VH sequence of SEQ ID NO: 52 and a VL sequence of SEQ ID NO: 53;
a VH sequence of SEQ ID NO: 58;
a VH sequence of SEQ ID NO: 59; and
a VH sequence of SEQ ID NO: 60.

26. The polypeptide according to any of paragraphs 1 to 25, which is pegylated.

27. A nucleic acid encoding the polypeptide of any of paragraphs 1 to 25.

28. An expression cassette comprising the nucleic acid according to paragraph 27.

29. A vector comprising the nucleic acid according to paragraph 27 or the expression cassette according to paragraph 28.

30. A cell comprising the nucleic acid according to paragraph 27, the expression cassette according to paragraph 28, or the vector according to paragraph 29.

31. A method for making a polypeptide according to any of paragraphs 1 to 25 by culturing a cell according to paragraph 30 and purifying the polypeptide from the supernatant.

32. A pharmaceutical composition comprising the polypeptide according to any of paragraphs 1 to 26, or the nucleic acid according to paragraph 27, or the expression cassette according to paragraph 28, or the vector according to paragraph 29; and a pharmaceutically acceptable carrier, diluent, excipient or adjuvant.

33. A polypeptide according to any of paragraphs 1 to 26 or a pharmaceutical composition according to paragraph 32 for use in medicine.

34. A polypeptide according to any of paragraphs 1 to 26 or a pharmaceutical composition according to paragraph 32 for use in the treatment of a coronavirus infection or a condition or disorder resulting from this infection.

35. Use of a polypeptide according to any of paragraphs 1 to 26 or a pharmaceutical composition according to paragraph 32 in the manufacturing of a medicament for the treatment of a coronavirus infection or a condition or disorder resulting from this infection.

36. Method for treating a coronavirus infection or a condition or disorder resulting from this infection in a subject in need thereof, comprising a step of administering a polypeptide according to any of paragraphs 1 to 26 or a pharmaceutical composition according to paragraph 32 to the subject.

37. Method for treating a subject having COVID-19 of unknown SARS-CoV-2 strain, comprising a step of administering a polypeptide according to any of paragraphs 1 to 26 or a pharmaceutical composition according to paragraph 32 to the subject.

38. Method for treating a subject previously immunised with a vaccine based on S protein depicted under Uniprot accession number P0DTC2, comprising a step of administering a polypeptide according to any of paragraphs 1 to 26 or a pharmaceutical composition according to paragraph 32 to the subject.

39. Method for treating a subject previously treated with antibodies specific to S protein depicted under Uniprot accession number P0DTC2, comprising a step of administering a polypeptide according to any of paragraphs 1 to 26 or a pharmaceutical composition according to paragraph 32 to the subject.

40. Method for treating a subject previously infected with a first SARS-CoV-2 strain who is currently infected with a second SARS-CoV-2 strain, wherein the first and second SARS-CoV-2 strains are different, comprising a step of administering a polypeptide according to any of paragraphs 1 to 26 or a pharmaceutical composition according to paragraph 32 to the subject.

41. Method for treating a coronavirus infection of one SARS-CoV-2 strain selected from wild-type, variant D614G, variant A222V, variant S477N, variant B.1.1.7, variant B.1.351, and variant B.1.1.28, comprising a step of administering a polypeptide according to any of paragraphs 1 to 26 or a pharmaceutical composition according to paragraph 32 to the subject.

42. A method of neutralising a coronavirus infection, comprising a step of contacting a polypeptide according to any of paragraphs 1 to 26 or a pharmaceutical composition according to paragraph 32 with a cell infected with said coronavirus.

43. A polypeptide or pharmaceutical composition for use according to paragraph 34, or the use according to paragraph 35, or the method according to paragraphs 36, 37 or 42, wherein the coronavirus is SARS-CoV-2.

### DESCRIPTION OF THE FIGURES

FIG. 1. X-ray crystal structure of the ACE2-S1 complex. ACE2 (bottom), S1 (top) complex X-ray crystallography (6M0J).

FIG. 2. ACE2-Fc fusion constructs.

FIG. 3. ACE2-scFv-Fc fusion constructs.

FIG. 4. X-ray crystal structure of Domain 1 and Domain 2 of CD147. Domain 1 spanning aa 140-219 (top); Domain 2 aa 220-320 (bottom). X-ray crystal structure (3B5H).

FIG. 5. CD147-Fc and CD147-ACE2-Fc fusion constructs.

FIG. 6. Fusion proteins based on anti-S protein binders. A) dAb and scFv-Fc fusion protein homodimers. B) Heterodimers of dAb and scFv-based fusion constructs using the “knob-into-hole” strategy. C) Heterodimers of dAb and scFv-based fusion constructs using the bispecific tetravalent Fc fusions strategy.

FIG. 7. Binding of hACE2-IgG Fc and hACE2-IgM Fc to the S1 protein of SARS-CoV-2 and SARS-CoV by ELISA.

A) Binding to S1 protein of SARS-CoV-2. B) Binding to S1 protein of SARS-CoV. C) Non-specific binding control to unrelated HepB peptides.

FIG. 8. Binding of hACE2-IgGFc-CD147D1 to the S1 protein of SARS-CoV-2 and SARS-CoV by ELISA.

A) Binding to S1 protein of SARS-CoV-2. B) Binding to S1 protein of SARS-CoV. C) Non-specific binding control to unrelated HepB peptides.

FIG. 9. Screening of dAb binders specific for S1 protein of SARS-CoV-2 by ELISA.

FIG. 10. Characterisation of the binding specificity of dAbs specific for the S protein of SARS-CoV-2 by ELISA.

A) Binding to S1 protein of SARS-CoV-2. B) Binding to S1 protein of SARS-CoV. C) Non-specific binding control to unrelated HepB peptides.

FIG. 11. Binding of SARS-CoV neutralising antibodies to SARS-CoV-2 S1.

Recombinant anti-SARS antibodies in scFv-Fc format were tested by ELISA as supernatant at approximately 10 μg/ml against SARS-CoV-2 S1 subunit. Clones are 80R (74648), S230.15 (74649), M396 (74650), F26G19 (74651), F26G8 (74652), F26G8.2 (74653), F26G18 (74654), 92N (74655), 91M (75656), 27D (74657), 26H (74658), 12E (74659), 8C (74660), CR3009 (74661), CR3006 (74662), CR3018 (74663), CR3013 (74664), CR3014 (74665), AS3-3 (74667), CR3022 (74668), and B1 (74669). Dotted line represents baseline cut-off (3× background signal).

FIG. 12. Binding profile of CR3022 scFv-Fc, ACE2-CR3022 scFv-Fc, CR3022 scFv-ACE2-Fc to recombinant full length spike protein in ELISA.

FIG. 13. ACE2 enzymatic activity.

Enzymatic activity of active and inactive ACE2 (HH:NN)-Fc on fluorogenic substrate Mca-APK(Dnp) showing no activity for the mutated ACE2 (18-740)-Fc.

FIG. 14. Binding of ACE2 to Angiotensin II by surface plasmon resonance and ELISA.

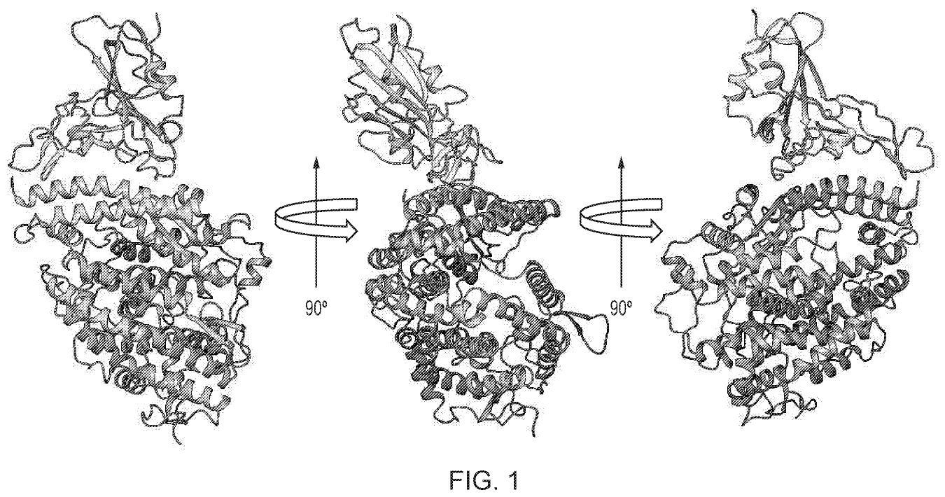

(A) Surface plasmon resonance (SPR) sensograms of binding kinetics between active ACE2-Fc and inactive ACE2 (HH:NN)-Fc for angiotensin 2. Angiotensin II concentration range was from 1 μM to 15.625 nM. Kinetic affinity, expressed as KD (M), was measured at 117 nM for active ACE2-Fc and at 1.3 μM for inactive ACE2-Fc, when fitted with a Langmuir 1:1 binding model. (B) SPR kinetic affinity of ACE2-Fc WT (left) and HH:NN mutated (inactive) (right), on SARS-CoV-2 spike S1 domain, showing comparable kinetic profiles. (C) ELISA of SARS-CoV-2 active spike trimer (left) or S1 domain (centre) against WT active and ACE2 (HH:NN)-Fc (inactive), showing comparable binding capacity. No binding detected with control antigen (right) or negative control antibody (Mean±SD). Unpaired t-test of AUC (left t=1.086, df=42; centre t=1.79, df=42).

FIG. 15. Biophysical characterisation of ACE2-Fc LALA and ACE2-Fc LALA-PG.

(A) Size exclusion chromatography on a Superdex 200 increase 5-150 GL in comparison to ACE2(18-740)-Fc. (B) Representative thermal stability scan on nanoDSF showing first derivative of 350 nm/330 nm absorbance ratio, in comparison to ACE2(18-740)-Fc.

FIG. 16. Characterisation of Fc effector functions.

(A) ELISA of SARS-CoV-2 active spike trimer (left) or S1 domain (right) with ACE2(HH:NN) WT Fc (square), LALA Fc (triangle) or LALA-PG Fc (rhomboid), showing comparable binding capacity (Mean±SD). One-way ANOVA of AUC with Turkey's multiple comparison (left F=0.3121, df=2, 96; right F=34.17, df=2, 96, compared to square). (B) Binding capacity of SupT1 cell line expressing codon optimised SARS-CoV-2 full length Spike, by flow cytometry against ACE2(HH:NN) WT Fc (square), LALA Fc (triangle) or LALA-PG Fc (rhomboid). (Mean±SD). One-way ANOVA of AUC with Turkey's multiple comparison (square vs triangle, F=3.986, df=2, 69). (C) Fc-mediated binding capacity to U937, K562 and SupT1 of ACE2(HH:NN) WT Fc (square), LALA Fc (triangle) or LALA-PG Fc (rhomboid), detected with biotinylated SARS-CoV-2 S1 and streptavidin conjugated secondary agent. No binding was detected with ACE2-Fc constructs carrying the LALA or LALA-PG mutations (Mean±SD). (D) Representative flow cytometry plot of Fc-mediated binding of ACE2 (HH:NN) WT Fc (top) and LALA-PG Fc (middle) on human monocyte-derived M1 macrophages. No binding detected with Fc carrying the LALA-PG mutation (n=4). (E) SPR binding kinetic of ACE2 (HH:NN) WT Fe, LALA Fe or LALA-PG Fe on human FcγRIa, FcγRIIa, FcγRIIb, FcγRIIIa and FcγRIIIb. LALA-PG mutations mediated a complete abrogation of FcγR interaction.

FIG. 17. Virus neutralisation assay.

Virus neutralisation assay on live SARS-CoV-2 virus (A) and lentiviral pseudotyped virus (B) using ACE2-Fe, ACE2-Fc LALA or ACE2-Fc LALA-PG constructs. Virus neutralisation was measured at IC50 of 5.2, 11.7 and 4.1 nM for ACE2-Fe, ACE2-Fc LALA and ACE2-Fc LALA-PG, respectively for live virus. Neutralisation on pseudotyped virus was determined at IC50 of 0.3 nM for ACE2-Fc and ACE-Fc LALA, and 0.1 nM for ACE2-Fc LALA-PG.

FIG. 18. ACE2-Fc formulation optimisation.

(A) Thermal stability analysis via nanoDSF of ACE2 (HH:NN)-Fc in PBS at pH 7.4 or in 20 mM His buffer with pH range of 3.5-7. Higest stability obtained with 20 mM His pH 6.5. (B) Thermal stability of ACE2-FX in 20 mM His pH 6.5 (dashed line) and 3.5 (thick solid line) following 2 h incubation at RT. Full stability could be recovered following buffer exchange of ACE2-FX from pH 3.5 to pH 6.5 (line). (C) Particle size distribution analysis via MADLS of ACE2-FX at 1 mg/ml in PBS pH 7.4, 20 mM His pH3.5, 6.5 or buffer exchanged from pH 3.5 to 6.5. Increase of particle size of sample at pH 3.5 was partially recovered upon buffer exchange in 20 mM His pH 6.5 (Mean±SD). (D) ACE2 (HH:NN)-Fc binding capacity for SARS-CoV-2 S1 in ELISA in PBS pH 7.4 (circle), 20 mM His pH 6.5 (triangle) or 20 mM His pH3.5 followed by buffer exchange in 20 mM His pH 6.5 (square). (Mean±SD). (E) Thermal stability analysis via nanoDSF of ACE2-FX in PBS at pH 7.4 (dashed line) or in 20 mM His pH 6.5 (solid line) showing a 3.9° C. Tm shift. (F) Particle size distribution analysis via MADLS of ACE2-FX at 1 mg/ml in PBS pH 7.4 (dashed line) and 20 mM His pH 6.5 (solid line) showing comparable profile (Mean±SD). (G) Particle size distribution analysis via MADLS of ACE2 (HH:NN)-Fc (LALA-PG) at 20 mg/ml in 20 mM His pH 6.5 with polisorbate 80 range 0-1% (Mean±SD). One way ANOVA with Turkey's multiple comparisons (F=9.384, df=6, 14).

FIG. 19. Biophysical characterisation of low pH exposed ACE2-Fc.

Biophysical characterisation of ACE2-Fc upon incubation at low pH, via DSF (A), SEC (B) and ELISA against full length SARS-CoV-2 spike protein (C). Change of pH from 3.5 to 7 resulted in a complete recovery of binding capacity and thermal stability of the construct.

FIG. 20. Formulation optimisation for ACE2-Fc LALA and LALA-PG.

Formulation optimisation of ACE2-Fc LALA and LALA-PG. Proteins dialysed in 20 mM His buffer at a pH 6.5 were analysed by SEC (A) and thermal stability determined by nanoDSF (B). 20 mM His pH 6.5 was able to increase Tm for both constructs by 3.9° C. compared to PBS pH 7.4 formulation.

FIG. 21. Comparison of virus neutralisation assay.

Neutralisation assay of SARS-CoV-2 pseudotype (left) or live virus (right) with ACE2 (HH:NN)-Fc (LALA-PG) in PBS pH 7.4 (triangle) and 20 mM His pH 6.5 (open square). Both formulations show comparable neutralisation efficiencies (Mean±SD). Unpaired t test of AUC (left t=0.000, df=40; right t=1.317, df=28).

FIG. 22. ACE2-Fc (LALA-PG) specificity.

A) Cell microarray screening of human cell-membrane proteome with ACE2-Fc (LALA-PG), control Fc (LALA-PG), CTLA4-hFc or PBS. Depicted is a selection of antigens (key legend on the right panel). ACE2-Fc (LALA-PG) shows strong specific interaction with SARS-CoV-2 spike protein only. B) SPR binding kinetics of ACE2 (HH:NN) WT Fc, LALA-PG Fc, mAb #A, mAb #B and mAb #C against SARS-CoV-1 S1, SARS-CoV-2 S1, SARS-CoV-2 S1 D614G and HCoV-NL63 S1 domains. ACE2 (HH:NN) Fc and ACE2 (HH:NN) Fc (LALA-PG) were able to efficiently bind all spike protein tested. All sensograms were fitted with Langmuir 1:1 binding model, except for SARS-CoV-1 S1 kinetics which were fitted with two-state kinetics. Two-fold serial dilutions starting from 500 nM for HCoV-NL63 S1, 250 nM for SARS-CoV-1 and SARS-CoV-2 S1, 125 nM for SARS-CoV-2 S1 D614G.

FIG. 23. SARS-CoV-2 neutralisation efficiency.

A) Neutralisation assay of live SARS-CoV-2 virus by ACE2 (HH:NN) WT Fc (blue) and LALA-PG Fc (orange). Both variants show comparable neutralisation efficiencies (Mean±SD). Unpaired t-test of AUC (t=1.695, df=28). B) Infectious viral titre (left) and physical particle number as determined by p24 ELISA (right) comparison of SARS-CoV-1, SARS-CoV-2 WT, SARS-CoV-2 D614G, SARS-CoV-2 B.1.1.7 and SARS-CoV-2 B.1.351 pseudotyped vectors, showing comparable particle concentration but diverse infective capacity (Mean±SD). One way ANOVA Dunnett's multiple comparison (F=105.2, df=4, 10). C) Neutralisation assay of SARS-CoV-1, SARS-CoV-2, SARS-CoV-2 D614G, SARS-CoV-2 B.1.1.7, and SARS-CoV-2 B.1.351 pseudotyped vectors against ACE2 (HH:NN)-Fc (LALA-PG), mAb #A, mAb #B+C, mAb #B and mAb #C. Marked decrease of neutralisation capacity for SARS-CoV-2 B1.351 detected for mAb #A, mAb #B+C, and mAb #B and mAb #C individually. No loss of neutralisation capacity detected for ACE2 (HH:NN)-Fc (LALA-PG) receptor decoy (Mean±SD). One-way ANOVA of AUC with Dunnett's multiple comparison to blue (F=369.2, df=4,100). Bottom right panel, fold change of neutralisation capacity based on IC₅₀ values.

FIG. 24. In vivo SARS-CoV-2 neutralisation.

Syrian hamsters intranasally challenged with live SARS-CoV-2. ACE2 (HH:NN)-Fc (LALA-PG) administered i.p. at day 1 post-challenge at 5 mg/kg and 50 mg/kg or placebo (PBS) (n=6 per group). A) Body weight change (%) relative to the day of viral inoculation. Day of therapeutic administration marked with arrow. Significant reduction of body weight change compared to placebo, detected for both treatment regimens (Mean±SD). Individual day comparison placebo vs. 50 mg/kg dose two-way ANOVA with Sidak's multiple comparison compared to placebo group * p=0.01, ** p=0.004, *** p=0.0001, **** p<0.0001. One-way ANOVA of AUC with Turkey's multiple comparison (F=9.379, df=2, 249). B) Subgenomic RNA PCR swab test. Limit of detection 2.88 RNA copies, samples with undetectable RNA were assigned a value of 1 (Mean±SD). Two-way ANOVA with Dunnett's multiple comparison, one-way ANOVA of AUC with Dunnett's multiple comparison (F=3.247, df=2.69). C) Necropsy pathology lung score (categories 1-3) showing reduction in lung damage for ACE2(HH:NN)-Fc (LALA-PG) treated groups. Bottom, representative lung damage for grade score 1, 2 and 3. D) Human IgG1 Fc concentration in hamster sera at day −5 and day 7 relative to viral inoculation. Limit of detection 4 ng/ml. Samples with undetectable levels were assigned a value of 1 (Mean±SD). Two-way ANOVA with Sidak's multiple comparison (F=39.2, df=2, 22).

FIG. 25. Immunoglobulin-based architectures.

A) IgG and IgG-based molecules. B) Dual variable domain (DVD) and DVD-based molecules. C) scFv4-Fc and scFv4-Fc-based molecules.

### DETAILED DESCRIPTION OF THE INVENTION

The present invention provides recombinant fusion proteins which have the ability to neutralise coronavirus, and SARS-CoV-2 virus in particular, and viral re-entry. These fusion proteins are based on ACE2, CD147 and antibodies binding to the coronavirus spike protein (S protein).

1. Coronavirus Spike Protein (S Protein)

Three coronaviruses have crossed the species barrier to cause deadly pneumonia in humans since the beginning of the 21st century: severe acute respiratory syndrome coronavirus (SARS-CoV), Middle East respiratory syndrome coronavirus (MERS-CoV), and SARS-CoV-2. In 2002-2003, SARS-CoV, a lineage B beta-CoV, emerged from bat and palm civet, and infected over 8,000 people and caused about 800 deaths. In 2012, MERS-CoV, a lineage C beta-CoV, was discovered as the causative agent of a severe respiratory syndrome in Saudi Arabia, currently with 2,494 confirmed cases and 858 deaths, it remains endemic in Middle East, and dromedary camel is considered as the zoonotic reservoir host of MERS-CoV. At the end of 2019, a novel coronavirus, named SARS-CoV-2, was found in patients with severe pneumonia in Wuhan, China. Viruses were isolated from patients and sequenced. Phylogenetical analysis revealed that it is a lineage B beta-CoV and closely related to a SARS-like (SL) CoV, RaTG13, discovered in a cave of Yunnan, China, in 2013. They share about 96% nucleotide sequence identities, suggesting that SARS-CoV-2 might have emerged from a Bat SL-CoV. However, the intermediate host or whether there is an intermediate host remains to be determined.

In addition to the highly pathogenic zoonotic pathogens SARS-CoV, MERS-CoV, and SARS-CoV-2, all belonging to the β-coronavirus genus, four low-pathogenicity coronaviruses are endemic in humans: HCoV-OC43, HCoV-HKU1, HCoV-NL63, and HCoV-229E.

The coronaviruses (order Nidovirales, family Coronaviridae, genus Coronavirus) are a diverse group of large RNA viruses that cause varieties of diseases in humans and other animals, including respiratory, enteric, renal, and neurological diseases. Coronaviruses are enveloped viruses that contain a large single-stranded RNA genome of positive polarity. At ˜30,000 nucleotides (nt), their genome is the largest found in any of the RNA viruses. Their envelope accommodates three or four membrane proteins of which the membrane (M), envelope (E), and spike (S) proteins are common to all. The S protein is a relatively large, about 180 kDa type I glycoprotein, trimers of which form the petal-shaped projections on the surface of the virion that give rise to the characteristic corona solis-like appearance. It has been suggested that the S1 subunit constitutes the globular head, while the S2 subunit forms the stalk-like region of the spike.

The two functions of the coronavirus S protein appear to be spatially separated. The S1 subunit (or the equivalent part in viruses with uncleaved S protein) is responsible for receptor binding, and the S2 subunit is responsible for membrane fusion. In the structure, N- and C-terminal portions of S1 fold as two independent domains, N-terminal domain (NTD) and C-terminal domain (C-domain). Depending on the virus, either NTD or C-domain can serve as the receptor-binding domain (RBD). While RBD of mouse hepatitis virus (MHV) is located at the NTD, most of other CoVs, including SARS-CoV and MERS-CoV use C-domain to bind their receptors. MHV uses mouse carcinoembryonic antigen related cell adhesion molecule 1a (mCEACAM1a) as the receptor, and the receptors for SARS-CoV and MERS-CoV are human angiotensin-converting enzyme 2 (hACE2) and dipeptidyl peptidase 4 (DPP4), respectively. In terms of sequence identity, S proteins of SARS-CoV-2 share about 76% and 97% of amino acid identity with SARS-CoV and RaTG13, respectively, and the amino acid sequence of potential RBD of SARS-CoV-2 is about 74% and 90.1% homologous to that of SARS-CoV and RaTG13, respectively.

The ectodomain of the S2 subunit contains two heptad repeat (HR) regions, HR1 and HR2, characteristic of coiled coils, while the fusion peptide (FP) is predicted to be located amino terminally of the first HR region (HR1). Binding of the S1 subunit to the (soluble) receptor has been shown to trigger conformational changes that supposedly facilitate virus entry by activation of the fusion function of the S2 subunit. The conformational changes are thought to expose the fusion peptide and to lead to the formation of a heterotrimeric six-helix bundle by the two HR regions, a characteristic of class I viral fusion proteins, resulting in the close locations of the fusion peptide and the transmembrane domain in the process of membrane fusion.

Coronavirus S proteins are typical class I viral fusion proteins, and protease cleavage is required for activation of the fusion potential of S protein. A two-step sequential protease cleavage model has been proposed for activation of S proteins of SARS-CoV and MERS-CoV, priming cleavage between S1 and S2 and activating cleavage on S2′ site. Depending on virus strains and cell types, CoV S proteins may be cleaved by one or several host proteases, including furin, trypsin, cathepsins, transmembrane protease serine protease-2 (TMPRSS-2), TMPRSS-4, or human airway trypsin-like protease (HAT). Availability of these proteases on target cells largely determines whether coronaviruses enter cells through plasma membrane or endocytosis. It has been reported that SARS-CoV-2 S protein is capable of triggering protease-independent and receptor-dependent syncytium formation. Such a mechanism might enhance virus spreading through cell-cell fusion and this might partially explain rapid progress of the disease.

The coronavirus S protein may be the S protein of one of the following coronavirus: SARS-CoV-2, SARS-CoV, SARS-like CoV RaTG13, MERS-CoV, HCoV-OC43, HCoV-HKU1, HCoV-NL63, and HCoV-229E.

The coronavirus S protein may be the S protein of SARS-CoV-2, SARS-CoV, or SARS-like CoV RaTG13.

The coronavirus S protein may be the S protein of SARS-CoV-2 depicted under Uniprot accession number P0DTC2 (sequence version 1, as of 22 Apr. 2020).

The sequences of S protein (SEQ ID NO: 73; signal sequence underlined), subunit S1 (SEQ ID NO: 74) and subunit S2 (SEQ ID NO: 75; HR1 region is underlined and HR2 region is in bold) of coronavirus SARS-CoV-2 are shown below.

SARS-COV-2 S protein (SEQ ID NO: 73):
MFVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGFNCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCL
IGAEHVNNSYECDIPIGAGICASYQTQTNSPRRARSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQTYKTPPIKDFGGF
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQD
VVNQNAQALNTLVKQLSSNFGAISSVLNDILSRLDKVEAEVQIDRLITGR
LQSLQTYVTQQLIRAAEIRASANLAATKMSECVLGQSKRVDFCGKGYHLM
SFPQSAPHGVVFLHVTSGNCDVVIGIVNNTVYDPLQPELDSFKEELDKYF
KNHTSPDVDLGDISGINASVVNIQKEIDRLNEVAKNLNESLIDLQELGKY
EQYIKWPWYIWLGFIAGLIAIVMVTIMLCCMTSCCSCLKGCCSCGSCCKF
DEDDSEPVLKGVKLHYT
SARS-COV-2 Spike protein, S1 subunit
(SEQ ID NO: 74):
QCVNLTTRTQLPPAYTNSFTRGVYYPDKVFRSSVLHSTQDLFLPFFSNVT
WFHAIHVSGTNGTKRFDNPVLPFNDGVYFASTEKSNIIRGWIFGTTLDSK
TQSLLIVNNATNVVIKVCEFQFCNDPFLGVYYHKNNKSWMESEFRVYSSA
NNCTFEYVSQPFLMDLEGKQGNFKNLREFVFKNIDGYFKIYSKHTPINLV
RDLPQGFSALEPLVDLPIGINITRFQTLLALHRSYLTPGDSSSGWTAGAA
AYYVGYLQPRTFLLKYNENGTITDAVDCALDPLSETKCTLKSFTVEKGIY
QTSNFRVQPTESIVRFPNITNLCPFGEVFNATRFASVYAWNRKRISNCVA
DYSVLYNSASFSTFKCYGVSPTKLNDLCFTNVYADSFVIRGDEVRQIAPG
QTGKIADYNYKLPDDFTGCVIAWNSNNLDSKVGGNYNYLYRLFRKSNLKP
FERDISTEIYQAGSTPCNGVEGFNCYFPLQSYGFQPTNGVGYQPYRVVVL
SFELLHAPATVCGPKKSTNLVKNKCVNFNFNGLTGTGVLTESNKKFLPFQ
QFGRDIADTTDAVRDPQTLEILDITPCSFGGVSVITPGTNTSNQVAVLYQ
DVNCTEVPVAIHADQLTPTWRVYSTGSNVFQTRAGCLIGAEHVNNSYECD
IPIGAGICASYQTQTNSP
SARS-COV-2 Spike protein, S2 subunit
(SEQ ID NO: 75):
RRARSVASQSIIAYTMSLGAENSVAYSNNSIAIPTNFTISVTTEILPVSM
TKTSVDCTMYICGDSTECSNLLLQYGSFCTQLNRALTGIAVEQDKNTQEV
FAQVKQIYKTPPIKDFGGFNFSQILPDPSKPSKRSFIEDLLFNKVTLADA
GFIKQYGDCLGDIAARDLICAQKFNGLTVLPPLLTDEMIAQYTSALLAGT
ITSGWTFGAGAALQIPFAMQMAYRFNGIGVTQNVLYENQKLIANQFNSAI
GKIQDSLSSTASALGKLQDVVNQNAQALNTLVKQLSSNFGAISSVLNDIL
SRLDKVEAEVQIDRLITGRLQSLQTYVTQQLIRAAEIRASANLAATKMSE
CVLGQSKRVDFCGKGYHLMSFPQSAPHGVVFLHVTYVPAQEKNGIVNNTV
YDPLQPELDSFKEELDKYFKNHTSPDVDLGDISGINASVVNIQKEIDRLN
EVAKNLNESLIDLQELGKYEQYIKWPWYIWLGFIAGLIAIVMVTIMLCCM
TSCCSCLKGCCSCGSCCKFDEDDSEPVLKGVKLHYT

Several variants carrying mutations in S-protein, including in its receptor-binding domain (RBD), have emerged, likely due to the rapid dissemination of the virus coupled with pressure from the patients' immune response. Of note is the identification of the D614G (Nextstrain clade 20A) in early March 2020 that has rapidly become the dominant strain globally. Additional variants have also gained partial dominance in different regions of the globe. The variants A222V (Nextstrain clade 20A.EU1) and S477N (Nextstrain clade 20A.EU2) have emerged in the summer of 2020 in Spain and have rapidly shown diffusion within Europe. Recently, a new variant (clade 20B/501Y.V1, B.1.1.7) characterised by multiple mutations in S-protein (deletion 69-70, deletion 144, N501Y, A570D, D614G, P681H, T716I, S982A, D1118H) has been associated with a rapid surge in COVID-19 cases in the UK between December 2020 and January 2021. In the same period, a new variant in South Africa (clade 20C/501Y.V2, B.1.351), also carrying the N501Y mutation in the RBD (L18F, D80A, D215G, R246I, K417N, E484K, N501Y, D614G and A701V), has been associated with increased transmissibility and reduction of serum neutralisation capacity. Finally, a variant that emerged in Brazil (B.1.1.28) contains mutational hallmarks of both the UK and South Africa variants (L18F, T20N, P26S, D138Y, R190S, K417T, E484K, N501Y, H655Y, T1027I), suggesting convergent evolution in SARS-CoV-2 due to similar selective pressures. Further variants are expected to emerge. The mutation numbering refers to the SARS-CoV-2 S protein as shown in SEQ ID NO: 73.

The coronavirus S protein may be the S protein of wild type SARS-CoV-2 (SEQ ID NO: 73) or a variant thereof having one or more mutations from the sequence shown as SEQ ID NO: 73. Variants of one the S protein of wild type SARS-CoV-2 include, without limitation, variants D614G; A222V; S477N; clade 20B/501Y.V1 or UK variant B.1.1.7; and clade 20C/501Y.V2, B.1.351 or South African variant; Brazilian variant B.1.1.28.

Variants of wild type SARS-CoV-2 include, without limitation, variants D614G; A222V; S477N; clade 20B/501Y.V1 or UK variant B.1.1.7; and clade 20C/501Y.V2, B.1.351 or South African variant; Brazilian variant B.1.1.28.

It will be immediately understood that the present invention is useful with any other SARS-CoV-2 variants existent at the time of filing or with any future variants that may emerge. Thus, the coronavirus S protein may be the S protein of any of these variants.

2. Polypeptide Based on ACE2

The present inventors have designed and generated fusion proteins based on the ectodomain of ACE2, which is used by a number of coronaviruses as a receptor to infect cells. As shown in Examples 12 to 15, these molecules can efficiently neutralise the virus by acting as a receptor decoy for the spike protein of SARS-CoV-2. Surprisingly, these molecules appear to be insensitive to coronavirus mutational drift, as demonstrated in Example 14 with different SARS-CoV-2 variants. Such drift can alter epitopes on the spike protein rendering antibody-based passive immunisations less effective or entirely ineffective. Consequently, immunisation achieved by infection with an earlier form of the virus, or vaccination with an earlier form of the spike protein may limit or lose their protective effect. The present inventors have hypothesised that fusion proteins based on ACE2 may reduce the deleterious impact of the infection irrespective of the viral strain for as long as the virus binds to human ACE2 protein.

In a first aspect, the present invention provides a polypeptide, hereinafter “the first polypeptide of the invention”, comprising:
a) a domain A which comprises the ectodomain of human angiotensin converting enzyme type 2 (hACE2), or a fragment thereof comprising the amino acid sequence shown as SEQ ID NO: 3, or a variant thereof which maintains the capacity of hACE2 to interact with the S protein of coronavirus, and
b) a domain O which comprises an oligomerisation domain,

wherein the oligomerisation domain is selected from an IgG Fc region or a variant thereof which does not interact with FcγRI, FcγRIIa and FcγRIII, an IgM Fc region, an IgA Fc region, a collagen XVIII trimerizing structural element, a collagen XV trimerizing structural element, a foldon domain, a TenC domain and a coiled coil domain;

with the proviso that if the domain A consists of the ectodomain of hACE2 then the domain O is not an IgG1 Fc region.

The term polypeptide, as used herein, refers to natural, synthetic, and recombinant proteins or peptides generally having more than 10 amino acids.

2.1. Domain A

The first polypeptide of the invention comprises a domain A, which comprises the ectodomain of human angiotensin converting enzyme type 2 (hACE2) or a fragment thereof comprising the amino acid sequence shown as SEQ ID NO: 3, or a variant thereof which maintains the capacity of hACE2 to interact with the S protein of coronavirus.

The terms “angiotensin converting enzyme type 2”, “ACE2”, “hACE2”, “ACE-related carboxypeptidase”, “angiotensin-converting enzyme homolog”, “ACEH”, “metalloprotease MPROT15”, and “processed angiotensin-converting enzyme 2” are used indistinctly in the present invention. Human ACE2 is depicted under Accession No. Q9BYF1 in the Uniprot database on 30 Mar. 2020. hACE2 is an 805 aa transmembrane protein with a processed ectodomain that spans aa 18-740 of the sequence shown under Uniprot Accession No. Q9BYF1.

Human ACE2 has been identified as a functional receptor for the S protein of human coronavirus NL63 (HCoV-NL63) and of SARS coronavirus (SARS-CoV). More recently, it has also been shown to be a receptor for SARS-CoV-2.

ACE2 is a metalloprotease involved in the Renin-Angiotensin System (RAS), which controls blood pressure, electrolytes and intravascular fluid volume. A key function of hACE2 is believed to be the cleavage of Angiotensin II (Ang II) to Ang (1-7), which have opposing physiological roles. Elevated levels of Ang II are associated with vasoconstriction, inflammation, fibrosis, vascular leak, and sodium absorption. Ang (1-7) appears to be a counterregulatory protein in the RAS; associated with vasodilation, anti-proliferation, antiinflammation, and reduced vascular leak. hACE2 has also been reported to have a protective role in acute lung injury, providing a molecular explanation for the severe lung failure and death due to SARS-CoV infections.

In a normal adult human lung, hACE2 is expressed primarily in alveolar epithelial type II cells, which can serve as a viral reservoir. These cells produce surfactant which reduces surface tension, thus preventing alveoli from collapsing, and hence are critical to the gas exchange function of the lung. Injury to these cells could explain the severe lung injury observed in COVID-19 patients. hACE2 is also expressed in multiple extrapulmonary tissues including heart, kidneys, blood vessels, and intestine. The ACE2 tissue distribution in these organs may explain the multiorgan dysfunction observed in patients. There have been reports of patients presenting pulmonary embolism and kidney damage, probably caused by blood clots damaging the pulmonary and renal capillaries, as well as myocarditis, increased blood pressure, abdominal pain, diarrhoea and nausea.

Domain A of the first polypeptide of the invention may comprise the ectodomain of hACE2 or a fragment thereof comprising the amino acid sequence shown as SEQ ID NO: 3. hACE2 may comprise or consist of the processed ectodomain that spans aa 18-740 (SEQ ID NO:1), or the full ectodomain that spans aa 1-740 (SEQ ID NO: 2). Domain A may consist of the amino acid sequence shown as SEQ ID NO: 3.

Processed ACE2 ectodomain (SEQ ID NO: 1):
QSTIEEQAKTFLDKFNHEAEDLFYQSSLASWNYNTNITEENVQNMNNAGD
KWSAFLKEQSTLAQMYPLQEIQNLTVKLQLQALQQNGSSVLSEDKSKRLN
TILNTMSTIYSTGKVCNPDNPQECLLLEPGLNEIMANSLDYNERLWAWES
WRSEVGKQLRPLYEEYVVLKNEMARANHYEDYGDYWRGDYEVNGVDGYDY
SRGQLIEDVEHTFEEIKPLYEHLHAYVRAKLMNAYPSYISPIGCLPAHLL
GDMWGRFWTNLYSLTVPFGQKPNIDVTDAMVDQAWDAQRIFKEAEKFFVS
VGLPNMTQGFWENSMLTDPGNVQKAVCHPTAWDLGKGDFRILMCTKVTMD
DFLTAHHEMGHIQYDMAYAAQPFLLRNGANEGFHEAVGEIMSLSAATPKH
LKSIGLLSPDFQEDNETEINFLLKQALTIVGTLPFTYMLEKWRWMVFKGE
IPKDQWMKKWWEMKREIVGVVEPVPHDETYCDPASLFHVSNDYSFIRYYT
RTLYQFQFQEALCQAAKHEGPLHKCDISNSTEAGQKLFNMLRLGKSEPWT
LALENVVGAKNMNVRPLLNYFEPLFTWLKDQNKNSFVGWSTDWSPYADQS
IKVRISLKSALGDKAYEWNDNEMYLFRSSVAYAMRQYFLKVKNQMILFGE
EDVRVANLKPRISFNFFVTAPKNVSDIIPRTEVEKAIRMSRSRINDAFRL
NDNSLEFLGIQPTLGPPNQPPVS
Full ACE2 ectodomain (SEQ ID NO: 2):
MSSSSWLLLSLVAVTAAQSTIEEQAKTFLDKFNHEAEDLFYQSSLASWNY
NTNITEENVQNMNNAGDKWSAFLKEQSTLAQMYPLQEIQNLTVKLQLQAL
QQNGSSVLSEDKSKRLNTILNTMSTIYSTGKVCNPDNPQECLLLEPGLNE
IMANSLDYNERLWAWESWRSEVGKQLRPLYEEYVVLKNEMARANHYEDYG
DYWRGDYEVNGVDGYDYSRGQLIEDVEHTFEEIKPLYEHLHAYVRAKLMN
AYPSYISPIGCLPAHLLGDMWGRFWTNLYSLTVPFGQKPNIDVTDAMVDQ
AWDAQRIFKEAEKFFVSVGLPNMTQGFWENSMLTDPGNVQKAVCHPTAWD
LGKGDFRILMCTKVTMDDFLTAHHEMGHIQYDMAYAAQPFLLRNGANEGF
HEAVGEIMSLSAATPKHLKSIGLLSPDFQEDNETEINFLLKQALTIVGTL
PFTYMLEKWRWMVFKGEIPKDQWMKKWWEMKREIVGVVEPVPHDETYCDP
ASLFHVSNDYSFIRYYTRTLYQFQFQEALCQAAKHEGPLHKCDISNSTEA
GQKLFNMLRLGKSEPWTLALENVVGAKNMNVRPLLNYFEPLFTWLKDQNK
NSFVGWSTDWSPYADQSIKVRISLKSALGDKAYEWNDNEMYLFRSSVAYA
MRQYFLKVKNQMILFGEEDVRVANLKPRISFNFFVTAPKNVSDIIPRTEV
EKAIRMSRSRINDAFRLNDNSLEFLGIQPTLGPPNQPPVS
Truncated ACE2, aa 18-605 (SEQ ID NO: 3):
QSTIEEQAKTFLDKFNHEAEDLFYQSSLASWNYNTNITEENVQNMNNAGD
KWSAFLKEQSTLAQMYPLQEIQNLTVKLQLQALQQNGSSVLSEDKSKRLN
TILNTMSTIYSTGKVCNPDNPQECLLLEPGLNEIMANSLDYNERLWAWES
WRSEVGKQLRPLYEEYVVLKNEMARANHYEDYGDYWRGDYEVNGVDGYDY
SRGQLIEDVEHTFEEIKPLYEHLHAYVRAKLMNAYPSYISPIGCLPAHLL
GDMWGRFWTNLYSLTVPFGQKPNIDVTDAMVDQAWDAQRIFKEAEKFFVS
VGLPNMTQGFWENSMLTDPGNVQKAVCHPTAWDLGKGDFRILMCTKVTMD
DFLTAHHEMGHIQYDMAYAAQPFLLRNGANEGFHEAVGEIMSLSAATPKH
LKSIGLLSPDFQEDNETEINFLLKQALTIVGTLPFTYMLEKWRWMVFKGE
IPKDQWMKKWWEMKREIVGVVEPVPHDETYCDPASLFHVSNDYSFIRYYT
RTLYQFQFQEALCQAAKHEGPLHKCDISNSTEAGQKLFNMLRLGKSEPWT
LALENVVGAKNMNVRPLLNYFEPLFTWLKDQNKNSFVG

For reference, the sequence of hACE2 depicted under Accession No. Q9BYF1 in the Uniprot database on 30 Mar. 2020 is shown as SEQ ID NO: 137. In this sequence the leader peptide spans aa 1-17, the processed ectodomain spans aa 18-740, the transmembrane domain spans aa 741-761, and the cytoplasmic domain spans 762-805.

Human ACE2 (full-length)
(SEQ ID NO: 137)
MSSSSWLLLSLVAVTAAQSTIEEQAKTFLDKFNHEAEDLFYQSSLASWNY
NTNITEENVQNMNNAGDKWSAFLKEQSTLAQMYPLQEIQNLTVKLQLQAL
QQNGSSVLSEDKSKRLNTILNTMSTIYSTGKVCNPDNPQECLLLEPGLNE
IMANSLDYNERLWAWESWRSEVGKQLRPLYEEYVVLKNEMARANHYEDYG
DYWRGDYEVNGVDGYDYSRGQLIEDVEHTFEEIKPLYEHLHAYVRAKLMN
AYPSYISPIGCLPAHLLGDMWGRFWTNLYSLTVPFGQKPNIDVTDAMVDQ
AWDAQRIFKEAEKFFVSVGLPNMTQGFWENSMLTDPGNVQKAVCHPTAWD
LGKGDFRILMCTKVTMDDFLTAHHEMGHIQYDMAYAAQPFLLRNGANEGF
HEAVGEIMSLSAATPKHLKSIGLLSPDFQEDNETEINFLLKQALTIVGTL
PFTYMLEKWRWMVFKGEIPKDQWMKKWWEMKREIVGVVEPVPHDETYCDP
ASLFHVSNDYSFIRYYTRTLYQFQFQEALCQAAKHEGPLHKCDISNSTEA
GQKLFNMLRLGKSEPWTLALENVVGAKNMNVRPLLNYFEPLFTWLKDQNK
NSFVGWSTDWSPYADQSIKVRISLKSALGDKAYEWNDNEMYLFRSSVAYA
MRQYFLKVKNQMILFGEEDVRVANLKPRISFNFFVTAPKNVSDIIPRTEV
EKAIRMSRSRINDAFRLNDNSLEFLGIQPTLGPPNQPPVSIWLIVFGVVM
GVIVVGIVILIFTGIRDRKKKNKARSGENPYASIDISKGENNPGFQNTDD
VQTSF

The terms ectodomain of hACE2 or a fragment thereof comprising the amino acid sequence shown as SEQ ID NO: 3 are also intended to embrace functionally equivalent variants of the ectodomain of hACE2 or of a fragment thereof comprising the amino acid sequence shown as SEQ ID NO: 3, variants which have been modified in the amino acid sequence without adversely affecting, to any substantial degree, the capacity of hACE2 to interact with the S protein of coronavirus relative to those of the native hACE2 molecule. Said modifications include, the conservative (or non-conservative) substitution of one or more amino acids for other amino acids, the insertion and/or the deletion of one or more amino acids, provided that the capacity to interact with the S protein of coronavirus of the variant is substantially maintained, i.e., the variant maintains the ability (capacity) to interact with the S protein of coronavirus at physiological conditions.

The term “variant” or “mutant”, as used herein, refers to a polypeptide differing from a specifically recited polypeptide, i.e. reference or parent polypeptide by amino acid insertions, deletions, and/or substitutions, created using, for example, recombinant DNA techniques or by de novo synthesis. Variant and mutant are used indistinctly in the context of the present invention. The variants or mutants of the ectodomain of hACE2 or of a fragment thereof comprising the amino acid sequence shown as SEQ ID NO: 3 may have at least 70%, or at least 80%, or at least 90%, or at least 95%, or at least 96%, or at least 97%, or at least 98%, or at least 99% sequence identity with the sequences shown as SEQ ID NO: 1, 2 or 3, provided that the capacity to interact with the S protein of coronavirus of the variant is substantially maintained. Regions corresponding to positions 19-41, 82-84 and 353-357 of the full ACE2 ectodomain (SEQ ID NO: 2) may be kept unchanged.

Variants of the ectodomain of hACE2 or of a fragment thereof comprising the amino acid sequence shown as SEQ ID NO: 3 typically have at least about 75%, for example at least about 80%, 90%, 95%, 96%, 97%, 98% or 99% sequence identity with the sequences given as SEQ ID NO: 1-3.

Typically, variants may contain one or more conservative amino acid substitutions compared to the original amino acid or nucleic acid sequence. Conservative substitutions are those substitutions that do not substantially affect or decrease the affinity of a hACE2 variant to bind coronavirus S protein. For example, a hACE2 variant that specifically binds coronavirus S protein may include up to 1, up to 2, up to 5, up to 10, or up to 15 conservative substitutions compared to any of the sequences given as SEQ ID NO: 1-3 and retain specific binding to coronavirus S protein.

Functionally similar amino acids which may be exchanged by way of conservative substitution are well known to one of ordinary skill in the art. The following six groups are examples of amino acids that are considered to be conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W).

The percentage of sequence identity may be determined by comparing two optimally aligned sequences over a comparison window. The aligned sequences may be polynucleotide sequences or polypeptide sequences. For optimal alignment of the two sequences, the portion of the polynucleotide or amino acid sequence in the comparison window may comprise insertions or deletions (i.e., gaps) as compared to the reference sequence (that does not comprise insertions or deletions). The percentage of sequence identity is calculated by determining the number of positions at which the identical nucleotide residues, or the identical amino acid residues, occurs in both compared sequences to yield the number of matched positions, then dividing the number of matched positions by the total number of positions in the window of comparison and multiplying the result by 100 to yield the percentage of sequence identity. Sequence identity between two polypeptide sequences or two polynucleotide sequences can be determined, for example, by using the Gap program in the WISCONSIN PACKAGE version 10.0-UNIX from Genetics Computer Group, Inc. based on the method of Needleman and Wunsch (1970, J Mol Biol 48:443-53) using the set of default parameters for pairwise comparison (for amino acid sequence comparison: Gap Creation Penalty=8, Gap Extension Penalty=2; for nucleotide sequence comparison: Gap Creation Penalty=50; Gap Extension Penalty=3), or using the TBLASTN program in the BLAST 2.2.1 software suite (Altschul et al., 1997, Nucleic Acids Res 25:3389-402), using BLOSUM62 matrix (Henikoff & Henikoff, 1992, Proc Natl Acad Sci USA 89:10915-9) and the set of default parameters for pair-wise comparison (gap creation cost=l 1, gap extension cost=l).

The percentage of sequence identity between polypeptides and their corresponding functions may be determined, for example, using a variety of homology-based search algorithms that are available to compare a query sequence, to a protein database, including for example, BLAST, FASTA, and Smith-Waterman. BLASTX and BLASTP algorithms may be used to provide protein function information. A number of values are examined in order to assess the confidence of the function assignment. Useful measurements include “E-value” (also shown as “hit_p”), “percent identity”, “percent query coverage”, and “percent hit coverage”. In BLAST, the E-value, or the expectation value, represents the number of different alignments with scores equivalent to or better than the raw alignment score, S, that are expected to occur in a database search by chance. Hence, the lower the E value, the more significant the match. Since database size is an element in E-value calculations, the E-values obtained by doing a BLAST search against public databases, such as GenBank, have generally increased over time for any given query/entry match. Thus, in setting criteria for confidence of polypeptide function prediction, a “high” BLASTX match is considered as having an E-value for the top BLASTX hit of less than 1E-30; a medium BLASTX is considered as having an E-value of 1E-30 to 1E-8; and a low BLASTX is considered as having an E-value of greater than 1E-8. Percent identity refers to the percentage of identically matched amino acid residues that exist along the length of that portion of the sequences which is aligned by the BLAST algorithm. In setting criteria for confidence of polypeptide function prediction, a “high” BLAST match is considered as having percent identity for the top BLAST hit of at least 70%; a medium percent identity value is considered from 35% to 70%; and a low percent identity is considered of less than 35%. Of particular interest in protein function assignment is the use of combinations of E-values, percent identity, query coverage and hit coverage. Query coverage refers to the percent of the query sequence that is represented in the BLAST alignment, whereas hit coverage refers to the percent of the database entry that is represented in the BLAST alignment. For the purpose of defining the polypeptides functionally covered by the present invention, the function of a polypeptide is deduced from the function of a protein homolog, such as SEQ ID NO: 1, SEQ ID NO: 2 or SEQ ID NO: 3, wherein a polypeptide of the invention is one that either (1) results in hit_p<le-30 or % identity >35% AND query_coverage>50% AND hit_coverage>50%, or (2) results in hit_p<le-8 AND query_coverage>70% AND hit_coverage>70%.

Variants of the ectodomain of hACE2 or of a fragment thereof comprising the amino acid sequence shown as SEQ ID NO: 3 may maintain at least about 75%, for example at least about 80%, 90%, 95%, 96%, 97%, 98%, 99% or 100% of the capacity to interact with the S protein of coronavirus of the wild type hACE2. Variants of the ectodomain of hACE2 or of a fragment thereof comprising the amino acid sequence shown as SEQ ID NO: 3 may have an increased capacity to interact with the S protein of coronavirus of about 105%, for example at least about 110%, 115%, 120%, 125%, 130%, 140%, 150% or more compared with that of the wild type hACE2.

The interaction between ACE2 and coronavirus S protein can be determined by conventional methods. For example, in vitro binding of ACE2 to the S protein may be determined by ELISA, surface plasmon resonance (SPR), or by flow cytometry using ACE2- or S protein-expressing cells. Additionally, the virus neutralisation ability of ACE2-based fusion proteins may be determined by incubating the fusion proteins with relevant virus, e.g. lentiviral vectors pseudotyped with coronavirus S protein, and cultured onto ACE2-expressing cells. These methods are further described in Example 2.

Since hACE2 has a key role in the Renin-Angiotensin System (RAS), as previously explained, the present inventors have hypothesised that the use of hACE2 mutants having a partial or complete obliteration of its catalytic activity will prevent any deleterious effect associated with an increased systemic presence of hACE2. Angiotensin 1-8 (Angiotensin II) is processed by ACE2 enzyme to form Angiotensin 1-7 which mediates vasodilation, diuresis, anti-inflammatory and anti-proliferative activity via interaction with MES receptors (Lovren et al. Am J Physiol Heart Circ Physiol 2008). Tilting the ACE/ACE2 balance on one side can cause hypertension, cardiac dysfunction and pro-inflammatory activity, on the other hand, systemic ACE2 activity and downregulation of angiotensin 1-8 can result in lower blood pressure, myocardial disturbance and immunosuppression (Tikellis and Thomas, Int J Pept 2012)

Thus, the variant or mutant of the ectodomain of hACE2 or of a fragment thereof comprising the amino acid sequence shown as SEQ ID NO: 3 may comprise one or more mutations in residues at positions that are involved in the enzymatic activity of hACE2. These mutations will advantageously decrease or eliminate completely (i.e. inactivate) the catalytic activity of hACE2. Moreover, the variant or mutant of the ectodomain of hACE2 or of a fragment thereof comprising the amino acid sequence shown as SEQ ID NO: 3 comprising one or more of these mutations will maintain the capacity of wild type hACE2 to interact with the S protein of coronavirus. The variant or mutant of the ectodomain of hACE2 or of a fragment thereof comprising the amino acid sequence shown as SEQ ID NO: 3 may comprise two or more mutations in residues at positions that are involved in the enzymatic activity of hACE2. Amino acids involved in the enzymatic activity of hACE2 include Arg 169, Arg 273, His 345, Pro 346, Thr 371, His 374, Glu 375, His 378, Glu 402, Trp 477, Lys 481, His 505, and Tyr 515, with respect of the sequence of the full, unprocessed hACE2 ectodomain shown as SEQ ID NO: 2.

The variant or mutant of the ectodomain of hACE2 or of a fragment thereof comprising the amino acid sequence shown as SEQ ID NO: 3 may comprise mutations at His 374 and His 378. The terms inactive ACE2 and ACE2(HH:NN) are used in this document to refer to this molecule.

The variant of the ectodomain of hACE2 may comprise or consist of the sequence shown as SEQ ID NO: 104. The variant of a fragment thereof comprising the amino acid sequence shown as SEQ ID NO: 3 may comprise or consist of the sequence shown as SEQ ID NO: 105. These two variants contain mutations at His 374 and His 378.

Processed ACE2 ectodomain variant
(SEQ ID NO: 104):
QSTIEEQAKTFLDKFNHEAEDLFYQSSLASWNYNTNITEENVQNMNNAGD
KWSAFLKEQSTLAQMYPLQEIQNLTVKLQLQALQQNGSSVLSEDKSKRLN
TILNTMSTIYSTGKVCNPDNPQECLLLEPGLNEIMANSLDYNERLWAWES
WRSEVGKQLRPLYEEYVVLKNEMARANHYEDYGDYWRGDYEVNGVDGYDY
SRGQLIEDVEHTFEEIKPLYEHLHAYVRAKLMNAYPSYISPIGCLPAHLL
GDMWGRFWTNLYSLTVPFGQKPNIDVTDAMVDQAWDAQRIFKEAEKFFVS
VGLPNMTQGFWENSMLTDPGNVQKAVCHPTAWDLGKGDFRILMCTKVTMD
DFLTAHNEMGNIQYDMAYAAQPFLLRNGANEGFHEAVGEIMSLSAATPKH
LKSIGLLSPDFQEDNETEINFLLKQALTIVGTLPFTYMLEKWRWMVFKGE
IPKDQWMKKWWEMKREIVGVVEPVPHDETYCDPASLFHVSNDYSFIRYYT
RTLYQFQFQEALCQAAKHEGPLHKCDISNSTEAGQKLFNMLRLGKSEPWT
LALENVVGAKNMNVRPLLNYFEPLFTWLKDQNKNSFVGWSTDWSPYADQS
IKVRISLKSALGDKAYEWNDNEMYLFRSSVAYAMRQYFLKVKNQMILFGE
EDVRVANLKPRISFNFFVTAPKNVSDIIPRTEVEKAIRMSRSRINDAFRL
NDNSLEFLGIQPTLGPPNQPPVS
Truncated ACE2 variant, aa 18-605
(SEQ ID NO: 105):
QSTIEEQAKTFLDKFNHEAEDLFYQSSLASWNYNTNITEENVQNMNNAGD
KWSAFLKEQSTLAQMYPLQEIQNLTVKLQLQALQQNGSSVLSEDKSKRLN
TILNTMSTIYSTGKVCNPDNPQECLLLEPGLNEIMANSLDYNERLWAWES
WRSEVGKQLRPLYEEYVVLKNEMARANHYEDYGDYWRGDYEVNGVDGYDY
SRGQLIEDVEHTFEEIKPLYEHLHAYVRAKLMNAYPSYISPIGCLPAHLL
GDMWGRFWTNLYSLTVPFGQKPNIDVTDAMVDQAWDAQRIFKEAEKFFVS
VGLPNMTQGFWENSMLTDPGNVQKAVCHPTAWDLGKGDFRILMCTKVTMD
DFLTAHNEMGNIQYDMAYAAQPFLLRNGANEGFHEAVGEIMSLSAATPKH
LKSIGLLSPDFQEDNETEINFLLKQALTIVGTLPFTYMLEKWRWMVFKGE
IPKDQWMKKWWEMKREIVGVVEPVPHDETYCDPASLFHVSNDYSFIRYYT
RTLYQFQFQEALCQAAKHEGPLHKCDISNSTEAGQKLFNMLRLGKSEPWT
LALENVVGAKNMNVRPLLNYFEPLFTWLKDQNKNSFVG

The variant or mutant of the ectodomain of hACE2 or of a fragment thereof comprising the amino acid sequence shown as SEQ ID NO: 3 having one or more mutations in residues at positions that are involved in the enzymatic activity of hACE2 may show a decreased catalytic activity compared to that of wild-type hACE2. The decreased catalytic activity may be of at least about 50% o, for example at least about 40%, 30%, 25% o, 20%, 15% o, 10%, 500 or 00% of the catalytic activity the wild type hACE2.

The enzymatic or catalytic activity of hACE2 (wild type or a variant, mutant or fragment thereof) may be evaluated by any means known in the art. For example, by incubating the protein with a surrogate fluorogenic substrate for ACE2, such as Mca-APK(Dnp) (Example 12), or with a positive control peptide and monitoring the fluorescence over time.

A domain A comprising more than one ectodomains of hACE2 or a fragment thereof comprising the amino acid sequence shown as SEQ ID NO: 3, or a variant thereof, is also within the scope of the present invention. Such a domain A will comprise two or more ectodomains of hACE2, or a fragment thereof comprising the amino acid sequence shown as SEQ ID NO: 3, or a variant thereof which maintains the capacity of hACE2 to interact with the S protein of coronavirus and, optionally, which has a decreased catalytic activity compared to that of wild-type hACE2, in tandem, optionally separated by a flexible linker, as will be described in following sections of the present application.

2.2. Domain O

In addition to a domain A, the first polypeptide of the invention also comprises a domain O, which comprises an oligomerisation domain. The resulting molecule will self-assemble into an oligomer. Oligomeric proteins have the advantage of an increased valency and, potentially, also display an extended serum half-life.

The term “oligomerisation domain”, as used herein, refers to a protein sequence, polypeptide or oligopeptide that self-assembles to form an oligomer. The oligomer may be a dimer, trimer, tetramer, pentamer, hexamer and so on depending on the number of monomers that assemble together, i.e. two, three, four, five, six and so on, respectively. Oligomers can be homooligomers, when all the monomers are the same, or heterooligomers, when the monomers are different.

Homooligomer

The oligomer may be a homooligomer. The oligomerisation domain of the homooligomer may be any homooligomerisation domain that is suitable for making fusion proteins.

The oligomerisation domain of the homooligomer may be selected from an IgG Fc region or a variant thereof which does not interact with FcγRI, FcγRIIa and FcγRIII, an IgM Fc region, an IgA Fc region, a collagen XVIII trimerizing structural element, a collagen XV trimerizing structural element, a foldon domain, a TenC domain and a homooligomerising coiled-coil domain.

The Fc region is the tail region of an antibody that is formed by the CH2 and the CH3 domains of an antibody. There are several different Fc regions, according to the antibody isotype and subclass, and these are the Fc regions of an IgG1, an IgG2, an IgG3, an IgG4, an IgM, an IgA, an IgE, and an IgD. The Fc regions dimerise, but in the case of IgM and IgA these dimers additionally form pentamers or further dimers, respectively. The additional oligomerisation may be particularly advantageous for increasing the valency of the first polypeptide of the invention, or when an avidity effect is to be obtained. The Fc region may be the Fc region of an IgG1, an IgG2, an IgG3, an IgG4, an IgM, or an IgA.

The Fc region may be the Fc region of an IgG1 depicted under Uniprot Accession No. P01857 as of 8 Apr. 2020 or a sequence shown as SEQ ID NO: 61. The Fc region may be the Fc region of an IgG2 (SEQ ID NO: 62).

The Fc region may comprise the hinge region. The Fc region may not comprise the hinge region.

The domain O may comprise the sequence shown as SEQ ID NO: 61 or SEQ ID NO: 62, shown below (hinge region is underlined):

Hinge-IgG1 Fc region (SEQ ID NO: 61):
EPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKS
RWQQGNVFSCSVMHEALHNHYTQKSLSLSPGK
IgG2 Fc (SEQ ID NO: 62):
ERKCCVECPPCPAPPVAGPSVFLFPPKPKDTLMISRTPEVTCVVVDVSHE
DPEVQFNWYVDGVEVHNAKTKPREEQFNSTFRVVSVLTVVHQDWLNGKEY
KCKVSNKGLPAPIEKTISKTKGQPREPQVYTLPPSREEMTKNQVSLTCLV
KGFYPSDISVEWESNGQPENNYKTTPPMLDSDGSFFLYSKLTVDKSRWQQ
GNVFSCSVMHEALHNHYTQKSLSLSPGK

Reports from SARS-CoV have highlighted that the natural immune response and sero-conversion in affected patients correlated with decreasing viral loads from day 10. However, it was also associated to severe clinical worsening, probably due to an overexuberant host response. Similarly, there is evidence of a detrimental role of anti-S protein antibodies in SARS-CoV infection, causing lung injuries, abrogating TGFβ production and promoting inflammatory macrophage accumulation. This activity could be counteracted by FcγR blockade.

Moreover, reports revealed that, in addition to infection of the respiratory tract, SARS-CoV may also directly infect immune cells which do not ACE2. The occurrence of antibody-dependent enhancement (ADE) of infection was established in different cell lines and circulating immune cell types, among which the monocytic lineage (CD68⁺ cells) was the primary target. In addition to monocytes, human macrophages were also infected by SARS-CoV in the presence of anti-spike antibodies. ADE is thought to prompt the massive release of inflammatory and vasoactive mediators that ultimately may contribute to the cytokine release syndrome and disease severity observed in coronavirus infections by SARS-CoV, SARS-CoV-2 and MERS-CoV. Therefore, interaction of the Fc region of IgG with its cognate receptors (FcγRI, FcγRIIa and/or FcγRIII) may be undesirable as it may lead to antibody-dependent cellular phagocytosis (ADCP) and ADE.

Thus, the present invention also contemplates using a variant of an Fc region of an IgG which does not interact with FcγRI, FcγRIIa and FcγRIII. Mutations that abrogate the effector function of the Fc region have been extensively investigated and are well-known in the art. For example, the Fc region of an IgG may contain one or more of the following mutations or mutation combinations:
Leu235Glu;
Leu234Ala and Leu235Ala (LALA);
Leu234Ala, Leu235Ala (LALA) and Pro329Gly (PG);
Pro331Ser, Leu234Glu and Leu235Phe;
Asp265Ala;
Gly237Ala;
Glu318Ala;
Glu233Pro;
Gly236Arg and Leu328Arg;
Ala330Leu;
Asp270Ala;
Lys322Ala;
Pro329Ala;
Pro331Ala;
Val264Ala;
Phe241Ala;
Asn297Ala;
Asn297Gly; and
Asn297Gln.

HuIgGFc with LALA mutations
(SEQ ID NO: 120)
EPKSCDKTHTCPPCPAPEAAGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKS
RWQQGNVFSCSVMHEALHNHYTQKSLSLSPGK
HuIgGFc with LALA and PG mutations
(SEQ ID NO: 121)
EPKSCDKTHTCPPCPAPEAAGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALGAPIEKTISKAKGQPREPQVYTLPPSREEMTKNQVSL
TCLVKGFYPSDIAVEWESNGQPENNYKTTPPVLDSDGSFFLYSKLTVDKS
RWQQGNVFSCSVMHEALHNHYTQKSLSLSPGK

The first polypeptide of the invention may comprise:
a) a domain A which comprises the ectodomain of ACE2 having a sequence of SEQ ID NO: 1, and
b) a domain O which comprises the Fc region of an IgG which contains the mutations Leu234Ala and Leu235Ala (LALA).

The first polypeptide of the invention may comprise:
a) a domain A which comprises the ectodomain of ACE2 having a sequence of SEQ ID NO: 1, and
b) a domain O which comprises the Fc region of an IgG which contains the mutations Leu234Ala, Leu235Ala (LALA) and Pro329Gly (PG).

The first polypeptide of the invention may comprise:
a) a domain A which comprises a fragment of the ectodomain of ACE2 comprising the amino acid sequence shown as SEQ ID NO: 3, and
b) a domain O which comprises the Fc region of an IgG which contains the mutations Leu234Ala and Leu235Ala (LALA).

The first polypeptide of the invention may comprise:
a) a domain A which comprises a fragment of the ectodomain of ACE2 comprising the amino acid sequence shown as SEQ ID NO: 3, and
b) a domain O which comprises the Fc region of an IgG which contains the mutations Leu234Ala, Leu235Ala (LALA) and Pro329Gly (PG).

The first polypeptide of the invention may comprise:
a) a domain A which comprises a variant of the ectodomain of ACE2 having a sequence of SEQ ID NO: 104, and
b) a domain O which comprises the Fc region of an IgG which contains the mutations Leu234Ala, Leu235Ala (LALA).

The first polypeptide of the invention may comprise:
a) a domain A which comprises a variant of the ectodomain of ACE2 having a sequence of SEQ ID NO: 104, and
b) a domain O which comprises the Fc region of an IgG which contains the mutations Leu234Ala, Leu235Ala (LALA) and Pro329Gly (PG).

The first polypeptide of the invention may comprise:
a) a domain A which comprises a fragment of a variant of the ectodomain of ACE2 comprising the amino acid sequence shown as SEQ ID NO: 105, and
b) a domain O which comprises the Fc region of an IgG which contains the mutations Leu234Ala, Leu235Ala (LALA).

The first polypeptide of the invention may comprise:
a) a domain A which comprises a fragment of a variant of the ectodomain of ACE2 comprising the amino acid sequence shown as SEQ ID NO: 105, and
b) a domain O which comprises the Fc region of an IgG which contains the mutations Leu234Ala, Leu235Ala (LALA) and Pro329Gly (PG).

These embodiments are particularly beneficial compared to standard antibody-mediated and vaccine approaches because of the avoidance of ADE, i.e. Fc receptor mediated uptake of virus in FcR-expressing cells. Furthermore, the present inventors found that polypeptides according to the first aspect of the invention comprising a IgG Fc having the LALA and PG mutations showed an improved virus neutralisation efficiency against SARS-CoV-2 (live virus and lentiviral pseudotyped virus expressing SARS-CoV-2 spike protein) compared with a polypeptide comprising the wild type Fc region (Example 13). The fact that variations in the Fc domain influence the neutralisation capacity of the therapeutic fusion protein is surprising because the Fc domain is not involved in the interaction between the SARS-CoV-2 spike protein and ACE2 and, moreover, the mutations in the Fc domain would be expected to have an effect solely on the interaction with the Fc receptors.

The Fc region may comprise the hinge region. The Fc region may lack the hinge region.

| | IgG1 hinge |
|---|---|
| (SEQ ID NO: 126) | |
| | EPKSCDKTHTCP |

The Fc region may be truncated. The truncated Fc region may comprise the CH3 domain. The truncated Fc region may comprise the hinge region and, optionally, a flexible linker. The truncated Fc region may comprise the sequence shown as SEQ ID NO: 118 or SEQ ID NO: 119.

Truncated Fc region (Hinge-CH3)
(SEQ ID NO: 118)
EPKSCDKTHTCPPCGQPREPQVYTLPPSREEMTKNQVSLTCLVKGFYPSD
IAVEWESNGQPENNYKTTPPVLDSDGSFFLYSKLTVDKSRWQQGNVFSCS
VMHEALHNHYTQKSLSLSPGK
Truncated Fc region (Hinge-CH3)
(SEQ ID NO: 119)
EPKSCDKTHTCPPCGGGSSGGGSGGQPREPQVYTLPPSREEMTKNQVSLT
CLVKGFYPSDIAVEWESNGQPENNYKTTPPVLDSDGSFFLYSKLTVDKSR
WQQGNVFSCSVMHEALHNHYTQKSLSLSPGK

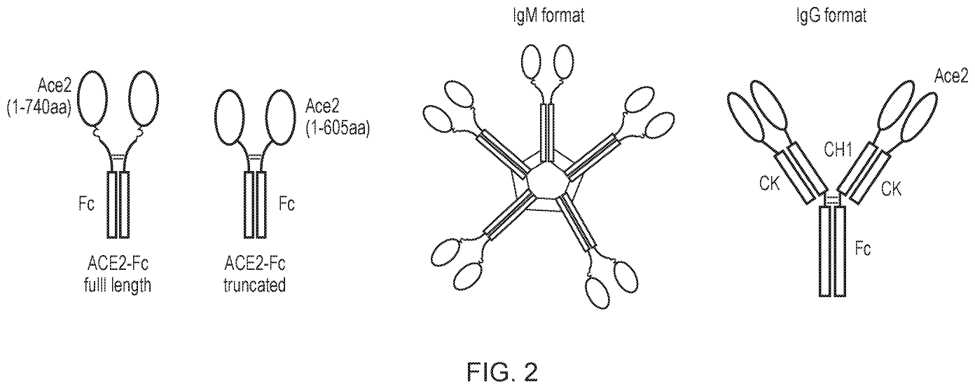

Non-limiting examples of fusion proteins comprising domain A and the Fc region in different orientations are depicted in FIG. 2.

The first polypeptide of the invention may comprise or consist of the amino acid sequence shown as SEQ ID NO: 76 to 79 and 125.

hACE2_18-740_HuIgG1Fc (SEQ ID NO: 76):
QSTIEEQAKTFLDKFNHEAEDLFYQSSLASWNYNTNITEENVQNMNNAGD
KWSAFLKEQSTLAQMYPLQEIQNLTVKLQLQALQQNGSSVLSEDKSKRLN
TILNTMSTIYSTGKVCNPDNPQECLLLEPGLNEIMANSLDYNERLWAWES
WRSEVGKQLRPLYEEYVVLKNEMARANHYEDYGDYWRGDYEVNGVDGYDY
SRGQLIEDVEHTFEEIKPLYEHLHAYVRAKLMNAYPSYISPIGCLPAHLL
GDMWGRFWTNLYSLTVPFGQKPNIDVTDAMVDQAWDAQRIFKEAEKFFVS
VGLPNMTQGFWENSMLTDPGNVQKAVCHPTAWDLGKGDFRILMCTKVTMD
DFLTAHNEMGNIQYDMAYAAQPFLLRNGANEGFHEAVGEIMSLSAATPKH
LKSIGLLSPDFQEDNETEINFLLKQALTIVGTLPFTYMLEKWRWMVFKGE
IPKDQWMKKWWEMKREIVGVVEPVPHDETYCDPASLFHVSNDYSFIRYYT
RTLYQFQFQEALCQAAKHEGPLHKCDISNSTEAGQKLFNMLRLGKSEPWT
LALENVVGAKNMNVRPLLNYFEPLFTWLKDQNKNSFVGWSTDWSPYADQS
IKVRISLKSALGDKAYEWNDNEMYLFRSSVAYAMRQYFLKVKNQMILFGE
EDVRVANLKPRISFNFFVTAPKNVSDIIPRTEVEKAIRMSRSRINDAFRL
NDNSLEFLGIQPTLGPPNQPPVSEPKSCDKTHTCPPCPAPELLGGPSVFL
FPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYK
TTPPVLDSDGSFFLYSKLTVDKSRWQQGNVFSCSVMHEALHNHYTQKSLS
LSPGK
hACE2_18-740_HuIgMFc (SEQ ID NO: 77):
QSTIEEQAKTFLDKFNHEAEDLFYQSSLASWNYNTNITEENVQNMNNAGD
KWSAFLKEQSTLAQMYPLQEIQNLTVKLQLQALQQNGSSVLSEDKSKRLN
TILNTMSTIYSTGKVCNPDNPQECLLLEPGLNEIMANSLDYNERLWAWES
WRSEVGKQLRPLYEEYVVLKNEMARANHYEDYGDYWRGDYEVNGVDGYDY
SRGQLIEDVEHTFEEIKPLYEHLHAYVRAKLMNAYPSYISPIGCLPAHLL
GDMWGRFWTNLYSLTVPFGQKPNIDVTDAMVDQAWDAQRIFKEAEKFFVS
VGLPNMTQGFWENSMLTDPGNVQKAVCHPTAWDLGKGDFRILMCTKVTMD
DFLTAHNEMGNIQYDMAYAAQPFLLRNGANEGFHEAVGEIMSLSAATPKH
LKSIGLLSPDFQEDNETEINFLLKQALTIVGTLPFTYMLEKWRWMVFKGE
IPKDQWMKKWWEMKREIVGVVEPVPHDETYCDPASLFHVSNDYSFIRYYT
RTLYQFQFQEALCQAAKHEGPLHKCDISNSTEAGQKLFNMLRLGKSEPWT
LALENVVGAKNMNVRPLLNYFEPLFTWLKDQNKNSFVGWSTDWSPYADQS
IKVRISLKSALGDKAYEWNDNEMYLFRSSVAYAMRQYFLKVKNQMILFGE
EDVRVANLKPRISFNFFVTAPKNVSDIIPRTEVEKAIRMSRSRINDAFRL
NDNSLEFLGIQPTLGPPNQPPVSSGGGSIAELPPKVSVFVPPRDGFFGNP
RSKSKLICQATGFSPRQIQVSWLREGKQVGSGVTTDQVQAEAKESGPTTY
KVTSTLTIKESDWLSQSMFTCRVDHRGLTFQQNASSMCVPDQDTAIRVFA
IPPSFASIFLTKSTKLTCLVTDLTTYDSVTISWTRQNGEAVKTHTNISES
HPNATFSAVGEASICEDDWNSGERFTCTVTHTDLPSPLKQTISRPKGVAL
HRPDVYLLPPAREQLNLRESATITCLVTGFSPADVFVQWMQRGQPLSPEK
YVTSAPMPEPQAPGRYFAHSILTVSEEEWNTGETYTCVVAHEALPNRVTE
RTVDKST
ACE2_18-605_HuIgMFc (SEQ ID NO: 78):
QSTIEEQAKTFLDKFNHEAEDLFYQSSLASWNYNTNITEENVQNMNNAGD
KWSAFLKEQSTLAQMYPLQEIQNLTVKLQLQALQQNGSSVLSEDKSKRLN
TILNTMSTIYSTGKVCNPDNPQECLLLEPGLNEIMANSLDYNERLWAWES
WRSEVGKQLRPLYEEYVVLKNEMARANHYEDYGDYWRGDYEVNGVDGYDY
SRGQLIEDVEHTFEEIKPLYEHLHAYVRAKLMNAYPSYISPIGCLPAHLL
GDMWGRFWTNLYSLTVPFGQKPNIDVTDAMVDQAWDAQRIFKEAEKFFVS
VGLPNMTQGFWENSMLTDPGNVQKAVCHPTAWDLGKGDFRILMCTKVTMD
DFLTAHNEMGNIQYDMAYAAQPFLLRNGANEGFHEAVGEIMSLSAATPKH
LKSIGLLSPDFQEDNETEINFLLKQALTIVGTLPFTYMLEKWRWMVFKGE
IPKDQWMKKWWEMKREIVGVVEPVPHDETYCDPASLFHVSNDYSFIRYYT
RTLYQFQFQEALCQAAKHEGPLHKCDISNSTEAGQKLFNMLRLGKSEPWT
LALENVVGAKNMNVRPLLNYFEPLFTWLKDQNKNSFVGSGGGSIAELPPK
VSVFVPPRDGFFGNPRSKSKLICQATGFSPRQIQVSWLREGKQVGSGVTT
DQVQAEAKESGPTTYKVTSTLTIKESDWLSQSMFTCRVDHRGLTFQQNAS
SMCVPDQDTAIRVFAIPPSFASIFLTKSTKLTCLVTDLTTYDSVTISWTR
QNGEAVKTHTNISESHPNATFSAVGEASICEDDWNSGERFTCTVTHTDLP
SPLKQTISRPKGVALHRPDVYLLPPAREQLNLRESATITCLVTGFSPADV
FVQWMQRGQPLSPEKYVTSAPMPEPQAPGRYFAHSILTVSEEEWNTGETY
TCVVAHEALPNRVTERTVDKST
ACE2_18-605_HuIgG1Fc (SEQ ID NO: 79):
QSTIEEQAKTFLDKFNHEAEDLFYQSSLASWNYNTNITEENVQNMNNAGD
KWSAFLKEQSTLAQMYPLQEIQNLTVKLQLQALQQNGSSVLSEDKSKRLN
TILNTMSTIYSTGKVCNPDNPQECLLLEPGLNEIMANSLDYNERLWAWES
WRSEVGKQLRPLYEEYVVLKNEMARANHYEDYGDYWRGDYEVNGVDGYDY
SRGQLIEDVEHTFEEIKPLYEHLHAYVRAKLMNAYPSYISPIGCLPAHLL
GDMWGRFWTNLYSLTVPFGQKPNIDVTDAMVDQAWDAQRIFKEAEKFFVS
VGLPNMTQGFWENSMLTDPGNVQKAVCHPTAWDLGKGDFRILMCTKVTMD
DFLTAHNEMGNIQYDMAYAAQPFLLRNGANEGFHEAVGEIMSLSAATPKH
LKSIGLLSPDFQEDNETEINFLLKQALTIVGTLPFTYMLEKWRWMVFKGE
IPKDQWMKKWWEMKREIVGVVEPVPHDETYCDPASLFHVSNDYSFIRYYT
RTLYQFQFQEALCQAAKHEGPLHKCDISNSTEAGQKLFNMLRLGKSEPWT
LALENVVGAKNMNVRPLLNYFEPLFTWLKDQNKNSFVGEPKSCDKTHTCP
PCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDI
AVEWESNGQPENNYKTTPPVLDSDGSFFLYSKLTVDKSRWQQGNVFSCSV
MHEALHNHYTQKSLSLSPGK
Inactive ACE2-Fc (LALA-PG) (ACE2(HH:NN)-
Fc LALA-PG) (SEQ ID NO: 125):
QSTIEEQAKTFLDKFNHEAEDLFYQSSLASWNYNTNITEENVQNMNNAGD
KWSAFLKEQSTLAQMYPLQEIQNLTVKLQLQALQQNGSSVLSEDKSKRLN
TILNTMSTIYSTGKVCNPDNPQECLLLEPGLNEIMANSLDYNERLWAWES
WRSEVGKQLRPLYEEYVVLKNEMARANHYEDYGDYWRGDYEVNGVDGYDY
SRGQLIEDVEHTFEEIKPLYEHLHAYVRAKLMNAYPSYISPIGCLPAHLL
GDMWGRFWTNLYSLTVPFGQKPNIDVTDAMVDQAWDAQRIFKEAEKFFVS
VGLPNMTQGFWENSMLTDPGNVQKAVCHPTAWDLGKGDFRILMCTKVTMD
DFLTAHNEMGNIQYDMAYAAQPFLLRNGANEGFHEAVGEIMSLSAATPKH
LKSIGLLSPDFQEDNETEINFLLKQALTIVGTLPFTYMLEKWRWMVFKGE
IPKDQWMKKWWEMKREIVGVVEPVPHDETYCDPASLFHVSNDYSFIRYYT
RTLYQFQFQEALCQAAKHEGPLHKCDISNSTEAGQKLFNMLRLGKSEPWT
LALENVVGAKNMNVRPLLNYFEPLFTWLKDQNKNSFVGWSTDWSPYADQS
IKVRISLKSALGDKAYEWNDNEMYLFRSSVAYAMRQYFLKVKNQMILFGE
EDVRVANLKPRISFNFFVTAPKNVSDIIPRTEVEKAIRMSRSRINDAFRL
NDNSLEFLGIQPTLGPPNQPPVSEPKSCDKTHTCPPCPAPEAAGGPSVFL
FPPKPKDTLMISRTPEVTCVVVDVSHEDPEVKFNWYVDGVEVHNAKTKPR
EEQYNSTYRVVSVLTVLHQDWLNGKEYKCKVSNKALGAPIEKTISKAKGQ
PREPQVYTLPPSREEMTKNQVSLTCLVKGFYPSDIAVEWESNGQPENNYK
TTPPVLDSDGSFFLYSKLTVDKSRWQQGNVFSCSVMHEALHNHYTQKSLS
LSPGK

The present invention also contemplates using a variant of the Fc region of an IgG which does not interact with FcγRI, FcγRIIa and FcγRIII and displays improved circulation or serum half-life. One or more of the following mutations or mutation combinations may be combined to the previously described silencing mutations (i.e. mutations that abrogate the effector function of the Fc region) in the IgG Fc region:
Arg435His;
Asn434Ala;
Met252Tyr, Ser 254Thr and Thr256Glu;
Met428Leu and Asn434Ser;
Thr252Leu, Thr253Ser and Thr254Phe;
Glu294delta, Thr307Pro and Asn434Tyr;
Thr256Asn, Ala378Val, Ser383Asn, and Asn434Tyr; and
Glu294delta.

The variant of the Fc region of an IgG which does not interact with FcγRI, FcγRIIa and FcγRIII and displays improved circulation or serum half-life may comprise mutations Met252Tyr, Ser 254Thr and Thr256Glu (YTE).

The variant of the Fc region of an IgG which does not interact with FcγRI, FcγRIIa and FcγRIII and displays improved circulation or serum half-life may comprise the sequence shown as SEQ ID NO: 128.

HulgG1Fc with LALA, PG and YTE mutations
(SEQ ID NO: 128)
EPKSCDKTHTCPPCPAPEAAGGPSVFLFPPKPKDTLYITREPEVTCVVV
DVSHEDPEVKFNWYVDGVEVHNAKTKPREEQYNSTYRVVSVLTVLHQDW
LNGKEYKCKVSNKALGAPIEKTISKAKGQPREPQVYTLPPSREEMTKNQ
VSLTCLVKGFYPSDIAVEWESNGQPENNYKTTPPVLDSDGSFFLYSKLT
VDKSRWQQGNVFSCSVMHEALHNHYTQKSLSLSPGK

The first polypeptide of the invention may comprise:
a) a domain A which comprises the ectodomain of ACE2 having a sequence of SEQ ID NO: 1, and
b) a domain O which comprises the Fc region of an IgG which contains the mutations Leu234Ala and Leu235Ala (LALA), and Met252Tyr, Ser 254Thr and Thr256Glu (YTE).

The first polypeptide of the invention may comprise:
a) a domain A which comprises the ectodomain of ACE2 having a sequence of SEQ ID NO: 1, and
b) a domain O which comprises the Fc region of an IgG which contains the mutations Leu234Ala, Leu235Ala (LALA) and Pro329Gly (PG), and Met252Tyr, Ser 254Thr and Thr256Glu (YTE).

The first polypeptide of the invention may comprise:
a) a domain A which comprises a fragment of the ectodomain of ACE2 comprising the amino acid sequence shown as SEQ ID NO: 3, and
b) a domain O which comprises the Fc region of an IgG which contains the mutations Leu234Ala and Leu235Ala (LALA), and Met252Tyr, Ser 254Thr and Thr256Glu (YTE).

The first polypeptide of the invention may comprise:
a) a domain A which comprises a fragment of the ectodomain of ACE2 comprising the amino acid sequence shown as SEQ ID NO: 3, and
b) a domain O which comprises the Fc region of an IgG which contains the mutations Leu234Ala, Leu235Ala (LALA) and Pro329Gly (PG), and Met252Tyr, Ser 254Thr and Thr256Glu (YTE).

The first polypeptide of the invention may comprise:
a) a domain A which comprises a variant of the ectodomain of ACE2 having a sequence of SEQ ID NO: 104, and
b) a domain O which comprises the Fc region of an IgG which contains the mutations Leu234Ala, Leu235Ala (LALA), and Met252Tyr, Ser 254Thr and Thr256Glu (YTE).

The first polypeptide of the invention may comprise:
a) a domain A which comprises a variant of the ectodomain of ACE2 having a sequence of SEQ ID NO: 104, and
b) a domain O which comprises the Fc region of an IgG which contains the mutations Leu234Ala, Leu235Ala (LALA) and Pro329Gly (PG), and Met252Tyr, Ser 254Thr and Thr256Glu (YTE).

The first polypeptide of the invention may comprise:
a) a domain A which comprises a fragment of a variant of the ectodomain of ACE2 comprising the amino acid sequence shown as SEQ ID NO: 105, and
b) a domain O which comprises the Fc region of an IgG which contains the mutations Leu234Ala, Leu235Ala (LALA), and Met252Tyr, Ser 254Thr and Thr256Glu (YTE).

The first polypeptide of the invention may comprise:
a) a domain A which comprises a fragment of a variant of the ectodomain of ACE2 comprising the amino acid sequence shown as SEQ ID NO: 105, and
b) a domain O which comprises the Fc region of an IgG which contains the mutations Leu234Ala, Leu235Ala (LALA) and Pro329Gly (PG), and Met252Tyr, Ser 254Thr and Thr256Glu (YTE).

The first polypeptide of the invention may comprise or consist of the amino acid sequence shown as SEQ ID NOs: 129 or 130.

hACE2_18-740_HulgG1Fc (LALA-PG, YTE)
(SEQ ID NO: 129):
QSTIEEQAKTFLDKFNHEAEDLFYQSSLASWNYNTNITEENVQNMNNAG
DKWSAFLKEQSTLAQMYPLQEIQNLTVKLQLQALQQNGSSVLSEDKSKR
LNTILNTMSTIYSTGKVCNPDNPQECLLLEPGLNEIMANSLDYNERLWA
WESWRSEVGKQLRPLYEEYVVLKNEMARANHYEDYGDYWRGDYEVNGVD
GYDYSRGQLIEDVEHTFEEIKPLYEHLHAYVRAKLMNAYPSYISPIGCL
PAHLLGDMWGRFWTNLYSLTVPFGQKPNIDVTDAMVDQAWDAQRIFKEA
EKFFVSVGLPNMTQGFWENSMLTDPGNVQKAVCHPTAWDLGKGDFRILM
CTKVTMDDFLTAHNEMGNIQYDMAYAAQPFLLRNGANEGFHEAVGEIMS
LSAATPKHLKSIGLLSPDFQEDNETEINFLLKQALTIVGTLPFTYMLEK
WRWMVFKGEIPKDQWMKKWWEMKREIVGVVEPVPHDETYCDPASLFHVS
NDYSFIRYYTRTLYQFQFQEALCQAAKHEGPLHKCDISNSTEAGQKLFN
MLRLGKSEPWTLALENVVGAKNMNVRPLLNYFEPLFTWLKDQNKNSFVG
WSTDWSPYADQSIKVRISLKSALGDKAYEWNDNEMYLFRSSVAYAMRQY
FLKVKNQMILFGEEDVRVANLKPRISFNFFVTAPKNVSDIIPRTEVEKA
IRMSRSRINDAFRLNDNSLEFLGIQPTLGPPNQPPVSEPKSCDKTHTCP
PCPAPEAAGGPSVFLFPPKPKDTLYITREPEVTCVVVDVSHEDPEVKFN
WYVDGVEVHNAKTKPREEQYNSTYRVVSVLTVLHQDWLNGKEYKCKVSN
KALGAPIEKTISKAKGQPREPQVYTLPPSREEMTKNQVSLTCLVKGFYP
SDIAVEWESNGQPENNYKTTPPVLDSDGSFFLYSKLTVDKSRWQQGNVF
SCSVMHEALHNHYTQKSLSLSPGK
Inactive ACE2-Fc (LALA-PG, YTE) (ACE2(HH:NN)-Fc
LALA-PG, YTE) (SEQ ID NO: 130):
QSTIEEQAKTFLDKFNHEAEDLFYQSSLASWNYNTNITEENVQNMNNAG
DKWSAFLKEQSTLAQMYPLQEIQNLTVKLQLQALQQNGSSVLSEDKSKR
LNTILNTMSTIYSTGKVCNPDNPQECLLLEPGLNEIMANSLDYNERLWA
WESWRSEVGKQLRPLYEEYVVLKNEMARANHYEDYGDYWRGDYEVNGVD
GYDYSRGQLIEDVEHTFEEIKPLYEHLHAYVRAKLMNAYPSYISPIGCL
PAHLLGDMWGRFWTNLYSLTVPFGQKPNIDVTDAMVDQAWDAQRIFKEA
EKFFVSVGLPNMTQGFWENSMLTDPGNVQKAVCHPTAWDLGKGDFRILM
CTKVTMDDFLTAHNEMGNIQYDMAYAAQPFLLRNGANEGFHEAVGEIMS
LSAATPKHLKSIGLLSPDFQEDNETEINFLLKQALTIVGTLPFTYMLEK
WRWMVFKGEIPKDQWMKKWWEMKREIVGVVEPVPHDETYCDPASLFHVS
NDYSFIRYYTRTLYQFQFQEALCQAAKHEGPLHKCDISNSTEAGQKLFN
MLRLGKSEPWTLALENVVGAKNMNVRPLLNYFEPLFTWLKDQNKNSFVG
WSTDWSPYADQSIKVRISLKSALGDKAYEWNDNEMYLFRSSVAYAMRQY
FLKVKNQMILFGEEDVRVANLKPRISFNFFVTAPKNVSDIIPRTEVEKA
IRMSRSRINDAFRLNDNSLEFLGIQPTLGPPNQPPVSEPKSCDKTHTCP
PCPAPEAAGGPSVFLFPPKPKDTLYITREPEVTCVVVDVSHEDPEVKFN
WYVDGVEVHNAKTKPREEQYNSTYRVVSVLTVLHQDWLNGKEYKCKVSN
KALGAPIEKTISKAKGQPREPQVYTLPPSREEMTKNQVSLTCLVKGFYP
SDIAVEWESNGQPENNYKTTPPVLDSDGSFFLYSKLTVDKSRWQQGNVF
SCSVMHEALHNHYTQKSLSLSPGK

The oligomerisation domain may be a collagen XVIII trimerizing structural element. The term “collagen XVIII trimerizing structural element” or “XVIIITSE”, as used herein, refers to the portion of collagen XVIII which is responsible for trimerization between monomers of collagen XVIII. The term is also intended to embrace functionally equivalent variants of a XVIIITSE of a naturally occurring collagen XVIII, variants which have been modified in the amino acid sequence without adversely affecting, to any substantial degree, the trimerization properties relative to those of the native collagen XVIII molecule. Said modifications include, the conservative (or non-conservative) substitution of one or more amino acids for other amino acids, the insertion and/or the deletion of one or more amino acids, provided that the trimerization properties of the native collagen XVIII protein is substantially maintained, i.e., the variant maintains the ability (capacity) of forming trimers with other peptides having the same sequence at physiological conditions.

The XVIIITSE may be a polypeptide having the amino acid sequence shown in SEQ ID NO: 4.

XVIIITSE (SEQ ID NO: 4):
SGVRLWATRQAMLGQVHEVPEGWLIFVAEQEELYVRVQNGFRKVQLEAR
TPLPRGTDNE

The oligomerisation domain may be a collagen XV trimerizing structural element. The term “collagen XV trimerizing structural element” or “XVTSE”, as used herein, refers to the portion of collagen XV which is responsible for trimerization between monomers of collagen XV. The term is also intended to embrace functionally equivalent variants of a XVTSE of a naturally occurring collagen XV, variants which have been modified in the amino acid sequence without adversely affecting, to any substantial degree, the trimerization properties relative to those of the native collagen XV molecule. Said modifications include, the conservative (or non-conservative) substitution of one or more amino acids for other amino acids, the insertion and/or the deletion of one or more amino acids, provided that the trimerization properties of the native collagen XV protein is substantially maintained, i.e., the variant maintains the ability (capacity) of forming trimers with other peptides having the same sequence at physiological conditions.

The XVTSE may be a polypeptide having the amino acid sequence shown in SEQ ID NO: 5.

XVTSE (SEQ ID NO: 5):
VTAFSNMDDMLQKAHLVIEGTFIYLRDSTEFFIRVRDGWKKLQLGELIP
IPADSPPPPALSSNP

The oligomerisation domain may be a foldon domain. The term “foldon domain”, “foldon T4” or “foldon T4 domain”, as used herein, refers to the C-terminal amino acid residues of the trimeric protein fibritin from bacteriophage T4 (SEQ ID NO: 63). The foldon domain promotes folding and trimerisation of fibritin. This feature has been exploited to trimerise other molecules.

Foldon T4 (SEQ ID NO: 63):
GYIPEAPRDGQAYVRKDGEWVLLSTFL

The oligomerisation domain may be a TenC domain. The term “TenC domain”, as used herein, refers to the oligomerisation domain located at the N-terminus of Tenascin C (TN-C). The TenC domain may be human (SEQ ID NO: 64) or from chicken (SEQ ID NO: 65). The TenC domain forms trimers.

Human TenC domain (SEQ ID NO: 64):
ACGCAAAPDVKELLSRLEELENLVSSLREQ
Chicken TenC domain (SEQ ID NO: 65):
ACGCAAAPDVKDLLSRLEELEGLVSSLREQ

The oligomerisation domain may be a coiled coil domain. A “coiled coil” is a structural motif in which two to seven alpha helices are wrapped together like the strands of a rope. Many endogenous proteins incorporate coiled coil domains. The coiled coil domain may be involved in protein folding (e.g. it interacts with several alpha helical motifs within the same protein chain) or responsible for protein-protein interaction. In the latter case, the coiled coil can initiate homo or hetero oligomer structures.

The structure of coiled coil domains is well known in the art. For example, as described by Lupas & Gruber (2007, Advances in Protein Chemistry 70:37-8). Coiled coils usually contain a repeated pattern, hxxhcxc, of hydrophobic (h) and charged (c) amino-acid residues, referred to as a heptad repeat. The positions in the heptad repeat are usually labelled abcdefg, where a and d are the hydrophobic positions, often being occupied by isoleucine, leucine, or valine.

Examples of proteins which contain a homooligomerising coiled coil domain include, but are not limited to, cartilage-oligomeric matrix protein (COMP), kinesin motor protein, hepatitis D delta antigen, archaeal box C/D sRNP core protein, mannose-binding protein A, coiled-coil serine-rich protein 1, polypeptide release factor 2, SNAP-25, SNARE, Lac repressor or apolipoprotein E.

The sequence of various coiled coil domains is shown below:

Cartilage oligomeric matrix protein (COMP)
homopentamer
(SEQ ID NO: 66)
AGSDLGPQMLRELQETNAALQDVRELLRQQVREITFLKNTVMECDACGS
GKKDPK
Kinesin motor protein: parallel homodimer
(SEQ ID NO: 106)
MHAALSTEVVHLRQRTEELLRCNEQQAAELETCKEQLFQSNMERKELHN
TVMDLRGN
Hepatitis D delta antigen: parallel homodimer
(SEQ ID NO: 107)
GREDILEQWVSGRKKLEELERDLRKLKKKIKKLEEDNPWLGNIKGIIGK
Y
Archaeal box C/D sRNP core protein: anti-parallel
heterodimer
(SEQ ID NO: 108)
RYVVALVKALEEIDESINMLNEKLEDIRAVKESEITEKFEKKIRELREL
RRDVEREIEEVM
Mannose-binding protein A: parallel homotrimer
(SEQ ID NO: 109)
AIEVKLANMEAEINTLKSKLELTNKLHAFSM
Coiled-coil serine-rich protein 1: parallel
homotrimer
(SEQ ID NO: 110)
EWEALEKKLAALESKLQALEKKLEALEHG

The oligomerisation domain may be a p53 oligomerisation domain. The term “p53 oligomerisation domain” or “p53 domain”, as used herein, refers to the oligomerisation domain located at the C-terminus of p53. The TenC domain may comprise the sequence shown as SEQ ID NO: 136. The TenC domain forms tetramers.

p53 oligomerisation domain
(SEQ ID NO: 136)
KKKPLDGEYFTLQIRGRERFEMFRELNEALELKDAQAGKEPG

Heterooligomer

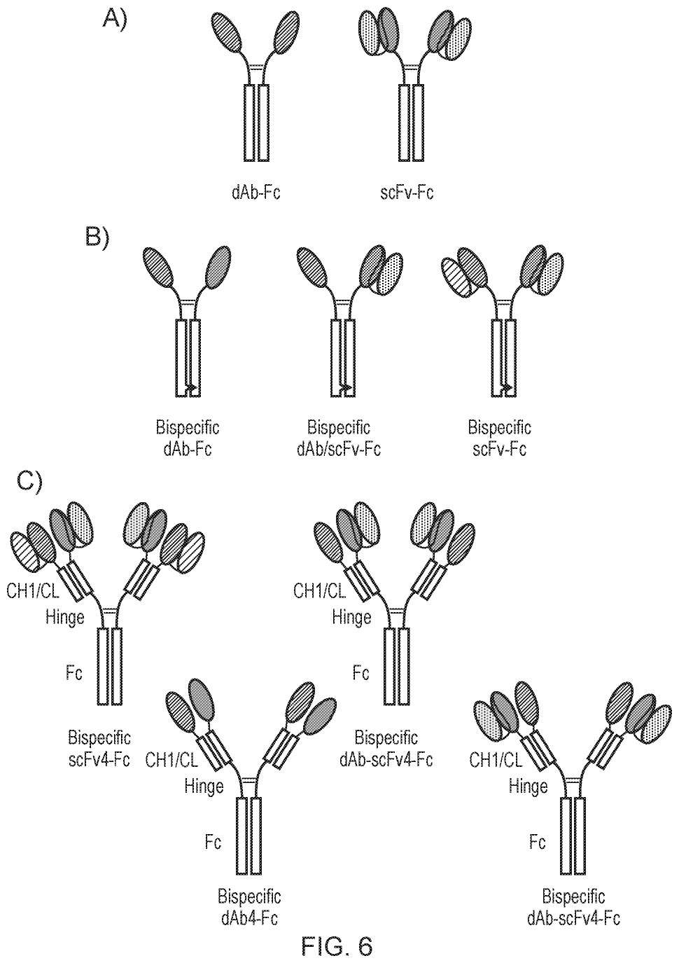

The oligomer may be a heterooligomer. The use of heterooligomerisation domains may be advantageous when it is intended that the polypeptide of the invention contains two or more different domains A, as described previously, or two or more different domains C, domains ABD or domains ALB, as will be described in subsequent sections of the present application. For example, the use of a heterooligomerisation domain, such as a heterodimerization domain, enables the fusion of an antibody binding domain having different specificity to each of the heteromonomers. This results in bispecific molecules, such as the general bispecific scFv-based fusion proteins depicted in FIGS. 6B, 6C and 25.

Different heterooligomerisation domains may be used in the context of the present invention. Non-limiting examples of heterooligomerisation domains are described in Brinkmann & Kontermann (2017, MAbs 9:182-212), and include the dock-and-lock (DNL) modules, knobs-into-holes modified CH3 domains, SEEDbodies, bispecific tetravalent Fc fusions, dual variable domain Ig (DVD), the barnase-barstar domains, and heterooligomerising coiled coil domains.

The basis of the DNL method is the exploitation of the specific protein-protein interactions occurring in nature between the regulatory (R) subunits of protein kinase (PKA) and the anchoring domain (AD) of A-kinase anchoring proteins (AKAPs). The dimerization domain and AKAP binding domain of human RIIa are both located within the same amino-terminal 44-amino acid sequence, which is termed the dimerization and docking domain (DDD) This platform technology exploits the DDD of human RIIa and the AD of a certain amino acid sequence as a pair of linker modules for docking any two entities into a noncovalent complex, which could be further locked into a stably tethered structure through the introduction of cysteine residues into both the DDD and the AD at strategic positions to facilitate the formation of disulfide bonds.

The general methodology of the DNL approach is as follows. A recombinant protein is constructed by linking a DDD sequence to the compound of interest, for example the ectodomain of hACE2. Because the DDD sequence effects the spontaneous formation of a dimer, the resulting recombinant protein is a divalent compound, for example a divalent ectodomain of hACE2. To make the end product bispecific, a second recombinant protein is prepared by fusing an AD sequence. This second recombinant protein may comprise domain C, domain ABD, or domain ALB, which will be described in more detail in following sections of present invention. The dimeric motif of DDD in the first recombinant protein creates a docking site for binding to the AD sequence, thus facilitating a ready association of the dimeric ectodomain of hACE2 construct with the monomeric domain C, domain ABD, or domain ALB to form a binary, trimeric complex. This binding event is made irreversible with a subsequent reaction to secure the 2 entities covalently via disulfide bridges between the inserted cysteine residues. This reaction occurs very efficiently, because the initial binding interactions bring the reactive thiol groups on both the DDD and AD into proximity to ligate site-specifically. By attaching the DDD and AD away from the functional groups of the 2 precursors, such site-specific ligations preserve the original activities of the 2 precursors. The DNL method was disclosed in U.S. provisional application 60/751,196, which is incorporated herein by reference in its entirety.

“Knobs-into-holes” is a design strategy for engineering antibody heavy chain homodimers for heterodimerization. In this approach, a ‘knob’ variant was first obtained by replacement of a small amino acid with a larger one in the CH3 domain of an IgG: T366Y. The knob was designed to insert into a ‘hole’ in the CH3 domain of a different IgG created by judicious replacement of a large residue with a smaller one: Y407T. Another example of knobs-into-holes structure (CW-CSAV) comprises mutations S354C, T366W in the CH3 domain of one IgG chain, and Y349C, T366S, L368A, and Y407V in the CH3 domain of other IgG chain. Other paired variant combinations have been developed. Knobs-into-holes fusion proteins consist of [IgG1 hinge]-CH2-[Knobs-into-holes CH3], that may be genetically linked to one or more fusion partners. This results in bispecific molecules, such as the general bispecific scFv-based fusion proteins depicted in FIG. 6B.

By designing strand-exchange engineered domain (SEED) heterodimers, another way of achieving complementarity in the CH3 interface allowing for a heterodimeric assembly of Fc chains was developed. These SEED CH3 domains or SEEDbodies are composed of alternating segments derived from human IgA and IgG CH3 sequences (AG SEED CH3 and GA SEED CH3) and were used to generate so-called SEEDbodies. Because molecular models suggested that interaction with FcRn is impaired in the AG SEED CH3, residues at the CH2-CH3 junction were returned to IgG sequences. Pharmacokinetic studies confirmed that the half-life of SEEDbodies was comparable to other Fc fusion proteins and IgG1. SEEDbody fusion proteins consist of [IgG1 hinge]-CH2-[SEED CH3], that may be genetically linked to one or more fusion partners.

Another immunoglobulin-based architecture that may be used in the context of the present invention consists in fusing two antigen binding domains (e.g. scFv or dAb) of different specificity to the constant domain of human κchain (CL) and the first constant domain of human heavy chain (CH1) to form two polypeptides, (ABD1)-CL and (ABD2)-CH1-CH2-CH3, respectively. These molecules are termed bispecific tetravalent Fc fusions. The two polypeptides are co-expressed in cells. Association between the heavy and the light chains forms a covalently linked hetero-tetramer with dual specificity. This approach yields a homogeneous bispecific IgG-like antibody product with each molecule containing four antigen binding sites, two for each of its target antigen. An example of these molecules is the bispecific scFv4-Fc protein depicted in FIG. 6C). The BsAb retains not only antigen binding efficiency but also the biological activity of its component antibodies.

Human IgG1 CH1
(SEQ ID NO: 127)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
Human IgG1 constant kappa
(SEQ ID NO: 131)
VAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTK
SFNRGEC

It will be immediately appreciated that, in the context of the present invention, this immunoglobulin-based architecture may be exploited to increase the valency and avidity of other antigen binding domains, such as Domain A or Domain C (which is described in later aspects of the invention). For example, a fusion of Domain A to the constant domain of human κchain (CL) and the first constant domain of human heavy chain (CH1) to form two polypeptides, (Domain A)-CL and (Domain A)-CH1-CH2-CH3, respectively, which result in a quadri- or tetravalent ACE2 molecule (fourth panel, FIG. 2). The variants of an Fc region of an IgG which does not interact with FcγRI, FcγRIIa and FcγRIII described previously, including all the mutation and mutation combinations, may be used in this molecule.

The tetravalent Domain A based on this immunoglobulin architecture may comprise the sequences shown as SEQ ID NOs: 132 to 135.

Inactive ACE2 (HH:NN)-Heavy chain
(CH1-hinge-CH2-CH3)
(SEQ ID NO: 132)
QSTIEEQAKTFLDKFNHEAEDLFYQSSLASWNYNTNITEENVQNMNNAG
DKWSAFLKEQSTLAQMYPLQEIQNLTVKLQLQALQQNGSSVLSEDKSKR
LNTILNTMSTIYSTGKVCNPDNPQECLLLEPGLNEIMANSLDYNERLWA
WESWRSEVGKQLRPLYEEYVVLKNEMARANHYEDYGDYWRGDYEVNGVD
GYDYSRGQLIEDVEHTFEEIKPLYEHLHAYVRAKLMNAYPSYISPIGCL
PAHLLGDMWGRFWTNLYSLTVPFGQKPNIDVTDAMVDQAWDAQRIFKEA
EKFFVSVGLPNMTQGFWENSMLTDPGNVQKAVCHPTAWDLGKGDFRILM
CTKVTMDDFLTAHNEMGNIQYDMAYAAQPFLLRNGANEGFHEAVGEIMS
LSAATPKHLKSIGLLSPDFQEDNETEINFLLKQALTIVGTLPFTYMLEK
WRWMVFKGEIPKDQWMKKWWEMKREIVGVVEPVPHDETYCDPASLFHVS
NDYSFIRYYTRTLYQFQFQEALCQAAKHEGPLHKCDISNSTEAGQKLFN
MLRLGKSEPWTLALENVVGAKNMNVRPLLNYFEPLFTWLKDQNKNSFVG
WSTDWSPYADQSIKVRISLKSALGDKAYEWNDNEMYLFRSSVAYAMRQY
FLKVKNQMILFGEEDVRVANLKPRISFNFFVTAPKNVSDIIPRTEVEKA
IRMSRSRINDAFRLNDNSLEFLGIQPTLGPPNQPPVSSSASTKGPSVFP
LAPSSKSTSGGTAALGCLVKDYFPEPVTVSWNSGALTSGVHTFPAVLQS
SGLYSLSSVVTVPSSSLGTQTYICNVNHKPSNTKVDKKVEPKSCDKTHT
CPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKV
SNKALPAPIEKTISKAKGQPREPQVYTLPPSREEMTKNQVSLTCLVKGF
YPSDIAVEWESNGQPENNYKTTPPVLDSDGSFFLYSKLTVDKSRWQQGN
VFSCSVMHEALHNHYTQKSLSLSPGK
Inactive ACE2 (HH:NN)-Heavy chain (IgG1 LALA-PG)
(SEQ ID NO: 133)
QSTIEEQAKTFLDKFNHEAEDLFYQSSLASWNYNTNITEENVQNMNNAG
DKWSAFLKEQSTLAQMYPLQEIQNLTVKLQLQALQQNGSSVLSEDKSKR
LNTILNTMSTIYSTGKVCNPDNPQECLLLEPGLNEIMANSLDYNERLWA
WESWRSEVGKQLRPLYEEYVVLKNEMARANHYEDYGDYWRGDYEVNGVD
GYDYSRGQLIEDVEHTFEEIKPLYEHLHAYVRAKLMNAYPSYISPIGCL
PAHLLGDMWGRFWTNLYSLTVPFGQKPNIDVTDAMVDQAWDAQRIFKEA
EKFFVSVGLPNMTQGFWENSMLTDPGNVQKAVCHPTAWDLGKGDFRILM
CTKVTMDDFLTAHNEMGNIQYDMAYAAQPFLLRNGANEGFHEAVGEIMS
LSAATPKHLKSIGLLSPDFQEDNETEINFLLKQALTIVGTLPFTYMLEK
WRWMVFKGEIPKDQWMKKWWEMKREIVGVVEPVPHDETYCDPASLFHVS
NDYSFIRYYTRTLYQFQFQEALCQAAKHEGPLHKCDISNSTEAGQKLFN
MLRLGKSEPWTLALENVVGAKNMNVRPLLNYFEPLFTWLKDQNKNSFVG
WSTDWSPYADQSIKVRISLKSALGDKAYEWNDNEMYLFRSSVAYAMRQY
FLKVKNQMILFGEEDVRVANLKPRISFNFFVTAPKNVSDIIPRTEVEKA
IRMSRSRINDAFRLNDNSLEFLGIQPTLGPPNQPPVSSSASTKGPSVFP
LAPSSKSTSGGTAALGCLVKDYFPEPVTVSWNSGALTSGVHTFPAVLQS
SGLYSLSSVVTVPSSSLGTQTYICNVNHKPSNTKVDKKVEPKSCDKTHT
CPPCPAPEAAGGPSVFLFPPKPKDTLMISRTPEVTCVVVDVSHEDPEVK
FNWYVDGVEVHNAKTKPREEQYNSTYRVVSVLTVLHQDWLNGKEYKCKV
SNKALGAPIEKTISKAKGQPREPQVYTLPPSREEMTKNQVSLTCLVKGF
YPSDIAVEWESNGQPENNYKTTPPVLDSDGSFFLYSKLTVDKSRWQQGN
VFSCSVMHEALHNHYTQKSLSLSPGK
Inactive ACE2 (HH:NN)-Heavy chain
(IgG1 LALA-PG, YTE)
(SEQ ID NO: 134)
QSTIEEQAKTFLDKFNHEAEDLFYQSSLASWNYNTNITEENVQNMNNAG
DKWSAFLKEQSTLAQMYPLQEIQNLTVKLQLQALQQNGSSVLSEDKSKR
LNTILNTMSTIYSTGKVCNPDNPQECLLLEPGLNEIMANSLDYNERLWA
WESWRSEVGKQLRPLYEEYVVLKNEMARANHYEDYGDYWRGDYEVNGVD
GYDYSRGQLIEDVEHTFEEIKPLYEHLHAYVRAKLMNAYPSYISPIGCL
PAHLLGDMWGRFWTNLYSLTVPFGQKPNIDVTDAMVDQAWDAQRIFKEA
EKFFVSVGLPNMTQGFWENSMLTDPGNVQKAVCHPTAWDLGKGDFRILM
CTKVTMDDFLTAHNEMGNIQYDMAYAAQPFLLRNGANEGFHEAVGEIMS
LSAATPKHLKSIGLLSPDFQEDNETEINFLLKQALTIVGTLPFTYMLEK
WRWMVFKGEIPKDQWMKKWWEMKREIVGVVEPVPHDETYCDPASLFHVS
NDYSFIRYYTRTLYQFQFQEALCQAAKHEGPLHKCDISNSTEAGQKLFN
MLRLGKSEPWTLALENVVGAKNMNVRPLLNYFEPLFTWLKDQNKNSFVG
WSTDWSPYADQSIKVRISLKSALGDKAYEWNDNEMYLFRSSVAYAMRQY
FLKVKNQMILFGEEDVRVANLKPRISFNFFVTAPKNVSDIIPRTEVEKA
IRMSRSRINDAFRLNDNSLEFLGIQPTLGPPNQPPVSSSASTKGPSVFP
LAPSSKSTSGGTAALGCLVKDYFPEPVTVSWNSGALTSGVHTFPAVLQS
SGLYSLSSVVTVPSSSLGTQTYICNVNHKPSNTKVDKKVEPKSCDKTHT
CPPCPAPEAAGGPSVFLFPPKPKDTLYITREPEVTCVVVDVSHEDPEVK
FNWYVDGVEVHNAKTKPREEQYNSTYRVVSVLTVLHQDWLNGKEYKCKV
SNKALGAPIEKTISKAKGQPREPQVYTLPPSREEMTKNQVSLTCLVKGF
YPSDIAVEWESNGQPENNYKTTPPVLDSDGSFFLYSKLTVDKSRWQQGN
VFSCSVMHEALHNHYTQKSLSLSPGK
Inactive ACE2 (HH:NN)-Light chain
(constant kappa)
(SEQ ID NO: 135)
QSTIEEQAKTFLDKFNHEAEDLFYQSSLASWNYNTNITEENVQNMNNAG
DKWSAFLKEQSTLAQMYPLQEIQNLTVKLQLQALQQNGSSVLSEDKSKR
LNTILNTMSTIYSTGKVCNPDNPQECLLLEPGLNEIMANSLDYNERLWA
WESWRSEVGKQLRPLYEEYVVLKNEMARANHYEDYGDYWRGDYEVNGVD
GYDYSRGQLIEDVEHTFEEIKPLYEHLHAYVRAKLMNAYPSYISPIGCL
PAHLLGDMWGRFWTNLYSLTVPFGQKPNIDVTDAMVDQAWDAQRIFKEA
EKFFVSVGLPNMTQGFWENSMLTDPGNVQKAVCHPTAWDLGKGDFRILM
CTKVTMDDFLTAHNEMGNIQYDMAYAAQPFLLRNGANEGFHEAVGEIMS
LSAATPKHLKSIGLLSPDFQEDNETEINFLLKQALTIVGTLPFTYMLEK
WRWMVFKGEIPKDQWMKKWWEMKREIVGVVEPVPHDETYCDPASLFHVS
NDYSFIRYYTRTLYQFQFQEALCQAAKHEGPLHKCDISNSTEAGQKLFN
MLRLGKSEPWTLALENVVGAKNMNVRPLLNYFEPLFTWLKDQNKNSFVG
WSTDWSPYADQSIKVRISLKSALGDKAYEWNDNEMYLFRSSVAYAMRQY
FLKVKNQMILFGEEDVRVANLKPRISFNFFVTAPKNVSDIIPRTEVEKA
IRMSRSRINDAFRLNDNSLEFLGIQPTLGPPNQPPVSSSASVAAPSVFI
FPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC

Further immunoglobulin-based architectures that form heterooligomers include the dual variable domain (DVD or DVD-Ig) (Wu et al., 2007, Nat Biotechnol 25:1290-7). Like a conventional IgG, the DVD molecule is composed of two heavy chains and two light chains. Unlike IgG, however, both heavy and light chains of a DVD molecule contain an additional variable domain (VD) connected via a linker sequence at the N-termini of the VH and VL of an existing monoclonal antibody (mAb). Thus, when the heavy and the light chains combine, the resulting DVD molecule contains four antigen recognition sites (FIG. 25B). The outermost or N-terminal variable domain is termed VD1 and the innermost variable domain is termed VD2; the VD2 is proximal to the C-terminal CH1 or CL.

Non-limiting examples of other immunoglobulin-based architectures are depicted in FIG. 25. These architectures, including DVD and scFv4-Fc, enable the production of multivalent and multispecific heterooligomers. These are particularly useful in the aspect of the invention related to a polypeptide based on coronavirus SP-specific binders, which is described in subsequent aspects.

Variants of the Fc region of an IgG which do not interact with FcγRI, FcγRIIa and FcγRIII and/or which display improved circulation or serum half-life, which were described previously in the context of the homooligomer, are equally applicable to the immunoglobulin-based architectures of the heterooligomer.

The barnase-barstar system is a multimerisation module based on the tight interaction between barnase and barstar. Barnase is a 110 aa secreted ribonuclease from Bacillus amyloliquefaciens. Barstar is an 89 aa cytoplasmic barnase inhibitor with which the host protects itself. They rapidly form a complex with a KD of ˜10⁻¹⁴ M. Both the N- and C-termini of both proteins are accessible and available for fusions (Deyev et al., 2003, Nat Biotech 21:1486-92).

The coiled coil domain has been described in the context of the homooligomer of the invention, and its definition applies equally to the heterooligomer. Examples of proteins which contain a heterooligomerising coiled coil domain include, but are not limited to, polypeptide release factor 2, SNAP-25, SNARE, Lac repressor and apolipoprotein E. Non-limiting examples of heterooligomerising coiled coil domains include

Polypeptide release factor 2: anti-parallel
heterotrimer
Chain A:
(SEQ ID NO: 138)
INPVNNRIQDLTERSDVLRGYLDY
Chain B:
(SEQ ID NO: 111)
VVDTLDQMKQGLEDVSGLLELAVEADDEETFNEAVAELDALEEKLAQLE
FR
SNAP-25 and SNARE: parallel heterotetramer
Chain A:
(SEQ ID NO: 112)
IETRHSEIIKLENSIRELHDMFMDMAMLVESQGEMIDRIEYNVEHAVDY
VE
Chain B:
(SEQ ID NO: 113)
ALSEIETRHSEIIKLENSIRELHDMFMDMAMLVESQGEMIDRIEYNVEH
AVDYVERAVSDTKKAVKY
Chain C:
(SEQ ID NO: 114)
ELEEMQRRADQLADESLESTRRMLQLVEESKDAGIRTLVMLDEQGEQLE
RIEEGMDQINKDMKEAEKNL
Chain D:
(SEQ ID NO: 115)
IETRHSEIIKLENSIRELHDMFMDMAMLVESQGEMIDRIEYNVEHAVDY
VE
Lac repressor: parallel homotetramer
(SEQ ID NO: 116)
SPRALADSLMQLARQVSRLE
Apolipoprotein E: anti-parallel heterotetramer
(SEQ ID NO: 117)
SGQRWELALGRFWDYLRWVQTLSEQVQEELLSSQVTQELRALMDETMKE
LKAYKSELEEQLTARLSKELQAAQARLGADMEDVCGRLVQYRGEVQAML
GQSTEELRVRLASHLRKLRKRLLRDADDLQKRLAVYQA

The ability of the first polypeptide of the invention to form dimers, trimers or higher oligomers can be determined by conventional methods known by the skilled person in the art. For example, by way of a simple illustration, the ability of a domain A fused to a domain O comprising a trimerising domain, or functionally equivalent variant thereof, to form a trimer can be determined by using standard chromatographic techniques. Thus, the variant to be assessed is put under suitable trimerisation conditions and the complex is subjected to a standard chromatographic assay under non denaturing conditions so that the eventually formed complex (trimer) is not altered. If the variant trimerises properly, the molecular size of the complex would be three times heavier than the molecular size of a single molecule of the variant. The molecular size of the complex can be revealed by using standard methods such as analytical centrifugation, mass spectrometry, size-exclusion chromatography, sedimentation velocity, etc.

Combinations of two or more type of homo-oligomerisation domain in the same polypeptide, or two or more type of hetero-oligomerisation domain in the same polypeptide, or one or more homo-oligomerisation domain and one or more hetero-oligomerisation domain in the same polypeptide are within the scope of the present invention. A non-limiting example is shown in the fourth panel of FIG. 25B.

The first polypeptide of the invention may have different configurations. Thus, the first polypeptide of the invention may comprise, from N-terminus to C-terminus:
domain A-domain O, and
domain O-domain A.

Domain A and domain O may be joined by a linker.

The terms “linker” and “spacer” are used indistinctively in the present application. A linker provides spatial separation between domain A and domain O. The linker may be a flexible linker. This type of linkers allows for torsion of domain A respective of domain O, which may be beneficial when domain A interacts with the S protein of a coronavirus. Non-limiting examples of flexible linkers that may be used in the first polypeptide of the invention include:

(Gly₄Ser)₂ (SEQ ID NO: 67: GGGGSGGGGS);
(Gly₄Ser)₃ (SEQ ID NO: 6: GGGGSGGGGSGGGGS);
(Gly₄Ser)₄ (SEQ ID NO: 68: GGGGSGGGGSGGGGSGGGGS);
(Gly₄Ser)₅ (SEQ ID NO: 69: GGGGSGGGGSGGGGSGGGGSGG
GGS);
(SEQ ID NO: 70)
SGGGGSGGGGSGGGGS;
(SEQ ID NO: 71)
GGGGSGGGGSGGGGS;
and
(SEQ ID NO: 72)
GGGGSGGGGSGGGAS.

The linker may be the (Gly4Ser)3 linker (SEQ ID NO: 6).

Where possible, the sequences used in domain O may be of human origin. This is convenient in order to prevent immunogenicity.

Where the first polypeptide of the invention consists of one domain A which consists of the ectodomain of hACE2 having a sequence of SEQ ID NO: 1 or 2, then the domain O may not comprise the human IgG1 Fc region.

The first polypeptide of the invention may further comprise a domain C, wherein the domain C comprises CD147 or a fragment thereof comprising the amino acid sequence shown as SEQ ID NO: 2 or a fragment thereof comprising the amino acid sequence shown as SEQ ID NO: 3.

2.3. Domain C

A high mutation rate is expected for SARS-CoV-2 as a result of selective pressure. Since the binding between ACE2 and S protein of SARS-CoV and SARS-CoV-2 has been described as a low affinity interaction, a second interaction with the coronavirus virion may have potential benefit. CD147 has been reported to be involved coronavirus entry into the host cell, although the mechanism is still unknown. Thus, a fusion protein formed with ACE2 and CD147 may result in an avidity maximisation and enhanced overall neutralisation potential.

Therefore, the first polypeptide of the invention may further comprise a domain C.

Thus, the first polypeptide of the invention may comprise:
a) a domain A which comprises the ectodomain of human angiotensin converting enzyme type 2 (hACE2), or a fragment thereof comprising the amino acid sequence shown as SEQ ID NO: 3, or a variant thereof which maintains the capacity of hACE2 to interact with the S protein of coronavirus,
b) a domain O which comprises an oligomerisation domain, and
c) a domain C which comprises the ectodomain of CD147 or a fragment thereof comprising the amino acid sequence shown as SEQ ID NO: 8 or SEQ ID NO: 9.

The terms “polypeptide”, “domain A”, “hACE2”, “domain O”, and “oligomerisation domain” have been described in detail in previous sections of this specification, and their definitions and particular features apply equally herein.

Importantly, the proviso defining that “where the first polypeptide of the invention comprises one domain A which consists of the ectodomain of hACE2 having a sequence of SEQ ID NO: 1 or 2, then the domain O may not comprise the human IgG1 Fc region” does not apply to polypeptides of the invention comprising a domain A, a domain O and a domain C.

Domain C comprises the ectodomain of CD147 or a fragment thereof comprising the amino acid sequence shown as SEQ ID NO: 8 or a fragment thereof comprising the amino acid sequence shown as SEQ ID NO: 9.

The term “CD147” or “hCD147” or “cluster of differentiation 147” or “basigin” or “BSG” or “5F7” or “collagenase stimulatory factor” or “extracellular matrix metalloproteinase inducer (EMMPRIN)” or “leukocyte activation antigen M6” or “OK blood group antigen” or “tumour cell-derived collagenase stimulatory factor (TCSF)” refers is a 385 aa highly glycosylated type I transmembrane receptor with three immunoglobulin-like domains. Human CD147 is depicted under Accession No. P35613 in the Uniprot database on 30 Mar. 2020. It is widely expressed on various tissues and cell types and is involved in many physiological and pathological processes, such as various immunologic phenomena, differentiation, and development. CD147 is expressed by many cell types, including epithelial cells, endothelial cells and leukocytes.

CD147 has many ligands, including the cyclophilin (CyP) proteins Cyp-A and CyP-B and certain integrins. It is the main receptor for CyPA, which is a ubiquitously expressed cellular protein involved in protein folding. CyPA has been shown to incorporate into HIV-1 virions by a specific interaction with the viral capsid protein. Through its binding to CD147, CyPA plays an essential role in early stage HIV-1 infection by modulating the translocation of HIV-1 core protein and initiation of viral reverse transcription. CD147/CyPA have been implicated in the regulation of viral infectivity of other viruses such as measles virus, influenza virus and, relevantly, SARS-CoV. In SARS-CoV-2, CD147 has been shown to bind to the S protein.

The processed mature protein spans aa 21-385 of the sequence shown under Uniprot Accession No. P35613, and contains three extracellular immunoglobulin domains spanning aa 37-120, aa 138-219, and aa 221-315, respectively. There are 4 isoforms of CD147: the one just described is known as Isoform 1 or long or basigin-2 (Uniprot Accession No. P35613-1); Isoform 2 or short or basigin-1 (Uniprot Accession No. P35613-2); Isoform 3 or basigin-3 (Uniprot Accession No. P35613-3); and Isoform 4 or basigin-4 (Uniprot Accession No. P35613-4).

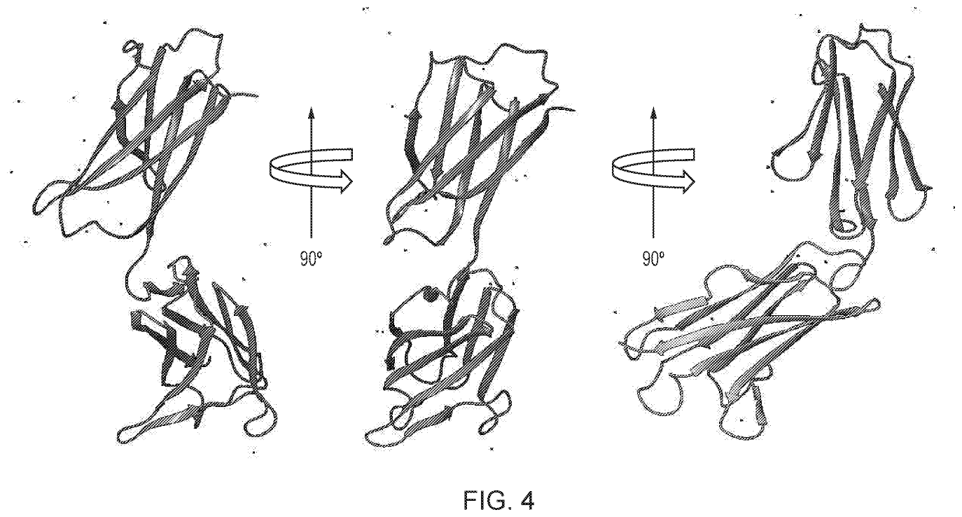

Domain C of the first polypeptide of the invention may comprise the ectodomain of CD147 having the sequence of SEQ ID NO: 7, or Domain 1 of CD147 having the sequence of SEQ ID NO: 8, or Domain 2 of CD147 having the sequence of SEQ ID NO: 9. The structure of Domain 1 and Domain 2 of CD147 is depicted in FIG. 4.

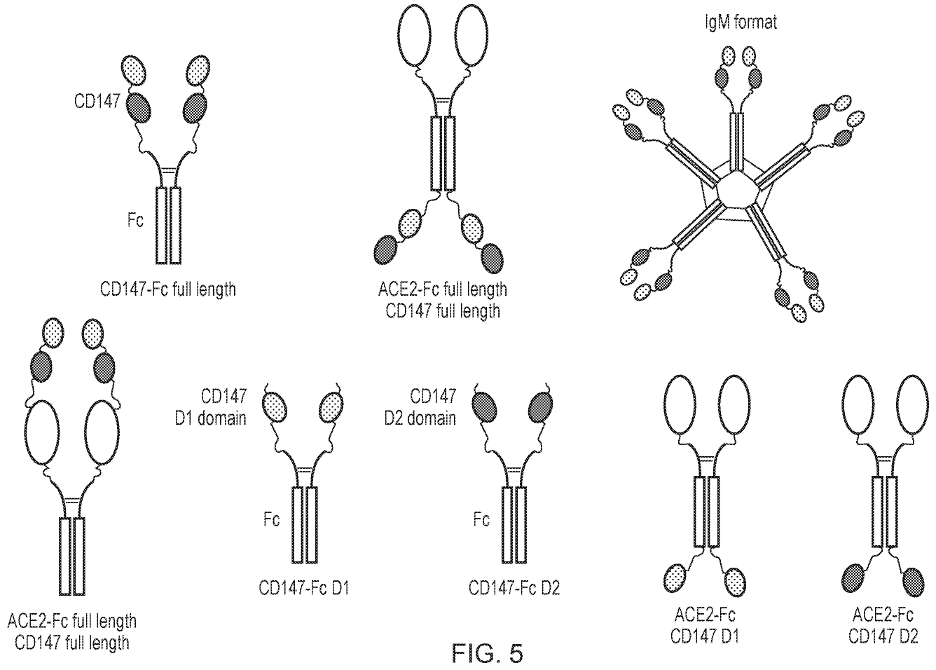

Non-limiting examples domain A, domain O comprising an Fc region, and domain C fusion proteins in different configurations are depicted in FIG. 5.

Processed extracellular domain of CD147
(SEQ ID NO: 7):
AAGFVQAPLSQQRWVGGSVELHCEAVGSPVPEIQWWFEGQGPNDTCSQL
WDGARLDRVHIHATYHQHAASTISIDTLVEEDTGTYECRASNDPDRNHL
TRAPRVKWVRAQAVVLVLEPGTVFTTVEDLGSKILLTCSLNDSATEVTG
HRWLKGGVVLKEDALPGQKTEFKVDSDDQWGEYSCVFLPEPMGTANIQL
HGPPRVKAVKSSEHINEGETAMLVCKSESVPPVTDWAWYKITDSEDKAL
MNGSESRFFVSSSQGRSELHIENLNMEADPGQYRCNGTSSKGSDQAIIT
LRVRS
Domain 1 of CD147 (SEQ ID NO: 8):
AAGFVQAPLSQQRWVGGSVELHCEAVGSPVPEIQWWFEGQGPNDTCSQL
WDGARLDRVHIHATYHQHAASTISIDTLVEEDTGTYECRASNDPDRNHL
TRAPRVKWVRAQAVVLVLEPGTVFTTVEDLGSKILLTCSLNDSATEVTG
HRWLKGGVVLKEDALPGQKTEFKVDSDDQWGEYSCVFLPEPMGTANIQL
HG
Domain 2 of CD147 (SEQ ID NO: 9):
GPPRVKAVKSSEHINEGETAMLVCKSESVPPVTDWAWYKITDSEDKALM
NGSESRFFVSSSQGRSELHIENLNMEADPGQYRCNGTSSKGSDQAIITL
RVRS

The terms CD147 of sequence SEQ ID NO: 7, or a fragment thereof comprising the amino acid sequence shown as SEQ ID NO: 8 or SEQ ID NO: 9 are also intended to embrace functionally equivalent variants which have been modified in the amino acid sequence without adversely affecting, to any substantial degree, the capacity of CD147 or one of its fragments to interact with the coronavirus virion relative to that of the native CD147 molecule. Said modifications include, the conservative (or non-conservative) substitution of one or more amino acids for other amino acids, the insertion and/or the deletion of one or more amino acids, provided that the capacity to interact with the coronavirus virion of the variant is substantially maintained, i.e., the variant maintains the ability (capacity) to interact with the coronavirus virion at physiological conditions.

The term “variant” or “mutant” has been described in the context of domain A and its definition and particular features apply equally to domain C. The variants or mutants of CD147 of sequence SEQ ID NO: 7, or a fragment thereof comprising the amino acid sequence shown as SEQ ID NO: 8 or SEQ ID NO: 9 may have at least 70%, or at least 80%, or at least 90%, or at least 95%, or at least 96%, or at least 97%, or at least 98%, or at least 99% sequence identity with the sequences shown as SEQ ID NO: 6, 7 or 8, provided that the capacity to interact with the coronavirus virion of the variant is substantially maintained.

The interaction between CD147 and coronavirus S protein can be determined by conventional methods. For example, in vitro binding of CD147 to the S protein may be determined by ELISA, surface plasmon resonance (SPR), or by flow cytometry using ACE2- or S protein-expressing cells. Additionally, the virus neutralisation ability of CD147-based fusion proteins may be determined by incubating the fusion proteins with relevant virus, e.g. lentiviral vectors pseudotyped with coronavirus S protein, and cultured onto ACE2-expressing cells. These methods are further described in Example 4.

Domain A, domain O and domain C may have any configuration in the first polypeptide of the invention. The structure of the polypeptide may be selected from one of the following, from N-terminus to C-terminus:
domain A-domain C-domain O,
domain C-domain A-domain O,
domain A-domain O-domain C,
domain C-domain O-domain A,
domain O-domain A-domain C, and
domain O-domain C-domain A.

Each of these domains may be joined by a linker. The linker may be a flexible linker. The linkers described in the context of the fusion between domain A and domain O may be equally used herein. The linker may be the (Gly₄Ser)₃ linker (SEQ ID NO: 6).

The first polypeptide of the invention may consist of an amino acid sequence selected from SEQ ID NO: 80 to 83.

hACE2_18-740_HuIgG1Fc_CD147_22-320
(SEQ ID NO: 80):
QSTIEEQAKTFLDKFNHEAEDLFYQSSLASWNYNTNITEENVQNMNNAG
DKWSAFLKEQSTLAQMYPLQEIQNLTVKLQLQALQQNGSSVLSEDKSKR
LNTILNTMSTIYSTGKVCNPDNPQECLLLEPGLNEIMANSLDYNERLWA
WESWRSEVGKQLRPLYEEYVVLKNEMARANHYEDYGDYWRGDYEVNGVD
GYDYSRGQLIEDVEHTFEEIKPLYEHLHAYVRAKLMNAYPSYISPIGCL
PAHLLGDMWGRFWTNLYSLTVPFGQKPNIDVTDAMVDQAWDAQRIFKEA
EKFFVSVGLPNMTQGFWENSMLTDPGNVQKAVCHPTAWDLGKGDFRILM
CTKVTMDDFLTAHNEMGNIQYDMAYAAQPFLLRNGANEGFHEAVGEIMS
LSAATPKHLKSIGLLSPDFQEDNETEINFLLKQALTIVGTLPFTYMLEK
WRWMVFKGEIPKDQWMKKWWEMKREIVGVVEPVPHDETYCDPASLFHVS
NDYSFIRYYTRTLYQFQFQEALCQAAKHEGPLHKCDISNSTEAGQKLFN
MLRLGKSEPWTLALENVVGAKNMNVRPLLNYFEPLFTWLKDQNKNSFVG
WSTDWSPYADQSIKVRISLKSALGDKAYEWNDNEMYLFRSSVAYAMRQY
FLKVKNQMILFGEEDVRVANLKPRISFNFFVTAPKNVSDIIPRTEVEKA
IRMSRSRINDAFRLNDNSLEFLGIQPTLGPPNQPPVSEPKSCDKTHTCP
PCPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVSN
KALPAPIEKTISKAKGQPREPQVYTLPPSRDELTKNQVSLTCLVKGFYP
SDIAVEWESNGQPENNYKTTPPVLDSDGSFFLYSKLTVDKSRWQQGNVF
SCSVMHEALHNHYTQKSLSLSPGKSGGGGSGGGGSGGGGSAAGFVQAPL
SQQRWVGGSVELHICEAVGSPVPEIQWWFEGQGPNDTCSQLWDGARLDR
VHIHATYHQHAASTISIDTLVEEDTGTYECRASNDPDRNHLTRAPRVKW
VRAQAVVLVLEPGTVFTTVEDLGSKILLTCSLNDSATEVTGHRWLKGGV
VLKEDALPGQKTEFKVDSDDQWGEYSCVFLPEPMGTANIQLHGPPRVKA
VKSSEHINEGETAMLVCKSESVPPVTDWAWYKITDSEDKALMNGSESRF
FVSSSQGRSELHIENLNMEADPGQYRCNGTSSKGSDQAIITLRVRS
hACE2_18-740_HuIgG1Fc_CD147_22-219
(SEQ ID NO: 81):
QSTIEEQAKTFLDKFNHEAEDLFYQSSLASWNYNTNITEENVQNMNNAG
DKWSAFLKEQSTLAQMYPLQEIQNLTVKLQLQALQQNGSSVLSEDKSKR
LNTILNTMSTIYSTGKVCNPDNPQECLLLEPGLNEIMANSLDYNERLWA
WESWRSEVGKQLRPLYEEYVVLKNEMARANHYEDYGDYWRGDYEVNGVD
GYDYSRGQLIEDVEHTFEEIKPLYEHLHAYVRAKLMNAYPSYISPIGCL
PAHLLGDMWGRFWTNLYSLTVPFGQKPNIDVTDAMVDQAWDAQRIFKEA
EKFFVSVGLPNMTQGFWENSMLTDPGNVQKAVCHPTAWDLGKGDFRILM
CTKVTMDDFLTAHNEMGNIQYDMAYAAQPFLLRNGANEGFHEAVGEIMS
LSAATPKHLKSIGLLSPDFQEDNETEINFLLKQALTIVGTLPFTYMLEK
WRWMVFKGEIPKDQWMKKWWEMKREIVGVVEPVPHDETYCDPASLFHVS
NDYSFIRYYTRTLYQFQFQEALCQAAKHEGPLHKCDISNSTEAGQKLFN
MLRLGKSEPWTLALENVVGAKNMNVRPLLNYFEPLFTWLKDQNKNSFVG
WSTDWSPYADQSIKVRISLKSALGDKAYEWNDNEMYLFRSSVAYAMRQY
FLKVKNQMILFGEEDVRVANLKPRISFNFFVTAPKNVSDIIPRTEVEKA
IRMSRSRINDAFRLNDNSLEFLGIQPTLGPPNQPPVSEPKSCDKTHTCP
PCPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVSN
KALPAPIEKTISKAKGQPREPQVYTLPPSREEMTKNQVSLTCLVKGFYP
SDIAVEWESNGQPENNYKTTPPVLDSDGSFFLYSKLTVDKSRWQQGNVF
SCSVMHEALHNHYTQKSLSLSPGKSGGGGSGGGGSGGGGSAAGFVQAPL
SQQRWVGGSVELHCEAVGSPVPEIQWWFEGQGPNDTCSQLWDGARLDRV
HIHATYHQHAASTISIDTLVEEDTGTYECRASNDPDRNHLTRAPRVKWV
RAQAVVLVLEPGTVFTTVEDLGSKILLTCSLNDSATEVTGHRWLKGGVV
LKEDALPGQKTEFKVDSDDQWGEYSCVFLPEPMGTANIQLHG
hACE2_18-740_HuIgG1Fc_CD147_219-320
(SEQ ID NO: 82):
QSTIEEQAKTFLDKFNHEAEDLFYQSSLASWNYNTNITEENVQNMNNAG
DKWSAFLKEQSTLAQMYPLQEIQNLTVKLQLQALQQNGSSVLSEDKSKR
LNTILNTMSTIYSTGKVCNPDNPQECLLLEPGLNEIMANSLDYNERLWA
WESWRSEVGKQLRPLYEEYVVLKNEMARANHYEDYGDYWRGDYEVNGVD
GYDYSRGQLIEDVEHTFEEIKPLYEHLHAYVRAKLMNAYPSYISPIGCL
PAHLLGDMWGRFWTNLYSLTVPFGQKPNIDVTDAMVDQAWDAQRIFKEA
EKFFVSVGLPNMTQGFWENSMLTDPGNVQKAVCHPTAWDLGKGDFRILM
CTKVTMDDFLTAHNEMGNIQYDMAYAAQPFLLRNGANEGFHEAVGEIMS
LSAATPKHLKSIGLLSPDFQEDNETEINFLLKQALTIVGTLPFTYMLEK
WRWMVFKGEIPKDQWMKKWWEMKREIVGVVEPVPHDETYCDPASLFHVS
NDYSFIRYYTRTLYQFQFQEALCQAAKHEGPLHKCDISNSTEAGQKLFN
MLRLGKSEPWTLALENVVGAKNMNVRPLLNYFEPLFTWLKDQNKNSFVG
WSTDWSPYADQSIKVRISLKSALGDKAYEWNDNEMYLFRSSVAYAMRQY
FLKVKNQMILFGEEDVRVANLKPRISFNFFVTAPKNVSDIIPRTEVEKA
IRMSRSRINDAFRLNDNSLEFLGIQPTLGPPNQPPVSEPKSCDKTHTCP
PCPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVSN
KALPAPIEKTISKAKGQPREPQVYTLPPSRDELTKNQVSLTCLVKGFYP
SDIAVEWESNGQPENNYKTTPPVLDSDGSFFLYSKLTVDKSRWQQGNVF
SCSVMHEALHNHYTQKSLSLSPGKSGGGGSGGGGSGGGGSGPPRVKAVK
SSEHINEGETAMLVCKSESVPPVTDWAWYKITDSEDKALMNGSESRFFV
SSSQGRSELHIENLNMEADPGQYRCNGTSSKGSDQAIITLRVRS
CD147_22-320_16gs_hACE2_18-740_HuIgG1Fc
(SEQ ID NO: 83):
AAGFVQAPLSQQRWVGGSVELHCEAVGSPVPEIQWWFEGQGPNDTCSQL
WDGARLDRVHIHATYHQHAASTISIDTLVEEDTGTYECRASNDPDRNHL
TRAPRVKWVRAQAVVLVLEPGTVFTTVEDLGSKILLTCSLNDSATEVTG
HRWLKGGVVLKEDALPGQKTEFKVDSDDQWGEYSCVFLPEPMGTANIQL
HGPPRVKAVKSSEHINEGETAMLVCKSESVPPVTDWAWYKITDSEDKAL
MNGSESRFFVSSSQGRSELHIENLNMEADPGQYRCNGTSSKGSDQAIIT
LRVRSSGGGGSGGGGSGGGGSQSTIEEQAKTFLDKFNHEAEDLFYQSSL
ASWNYNTNITEENVQNMNNAGDKWSAFLKEQSTLAQMYPLQEIQNLTVK
LQLQALQQNGSSVLSEDKSKRLNTILNTMSTIYSTGKVCNPDNPQECLL
LEPGLNEIMANSLDYNERLWAWESWRSEVGKQLRPLYEEYVVLKNEMAR
ANHYEDYGDYWRGDYEVNGVDGYDYSRGQLIEDVEHTFEEIKPLYEHLH
AYVRAKLMNAYPSYISPIGCLPAHLLGDMWGRFWTNLYSLTVPFGQKPN
IDVTDAMVDQAWDAQRIFKEAEKFFVSVGLPNMTQGFWENSMLTDPGNV
QKAVCHPTAWDLGKGDFRILMCTKVTMDDFLTAHNEMGNIQYDMAYAAQ
PFLLRNGANEGFHEAVGEIMSLSAATPKHLKSIGLLSPDFQEDNETEIN
FLLKQALTIVGTLPFTYMLEKWRWMVFKGEIPKDQWMKKWWEMKREIVG
VVEPVPHDETYCDPASLFHVSNDYSFIRYYTRTLYQFQFQEALCQAAKH
EGPLHKCDISNSTEAGQKLFNMLRLGKSEPWTLALENVVGAKNMNVRPL
LNYFEPLFTWLKDQNKNSFVGWSTDWSPYADQSIKVRISLKSALGDKAY
EWNDNEMYLFRSSVAYAMRQYFLKVKNQMILFGEEDVRVANLKPRISFN
FFVTAPKNVSDIIPRTEVEKAIRMSRSRINDAFRLNDNSLEFLGIQPTL
GPPNQPPVSEPKSCDKTHTCPPCPAPELLGGPSVFLFPPKPKDTLMISR
TPEVTCVVVDVSHEDPEVKFNWYVDGVEVHNAKTKPREEQYNSTYRVVS
VLTVLHQDWLNGKEYKCKVSNALPAPIEKTISKAKGQPREPQVYTLPPS
KRDELTKNQVSLTCLVKGFYPSDIAVEWESNGQPENNYKTTPPVLDSDG
SFFLYSKLTVDKSRWQQGNVFSCSVMHEALHNHYTQKSLSLSPGK

2.4. Domain ABD

As an alternative approach to enhance the interaction between the neutralising clamp of the invention and SARS-CoV-2, the ACE2 molecule can be combined with antibodies binding distinct epitopes on the coronavirus S protein, maximising avidity and overall neutralisation potential. Although viral escape mechanisms (through viral mutation) are more probable with non-endogenous ligands such as antibodies, the use of two or more binding events minimises the risk of such occurrence.

The first polypeptide of the invention may further comprise a domain ABD, wherein the domain ABD comprises an antigen-binding domain that binds specifically to a coronavirus spike protein (S protein).

Therefore, the first polypeptide of the invention may comprise:
a) a domain A which comprises the ectodomain of human angiotensin converting enzyme type 2 (hACE2), or a fragment thereof comprising the amino acid sequence shown as SEQ ID NO: 3, or a variant thereof which maintains the capacity of hACE2 to interact with the S protein of coronavirus,
b) a domain O which comprises an oligomerisation domain, and
c) a domain ABD, wherein the domain ABD comprises an antigen-binding domain that binds specifically to a coronavirus spike protein (S protein);
and, optionally,
d) a domain C which comprises the ectodomain of CD147 or a fragment thereof comprising the amino acid sequence shown as SEQ ID NO: 8 or SEQ ID NO: 9.

The first polypeptide of the invention may comprise:
a domain A, a domain O and a domain ABD, or
a domain A, a domain O, a domain C, and a domain ABD.

The terms “polypeptide”, “domain A”, “hACE2”, “domain O”, “oligomerisation domain”, “coronavirus S protein”, “domain C”, and “CD147” have been described in detail in previous sections of this specification, and their definitions and particular features apply equally herein.

Importantly, the proviso defining that “where the first polypeptide of the invention comprises one domain A which consists of the ectodomain of hACE2 having a sequence of SEQ ID NO: 1 or 2, then the domain O may not comprise the human IgG1 Fc region” does not apply to polypeptides of the invention comprising a domain A, a domain O and a domain ABD, and, where present, a domain C.

The first polypeptide of the invention may comprise a domain A, a domain O, a domain ABD, and, where present, a domain C, in any possible configuration. The skilled person will immediately identify all the different configurations that are possible for combinations of a domain A, a domain O, a domain ABD, and, where present, a domain C (from N- to C-terminus).

Each of domain A, domain O, domain ABD, and, where present, domain C, may be joined by a linker. The linker may be a flexible linker. The linkers described in the context of the fusion between domain A and domain O may be equally used herein. The linker may be the (Gly₄Ser)₃ linker (SEQ ID NO: 6).

The term “antigen-binding domain”, as used herein, refers to a polypeptide having an antigen binding site which comprises at least one complementarity determining region or CDR. The antigen-binding domain may comprise 3 CDRs and have an antigen binding site which is equivalent to that of a single domain antibody (dAb), heavy chain antibody (VHH) or a nanobody. Alternatively, the antibody may comprise 6 CDRs and have an antigen binding site which is equivalent to that of a classical antibody molecule. The remainder of the polypeptide may be any sequence which provides a suitable scaffold for the antigen binding site and displays it in an appropriate manner for it to bind the antigen.

A full-length antibody or immunoglobulin typically consists of four polypeptides: two identical copies of a heavy (H) chain polypeptide and two identical copies of a light (L) chain polypeptide. Each of the heavy chains contains one N terminal variable (VH) region and three C-terminal constant (CH₁, CH₂ and CH₃) regions, and each light chain contains one N-terminal variable (VL) region and one C-terminal constant (CL) region. The variable regions of each pair of light and heavy chains form the antigen binding site of an antibody. They are characterised by the same general structure constituted by relatively preserved regions called frameworks (FR) joined by three hyper-variable regions called complementarity determining regions (CDR) (Kabat et al., 1991, Sequences of Proteins of Immunological Interest, 5th Ed., NIH Publication No. 91-3242, Bethesda, MD.; Chothia & Lesk, 1987, J Mol Biol 196:901-17). The term “complementarity determining region” or “CDR”, as used herein, refers to the region within an antibody that complements an antigen's shape. Thus, CDRs determine the protein's affinity and specificity for specific antigens. The CDRs of the two chains of each pair are aligned by the framework regions, acquiring the function of binding a specific epitope. Consequently, in the case of VH and VL domains both the heavy chain and the light chain are characterised by three CDRs, respectively CDRH1, CDRH2, CDRH3 and CDRL1, CDRL2, CDRL3.

A number of definitions of the CDRs are commonly in use. The Kabat definition is based on sequence variability and is the most commonly used (see http://www.bioinf.org.uk/abs/). The ImMunoGeneTics information system (IMGT) (see http://www.imgt.org) can also be used. According to this system, a complementarity determining region (CDR-IMGT) is a loop region of a variable domain, delimited according to the IMGT unique numbering for V domain. There are three CDR-IMGT in a variable domain: CDR1-IMGT (loop BC), CDR2-IMGT (loop C′C″), and CDR3-IMGT (loop FG). Other definitions of the CDRs have also been developed, such as the Chothia, the AbM and the contact definitions (see http://www.imgt.org). Unless stated otherwise, the CDRs described herein are derived using the Kabat system.

The terms “antigen-binding fragment” and “antigen-binding portion” are used interchangeably herein and refer to one or more fragments or portions of an antibody that retain the ability to specifically bind to an antigen. The antibody fragment may comprise, for example, one or more CDRs, the variable region (or portions thereof), the constant region (or portions thereof), or combinations thereof. Examples of antibody fragments include, but are not limited to, a Fab fragment, a F(ab′)₂ fragment, an Fv fragment, a single chain Fv (scFv), a domain antibody (dAb or VH), a single domain antibody (sdAb), a VHH, and a nanobody.

The antigen-binding domain may be selected from a scFv, a domain antibody (dAb or VH), a single domain antibody (sdAb), a VHH, or a nanobody.

The domain ABD of the first polypeptide of the invention may comprise an antigen-binding domain which is based on a non-immunoglobulin scaffold, also known as antibody mimetic. These antibody-binding domains are also called antibody mimetics. Non-limiting examples of non-immunoglobulin antigen-binding domains include an affibody, a fibronectin artificial antibody scaffold, an anticalin, an affilin, a DARPin, a VNAR, an iBody, an affimer, a fynomeran, abdurin/nanoantibody, a centyrin, an alphabody, a nanofitin, and a D domain.

The antigen-binding domain may be non-human, such as murine, rat or camelid, chimeric, humanised or fully human. The antigen-binding domain may be synthetic.

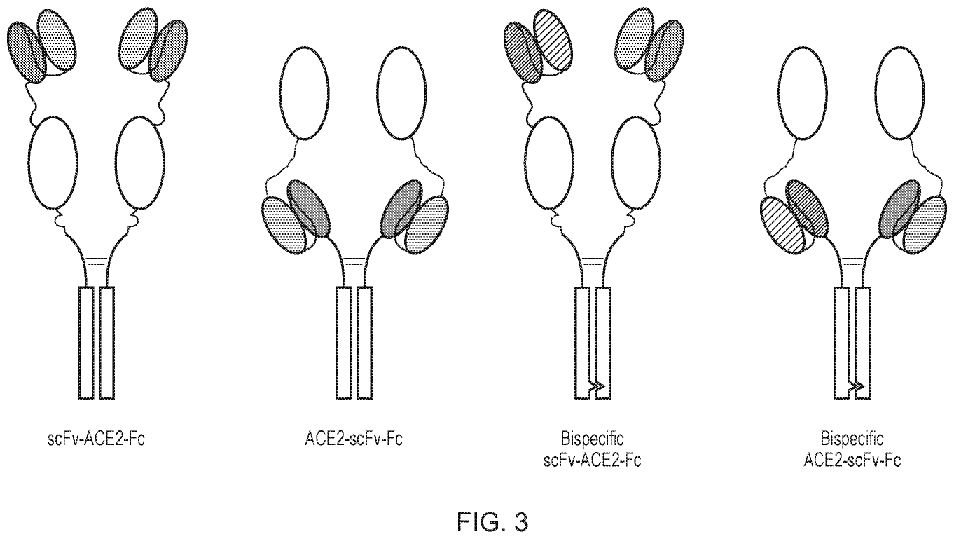

Non-limiting examples domain A, domain O and domain ABD fusion proteins in different orientations are depicted in FIG. 3.

Preparation of Antibodies

Preparation of antibodies may be performed using standard laboratory techniques. Antibodies may be obtained from animal serum, or, in the case of monoclonal antibodies or fragments thereof, produced in cell culture. Recombinant DNA technology may be used to produce the antibodies according to established procedure, in bacterial or mammalian cell culture.

Methods for the production of monoclonal antibodies are well known in the art. Briefly, monoclonal antibodies are typically made by fusing myeloma cells with the spleen cells from a mouse or rabbit that has been immunised with the desired antigen. Herein, the desired antigen is a coronavirus S protein, or subunit S1 or subunit S2 of a coronavirus S protein. The coronavirus may be SARS-CoV-2. The antigen-binding domain will be readily obtained from monoclonal antibodies by means of molecular biology techniques that are conventional in the art.

Alternatively, antibodies and related molecules, particularly scFvs, may be made outside the immune system by combining libraries of VH and VL chains in a recombinant manner. Libraries of VH, VHH, dAb and nanobodies may also be generated. Such libraries may be constructed and screened using phage-display technology as described in Example 9.

The antibody libraries may be immune or non-immune.

Approaches to generate antibody mimetics having a particular specificity are similar and well-known in the art. Methods to generate and screen antibody mimetics against a particular target are described, for example, in Binz et al., 2005 (Binz et al., 2005, Nat Biotech 23:1257-68).

Identification of Coronavirus S Protein-Specific Antibodies

Antibodies which are selective for a coronavirus S protein, or subunit S1 or subunit S2 of a coronavirus S protein, may be identified using methods which are standard in the art. Methods for determining the binding specificity of an antibody include, but are not limited to, enzyme-linked immunosorbent assay (ELISA), and competitive ELISA, western blot, immunofluorescent techniques such as immunohistochemistry (IHC), fluorescence microscopy, and flow cytometry; surface plasmon resonance (SPR), radioimmunoassay (RIA), Forster resonance energy transfer (FRET), phage display libraries, yeast two-hybrid screens, co-immunoprecipitation, bimolecular fluorescence complementation and tandem affinity purification. Additionally, the virus neutralisation ability of antibodies may be determined by incubating the antibodies with relevant virus, e.g. lentiviral vectors pseudotyped with coronavirus S protein, and cultured onto ACE2-expressing cells. These methods are further described in Example 10.

To identify an antibody which is selective for the S protein of SARS-CoV-2, or subunit S1 or subunit S2 thereof, and not for the S protein of another coronavirus, the binding of the antibody to these targets is assessed. Typically, the binding to either the S protein (or S1 or S2 subunits) of SARS-CoV-2 or of another coronavirus is determined.

The antigen-binding domain and ACE2 or a fragment thereof comprising the amino acid sequence shown as SEQ ID NO: 3 may bind to the same or to different epitopes on the coronavirus S protein.

Steric hindrance may prevent domain A and domain ABD from binding to coronavirus protein S at the same time. Thus, advantageously, the antigen-binding domain and ACE2 or a fragment thereof comprising the amino acid sequence shown as SEQ ID NO: 3 may bind to different epitopes on the coronavirus S protein. This will enhance the interaction of the first polypeptide of the invention with the S protein.

The antigen-binding domain may bind to the S1 subunit or the S2 subunit of the coronavirus S protein of one of the following coronavirus: SARS-CoV-2, SARS-CoV, SARS-like CoV RaTG13, MERS-CoV, HCoV-OC43, HCoV-HKU1, HCoV-NL63, and HCoV-229E.

Since the S protein of SARS-CoV-2 share about 76% and 97% amino acid sequence identity that of SARS-CoV and SARS-like CoV RaTG13, the antigen-binding domains of antibodies specific for the S protein of SARS-CoV and SARS-like CoV RaTG13 may be useful to enhance the interaction of the first polypeptide of the invention for the S protein of SARS-CoV-2. While the affinity of these cross-reacting antibodies may not be optimal, the avidity effect obtained may have a beneficial effect in the overall interaction of the first polypeptide of the invention and the S protein of SARS-CoV-2. Thus, the antigen-binding domain may bind to the S1 subunit or the S2 subunit of the coronavirus S protein of SARS-CoV-2, SARS-CoV, or SARS-like CoV RaTG13.

The antigen-binding domain of domain ABD may be specific for the S protein of SARS-CoV-2, or subunit S1 or subunit S2 thereof, of SARS-CoV-2.

The antigen-binding domain may bind to an epitope in the HR1 or the HR2 region of the S2 subunit of the S protein of SARS-CoV-2.

The antigen-binding domain of domain ABD may be specific for the S protein of SARS-like CoV RaTG13, or subunit S1 or subunit S2 thereof, of SARS-like CoV RaTG13.

The antigen-binding domain may comprise the CDR1, CDR2, and CDR3 from one of the following sequences:
a VH sequence of SEQ ID NO: 10 and a VL sequence of SEQ ID NO: 11;
a VH sequence of SEQ ID NO: 12 and a VL sequence of SEQ ID NO: 13;
a VH sequence of SEQ ID NO: 14 and a VL sequence of SEQ ID NO: 15;
a VH sequence of SEQ ID NO: 16 and a VL sequence of SEQ ID NO: 17;
a VH sequence of SEQ ID NO: 18 and a VL sequence of SEQ ID NO: 19;
a VH sequence of SEQ ID NO: 20 and a VL sequence of SEQ ID NO: 21;
a VH sequence of SEQ ID NO: 22 and a VL sequence of SEQ ID NO: 23;
a VH sequence of SEQ ID NO: 24 and a VL sequence of SEQ ID NO: 25;
a VH sequence of SEQ ID NO: 26 and a VL sequence of SEQ ID NO: 27;
a VH sequence of SEQ ID NO: 28 and a VL sequence of SEQ ID NO: 29;
a VH sequence of SEQ ID NO: 30 and a VL sequence of SEQ ID NO: 31;
a VH sequence of SEQ ID NO: 32 and a VL sequence of SEQ ID NO: 33;
a VH sequence of SEQ ID NO: 34 and a VL sequence of SEQ ID NO: 35;
a VH sequence of SEQ ID NO: 36 and a VL sequence of SEQ ID NO: 37;
a VH sequence of SEQ ID NO: 38 and a VL sequence of SEQ ID NO: 39;
a VH sequence of SEQ ID NO: 40 and a VL sequence of SEQ ID NO: 41;
a VH sequence of SEQ ID NO: 42 and a VL sequence of SEQ ID NO: 43;
a VH sequence of SEQ ID NO: 44 and a VL sequence of SEQ ID NO: 45;
a VH sequence of SEQ ID NO: 46 and a VL sequence of SEQ ID NO: 47;
a VH sequence of SEQ ID NO: 48 and a VL sequence of SEQ ID NO: 49;
a VH sequence of SEQ ID NO: 50 and a VL sequence of SEQ ID NO: 51;
a VH sequence of SEQ ID NO: 52 and a VL sequence of SEQ ID NO: 53;
a VH sequence of SEQ ID NO: 58;
a VH sequence of SEQ ID NO: 59; and
a VH sequence of SEQ ID NO: 60.

The antigen-binding domain may comprise or consist of one of the following sequences:
a VH sequence of SEQ ID NO: 10 and a VL sequence of SEQ ID NO: 11;
a VH sequence of SEQ ID NO: 12 and a VL sequence of SEQ ID NO: 13;
a VH sequence of SEQ ID NO: 14 and a VL sequence of SEQ ID NO: 15;
a VH sequence of SEQ ID NO: 16 and a VL sequence of SEQ ID NO: 17;
a VH sequence of SEQ ID NO: 18 and a VL sequence of SEQ ID NO: 19;
a VH sequence of SEQ ID NO: 20 and a VL sequence of SEQ ID NO: 21;
a VH sequence of SEQ ID NO: 22 and a VL sequence of SEQ ID NO: 23;
a VH sequence of SEQ ID NO: 24 and a VL sequence of SEQ ID NO: 25;
a VH sequence of SEQ ID NO: 26 and a VL sequence of SEQ ID NO: 27;
a VH sequence of SEQ ID NO: 28 and a VL sequence of SEQ ID NO: 29;
a VH sequence of SEQ ID NO: 30 and a VL sequence of SEQ ID NO: 31;
a VH sequence of SEQ ID NO: 32 and a VL sequence of SEQ ID NO: 33;
a VH sequence of SEQ ID NO: 34 and a VL sequence of SEQ ID NO: 35;
a VH sequence of SEQ ID NO: 36 and a VL sequence of SEQ ID NO: 37;
a VH sequence of SEQ ID NO: 38 and a VL sequence of SEQ ID NO: 39;
a VH sequence of SEQ ID NO: 40 and a VL sequence of SEQ ID NO: 41;
a VH sequence of SEQ ID NO: 42 and a VL sequence of SEQ ID NO: 43;
a VH sequence of SEQ ID NO: 44 and a VL sequence of SEQ ID NO: 45;
a VH sequence of SEQ ID NO: 46 and a VL sequence of SEQ ID NO: 47;
a VH sequence of SEQ ID NO: 48 and a VL sequence of SEQ ID NO: 49;
a VH sequence of SEQ ID NO: 50 and a VL sequence of SEQ ID NO: 51;
a VH sequence of SEQ ID NO: 52 and a VL sequence of SEQ ID NO: 53;
a VH sequence of SEQ ID NO: 58;
a VH sequence of SEQ ID NO: 59; and
a VH sequence of SEQ ID NO: 60.

VH domain clone 80R (SEQ ID NO: 10; CDR1, CDR2 and CDR3 underlined):
EVQLVQSGGGVVQPGKSLRLSCAASGFAFSSYAMHWVRQAPGKGLEWVAVISYD
GSNKYYADSVKGRFTISRDNSKNTLYLQMNSLRAEDTAVYYCARDRSYYLDYW
GQGTLVTVSS
VL domain clone 80R (SEQ ID NO: 11; CDR1, CDR2 and CDR3 underlined):
ETTLTQSPATLSLSPGERATLSCRASQSVRSNLAWYQQKPGQAPRPLIYDASTRAT
GIPDRFSGSGSGTDFTLTISRLEPEDFAVYYCQQRSNWPPTFGQGTKVEVK
VH domain clone S230.15(SEQ ID NO: 12; CDR1, CDR2 and CDR3 underlined):
QAQLVESGGALVQPGRSLRLSCAASGFTFRNYAMHWVRQAPATGLQWLAVITSD
GRNKFYADSVKGRFTISREDSKNTLYLQMDSLRGEDTAVYYCVTQRDNSRDYFP
HYFHDMDVWGQGTTVAVSS
VL domain clone S230.15(SEQ ID NO: 13; CDR1, CDR2 and CDR3 underlined):
DVVLTQSPLSLPVTLGQPASISCRSSQSLVYSDGDTYLNWFQQRPGQSPRRLIYQVS
NRDSGVPDRFSGSGSGTDFTLKISRVEAEDVGVYYCMQGSHWPPTFGQGTKVEIK
VH domain clone M396 (SEQ ID NO: 14; CDR1, CDR2 and CDR3 underlined):
QVQLQQSGAEVKKPGSSVKVSCKASGGTFSSYTISWVRQAPGQGLEWMGGITPIL
GIANYAQKFQGRVTITTDESTSTAYMELSSLRSEDTAVYYCARDTVMGGMDVWG
QGTTVTVSS
VL domain clone M396 (SEQ ID NO: 15; CDR1, CDR2 and CDR3 underlined):
SYELTQPPSVSVAPGKTARITCGGNNIGSKSVHWYQQKPGQAPVLVVYDDSDRPS
GIPERFSGSNSGNTATLTISRVEAGDEADYYCQVWDSSSDYVFGTGTKVTVL
VH domain clone F26G19 (SEQ ID NO: 16; CDR1, CDR2 and CDR3 underlined):
EVQLEESGTVLARPGASVKMSCKASGYTFTTYRMHWIKQRPGQGLEWIGAIYPGN
SDTTYNQKFKDKAKLTAVTSTSSAYMELSSLTNEDSAVYFCTREGIPQLLRTLDY
WGQGTSVTVSS
VL domain clone F26G19 (SEQ ID NO: 17; CDR1, CDR2 and CDR3 underlined):
DILMTQSPSSLSASLGERVSLTCRASQEISGYLSWLQEKPDGTIKRLIYAASTLDSG
VPKRFSGSRSGSDYSLTISSLESEDFADYYCLQYVSYPWTFGGGTKLEIK
VH domain clone F26G8 (SEQ ID NO: 18; CDR1, CDR2 and CDR3 underlined):
QVQLLESGAELVKPGASVKVSCKASGYTFTSYWIHWVKQRPGQGLEWIGEINPSN
GRTNYNGNFESKATLTVDKSSNTAYMHLSSLTYEDSAVYHCTRLDYWGQGTTLTVSS
VL domain clone F26G8 (SEQ ID NO: 19; CDR1, CDR2 and CDR3 underlined):
ELVMTQSPASLSVITGKKVTIRCISNTDIDDDLNWSQLKAGEPPKLLISEGNIFSPGV
PSRFSSSGNGTDFVFTIENTLSEDVANNYCFQSDNMPFTFGSGTKLEIK
VH domain clone F26G8.2 (SEQ ID NO: 20; CDR1, CDR2 and CDR3 underlined):
QVQLQQPGAELVKPGASVKVSCKASGYTFTNYWIHWVKQRPGQGLEWIGEINPG
NGRTNYNGNFMNKATLTVDKSSNTAYMQLSSLTSEDSAVYHCARLDYWGQGTT
LTVSS
VL domain clone F26G8.2 (SEQ ID NO: 21; CDR1, CDR2 and CDR3 underlined):
ELVMTQSPASLSVATGKKVTIRCISSTDIDDDMNWYQQKAGKPPKLLISEGNIFSPG
VPSRFSSSGNGTDFVFTVENTLSEDVADNYCLQSDNMPFTFGSGTKLGIK
VH domain clone F26G18 (SEQ ID NO: 22; CDR1, CDR2 and CDR3 underlined):
QLVQLEESGTVLPRPGASVKMSCKASGYTFTSYWMHWVKQRPGQGLEWIGAIYP
GNSDTNYNQKFKGRATLTAVTSTSTASMELSSLTNEDSAVYYCTRDGYGSLYYY
AMDFWGQGTSVTVSS
VL domain clone F26G18 (SEQ ID NO: 23; CDR1, CDR2 and CDR3 underlined):
ELVMTQSPSSLSASLGDRVTISCRASQDISNYLNWYQQKPDGTVKLLIYYTSRLHA
GVPSRFSGSGSGTDYSLTISNLEQEDIATYFCQQGYTLPYTFGGGTKLEIK
VH domain clone 92N (SEQ ID NO: 24; CDR1, CDR2 and CDR3 underlined):
QVQLVQSGAEVKKPGASVKVSCKASGYTFTTYGISWVRQAPGQGLEWMGWISA
YNGNTNYAQKLQGRVTMTTDTSTSTAYMELRSLRSDDTAVYYCARDDLSDYGE
WLGPDYWGQGTLVTVSS
VL domain clone 92N (SEQ ID NO: 25; CDR1, CDR2 and CDR3 underlined):
QPGLTQPPSASGTPGQRVTISCSGSRSNIGGNTVNWYQHVPGTAPKLLIYSNNQRP
SGVPDRFSGSKSGTSASLAISGLRSEDEAEYYCATWEDSLSGYVFGPGTKVTVL
VH domain clone 91M (SEQ ID NO: 26; CDR1, CDR2 and CDR3 underlined):
QVQLVQSGGGLVQPGGSLRLSCAASGFTFSSSWMSWVRQAPGKGLEWVAYIKQD
GSEKYYVDSVKGRFTISRDNAKNSLYLQMDSLRAEDTAVYYCARDQGWGWDGT
EYYSDYWGQGTLVTVSS
VL domain clone 91M (SEQ ID NO: 27; CDR1, CDR2 and CDR3 underlined):
QSALTQPASVSGSPGQSITISCTGTSSDVGGYNYVSWYQQHPGKAPKLMIYDVSKR
PSGVPDRFSGSKSGNTASLTISGLQAEDEADYFCSSYTITDIVVFGGGTKLTVL
VH domain clone 27D (SEQ ID NO: 28; CDR1, CDR2 and CDR3 underlined):
QVQLVQSGGGLIQPGGSLRLSCAASGFTVSSNYMSWVRQAPGKGLEWVSVIYSG
GSTYYADSVKGRFTISRDNSKNTLYLQMNSLRAEDTAVYYCARNFGEDFDYWGQ
GTLVTVSS
VL domain clone 27D (SEQ ID NO: 29; CDR1, CDR2 and CDR3 underlined):
QSALTQPASVSGSPGQSITISCTGTSSDLGGHNFVSWYQQHPGKAPKLMIYDVFNR
PSGVSSRFSGSKSGNTASLTISGLQAEDEADYFCSSYTITNIVVFGRGTKLTVL
VH domain clone 26H (SEQ ID NO: 30; CDR1, CDR2 and CDR3 underlined):
QVQLQESGGGLVKPGGSLRLSCAASGFTFSDYYMSWIRQAPGKGLEWVSYISSSG
STIYYADSVKGRFTISRDNAKNSLYLQMNSLRAEDTAVYYCARERWLQIGEDAFD
IWGQGTTVTVSS
VL domain clone 26H (SEQ ID NO: 31; CDR1, CDR2 and CDR3 underlined):
QTVVTQEPSFSVSPGGTITLTCDLNSGLVSSSHYPSWYQQTPGQAPRTLIYNTNIRS
SGVPDRFSGAILGNKAALTITGAQAEDESDYYCVLYMGSGISVFGGGTKLTVLGQ
VH domain clone 12E (SEQ ID NO: 32; CDR1, CDR2 and CDR3 underlined):
QVQLVQSGAEVKKPGASVKVSCKASGYTFTSYGISWVRQAPGQGLEWMGWISAY
NGNTNYAQKLQGRVTMTTDTSTSTAYMELRSLRSDDTAVYYCARVGYYGDYAW
GYYYYGMDVWGQGTLVTVSS
VL domain clone 12E (SEQ ID NO: 33; CDR1, CDR2 and CDR3 underlined):
SYELTQPPSASGTPGQRVTISCSGSSSSIGSETVNWYQQLPGTAPKLLIYSNNQRPSG
VPDRFSGSKSGTSASLAISGLQSEDEADYYCTAWDDTLNGRVIFGGGTKLTVL
VH domain clone 8C (SEQ ID NO: 34; CDR1, CDR2 and CDR3 underlined):
EVQLVQSGGGLVKPGGSLRLSCAASGFTFSDYYMSWIRQAPGKGLEWVSYISSSG
STIYYADSVKGRFTISRDNAKNSLYLQMNSLRAEDTGVYYCARDWGYSFGDWGQ
GTLVTVSS
VL domain clone 8C (SEQ ID NO: 35; CDR1, CDR2 and CDR3 underlined):
DIQMTQSPSSLSASVGDRVTITCRATQSISTHLNWYQQRGGKAPKLLIYGASTLES
GVPSRFSGSGSGTEFTLTISSLQPEDFATYYCLQHRSYPWTFGQGTNVEIK
VH domain clone CR3009 (SEQ ID NO: 36; CDR1, CDR2 and CDR3 underlined):
EVQLVESGGGVVQPGRSLRLSCAASGFTFSDYPMNWVRQAPGKGLEWVSSISGSG
GSTYYADSVKGRFTISRDNSKNTLYLQMNSLRAEDTAVYYCAKGLFMVTTYAFD
YWGQGTLVTVLE
VL domain clone CR3009 (SEQ ID NO: 37; CDR1, CDR2 and CDR3 underlined):
DIELTQSPSSLSASVGDRVTITCRASQSISSYLNWYQQKPGKAPKLLIYAASSLQSG
VPSRFSGSGSGTDFTLTISSLQPEDFATYYCQQSYSTPPTFGQGTKVEIK
VH domain clone CR3006 (SEQ ID NO: 38; CDR1, CDR2 and CDR3 underlined):
EVQLVESGGGLVQPGGSLRLSCAASGFTFSGYPMHWVRQAPGKGLEWVAVISYD
GSNKYYADSVKGRFTISRDNSKNTLYLQMNSLRAEDTAVYYCAKDGSPRTPSFDY
WGQGTLVTVLE
VL domain clone CR3006 (SEQ ID NO: 39; CDR1, CDR2 and CDR3 underlined):
DIQMTQSPHSLSASVGDRVTITCRASQGISNYLAWYQQKPGKVPKLLIYAASTLQS
GVPSRFSGSGSGTDFTLTISSLQPEDVGVYYCQQRFRTPVTFGQGTKLEIK
VH domain clone CR3018 (SEQ ID NO: 40; CDR1, CDR2 and CDR3 underlined):
EVQLVESGGGLVQPGGSLRLSCAASGFTFSSYAMSWVRQAPGKGLEWVSAISGSG
GSTYYADSVKGRFTISRDNSKNTLYLQMNSLRAEDTAVYYCAKFNPFTSFDYWG
QGTLVTVSS
VL domain clone CR3018 (SEQ ID NO: 41; CDR1, CDR2 and CDR3 underlined):
DIELTQSPSSLSASVGDRVTITCRASQSISSYLNWYQQKPGKAPKLLIYAASSLQSG
VPSRFSGSGSGTDFTLTISSLQPEDFATYYCQQSYSTPPTFGQGTKVEIK
VH domain clone CR3013 (SEQ ID NO: 42; CDR1, CDR2 and CDR3 underlined):
EVQLVESGGGLVQPGGSLRLSCAASGFTFSDHYMDWVRQAPGKGLEWVGRTRN
KANSYTTEYAASVKGRFTISRDDSKNSLYLQMNSLKTEDTAVYYCAKGLTPLYFD
YWGQGTLVTVSS
VL domain clone CR3013 (SEQ ID NO: 43; CDR1, CDR2 and CDR3 underlined):
DIELTQSPSSLSASVGDRVTITCRASQSISSYLNWYQQKPGKAPKLLIYAASSLQSG
VPSRFSGSGSGTDFTLTISSLQPEDFATYYCQQSYSTPPTFGQGTKVEIK
VH domain clone CR3014 (SEQ ID NO: 44; CDR1, CDR2 and CDR3 underlined):
EVQLVESGGGLVQPGGSLRLSCAASGFTFSDHYMDWVRQAPGKGLEWVGRTRN
KANSYTTEYAASVKGRFTISRDDSKNSLYLQMNSLKTEDTAVYYCARGISPFYFD
YWGQGTLVTVSS
VL domain clone CR3014 (SEQ ID NO: 45; CDR1, CDR2 and CDR3 underlined):
DIELTQSPSSLSASVGDRVTITCRASQSISSYLNWYQQKPGKAPKLLIYAASSLQSG
VPSRFSGSGSGTDFTLTISSLQPEDFATYYCQQSYSTPPTFGQGTKVEIK
VH domain clone CR3015 (SEQ ID NO: 46; CDR1, CDR2 and CDR3 underlined):
EVQLVESGGGVVRPGGSLRLSCAASGFTFDDYGMSWVRQAPGKGLEWVSGINW
NGGSTGYADSVKGRFTISRDNAKNSLYLQMNSLRAEDTAVYYCARGLSLRPWGQ
GTLVTVSR
VL domain clone CR3015 (SEQ ID NO: 47; CDR1, CDR2 and CDR3 underlined):
SELTQDPAVSVALGQTVRITCQGDSLRSYYASWYQQKPGQAPVLVIYGKNNRPSG
IPDRFSGSSSGNTASLTITGAQAEDEADYYCNSRDSSGNHVVFGGGTKLTVL
VH domain clone AS3-3 (SEQ ID NO: 48; CDR1, CDR2 and CDR3 underlined):
QVQLVQSGAEVKKPGESLKISCKGSGYSFANHWIAWVRQMPGKGLEWMGIFNPD
NSDTTYSPSFQGQVTFSADKSISIAYLHWSSLKASDTAIYYCARLALSDGWLHDFW
GQGTLVTVSS
VL domain clone AS3-3 (SEQ ID NO: 49; CDR1, CDR2 and CDR3 underlined):
EIVMTQSPLSLPVTLGQPASISCKSSQSLVDTNGNTYLIWFHQRPGQSPRRLIYQVS
KRDSGVPDRFSGSGSGTDFTLKISRVEAEDVGVYFCMQGTYRPPTLGQGTKVEIK
VH domain clone CR3022 (SEQ ID NO: 50; CDR1, CDR2 and CDR3 underlined):
QMQLVQSGTEVKKPGESLKISCKGSGYGFITYWIGWVRQMPGKGLEWMGIIYPGD
SETRYSPSFQGQVTISADKSINTAYLQWSSLKASDTAIYYCAGGSGISTPMDVWGQ
GTTVTVSS
VL domain clone CR3022 (SEQ ID NO: 51; CDR1, CDR2 and CDR3 underlined):
DIQLTQSPDSLAVSLGERATINCKSSQSVLYSSINKNYLAWYQQKPGQPPKLLIYW
ASTRESGVPDRFSGSGSGTDFTLTISSLQAEDVAVYYCQQYYSTPYTFGQGTKVEIK
VH domain clone B1 (SEQ ID NO: 52; CDR1, CDR2 and CDR3 underlined):
QVQLLESGAEVKKPGASAKVSCKASGCTFTSYGISWVRQAPGQGLEWMGWISAY
NGNTNYAQKLQGRVTMTTDTSTSTAYMELRSLRSDDTAVYYCARGVAVAGTWD
WFDPWGQGTLVTVSS
VL domain clone B1 (SEQ ID NO: 53; CDR1, CDR2 and CDR3 underlined):
DIVLTQTPGTLSLSPGERATLSCRASQSVRTYLAWYQQKPGQVPRLLIYGASSRAT
GIPDRFSGSGSGTDFTLTISRLEPEDFAVYYCQQYGSSLWTFGQGTRLEIKR
dAb clone 5 (SEQ ID NO: 58; CDR1, CDR2 and CDR3 underlined):
QVQLQESGGGLVQAGGSLRLSCVDSGRTFSGGTMAWFRQAPGKAREFVAADRGF
TSTYYADAVKGRFTISRDNAENAAENAVYLQMNSLKPEDTAVYYCAARPTVAFS
TDEIFYKFWGQGTQVTVSS
dAb clone 7 (SEQ ID NO: 59; CDR1, CDR2 and CDR3 underlined):
QVQLQESGGGLVEAGGSLRLSCVASGFTFEDYNIGWFRQAPGKEREGVSYIRANG
GTTYYTDSVKGRFTISADNAKNTVYLQMSNLKSEDTAVYYCAADGSSWPIDLPVY
EYDYWGQGTQVTVSS
dAb clone 9 (SEQ ID NO: 60; CDR1, CDR2 and CDR3 underlined):
QVQLQESGGGLVQAGDSLRLSCAASGRTSDTMTIGWFRQAPGKEREFVASINWSG
STTYYADSVNERFTISRDDAKNTVYLQMNSLKPEDTAVYYCAAKRGVWTIKSAA
ADFASWGQGTQVTVSS

Variants of the above amino acid sequences may also be used in the present invention, provided that the resulting antibody binds coronavirus S protein. Typically, such variants have a high degree of sequence identity with one of the sequences specified above.

The terms “identity”, “identical” or “percent identity” have been described in previous sections of this specification and their definitions and particular features apply equally herein.

Variants of the VL or VH domain or scFv typically have at least about 75%, for example at least about 80%, 90%, 95%, 96%, 97%, 98% or 99% sequence identity with the sequences given as SEQ ID NO: 10-53.

Typically, variants may contain one or more conservative amino acid substitutions compared to the original amino acid or nucleic acid sequence. Conservative substitutions are those substitutions that do not substantially affect or decrease the affinity of an antibody to bind coronavirus S protein. For example, a human antibody that specifically binds coronavirus S protein may include up to 1, up to 2, up to 5, up to 10, or up to 15 conservative substitutions in either or both of the VH or VL compared to any of the sequences given as SEQ ID No. 10-53 and retain specific binding to coronavirus S protein.

The first polypeptide of the invention may further comprise an additional domain ABD which comprises an antigen-binding domain that binds specifically to a coronavirus S protein.

The term “domain ABD” has been described previously and all the definitions and particular features of the first domain ABD apply equally to the additional domain ABD.

Steric hindrance may prevent domain A and domain ABD from binding to coronavirus S protein at the same time. In order to avoid this, the ectodomain of hACE2 or a fragment thereof comprising the amino acid sequence shown as SEQ ID NO: 3, the antigen-binding domain of domain ABD and the antigen-binding domain of the additional domain ABD may bind to different epitopes on the coronavirus S protein.

The coronavirus S protein may be the spike protein of SARS-CoV-2 Coronavirus.

Internal flexibility is also a factor contributing to multivalent binding. Thus, each of domain A, domain O, domain ABD, additional domain ABD, and, where present, domain C, may be joined by a linker. The linker may be a flexible linker. The linker may be the (Gly₄Ser)₃ linker (SEQ ID NO: 6).

The first polypeptide of the invention may consist of one of the amino acid sequences shown as SEQ ID NO: 84 to 87 and 96 to 99.

hACE2_18-605-L-SARS_aCoV_CR3022_
ScFv_HL-HuIgG1Fc
(SEQ ID NO: 84)
QSTIEEQAKTFLDKFNHEAEDLFYQSSLASWNYNTNITEE
NVQNMNNAGDKWSAFLKEQSTLAQMYPLQEIQNLTVKLQL
QALQQNGSSVLSEDKSKRLNTILNTMSTIYSTGKVCNPDN
PQECLLLEPGLNEIMANSLDYNERLWAWESWRSEVGKQLR
PLYEEYVVLKNEMARANHYEDYGDYWRGDYEVNGVDGYDY
SRGQLIEDVEHTFEEIKPLYEHLHAYVRAKLMNAYPSYIS
PIGCLPAHLLGDMWGRFWTNLYSLTVPFGQKPNIDVTDAM
VDQAWDAQRIFKEAEKFFVSVGLPNMTQGFWENSMLTDPG
NVQKAVCHPTAWDLGKGDFRILMCTKVTMDDFLTAHNEMG
NIQYDMAYAAQPFLLRNGANEGFHEAVGEIMSLSAATPKH
LKSIGLLSPDFQEDNETEINFLLKQALTIVGTLPFTYMLE
KWRWMVFKGEIPKDQWMKKWWEMKREIVGVVEPVPHDETY
CDPASLFHVSNDYSFIRYYTRTLYQFQFQEALCQAAKHEG
PLHKCDISNSTEAGQKLFNMLRLGKSEPWTLALENVVGAK
NMNVRPLLNYFEPLFTWLKDQNKNSFVGGGGGSGGGGSGG
GASQMQLVQSGTEVKKPGESLKISCKGSGYGFITYWIGWV
RQMPGKGLEWMGIIYPGDSETRYSPSFQGQVTISADKSIN
TAYLQWSSLKASDTAIYYCAGGSGISTPMDVWGQGTTVTV
SSGGGGSGGGGSGGGGSDIQLTQSPDSLAVSLGERATINC
KSSQSVLYSSINKNYLAWYQQKPGQPPKLLIYWASTRESG
VPDRFSGSGSGTDFTLTISSLQAEDVAVYYCQQYYSTPYT
FGQGTKVEIKGSEPKSCDKTHTCPPCPAPELLGGPSVFLF
PPKPKDTLMISRTPEVTCVVVDVSHEDPEVKFNWYVDGVE
VHNAKTKPREEQYNSTYRVVSVLTVLHQDWLNGKEYKCKV
SNKALPAPIEKTISKAKGQPREPQVYTLPPSRDELTKNQV
SLTCLVKGFYPSDIAVEWESNGQPENNYKTTPPVLDSDGS
FFLYSKLTVDKSRWQQGNVFSCSVMHEALHNHYTQKSLSL
SPGK
hACE2_18-740-L-SARS_aCoV_CR3022_
ScFv_HL-HuIgG1Fc
(SEQ ID NO: 85)
QSTIEEQAKTFLDKFNHEAEDLFYQSSLASWNYNTNITEE
NVQNMNNAGDKWSAFLKEQSTLAQMYPLQEIQNLTVKLQL
QALQQNGSSVLSEDKSKRLNTILNTMSTIYSTGKVCNPDN
PQECLLLEPGLNEIMANSLDYNERLWAWESWRSEVGKQLR
PLYEEYVVLKNEMARANHYEDYGDYWRGDYEVNGVDGYDY
SRGQLIEDVEHTFEEIKPLYEHLHAYVRAKLMNAYPSYIS
PIGCLPAHLLGDMWGRFWTNLYSLTVPFGQKPNIDVTDAM
VDQAWDAQRIFKEAEKFFVSVGLPNMTQGFWENSMLTDPG
NVQKAVCHPTAWDLGKGDFRILMCTKVTMDDFLTAHNEMG
NIQYDMAYAAQPFLLRNGANEGFHEAVGEIMSLSAATPKH
LKSIGLLSPDFQEDNETEINFLLKQALTIVGTLPFTYMLE
KWRWMVFKGEIPKDQWMKKWWEMKREIVGVVEPVPHDETY
CDPASLFHVSNDYSFIRYYTRTLYQFQFQEALCQAAKHEG
PLHKCDISNSTEAGQKLFNMLRLGKSEPWTLALENVVGAK
NMNVRPLLNYFEPLFTWLKDQNKNSFVGWSTDWSPYADQS
IKVRISLKSALGDKAYEWNDNEMYLFRSSVAYAMRQYFLK
VKNQMILFGEEDVRVANLKPRISFNFFVTAPKNVSDIIPR
TEVEKAIRMSRSRINDAFRLNDNSLEFLGIQPTLGPPNQP
PVSGGGGSGGGGSGGGASQMQLVQSGTEVKKPGESLKISC
KGSGYGFITYWIGWVRQMPGKGLEWMGIIYPGDSETRYSP
SFQGQVTISADKSINTAYLQWSSLKASDTAIYYCAGGSGI
STPMDVWGQGTTVTVSSGGGGSGGGGSGGGGSDIQLTQSP
DSLAVSLGERATINCKSSQSVLYSSINKNYLAWYQQKPGQ
PPKLLIYWASTRESGVPDRFSGSGSGTDFTLTISSLQAED
VAVYYCQQYYSTPYTFGQGTKVEIKGSEPKSCDKTHTCPP
CPAPELLGGPSVFLFPPKPKDTLMISRTPEVTCVVVDVSH
EDPEVKFNWYVDGVEVHNAKTKPREEQYNSTYRVVSVLTV
LHQDWLNGKEYKCKVSNKALPAPIEKTISKAKGQPREPQV
YTLPPSRDELTKNQVSLTCLVKGFYPSDIAVEWESNGQPE
NNYKTTPPVLDSDGSFFLYSKLTVDKSRWQQGNVFSCSVM
HEALHNHYTQKSLSLSPGK
hACE2_18-605-L-SARS_aCoV-S2_B1_ScFv_
HL-HuIgG1Fc
(SEQ ID NO: 86)
QSTIEEQAKTFLDKFNHEAEDLFYQSSLASWNYNTNITEE
NVQNMNNAGDKWSAFLKEQSTLAQMYPLQEIQNLTVKLQL
QALQQNGSSVLSEDKSKRLNTILNTMSTIYSTGKVCNPDN
PQECLLLEPGLNEIMANSLDYNERLWAWESWRSEVGKQLR
PLYEEYVVLKNEMARANHYEDYGDYWRGDYEVNGVDGYDY
SRGQLIEDVEHTFEEIKPLYEHLHAYVRAKLMNAYPSYIS
PIGCLPAHLLGDMWGRFWTNLYSLTVPFGQKPNIDVTDAM
VDQAWDAQRIFKEAEKFFVSVGLPNMTQGFWENSMLTDPG
NVQKAVCHPTAWDLGKGDFRILMCTKVTMDDFLTAHNEMG
NIQYDMAYAAQPFLLRNGANEGFHEAVGEIMSLSAATPKH
LKSIGLLSPDFQEDNETEINFLLKQALTIVGTLPFTYMLE
KWRWMVFKGEIPKDQWMKKWWEMKREIVGVVEPVPHDETY
CDPASLFHVSNDYSFIRYYTRTLYQFQFQEALCQAAKHEG
PLHKCDISNSTEAGQKLFNMLRLGKSEPWTLALENVVGAK
NMNVRPLLNYFEPLFTWLKDQNKNSFVGGGGGSGGGGSGG
GASQVQLLESGAEVKKPGASAKVSCKASGCTFTSYGISWV
RQAPGQGLEWMGWISAYNGNTNYAQKLQGRVTMTTDTSTS
TAYMELRSLRSDDTAVYYCARGVAVAGTWDWFDPWGQGTL
VTVSSGGGGSGGGGSGGGGSDIVLTQTPGTLSLSPGERAT
LSCRASQSVRTYLAWYQQKPGQVPRLLIYGASSRATGIPD
RFSGSGSGTDFTLTISRLEPEDFAVYYCQQYGSSLWTFGQ
GTRLEIKRGSEPKSCDKTHTCPPCPAPELLGGPSVFLFPP
KPKDTLMISRTPEVTCVVVDVSHEDPEVKFNWYVDGVEVH
NAKTKPREEQYNSTYRVVSVLTVLHQDWLNGKEYKCKVSN
KALPAPIEKTISKAKGQPREPQVYTLPPSRDELTKNQVSL
TCLVKGFYPSDIAVEWESNGQPENNYKTTPPVLDSDGSFF
LYSKLTVDKSRWQQGNVFSCSVMHEALHNHYTQKSLSLSP
GK
hACE2_18-740-L-SARS_aCoV-S2_B1_
ScFv_HL-HuIgG1Fc
(SEQ ID NO: 87)
QSTIEEQAKTFLDKFNHEAEDLFYQSSLASWNYNTNITEE
NVQNMNNAGDKWSAFLKEQSTLAQMYPLQEIQNLTVKLQL
QALQQNGSSVLSEDKSKRLNTILNTMSTIYSTGKVCNPDN
PQECLLLEPGLNEIMANSLDYNERLWAWESWRSEVGKQLR
PLYEEYVVLKNEMARANHYEDYGDYWRGDYEVNGVDGYDY
SRGQLIEDVEHTFEEIKPLYEHLHAYVRAKLMNAYPSYIS
PIGCLPAHLLGDMWGRFWTNLYSLTVPFGQKPNIDVTDAM
VDQAWDAQRIFKEAEKFFVSVGLPNMTQGFWENSMLTDPG
NVQKAVCHPTAWDLGKGDFRILMCTKVTMDDFLTAHNEMG
NIQYDMAYAAQPFLLRNGANEGFHEAVGEIMSLSAATPKH
LKSIGLLSPDFQEDNETEINFLLKQALTIVGTLPFTYMLE
KWRWMVFKGEIPKDQWMKKWWEMKREIVGVVEPVPHDETY
CDPASLFHVSNDYSFIRYYTRTLYQFQFQEALCQAAKHEG
PLHKCDISNSTEAGQKLFNMLRLGKSEPWTLALENVVGAK
NMNVRPLLNYFEPLFTWLKDQNKNSFVGWSTDWSPYADQS
IKVRISLKSALGDKAYEWNDNEMYLFRSSVAYAMRQYFLK
VKNQMILFGEEDVRVANLKPRISFNFFVTAPKNVSDIIPR
TEVEKAIRMSRSRINDAFRLNDNSLEFLGIQPTLGPPNQP
PVSGGGGSGGGGSGGGASQVQLLESGAEVKKPGASAKVSC
KASGCTFTSYGISWVRQAPGQGLEWMGWISAYNGNTNYAQ
KLQGRVTMTTDTSTSTAYMELRSLRSDDTAVYYCARGVAV
AGTWDWFDPWGQGTLVTVSSGGGGSGGGGSGGGGSDIVLT
QTPGTLSLSPGERATLSCRASQSVRTYLAWYQQKPGQVPR
LLIYGASSRATGIPDRFSGSGSGTDFTLTISRLEPEDFAV
YYCQQYGSSLWTFGQGTRLEIKRGSEPKSCDKTHTCPPCP
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAKGQPREPQVYT
LPPSRDELTKNQVSLTCLVKGFYPSDIAVEWESNGQPENN
YKTTPPVLDSDGSFFLYSKLTVDKSRWQQGNVFSCSVMHE
ALHNHYTQKSLSLSPGK
SARS_aCoV_CR3022_ScFv_HL-L_ACE2_
18-605_shuttle_HuIgG1Fc
(SEQ ID NO: 96)
QMQLVQSGTEVKKPGESLKISCKGSGYGFITYWIGWVRQM
PGKGLEWMGIIYPGDSETRYSPSFQGQVTISADKSINTAY
LQWSSLKASDTAIYYCAGGSGISTPMDVWGQGTTVTVSSG
GGGSGGGGSGGGGSDIQLTQSPDSLAVSLGERATINCKSS
QSVLYSSINKNYLAWYQQKPGQPPKLLIYWASTRESGVPD
RFSGSGSGTDFTLTISSLQAEDVAVYYCQQYYSTPYTFGQ
GTKVEIKGSGGGGSGGGGSGGGGSQSTIEEQAKTFLDKFN
HEAEDLFYQSSLASWNYNTNITEENVQNMNNAGDKWSAFL
KEQSTLAQMYPLQEIQNLTVKLQLQALQQNGSSVLSEDKS
KRLNTILNTMSTIYSTGKVCNPDNPQECLLLEPGLNEIMA
NSLDYNERLWAWESWRSEVGKQLRPLYEEYVVLKNEMARA
NHYEDYGDYWRGDYEVNGVDGYDYSRGQLIEDVEHTFEEI
KPLYEHLHAYVRAKLMNAYPSYISPIGCLPAHLLGDMWGR
FWTNLYSLTVPFGQKPNIDVTDAMVDQAWDAQRIFKEAEK
FFVSVGLPNMTQGFWENSMLTDPGNVQKAVCHPTAWDLGK
GDFRILMCTKVTMDDFLTAHNEMGNIQYDMAYAAQPFLLR
NGANEGFHEAVGEIMSLSAATPKHLKSIGLLSPDFQEDNE
TEINFLLKQALTIVGTLPFTYMLEKWRWMVFKGEIPKDQW
MKKWWEMKREIVGVVEPVPHDETYCDPASLFHVSNDYSFI
RYYTRTLYQFQFQEALCQAAKHEGPLHKCDISNSTEAGQK
LFNMLRLGKSEPWTLALENVVGAKNMNVRPLLNYFEPLFT
WLKDQNKNSFVGEPKSCDKTHTCPPCPAPELLGGPSVFLF
PPKPKDTLMISRTPEVTCVVVDVSHEDPEVKFNWYVDGVE
VHNAKTKPREEQYNSTYRVVSVLTVLHQDWLNGKEYKCKV
SNKALPAPIEKTISKAKGQPREPQVYTLPPSRDELTKNQV
SLTCLVKGFYPSDIAVEWESNGQPENNYKTTPPVLDSDGS
FFLYSKLTVDKSRWQQGNVFSCSVMHEALHNHYTQKSLSL
SPGK
SARS_aCoV_CR3022_ScFv_HL-L_ACE2_
18-740_shuttle_HuIgG1Fc
(SEQ ID NO: 97)
QMQLVQSGTEVKKPGESLKISCKGSGYGFITYWIGWVRQM
PGKGLEWMGIIYPGDSETRYSPSFQGQVTISADKSINTAY
LQWSSLKASDTAIYYCAGGSGISTPMDVWGQGTTVTVSSG
GGGSGGGGSGGGGSDIQLTQSPDSLAVSLGERATINCKSS
QSVLYSSINKNYLAWYQQKPGQPPKLLIYWASTRESGVPD
RFSGSGSGTDFTLTISSLQAEDVAVYYCQQYYSTPYTFGQ
GTKVEIKGSGGGGSGGGGSGGGGSQSTIEEQAKTFLDKFN
HEAEDLFYQSSLASWNYNTNITEENVQNMNNAGDKWSAFL
KEQSTLAQMYPLQEIQNLTVKLQLQALQQNGSSVLSEDKS
KRLNTILNTMSTIYSTGKVCNPDNPQECLLLEPGLNEIMA
NSLDYNERLWAWESWRSEVGKQLRPLYEEYVVLKNEMARA
NHYEDYGDYWRGDYEVNGVDGYDYSRGQLIEDVEHTFEEI
KPLYEHLHAYVRAKLMNAYPSYISPIGCLPAHLLGDMWGR
FWTNLYSLTVPFGQKPNIDVTDAMVDQAWDAQRIFKEAEK
FFVSVGLPNMTQGFWENSMLTDPGNVQKAVCHPTAWDLGK
GDFRILMCTKVTMDDFLTAHNEMGNIQYDMAYAAQPFLLR
NGANEGFHEAVGEIMSLSAATPKHLKSIGLLSPDFQEDNE
TEINFLLKQALTIVGTLPFTYMLEKWRWMVFKGEIPKDQW
MKKWWEMKREIVGVVEPVPHDETYCDPASLFHVSNDYSFI
RYYTRTLYQFQFQEALCQAAKHEGPLHKCDISNSTEAGQK
LFNMLRLGKSEPWTLALENVVGAKNMNVRPLLNYFEPLFT
WLKDQNKNSFVGWSTDWSPYADQSIKVRISLKSALGDKAY
EWNDNEMYLFRSSVAYAMRQYFLKVKNQMILFGEEDVRVA
NLKPRISFNFFVTAPKNVSDIIPRTEVEKAIRMSRSRIND
AFRLNDNSLEFLGIQPTLGPPNQPPVSEPKSCDKTHTCPP
CPAPELLGGPSVFLFPPKPKDTLMISRTPEVTCVVVDVSH
EDPEVKFNWYVDGVEVHNAKTKPREEQYNSTYRVVSVLTV
LHQDWLNGKEYKCKVSNKALPAPIEKTISKAKGQPREPQV
YTLPPSRDELTKNQVSLTCLVKGFYPSDIAVEWESNGQPE
NNYKTTPPVLDSDGSFFLYSKLTVDKSRWQQGNVFSCSVM
HEALHNHYTQKSLSLSPGK
SARS_aCoV-S2_B1_ScFv_HL-L_ACE2_
18-605_shuttle_HuIgG1Fc
(SEQ ID NO: 98)
QVQLLESGAEVKKPGASAKVSCKASGCTFTSYGISWVRQA
PGQGLEWMGWISAYNGNTNYAQKLQGRVTMTTDTSTSTAY
MELRSLRSDDTAVYYCARGVAVAGTWDWFDPWGQGTLVTV
SSGGGGSGGGGSGGGGSDIVLTQTPGTLSLSPGERATLSC
RASQSVRTYLAWYQQKPGQVPRLLIYGASSRATGIPDRFS
GSGSGTDFTLTISRLEPEDFAVYYCQQYGSSLWTFGQGTR
LEIKRGSGGGGSGGGGSGGGGSQSTIEEQAKTFLDKFNHE
AEDLFYQSSLASWNYNTNITEENVQNMNNAGDKWSAFLKE
QSTLAQMYPLQEIQNLTVKLQLQALQQNGSSVLSEDKSKR
LNTILNTMSTIYSTGKVCNPDNPQECLLLEPGLNEIMANS
LDYNERLWAWESWRSEVGKQLRPLYEEYVVLKNEMARANH
YEDYGDYWRGDYEVNGVDGYDYSRGQLIEDVEHTFEEIKP
LYEHLHAYVRAKLMNAYPSYISPIGCLPAHLLGDMWGRFW
TNLYSLTVPFGQKPNIDVTDAMVDQAWDAQRIFKEAEKFF
VSVGLPNMTQGFWENSMLTDPGNVQKAVCHPTAWDLGKGD
FRILMCTKVTMDDFLTAHNEMGNIQYDMAYAAQPFLLRNG
ANEGFHEAVGEIMSLSAATPKHLKSIGLLSPDFQEDNETE
INFLLKQALTIVGTLPFTYMLEKWRWMVFKGEIPKDQWMK
KWWEMKREIVGVVEPVPHDETYCDPASLFHVSNDYSFIRY
YTRTLYQFQFQEALCQAAKHEGPLHKCDISNSTEAGQKLF
NMLRLGKSEPWTLALENVVGAKNMNVRPLLNYFEPLFTWL
KDQNKNSFVGEPKSCDKTHTCPPCPAPELLGGPSVFLFPP
KPKDTLMISRTPEVTCVVVDVSHEDPEVKFNWYVDGVEVH
NAKTKPREEQYNSTYRVVSVLTVLHQDWLNGKEYKCKVSN
KALPAPIEKTISKAKGQPREPQVYTLPPSRDELTKNQVSL
TCLVKGFYPSDIAVEWESNGQPENNYKTTPPVLDSDGSFF
LYSKLTVDKSRWQQGNVFSCSVMHEALHNHYTQKSLSLSP
GK
SARS_aCoV-S2_B1_ScFv_HL-L_ACE2_18-
740_shuttle_HuIgG1Fc
(SEQ ID NO: 99)
QVQLLESGAEVKKPGASAKVSCKASGCTFTSYGISWVRQA
PGQGLEWMGWISAYNGNTNYAQKLQGRVTMTTDTSTSTAY
MELRSLRSDDTAVYYCARGVAVAGTWDWFDPWGQGTLVTV
SSGGGGSGGGGSGGGGSDIVLTQTPGTLSLSPGERATLSC
RASQSVRTYLAWYQQKPGQVPRLLIYGASSRATGIPDRFS
GSGSGTDFTLTISRLEPEDFAVYYCQQYGSSLWTFGQGTR
LEIKRGSGGGGSGGGGSGGGGSQSTIEEQAKTFLDKFNHE
AEDLFYQSSLASWNYNTNITEENVQNMNNAGDKWSAFLKE
QSTLAQMYPLQEIQNLTVKLQLQALQQNGSSVLSEDKSKR
LNTILNTMSTIYSTGKVCNPDNPQECLLLEPGLNEIMANS
LDYNERLWAWESWRSEVGKQLRPLYEEYVVLKNEMARANH
YEDYGDYWRGDYEVNGVDGYDYSRGQLIEDVEHTFEEIKP
LYEHLHAYVRAKLMNAYPSYISPIGCLPAHLLGDMWGRFW
TNLYSLTVPFGQKPNIDVTDAMVDQAWDAQRIFKEAEKFF
VSVGLPNMTQGFWENSMLTDPGNVQKAVCHPTAWDLGKGD
FRILMCTKVTMDDFLTAHNEMGNIQYDMAYAAQPFLLRNG
ANEGFHEAVGEIMSLSAATPKHLKSIGLLSPDFQEDNETE
INFLLKQALTIVGTLPFTYMLEKWRWMVFKGEIPKDQWMK
KWWEMKREIVGVVEPVPHDETYCDPASLFHVSNDYSFIRY
YTRTLYQFQFQEALCQAAKHEGPLHKCDISNSTEAGQKLF
NMLRLGKSEPWTLALENVVGAKNMNVRPLLNYFEPLFTWL
KDQNKNSFVGWSTDWSPYADQSIKVRISLKSALGDKAYEW
NDNEMYLFRSSVAYAMRQYFLKVKNQMILFGEEDVRVANL
KPRISFNFFVTAPKNVSDIIPRTEVEKAIRMSRSRINDAF
RLNDNSLEFLGIQPTLGPPNQPPVSEPKSCDKTHTCPPCP
APELLGGPSVFLFPPKPKDTLMISRTPEVTCVVVDVSHED
PEVKFNWYVDGVEVHNAKTKPREEQYNSTYRVVSVLTVLH
QDWLNGKEYKCKVSNKALPAPIEKTISKAKGQPREPQVYT
LPPSRDELTKNQVSLTCLVKGFYPSDIAVEWESNGQPENN
YKTTPPVLDSDGSFFLYSKLTVDKSRWQQGNVFSCSVMHE
ALHNHYTQKSLSLSPGK

The first polypeptide of the invention may comprise a domain A, a domain O, a domain ABD, additional domain ABD, and, where present, a domain C, in any configuration. The skilled person will immediately recognise all the possible different domain structures (from N- to C-terminus).

2.5. Domain ALB

The first polypeptide of the invention may be short-lived in the bloodstream or, otherwise, its pharmacokinetic properties may need to be enhanced. Plasma proteins and plasma protein binding proteins or peptides can be an effective means of improving the pharmacokinetic properties of any molecule. One of these plasma proteins is albumin, which has been extensively investigated for extending the half-life of therapeutic molecules in blood.

Human serum albumin (HSA) can be exploited in one of two ways. One approach is to directly couple the therapeutic protein to HSA, either genetically or chemically. A second approach is to use an albumin-binding domain.

The first polypeptide of the invention may further comprise a domain ALB which comprises albumin or an albumin domain, or an antigen-binding domain that binds specifically to albumin, or an albumin-binding-peptide, or an albumin-binding domain of a Streptococcus protein.

Therefore, the first polypeptide of the invention may comprise:
a) a domain A which comprises the ectodomain of human angiotensin converting enzyme type 2 (hACE2), or a fragment thereof comprising the amino acid sequence shown as SEQ ID NO: 3, or a variant thereof which maintains the capacity of hACE2 to interact with the S protein of coronavirus,
b) a domain O which comprises an oligomerisation domain, and
c) a domain ALB which comprises albumin or an albumin domain, or an antigen-binding domain that binds specifically to albumin, or an albumin-binding-peptide, or an albumin-binding domain of a Streptococcus protein.

Optionally, the first polypeptide of the invention may further comprise a domain ABD, wherein the domain ABD comprises an antigen-binding domain that binds specifically to a coronavirus S protein and/or a domain C which comprises the ectodomain of CD147 or a fragment thereof comprising the amino acid sequence shown as SEQ ID NO: 8 or SEQ ID NO: 9.

The terms “polypeptide”, “domain A”, “hACE2”, “domain O”, “oligomerisation domain”, “domain C”, “CD147”, “domain ABD”, “antigen-binding domain”, and “coronavirus S protein” have been described in detail in previous sections of this specification, and their definitions and particular features apply equally herein.

Importantly, the proviso defining that “where the first polypeptide of the invention comprises one domain A which consists of the ectodomain of hACE2 having a sequence of SEQ ID NO: 1 or 2, then the domain O may not comprise the human IgG1 Fc region” does not apply to polypeptides of the invention comprising a domain A, a domain O and a domain ALB, and, where present, a domain C and/or a domain ABD.

The first polypeptide of the invention may comprise a domain A, a domain O, and a domain ALB, and, where present, a domain C and/or a domain ABD, in any orientation. The skilled person will immediately recognise all the possible different domain structures (from N- to C-terminus).

Each of domain A, domain O, and a domain ALB, and, where present, a domain C and/or a domain ABD, may be joined by a linker. The linker may be a flexible linker. The linkers described in the context of the fusion between domain A and domain O may be equally used herein. The linker may be the (Gly₄Ser)₃ linker (SEQ ID NO: 6).

Albumin is the most abundant protein in plasma, present at 50 mg/ml (600 μM), and has a half-life of 19 days in humans. With a molecular mass of about 67 kDa, albumin serves to maintain plasma pH, contributes to colloidal blood pressure, functions as carrier of many metabolites and fatty acids, and serves as a major drug transport protein in plasma. Human albumin is depicted under Accession No. P02768 in the Uniprot database on 30 Mar. 2020. Albumin is formed by three domains, i.e. albumin domain 1 spanning aa 19-210, albumin domain 2 spanning aa 211-403, and albumin domain 3 spanning aa 404-601.

Domain ALB of the first polypeptide of the invention may comprise the sequence of the full albumin protein or the sequence of one or two of the albumin domains.

The amino acid sequence of HSA is shown as SEQ ID NO: 139. Alternatively, it may comprise a mutated HSA, such as the ones described in WO2010059315.

Wild-type human serum albumin;
processed protein
(SEQ ID NO: 139)
RGVFRRDAHKSEVAHRFKDLGEENFKALVLIAFAQYLQQC
PFEDHVKLVNEVTEFAKTCVADESAENCDKSLHTLFGDKL
CTVATLRETYGEMADCCAKQEPERNECFLQHKDDNPNLPR
LVRPEVDVMCTAFHDNEETFLKKYLYEIARRHPYFYAPEL
LFFAKRYKAAFTECCQAADKAACLLPKLDELRDEGKASSA
KQRLKCASLQKFGERAFKAWAVARLSQRFPKAEFAEVSKL
VTDLTKVHTECCHGDLLECADDRADLAKYICENQDSISSK
LKECCEKPLLEKSHCIAEVENDEMPADLPSLAADFVESKD
VCKNYAEAKDVFLGMFLYEYARRHPDYSVVLLLRLAKTYE
TTLEKCCAAADPHECYAKVFDEFKPLVEEPQNLIKQNCEL
FEQLGEYKFQNALLVRYTKKVPQVSTPTLVEVSRNLGKVG
SKCCKHPEAKRMPCAEDYLSVVLNQLCVLHEKTPVSDRVT
KCCTESLVNRRPCFSALEVDETYVPKEFNAETFTFHADIC
TLSEKERQIKKQTALVELVKHKPKATKEQLKAVMDDFAAF
VEKCCKADDKETCFAEEGKKLVAASQAALGL

The half-life extending domain may comprise one domain of HSA, i.e. domain I of HSA (residues 1-192 of SEQ ID NO: 139), domain II of HSA (residues 193-385 of SEQ ID NO: 139), or domain III of HSA (residues 386-591 of SEQ ID NO: 139). The half-life extending domain may comprise a combination of HSA domains, such as domains I and II, I and III, or II and III.

Domain ALB of the first polypeptide of the invention may comprise the sequence of an antigen-binding domain that binds specifically to albumin.

The term “antigen-binding domain” has been described previously, and its definition and particular features apply equally herein. Where necessary, the skilled person will readily be able to adapt the previous description to albumin.

Numerous anti-HSA antigen-binding domain have been described in the art, such as scFvs, single domain antibodies (Nanobody™, AlbudAb™) and Fabs, as well as albumin-binding domains based on antibody mimetics, such as anti-albumin DARPins. Anti-serum albumin binding single variable domains have been described, for example, in Holt et al., Protein Eng Des Sel 21:283-8, WO04003019, WO2008096158, WO05118642, WO20060591056, WO2011/006915.

Domain ALB of the first polypeptide of the invention may comprise the sequence of an albumin-binding-peptide. A non-limiting example of an albumin-binding-peptide includes peptides having the core sequence DICLPRWGCLW (SEQ ID NO: 54), which was generated using peptide phage display to specifically bind to albumin.

Domain ALB of the first polypeptide of the invention may comprise the sequence of an albumin-binding domain of a Streptococcus protein. The Streptococcal protein may be Protein G. The albumin-binding domain of Streptococcal protein G may be the albumin-binding domain B2A3 (BA) and/or B1A2B2A3 (BABA).

Other modifications used to extend half-life that are currently known in the art, or that will be developed in the future, also form part of the present invention. For example, the first polypeptide of the invention may be conjugated to polyethylene glycol (PEG), or pegylated.

3. Polypeptide Based on CD147

In another aspect, the present invention provides a polypeptide, hereinafter “the second polypeptide of the invention”, comprising
a) a domain C which comprises the ectodomain of CD147 or a fragment thereof comprising the amino acid sequence shown as SEQ ID NO: 2 or a fragment thereof comprising the amino acid sequence shown as SEQ ID NO: 3, and
b) a domain O which comprises an oligomerisation domain.

The terms “polypeptide”, “domain C”, “CD147”, “domain O”, and “oligomerisation domain” have been described in detail in the context of the first polypeptide of the invention, and their definitions and particular features apply equally to the second polypeptide of the invention.

The oligomerisation domain may be selected from an IgG Fc region or a variant thereof which does not interact with FcγRI, FcγRIIa and FcγRIII, an IgM Fc region, an IgA Fc region, a collagen XVIII trimerizing structural element, a collagen XV trimerizing structural element, a foldon domain, a TenC domain and a coiled coil domain.

Non-limiting examples domain C and domain O fusion proteins are depicted in FIG. 5.

The second polypeptide of the invention may comprise, from N-terminus to C-terminus:
domain C-domain O, and
domain O-domain C.

The second polypeptide of the invention may consist of an amino acid sequence selected from SEQ ID NO: 122-124.

hCD147-Fc
(SEQ ID NO: 122)
AAGFVQAPLSQQRWVGGSVELHCEAVGSPVPEIQWWFEGQ
GPNDTCSQLWDGARLDRVHIHATYHQHAASTISIDTLVEE
DTGTYECRASNDPDRNHLTRAPRVKWVRAQAVVLVLEPGT
VFTTVEDLGSKILLTCSLNDSATEVTGHRWLKGGVVLKED
ALPGQKTEFKVDSDDQWGEYSCVFLPEPMGTANIQLHGPP
RVKAVKSSEHINEGETAMLVCKSESVPPVTDWAWYKITDS
EDKALMNGSESRFFVSSSQGRSELHIENLNMEADPGQYRC
NGTSSKGSDQAIITLRVRSEPKSCDKTHTCPPCPAPELLG
GPSVFLFPPKPKDTLMISRTPEVTCVVVDVSHEDPEVKFN
WYVDGVEVHNAKTKPREEQYNSTYRVVSVLTVLHQDWLNG
KEYKCKVSNKALPAPIEKTISKAKGQPREPQVYTLPPSRE
EMTKNQVSLTCLVKGFYPSDIAVEWESNGQPENNYKTTPP
VLDSDGSFFLYSKLTVDKSRWQQGNVFSCSVMHEALHNHY
TQKSLSLSPGK
hCD147 D1-Fc
(SEQ ID NO: 123)
AAGFVQAPLSQQRWVGGSVELHCEAVGSPVPEIQWWFEGQ
GPNDTCSQLWDGARLDRVHIHATYHQHAASTISIDTLVEE
DTGTYECRASNDPDRNHLTRAPRVKWVRAQAVVLVLEPGT
VFTTVEDLGSKILLTCSLNDSATEVTGHRWLKGGVVLKED
ALPGQKTEFKVDSDDQWGEYSCVFLPEPMGTANIQLHGEP
KSCDKTHTCPPCPAPELLGGPSVFLFPPKPKDTLMISRTP
EVTCVVVDVSHEDPEVKFNWYVDGVEVHNAKTKPREEQYN
STYRVVSVLTVLHQDWLNGKEYKCKVSNKALPAPIEKTIS
KAKGQPREPQVYTLPPSREEMTKNQVSLTCLVKGFYPSDI
AVEWESNGQPENNYKTTPPVLDSDGSFFLYSKLTVDKSRW
QQGNVFSCSVMHEALHNHYTQKSLSLSPGK
CD147 D2-Fc
(SEQ ID NO: 124)
GPPRVKAVKSSEHINEGETAMLVCKSESVPPVTDWAWYKI
TDSEDKALMNGSESRFFVSSSQGRSELHIENLNMEADPGQ
YRCNGTSSKGSDQAIITLRVRSEPKSCDKTHTCPPCPAPE
LLGGPSVFLFPPKPKDTLMISRTPEVTCVVVDVSHEDPEV
KFNWYVDGVEVHNAKTKPREEQYNSTYRVVSVLTVLHQDW
LNGKEYKCKVSNKALPAPIEKTISKAKGQPREPQVYTLPP
SREEMTKNQVSLTCLVKGFYPSDIAVEWESNGQPENNYKT
TPPVLDSDGSFFLYSKLTVDKSRWQQGNVFSCSVMHEALH
NHYTQKSLSLSPGK

Domain C and domain O may be joined by a linker. The linker may be a flexible linker. The linkers described in the context of the fusion between domain C and domain O may be equally used herein. The linker may be the (Gly₄Ser)₃ linker (SEQ ID NO: 6).

As an alternative approach to enhance the interaction between the neutralising clamp of the invention and SARS-CoV-2, the CD147 molecule can be combined with antibodies binding distinct epitopes on the coronavirus S protein, maximising avidity and overall neutralisation potential. Although viral escape mechanisms (through viral mutation) are more probable with non-endogenous ligands such as antibodies, the use of two or more binding events minimises the risk of such occurrence.

Thus, the second polypeptide of the invention may further comprise a domain ABD, wherein the domain ABD comprises an antigen-binding domain that binds specifically to a coronavirus S protein.

The second polypeptide of the invention may comprise a domain C, a domain O and a domain ABD.

The second polypeptide of the invention may comprise a domain C, a domain O and a domain ABD in any orientation. The structure of the polypeptide may be selected from one of the following, from N-terminus to C-terminus:
domain C-domain ABD-domain O,
domain ABD-domain C-domain O,
domain C-domain O-domain ABD,
domain ABD-domain O-domain C,
domain O-domain C-domain ABD, and
domain O-domain ABD-domain C.

Each of domain C, domain O and domain ABD may be joined by a linker. The linker may be a flexible linker. The linkers described in the context of the fusion between domain A and domain O may be equally used herein. The linker may be the (Gly₄Ser)₃ linker (SEQ ID NO: 6).

The terms “ABD”, and “coronavirus S protein” have been described in detail in the context of the first polypeptide of the invention, and their definitions and particular features apply equally to the second polypeptide of the invention.

The antigen-binding domain of domain ABD may be specific for the S protein of SARS-CoV-2, or subunit S1 or subunit S2 thereof, of SARS-CoV-2.

The antigen-binding domain may bind to an epitope in the HR1 or the HR2 region of the S2 subunit of the S protein of SARS-CoV-2.

The antigen-binding domain of domain ABD may be specific for the S protein of SARS-like CoV RaTG13, or subunit S1 or subunit S2 thereof, of SARS-like CoV RaTG13.

The antigen-binding domain of domain ABD may be specific for the S protein of SARS-CoV, or subunit S1 or subunit S2 thereof, of SARS-CoV.

The antigen-binding domain is selected from a scFv or a domain antibody (dAb or VH).

The antigen-binding domain may comprise the CDR1, CDR2, and CDR3 from one of the following sequences:
a VH sequence of SEQ ID NO: 10 and a VL sequence of SEQ ID NO: 11;
a VH sequence of SEQ ID NO: 12 and a VL sequence of SEQ ID NO: 13;
a VH sequence of SEQ ID NO: 14 and a VL sequence of SEQ ID NO: 15;
a VH sequence of SEQ ID NO: 16 and a VL sequence of SEQ ID NO: 17;
a VH sequence of SEQ ID NO: 18 and a VL sequence of SEQ ID NO: 19;
a VH sequence of SEQ ID NO: 20 and a VL sequence of SEQ ID NO: 21;
a VH sequence of SEQ ID NO: 22 and a VL sequence of SEQ ID NO: 23;
a VH sequence of SEQ ID NO: 24 and a VL sequence of SEQ ID NO: 25;
a VH sequence of SEQ ID NO: 26 and a VL sequence of SEQ ID NO: 27;
a VH sequence of SEQ ID NO: 28 and a VL sequence of SEQ ID NO: 29;
a VH sequence of SEQ ID NO: 30 and a VL sequence of SEQ ID NO: 31;
a VH sequence of SEQ ID NO: 32 and a VL sequence of SEQ ID NO: 33;
a VH sequence of SEQ ID NO: 34 and a VL sequence of SEQ ID NO: 35;
a VH sequence of SEQ ID NO: 36 and a VL sequence of SEQ ID NO: 37;
a VH sequence of SEQ ID NO: 38 and a VL sequence of SEQ ID NO: 39;
a VH sequence of SEQ ID NO: 40 and a VL sequence of SEQ ID NO: 41;
a VH sequence of SEQ ID NO: 42 and a VL sequence of SEQ ID NO: 43;
a VH sequence of SEQ ID NO: 44 and a VL sequence of SEQ ID NO: 45;
a VH sequence of SEQ ID NO: 46 and a VL sequence of SEQ ID NO: 47;
a VH sequence of SEQ ID NO: 48 and a VL sequence of SEQ ID NO: 49;
a VH sequence of SEQ ID NO: 50 and a VL sequence of SEQ ID NO: 51;
a VH sequence of SEQ ID NO: 52 and a VL sequence of SEQ ID NO: 53;
a VH sequence of SEQ ID NO: 58;
a VH sequence of SEQ ID NO: 59; and
a VH sequence of SEQ ID NO: 60.

The antigen-binding domain may comprise one of the following sequences:
a VH sequence of SEQ ID NO: 10 and a VL sequence of SEQ ID NO: 11;
a VH sequence of SEQ ID NO: 12 and a VL sequence of SEQ ID NO: 13;
a VH sequence of SEQ ID NO: 14 and a VL sequence of SEQ ID NO: 15;
a VH sequence of SEQ ID NO: 16 and a VL sequence of SEQ ID NO: 17;
a VH sequence of SEQ ID NO: 18 and a VL sequence of SEQ ID NO: 19;
a VH sequence of SEQ ID NO: 20 and a VL sequence of SEQ ID NO: 21;
a VH sequence of SEQ ID NO: 22 and a VL sequence of SEQ ID NO: 23;
a VH sequence of SEQ ID NO: 24 and a VL sequence of SEQ ID NO: 25;
a VH sequence of SEQ ID NO: 26 and a VL sequence of SEQ ID NO: 27;
a VH sequence of SEQ ID NO: 28 and a VL sequence of SEQ ID NO: 29;
a VH sequence of SEQ ID NO: 30 and a VL sequence of SEQ ID NO: 31;
a VH sequence of SEQ ID NO: 32 and a VL sequence of SEQ ID NO: 33;
a VH sequence of SEQ ID NO: 34 and a VL sequence of SEQ ID NO: 35;
a VH sequence of SEQ ID NO: 36 and a VL sequence of SEQ ID NO: 37;
a VH sequence of SEQ ID NO: 38 and a VL sequence of SEQ ID NO: 39;
a VH sequence of SEQ ID NO: 40 and a VL sequence of SEQ ID NO: 41;
a VH sequence of SEQ ID NO: 42 and a VL sequence of SEQ ID NO: 43;
a VH sequence of SEQ ID NO: 44 and a VL sequence of SEQ ID NO: 45;
a VH sequence of SEQ ID NO: 46 and a VL sequence of SEQ ID NO: 47;
a VH sequence of SEQ ID NO: 48 and a VL sequence of SEQ ID NO: 49;
a VH sequence of SEQ ID NO: 50 and a VL sequence of SEQ ID NO: 51;
a VH sequence of SEQ ID NO: 52 and a VL sequence of SEQ ID NO: 53;
a VH sequence of SEQ ID NO: 58;
a VH sequence of SEQ ID NO: 59; and
a VH sequence of SEQ ID NO: 60.

The coronavirus S protein may be the spike protein of SARS-CoV-2 coronavirus.

Variants of the above amino acid sequences may also be used in the second polypeptide of the invention, provided that the resulting antibody binds coronavirus S protein. Typically, such variants have a high degree of sequence identity with one of the sequences specified above.

The terms “identity”, “identical” or “percent identity” have been described in the context of the first polypeptide of the invention and their definitions and particular features apply equally to the second polypeptide of the invention.

The second polypeptide of the invention may further comprise an additional domain ABD which comprises an antigen-binding domain that binds specifically to a Coronavirus spike protein.

The term “domain ABD” has been described previously and all the definitions and particular features of the first domain ABD apply equally to the additional domain ABD.

Steric hindrance may prevent domain C and domain ABD from binding to coronavirus protein S at the same time. In order to avoid this, CD147 or a fragment thereof comprising the amino acid sequence shown as SEQ ID NO: 2 or a fragment thereof comprising the amino acid sequence shown as SEQ ID NO: 3, the antigen-binding domain of domain ABD and the antigen-binding domain of the additional domain ABD may bind to different epitopes on the Coronavirus spike protein.

The coronavirus S protein may be the spike protein of SARS-CoV-2, SARS-CoV or SARS-like CoV RaTG13.

Internal flexibility is also a factor contributing to multivalent binding. Thus, each of domain C, domain O, domain ABD, and additional domain ABD may be joined by a linker. The linker may be a flexible linker. The linker may be the (Gly₄Ser)₃ linker (SEQ ID NO: 6).

The second polypeptide of the invention may comprise a domain C, a domain O, a domain ABD, and an additional domain ABD in any orientation. The skilled person will immediately recognise all the possible different domain structures (from N- to C-terminus).

The second polypeptide of the invention may be short-lived in the bloodstream or, otherwise, its pharmacokinetic properties may need to be enhanced. Plasma proteins and plasma protein binding can be an effective means of improving the pharmacokinetic properties of any molecule. One of these plasma proteins is albumin, which has been extensively investigated for extending the half-life of therapeutic molecules in blood.

Thus, the second polypeptide of the invention may further comprise a domain ALB which comprises albumin or an albumin domain, or an antigen-binding domain that binds specifically to albumin, or an albumin-binding-peptide, or an albumin-binding domain of a Streptococcus protein.

The terms “albumin”, “antigen-binding domain that binds specifically to albumin”, “albumin-binding-peptide”, and “albumin-binding domain of a Streptococcus protein” have been described in the context of the first polypeptide of the invention and their definitions and particular features apply equally to the second polypeptide of the invention.

Domain ALB may be joined by a linker. The linker may be a flexible linker. The linkers described in the context of the fusion between domain A and domain O may be equally used herein. The linker may be the (Gly₄Ser)₃ linker (SEQ ID NO: 6).

The second polypeptide of the invention may consist of the amino acid sequence shown as SEQ ID NO: 88 to 91.

CD147_22-320_HuIgMFc
(SEQ ID NO: 88)
AAGFVQAPLSQQRWVGGSVELHCEAVGSPVPEIQWWFEGQ
GPNDTCSQLWDGARLDRVHIHATYHQHAASTISIDTLVEE
DTGTYECRASNDPDRNHLTRAPRVKWVRAQAVVLVLEPGT
VFTTVEDLGSKILLTCSLNDSATEVTGHRWLKGGVVLKED
ALPGQKTEFKVDSDDQWGEYSCVFLPEPMGTANIQLHGPP
RVKAVKSSEHINEGETAMLVCKSESVPPVTDWAWYKITDS
EDKALMNGSESRFFVSSSQGRSELHIENLNMEADPGQYRC
NGTSSKGSDQAIITLRVRSSGGGSIAELPPKVSVFVPPRD
GFFGNPRSKSKLICQATGFSPRQIQVSWLREGKQVGSGVT
TDQVQAEAKESGPTTYKVTSTLTIKESDWLSQSMFTCRVD
HRGLTFQQNASSMCVPDQDTAIRVFAIPPSFASIFLTKST
KLTCLVTDLTTYDSVTISWTRQNGEAVKTHTNISESHPNA
TFSAVGEASICEDDWNSGERFTCTVTHTDLPSPLKQTISR
PKGVALHRPDVYLLPPAREQLNLRESATITCLVTGFSPAD
VFVQWMQRGQPLSPEKYVTSAPMPEPQAPGRYFAHSILTV
SEEEWNTGETYTCVVAHEALPNRVTERTVDKST
CD147-D1_22-219_HuIgG1Fc
(SEQ ID NO: 89)
AAGFVQAPLSQQRWVGGSVELHCEAVGSPVPEIQWWFEGQ
GPNDTCSQLWDGARLDRVHIHATYHQHAASTISIDTLVEE
DTGTYECRASNDPDRNHLTRAPRVKWVRAQAVVLVLEPGT
VFTTVEDLGSKILLTCSLNDSATEVTGHRWLKGGVVLKED
ALPGQKTEFKVDSDDQWGEYSCVFLPEPMGTANIQLHGEP
KSCDKTHTCPPCPAPELLGGPSVFLFPPKPKDTLMISRTP
EVTCVVVDVSHEDPEVKFNWYVDGVEVHNAKTKPREEQYN
STYRVVSVLTVLHQDWLNGKEYKCKVSNKALPAPIEKTIS
KAKGQPREPQVYTLPPSREEMTKNQVSLTCLVKGFYPSDI
AVEWESNGQPENNYKTTPPVLDSDGSFFLYSKLTVDKSRW
QQGNVFSCSVMHEALHNHYTQKSLSLSPGK
CD147-D2_219-320_HuIgG1Fc
(SEQ ID NO: 90)
GPPRVKAVKSSEHINEGETAMLVCKSESVPPVTDWAWYKI
TDSEDKALMNGSESRFFVSSSQGRSELHIENLNMEADPGQ
YRCNGTSSKGSDQAIITLRVRSEPKSCDKTHTCPPCPAPE
LLGGPSVFLFPPKPKDTLMISRTPEVTCVVVDVSHEDPEV
KFNWYVDGVEVHNAKTKPREEQYNSTYRVVSVLTVLHQDW
LNGKEYKCKVSNKALPAPIEKTISKAKGQPREPQVYTLPP
SREEMTKNQVSLTCLVKGFYPSDIAVEWESNGQPENNYKT
TPPVLDSDGSFFLYSKLTVDKSRWQQGNVFSCSVMHEALH
NHYTQKSLSLSPGK
CD147_22-320_HuIgG1Fc
(SEQ ID NO: 91)
AAGFVQAPLSQQRWVGGSVELHCEAVGSPVPEIQWWFEGQ
GPNDTCSQLWDGARLDRVHIHATYHQHAASTISIDTLVEE
DTGTYECRASNDPDRNHLTRAPRVKWVRAQAVVLVLEPGT
VFTTVEDLGSKILLTCSLNDSATEVTGHRWLKGGVVLKED
ALPGQKTEFKVDSDDQWGEYSCVFLPEPMGTANIQLHGPP
RVKAVKSSEHINEGETAMLVCKSESVPPVTDWAWYKITDS
EDKALMNGSESRFFVSSSQGRSELHIENLNMEADPGQYRC
NGTSSKGSDQAIITLRVRSEPKSCDKTHTCPPCPAPELLG
GPSVFLFPPKPKDTLMISRTPEVTCVVVDVSHEDPEVKFN
WYVDGVEVHNAKTKPREEQYNSTYRVVSVLTVLHQDWLNG
KEYKCKVSNKALPAPIEKTISKAKGQPREPQVYTLPPSRD
ELTKNQVSLTCLVKGFYPSDIAVEWESNGQPENNYKTTPP
VLDSDGSFFLYSKLTVDKSRWQQGNVFSCSVMHEALHNHY
TQKSLSLSPGK

The first polypeptide of the invention may comprise a domain C, a domain O, a domain ALB, and, where present, a domain ABD and/or an additional domain ABD, in any orientation. The skilled person will immediately recognise all the possible different domain structures (from N- to C-terminus).

Other modifications used to extend half-life that are currently known in the art, or that will be developed in the future, also form part of the present invention. For example, the first polypeptide of the invention may be conjugated to polyethylene glycol (PEG), or pegylated.

4. Polypeptide Based on Coronavirus SP-Specific Binders

In another aspect, the present invention provides a polypeptide, hereinafter “the third polypeptide of the invention”, comprising:
a) a domain ABD which comprises an antigen-binding domain that binds specifically to a Coronavirus spike protein, and
b) a domain O which comprises an oligomerisation domain.

The terms “polypeptide”, “domain ABD”, “antigen-binding domain”, “coronavirus spike protein”, “domain O”, and “oligomerisation domain” have been described in detail in the context of the first polypeptide of the invention, and their definitions and particular features apply equally to the third polypeptide of the invention.

The oligomerisation domain may be selected from an IgG Fc region or a variant thereof which does not interact with FcγRI, FcγRIIa and FcγRIII, an IgM Fc region, an IgA Fc region, a collagen XVIII trimerizing structural element, a collagen XV trimerizing structural element, a foldon domain, a TenC domain and a coiled coil domain.

Using a variant of an Fc region of an IgG which does not interact with FcγRI, FcγRIIa and FcγRIII is within the scope of the present invention. These variants are well-known in the art. For example, the Fc region of an IgG may contain one or more of the following mutations Leu234Ala, Leu235Ala (LALA).

Non-limiting examples domain ABD and domain O fusion proteins in different orientations are depicted in FIG. 6.

The third polypeptide of the invention may comprise, from N-terminus to C-terminus:
domain ABD-domain O, and
domain O-domain ABD.

Domain ABD and domain O may be joined by a linker. The linker may be a flexible linker. The linkers described in the context of the fusion between domain A and domain O may be equally used herein. The linker may be the (Gly₄Ser)₃ linker (SEQ ID NO: 6).

The antigen-binding domain may bind to the S1 subunit or the S2 subunit of the coronavirus S protein.

The antigen-binding domain of domain ABD may be specific for the S protein of SARS-CoV-2, or subunit S1 or subunit S2 thereof, of SARS-CoV-2.

The antigen-binding domain may bind to an epitope in the HR1 or the HR2 region of the S2 subunit of the S protein of SARS-CoV-2.

The antigen-binding domain of domain ABD may be specific for the S protein of SARS-like CoV RaTG13, or subunit S1 or subunit S2 thereof, of SARS-like CoV RaTG13.

The antigen-binding domain of domain ABD may be specific for the S protein of SARS-CoV, or subunit S1 or subunit S2 thereof, of SARS-CoV.

The antigen-binding domain is selected from a scFv or a domain antibody (dAb or VH).

The antigen-binding domain may comprise the CDR1, CDR2 and CDR3 from one of the following sequences:
a VH sequence of SEQ ID NO: 10 and a VL sequence of SEQ ID NO: 11;
a VH sequence of SEQ ID NO: 12 and a VL sequence of SEQ ID NO: 13;
a VH sequence of SEQ ID NO: 14 and a VL sequence of SEQ ID NO: 15;
a VH sequence of SEQ ID NO: 16 and a VL sequence of SEQ ID NO: 17;
a VH sequence of SEQ ID NO: 18 and a VL sequence of SEQ ID NO: 19;
a VH sequence of SEQ ID NO: 20 and a VL sequence of SEQ ID NO: 21;
a VH sequence of SEQ ID NO: 22 and a VL sequence of SEQ ID NO: 23;
a VH sequence of SEQ ID NO: 24 and a VL sequence of SEQ ID NO: 25;
a VH sequence of SEQ ID NO: 26 and a VL sequence of SEQ ID NO: 27;
a VH sequence of SEQ ID NO: 28 and a VL sequence of SEQ ID NO: 29;
a VH sequence of SEQ ID NO: 30 and a VL sequence of SEQ ID NO: 31;
a VH sequence of SEQ ID NO: 32 and a VL sequence of SEQ ID NO: 33;
a VH sequence of SEQ ID NO: 34 and a VL sequence of SEQ ID NO: 35;
a VH sequence of SEQ ID NO: 36 and a VL sequence of SEQ ID NO: 37;
a VH sequence of SEQ ID NO: 38 and a VL sequence of SEQ ID NO: 39;
a VH sequence of SEQ ID NO: 40 and a VL sequence of SEQ ID NO: 41;
a VH sequence of SEQ ID NO: 42 and a VL sequence of SEQ ID NO: 43;
a VH sequence of SEQ ID NO: 44 and a VL sequence of SEQ ID NO: 45;
a VH sequence of SEQ ID NO: 46 and a VL sequence of SEQ ID NO: 47;
a VH sequence of SEQ ID NO: 48 and a VL sequence of SEQ ID NO: 49;
a VH sequence of SEQ ID NO: 50 and a VL sequence of SEQ ID NO: 51;
a VH sequence of SEQ ID NO: 52 and a VL sequence of SEQ ID NO: 53;
a VH sequence of SEQ ID NO: 58;
a VH sequence of SEQ ID NO: 59; and
a VH sequence of SEQ ID NO: 60.

The antigen-binding domain may comprise one of the following sequences:
a VH sequence of SEQ ID NO: 10 and a VL sequence of SEQ ID NO: 11;
a VH sequence of SEQ ID NO: 12 and a VL sequence of SEQ ID NO: 13;
a VH sequence of SEQ ID NO: 14 and a VL sequence of SEQ ID NO: 15;
a VH sequence of SEQ ID NO: 16 and a VL sequence of SEQ ID NO: 17;
a VH sequence of SEQ ID NO: 18 and a VL sequence of SEQ ID NO: 19;
a VH sequence of SEQ ID NO: 20 and a VL sequence of SEQ ID NO: 21;
a VH sequence of SEQ ID NO: 22 and a VL sequence of SEQ ID NO: 23;
a VH sequence of SEQ ID NO: 24 and a VL sequence of SEQ ID NO: 25;
a VH sequence of SEQ ID NO: 26 and a VL sequence of SEQ ID NO: 27;
a VH sequence of SEQ ID NO: 28 and a VL sequence of SEQ ID NO: 29;
a VH sequence of SEQ ID NO: 30 and a VL sequence of SEQ ID NO: 31;
a VH sequence of SEQ ID NO: 32 and a VL sequence of SEQ ID NO: 33;
a VH sequence of SEQ ID NO: 34 and a VL sequence of SEQ ID NO: 35;
a VH sequence of SEQ ID NO: 36 and a VL sequence of SEQ ID NO: 37;
a VH sequence of SEQ ID NO: 38 and a VL sequence of SEQ ID NO: 39;
a VH sequence of SEQ ID NO: 40 and a VL sequence of SEQ ID NO: 41;
a VH sequence of SEQ ID NO: 42 and a VL sequence of SEQ ID NO: 43;
a VH sequence of SEQ ID NO: 44 and a VL sequence of SEQ ID NO: 45;
a VH sequence of SEQ ID NO: 46 and a VL sequence of SEQ ID NO: 47;
a VH sequence of SEQ ID NO: 48 and a VL sequence of SEQ ID NO: 49;
a VH sequence of SEQ ID NO: 50 and a VL sequence of SEQ ID NO: 51;
a VH sequence of SEQ ID NO: 52 and a VL sequence of SEQ ID NO: 53;
a VH sequence of SEQ ID NO: 58;
a VH sequence of SEQ ID NO: 59; and
a VH sequence of SEQ ID NO: 60.

The coronavirus S protein may be the spike protein of SARS-CoV-2 coronavirus.

Variants of the above amino acid sequences may also be used in the third polypeptide of the invention, provided that the resulting antibody binds coronavirus S protein. Typically, such variants have a high degree of sequence identity with one of the sequences specified above.

The terms “identity”, “identical” or “percent identity” have been described in the context of the first polypeptide of the invention and their definitions and particular features apply equally to the third polypeptide of the invention.

The third polypeptide of the invention may further comprise an additional domain ABD which comprises an antigen-binding domain that binds specifically to a coronavirus spike protein.

Steric hindrance may prevent domain ABD and the additional domain ABD from binding to coronavirus protein S at the same time. Thus, the antigen-binding domain of domain ABD and the antigen-binding domain of the additional domain ABD may bind to different epitopes on the coronavirus S protein.

The coronavirus S protein may be the S protein of SARS-CoV-2 Coronavirus.

Internal flexibility is also a factor contributing to multivalent binding. Thus, each of domain ABD, domain O, and additional domain ABD may be joined by a linker. The linker may be a flexible linker. The linkers described in the context of the fusion between domain A and domain O may be equally used herein. The linker may be the (Gly₄Ser)₃ linker (SEQ ID NO: 6).

The third polypeptide of the invention may comprise a domain C, a domain O, a domain ABD, and an additional domain ABD in any orientation. The skilled person will immediately recognise all the possible different domain structures (from N- to C-terminus).

The third polypeptide of the invention may be short-lived in the bloodstream or, otherwise, its pharmacokinetic properties may need to be enhanced. Plasma proteins and plasma protein binding can be an effective means of improving the pharmacokinetic properties of any molecule. One of these plasma proteins is albumin, which has been extensively investigated for extending the half-life of therapeutic molecules in blood.

Thus, the third polypeptide of the invention may further comprise a domain ALB which comprises an antigen-binding domain that binds specifically to albumin, or an albumin domain, or an antigen-binding domain that binds specifically to albumin, or an albumin-binding-peptide, or an albumin-binding domain of a Streptococcus protein.

The terms “albumin”, “antigen-binding domain that binds specifically to albumin”, “albumin-binding-peptide”, and “albumin-binding domain of a Streptococcus protein” have been described in the context of the first polypeptide of the invention and their definitions and particular features apply equally here.

Domain ALB may be joined by a linker. The linker may be a flexible linker. The linkers described in the context of the fusion between domain A and domain O may be equally used herein. The linker may be the (Gly₄Ser)₃ linker (SEQ ID NO: 6).

The third polypeptide of the invention may consist of the amino acid sequence shown as SEQ ID NO: 100 to 103.

SARS_aCoV_CR3022_scFv4-Ig_HL_
HC_mIgG2aFc
(SEQ ID NO: 100)
QMQLVQSGTEVKKPGESLKISCKGSGYGFITYWIGWVRQM
PGKGLEWMGIIYPGDSETRYSPSFQGQVTISADKSINTAY
LQWSSLKASDTAIYYCAGGSGISTPMDVWGQGTTVTVSSG
GGGSGGGGSGGGGSDIQLTQSPDSLAVSLGERATINCKSS
QSVLYSSINKNYLAWYQQKPGQPPKLLIYWASTRESGVPD
RFSGSGSGTDFTLTISSLQAEDVAVYYCQQYYSTPYTFGQ
GTKVEIKSSAKTTAPSVYPLAPVCGDTTGSSVTLGCLVKG
YFPEPVTLTWNSGSLSSGVHTFPAVLQSDLYTLSSSVTVT
SSTWPSQSITCNVAHPASSTKVDKKIEPRGPTIKPCPPCK
CPAPNLLGGPSVFIFPPKIKDVLMISLSPIVTCVVVDVSE
DDPDVQISWFVNNVEVHTAQTQTHREDYNSTLRVVSALPI
QHQDWMSGKEFKCKVNNKDLPAPIERTISKPKGSVRAPQV
YVLPPPEEEMTKKQVTLTCMVTDFMPEDIYVEWTNNGKTE
LNYKNTEPVLDSDGSYFMYSKLRVEKKNWVERNSYSCSVV
HEGLHNHHTTKSFSRTPGK
SARS_aCoV_CR3022_scFv4-Ig_HL_
LC_mKappa
(SEQ ID NO: 101)
QMQLVQSGTEVKKPGESLKISCKGSGYGFITYWIGWVRQM
PGKGLEWMGIIYPGDSETRYSPSFQGQVTISADKSINTAY
LQWSSLKASDTAIYYCAGGSGISTPMDVWGQGTTVTVSSG
GGGSGGGGSGGGGSDIQLTQSPDSLAVSLGERATINCKSS
QSVLYSSINKNYLAWYQQKPGQPPKLLIYWASTRESGVPD
RFSGSGSGTDFTLTISSLQAEDVAVYYCQQYYSTPYTFGQ
GTKVEIKADAAPTVSIFPPSSEQLTSGGASVVCFLNNFYP
KDINVKWKIDGSERQNGVLNSWTDQDSKDSTYSMSSTLTL
TKDEYERHNSYTCEATHKTSTSPIVKSFNRNEC
SARS_aCoV-S2_B1_scFv4-Ig_HL_
HC_mIgG2aFc
(SEQ ID NO: 102)
QVQLLESGAEVKKPGASAKVSCK
ASGCTFTSYGISWVRQAPGQGLEWMGWISAY
NGNTNYAQKLQGRVTMTTDTSTSTAYMELRSLRSDDTAVY
YCARGVAVAGTWDWFDPWGQGTLVTVSSGGGGSGGGGSGG
GGSDIVLTQTPGTLSLSPGERATLSCRASQSVRTYLAWYQ
QKPGQVPRLLIYGASSRATGIPDRFSGSGSGTDFTLTISR
LEPEDFAVYYCQQYGSSLWTFGQGTRLEIKRSSAKTTAPS
VYPLAPVCGDTTGSSVTLGCLVKGYFPEPVTLTWNSGSLS
SGVHTFPAVLQSDLYTLSSSVTVTSSTWPSQSITCNVAHP
ASSTKVDKKIEPRGPTIKPCPPCKCPAPNLLGGPSVFIFP
PKIKDVLMISLSPIVTCVVVDVSEDDPDVQISWFVNNVEV
HTAQTQTHREDYNSTLRVVSALPIQHQDWMSGKEFKCKVN
NKDLPAPIERTISKPKGSVRAPQVYVLPPPEEEMTKKQVT
LTCMVTDFMPEDIYVEWTNNGKTELNYKNTEPVLDSDGSY
FMYSKLRVEKKNWVERNSYSCSVVHEGLHNHHTTKSFSRT
PGK
SARS_aCoV-S2_B1_scFv4-Ig_
HL_LC_mKappa
(SEQ ID NO: 103)
QVQLLESGAEVKKPGASAKVSCKASGCTFTSYGISWVRQA
PGQGLEWMGWISAYNGNTNYAQKLQGRVTMTTDTSTSTAY
MELRSLRSDDTAVYYCARGVAVAGTWDWFDPWGQGTLVTV
SSGGGGSGGGGSGGGGSDIVLTQTPGTLSLSPGERATLSC
RASQSVRTYLAWYQQKPGQVPRLLIYGASSRATGIPDRFS
GSGSGTDFTLTISRLEPEDFAVYYCQQYGSSLWTFGQGTR
LEIKRADAAPTVSIFPPSSEQLTSGGASVVCFLNNFYPKD
INVKWKIDGSERQNGVLNSWTDQDSKDSTYSMSSTLTLTK
DEYERHNSYTCEATHKTSTSPIVKSFNRNEC

The third polypeptide of the invention may comprise a domain ABD, a domain O, a domain ALB, and, optionally, an additional domain ABD in any orientation. The skilled person will immediately recognise all the possible different domain structures (from N- to C-terminus).

Other modifications used to extend half-life that are currently known in the art, or that will be developed in the future, also form part of the present invention. For example, the first polypeptide of the invention may be conjugated to polyethylene glycol (PEG), or pegylated.

5. Signal Peptide

The first, second and third polypeptides of the present invention may comprise a signal peptide at their N-terminus so that when the polypeptide is expressed inside a cell, the nascent protein is directed to the endoplasmic reticulum and subsequently secreted.

The core of the signal peptide or leader sequence may contain a long stretch of hydrophobic amino acids that has a tendency to form a single alpha-helix. The signal peptide may begin with a short positively charged stretch of amino acids, which helps to enforce proper topology of the polypeptide during translocation. At the end of the signal peptide there is typically a stretch of amino acids that is recognised and cleaved by signal peptidase. Signal peptidase may cleave either during or after completion of translocation to generate a free signal peptide and a mature protein. The free signal peptides are then digested by specific proteases.

The signal peptide may be at the amino terminus of the molecule.

The signal peptide may comprise the SEQ ID NO: 55 to 57, 92 or 93 or a variant thereof having 5, 4, 3, 2 or 1 amino acid mutations (insertions, substitutions or additions) provided that the signal peptide still functions to cause cell surface expression of the protein.

SEQ ID NO: 55:
MGTSLLCWMALCLLGADHADG

The signal peptide of SEQ ID NO: 55 is compact and highly efficient. It is predicted to give about 95% cleavage after the terminal glycine, giving efficient removal by signal peptidase.

SEQ ID NO: 56:
MSLPVTALLLPLALLLHAARP

The signal peptide of SEQ ID NO: 56 is derived from IgG1.

SEQ ID NO: 57:
MAVPTQVLGLLLLWLTDARC

The signal peptide of SEQ ID NO: 57 is derived from CD8.

SEQ ID NO: 92:
METDTLLLWVLLLWVPGSTGD

The signal peptide of SEQ ID NO: 92 is derived from mouse Ig Kappa.

SEQ ID NO: 93:
MGWSCIILFLVATATGVHS

The signal peptide of SEQ ID NO: 93 is derived from human IgG2 heavy chain.

It will be appreciated that other signal peptides may be used for the purposes of this invention.

6. Nucleic Acid

In another aspect, the present invention also provides a nucleic acid encoding the first polypeptide of the invention, the second polypeptide of the invention or the third polypeptide of the invention, hereinafter “the nucleic acid of the invention”.

The nucleic acid of the invention may have one of the following structures:
a) where the nucleic acid encodes the first polypeptide of the invention:
A-O
or
A-C-O
or
A-ABD-O
or
A-C-ABD-O
or
A-O-ALB
or
A-C-O-ALB
or
A-ABD-O-ALB
or
A-C-ABD-O-ALB
b) where the nucleic acid encodes the second polypeptide of the invention:
C-O
or
C-ABD-O
or
C-O-ALB
or
C-ABD-O-ALB
c) where the nucleic acid encodes the third polypeptide of the invention:
ABD-O
or
ABD-O-ALB
in which:
   A is a nucleic acid encoding a domain A;
   O is a nucleic acid encoding the homooligomerisation domain;
   coexpr is a nucleic acid encoding a sequence enabling co-expression of the first and second polypeptides;
   C is a nucleic acid encoding a domain C;
   ABD is a nucleic acid encoding a domain ABD;
   ALB is a nucleic acid encoding a domain ALB.

These nucleic acid structures may optionally encode a spacer between any of domains, A, O, C, ABD and/or ALB. The skilled person will immediately recognise the nucleic acid structures that are needed to encode all the embodiments of the first, second and third polypeptide of the invention. For example, the nucleic acid of the invention may have one of the following structures:
a) where the nucleic acid encodes the first polypeptide of the invention:
A-spacer-O
or
A-spacer-C-spacer-O
or
A-spacer-ABD-spacer-O
or
A-spacer-C-spacer-ABD-spacer-O
or
A-spacer-O-spacer-ALB
or
A-spacer-C-spacer-O-spacer-ALB
or
A-spacer-ABD-spacer-O-spacer-ALB
or
A-spacer-C-spacer-ABD-spacer-O-spacer-ALB
b) where the nucleic acid encodes the second polypeptide of the invention:
C-spacer-O
or
C-spacer-ABD-spacer-O
or
C-spacer-O-spacer-ALB
or
C-spacer-ABD-spacer-O-spacer-ALB
c) where the nucleic acid encodes the third polypeptide of the invention:
ABD-spacer-O
or
ABD-spacer-O-spacer-ALB
in which:
   A is a nucleic acid encoding a domain A;
   spacer is a nucleic acid encoding a spacer;
   O is a nucleic acid encoding the homooligomerisation domain;
   coexpr is a nucleic acid encoding a sequence enabling co-expression of the first and second polypeptides;
   C is a nucleic acid encoding a domain C;
   ABD is a nucleic acid encoding a domain ABD;
   ALB is a nucleic acid encoding a domain ALB.

In particular embodiments where the domain O comprises a heterooligomeric domain (e.g. the polynucleotides shown in FIG. 3, right-two panels, and FIG. 6B), the nucleic acid construct encoding such a polynucleotide may have one of the following structures:
a) where the nucleic acid encodes the first polypeptide of the invention:
A-O1-coexpr-A-O2
or
A-C-O1-coexpr-A-C-O2
or
A-ABD1-O1-coexpr-A-ABD2-O2
or
A-C-ABD1-O1-coexpr-A-C-ABD2-O2
or
A-O1-ALB-coexpr-A-O2-ALB
or
A-C-O1-ALB-coexpr-A-C-O2-ALB
or
A-ABD1-O1-ALB-coexpr-A-ABD2-O2-ALB
or
A-C-ABD1-O1-ALB-coexpr-A-C-ABD2-O2-ALB
b) where the nucleic acid encodes the second polypeptide of the invention:
C-O1-coexpr-C-O2
or
C-ABD1-O1-coexpr-C-ABD2-O2
or
C-O1-ALB-coexpr-C-O2-ALB
or
C-ABD1-O1-ALB-coexpr-C-ABD2-O2-ALB
c) where the nucleic acid encodes the third polypeptide of the invention:
ABD1-O1-coexpr-ABD2-O2
or
ABD1-O1-ALB-coexpr-ABD2-O2-ALB
in which:
   A is a nucleic acid encoding a domain A;
   O1 is a nucleic acid encoding one of the heterooligomers;
   O2 is a nucleic acid encoding another one of the heterooligomers;
   coexpr is a nucleic acid encoding a sequence enabling co-expression of the first and second polypeptides;
   C is a nucleic acid encoding a domain C;
   ABD1 is a nucleic acid encoding a domain ABD;
   ABD2 is a nucleic acid encoding a domain ABD of different specificity to the one of ABD1;
   ALB is a nucleic acid encoding a domain ALB.

These nucleic acid structures may optionally encode a spacer between any of domains A, O, C, ABD and/or ALB. The skilled person will immediately recognise the nucleic acid structures that are needed to encode all the embodiments of the first, second and third polypeptide of the invention. For example, the nucleic acid of the invention may have one of the following structures:
a) where the nucleic acid encodes the first polypeptide of the invention:
A-spacer1-O1-coexpr-A-spacer2-O2
or
A-spacer1-C-spacer2-O1-coexpr-A-spacer3-C-spacer4-O2
or
A-spacer1-ABD1-spacer2-O1-coexpr-A-spacer3-ABD2-spacer4-O2
or
A-spacer1-C-spacer2-ABD1-spacer3-O1-coexpr-A-spacer4-C-spacer5-ABD2-spacer6-O2
or
A-spacer1-01 spacer2-ALB-coexpr-A-spacer3-O2-spacer 4-ALB
or
A-spacer1-C-spacer2-O1-spacer3-ALB-coexpr-A-spacer4-C-spacer5-O2-spacer6-ALB
or
A-spacer1-ABD1-spacer2-O1-spacer3-ALB-coexpr-A-spacer4-ABD2-spacer5-O2-spacer6-ALB
or
A-spacer1-C-spacer2-ABD1-spacer3-O1-spacer4-ALB-coexpr-A-spacer5-C-spacer6-ABD2-spacer7-O2-spacer8-ALB
b) where the nucleic acid encodes the second polypeptide of the invention:
C-spacer1-O1-coexpr-C-spacer2-O2
or
C-spacer1-ABD1-spacer2-O1-coexpr-C-spacer 3-ABD2-spacer4-O2
or
C-spacer1-O1-spacer2-ALB-coexpr-C-spacer3-O2-spacer4-ALB
or
C-spacer1-ABD1-spacer2-O1-spacer3-ALB-coexpr-C-spacer4-ABD2-spacer5-O2-spacer6-ALB
c) where the nucleic acid encodes the third polypeptide of the invention:
ABD1-spacer1-O1-coexpr-ABD2-spacer2-O2
or
ABD1-spacer1-O1-spacer2-ALB-coexpr-ABD2-spacer3-O2-spacer4-ALB
in which:
   A is a nucleic acid encoding a domain A;
   Spacer1, spacer2 and so on, which may be the same or different, are nucleic acids encoding a spacer1, spacer2 and so on;
   O1 is a nucleic acid encoding one of the heterooligomers;
   O2 is a nucleic acid encoding another one of the heterooligomers;
   coexpr is a nucleic acid encoding a sequence enabling co-expression of the first and second polypeptides;
   C is a nucleic acid encoding a domain C;
   ABD1 is a nucleic acid encoding a domain ABD;
   ABD2 is a nucleic acid encoding a domain ABD of different specificity to the one of ABD1;
   ALB is a nucleic acid encoding a domain ALB.

These structures encode heterodimers, but they can be easily adapted to higher forms of heterooligomers (e.g. heterotrimers, heterotetramers, and so on).

For the three groups of structures mentioned above, nucleic acid sequences encoding the two polypeptides may be in either order in the construct. Additionally, for the three groups of structures mentioned above, nucleic acid sequences encoding each of the different domains may be in any orientation.

There is also provided a nucleic acid construct encoding a third polypeptide of the invention such as the one shown in FIG. 6C. This nucleic acid may have the structure:
ABD1-spacer-CH1-hinge-CH2-CH3-coexpr-ABD2-CL
in which:
   ABD1 is a nucleic acid encoding a domain ABD;
   ABD2 is a nucleic acid encoding a domain ABD of different specificity to the one of ABD1;
   CH1 is a nucleic acid encoding a heavy chain constant domain 1;
   hinge is a nucleic acid encoding a hinge region;
   CH2 is a nucleic acid encoding a heavy chain constant domain 2;
   CH3 is a nucleic acid encoding a heavy chain constant domain 3
   CL is a nucleic acid encoding a light chain constant domain;
   spacer is a nucleic acid encoding a spacer or linker;
   hinge is a nucleic acid encoding a hinge region;
   coexpr is a nucleic acid encoding a sequence enabling co-expression of the first and second polypeptides.

For this structure, nucleic acid sequences encoding the two polypeptides forming the third polypeptide of the invention may be in any order in the construct.

The nucleic acid structures described above are not limiting in any way and, thus, the nucleic acid of the invention may have other different structures. The skilled person will immediately recognise the nucleic acid structures that are needed to encode all the embodiments of the first, second and third polypeptide of the invention.

The terms “first polypeptide of the invention”, “second polypeptide of the invention” and “third polypeptide of the invention” have been described in detail in the context of previous aspects of the invention and its features and embodiments apply equally to this aspect of the invention.

As used herein, the terms “polynucleotide”, “nucleotide”, and “nucleic acid” are intended to be synonymous with each other.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described here to reflect the codon usage of any particular host organism in which the polypeptides are to be expressed.

The nucleic acid sequences and constructs of the invention may contain alternative codons in regions of sequence encoding the same or similar amino acid sequences, in order to avoid homologous recombination.

Nucleic acids according to the invention may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the use as described herein, it is to be understood that the polynucleotides may be modified by any method available in the art. Such modifications may be carried out in order to enhance the in vivo activity or life span of polynucleotides of interest.

The terms “variant”, “homologue” or “derivative” in relation to a nucleotide sequence include any substitution of, variation of, modification of, replacement of, deletion of or addition of one (or more) nucleic acid from or to the sequence.

In the structure above, “coexpr” is a nucleic acid sequence enabling co-expression of two polypeptides as separate entities. It may be a sequence encoding a cleavage site, such that the nucleic acid construct produces both polypeptides, joined by a cleavage site(s). The cleavage site may be self-cleaving, such that when the polypeptide is produced, it is immediately cleaved into individual peptides without the need for any external cleavage activity.

The cleavage site may be any sequence which enables the two polypeptides to become separated.

The term “cleavage” is used herein for convenience, but the cleavage site may cause the peptides to separate into individual entities by a mechanism other than classical cleavage. For example, for the Foot-and-Mouth disease virus (FMDV) 2A self-cleaving peptide (see below), various models have been proposed for to account for the “cleavage” activity: proteolysis by a host-cell proteinase, autoproteolysis or a translational effect (Donnelly et al (2001) J. Gen. Virol. 82:1027-1041). The exact mechanism of such “cleavage” is not important for the purposes of the present invention, as long as the cleavage site, when positioned between nucleic acid sequences which encode proteins, causes the proteins to be expressed as separate entities.

The cleavage site may, for example be a furin cleavage site, a Tobacco Etch Virus (TEV) cleavage site or encode a self-cleaving peptide.

A ‘self-cleaving peptide’ refers to a peptide which functions such that when the polypeptide comprising the proteins and the self-cleaving peptide is produced, it is immediately “cleaved” or separated into distinct and discrete first and second polypeptides without the need for any external cleavage activity.

The self-cleaving peptide may be a 2A self-cleaving peptide from an aphtho- or a cardiovirus. The primary 2A/2B cleavage of the aptho- and cardioviruses is mediated by 2A “cleaving” at its own C-terminus. In apthoviruses, such as foot-and-mouth disease viruses (FMDV) and equine rhinitis A virus, the 2A region is a short section of about 18 amino acids, which, together with the N-terminal residue of protein 2B (a conserved proline residue) represents an autonomous element capable of mediating “cleavage” at its own C-terminus (Donelly et al (2001) as above).

“2A-like” sequences have been found in picornaviruses other than aptho- or cardioviruses, ‘picornavirus-like’ insect viruses, type C rotaviruses and repeated sequences within Trypanosoma spp and a bacterial sequence (Donnelly et al (2001) as above).

The cleavage site may comprise the 2A-like sequence shown as SEQ ID No.140 (RAEGRGSLLTCGDVEENPGP).

Alternatively, where the domain O comprises a heterooligomeric domain the different monomers may be encoded by separate nucleic acids.

The nucleic acid of the invention can contain a regulatory sequence operatively linked for the expression of the nucleotide sequence encoding the first polypeptide of the invention, thereby forming a gene construct, hereinafter the “gene construct of the invention”. As used herein, the term “operatively linked” means that the antibody encoded by the nucleic acid sequence of the invention is expressed in the correct reading frame under control of the expression control or regulating sequences. Therefore, in another aspect, the invention provides an expression cassette, hereinafter “the expression cassette of the invention”, comprising the gene construct of the invention operatively linked to an expression control sequence. The gene construct of the invention can be obtained through the use of techniques that are widely known in the art.

The expression cassette may comprise one or more control sequences. Control sequences are sequences that control and regulate transcription and, where appropriate, the translation of said antibody, and include promoter sequences, transcriptional regulators encoding sequences, ribosome binding sequences (RBS) and/or transcription terminating sequences. The expression cassette of the present invention may additionally include an enhancer, which may be adjacent to or distant from the promoter sequence and can function to increase transcription from the same. The expression control sequence may functional in prokaryotic cells or in eukaryotic cells and organisms, such as mammalian cells. The expression cassette may comprise a promoter. Any promoter may be used in this methodology.

It will be appreciated that different nucleic acids are required to encode the first, second or third polypeptides of the invention where the domain O comprises a hetero oligomerisation domain. The skilled person will readily know how to make the necessary modifications to obtain the different nucleic acids encoding for these heterooligomeric first, second or third polypeptides of the invention.

7. Vector

In another aspect, the present invention also provides a vector, or kit of vectors, which comprises a nucleic acid of the invention, or an expression cassette of the invention. Such a vector may be used to introduce the nucleic acid or expression cassette into a host cell so that it expresses the first polypeptide of the invention, the second polypeptide of the invention or the third polypeptide of the invention.

The terms “first polypeptide of the invention”, “second polypeptide of the invention”, “third polypeptide of the invention”, “nucleic acid of the invention” and “expression cassette or the invention” have been described in detail in the context of previous aspects of the invention and their features and embodiments apply equally to this aspect of the invention.

The vector may, for example, be a plasmid or a viral vector, such as a retroviral vector or a lentiviral vector, or a transposon-based vector or synthetic mRNA.

8. Cell

Another aspect of the present invention relates to a cell, hereinafter “the cell of the invention”, comprising the nucleic acid of the invention, the nucleic acid of the invention, the expression cassette of the invention, the vector of the invention, or the vector of the invention.

The cell may comprise a nucleic acid, or an expression cassette, or a vector according to the present invention.

The term “first polypeptide of the invention”, “second polypeptide of the invention”, “third polypeptide of the invention”, “nucleic acid of the invention”, “expression cassette or the invention”, and “vector of the invention” have been described in detail in the context of previous aspects of the invention and their features and embodiments apply equally to this aspect of the invention.

The cell may be prokaryotic or eukaryotic.

Cells suitable for performing the invention include, without limitation, mammalian, plant, insect, fungal and bacterial cells. Mammalian cells suitable for the present invention include epithelial cell lines, osteosarcoma cell lines, neuroblastoma cell lines, epithelial carcinomas, glial cells, hepatic cell lines, CHO (Chinese Hamster Ovary) cells, COS, BHK cells, HeLa cells, 911 cells, AT1080 cells, A549 cells, 293 and 293T cells, PER.C6 cells, NTERA-2 human ECCs cells, D3 cells of the mESCs line, human embryonic stem cells such as HS293, hMSCs and BGVO1, SHEF1, SHEF2 and HS181, NIH3T3 cells, REH and MCF-7 cells. Bacterial cells include, without limitation, cells from Gram positive bacteria such as species of the genus Bacillus, Streptomyces and Staphylococcus and Gram-negative bacterial cells such as cells of the genus Escherichia and Pseudomonas. Fungal cells preferably include yeast cells such as Saccharomyces, Pichia pastoris and Hansenula polymorpha. Insect cells include, without limitation, Drosophila cells and Sf9 cells. Plant cells include, among others, cells of crop plants such as cereals, medicinal, ornamental or bulbs.

The first, second or third polypeptide of the invention may be produced by culturing the host cells for a period of time sufficient to allow for expression of the polypeptide in the host cells or, more preferably, secretion of the polypeptide into the culture medium in which the host cells are grown. The first, second or third polypeptide of the invention can be recovered from the culture medium using standard protein purification methods.

Thus, the present invention also relates to a method for making the first, second or third polypeptide of the invention by culturing a cell of the invention and purifying the polypeptide from the supernatant.

9. Pharmaceutical Composition

In another aspect, the present invention also relates to a pharmaceutical composition containing the first polypeptide of the invention, the second polypeptide of the invention, the third polypeptide of the invention, the nucleic acid of the invention, the expression cassette of the invention, the vector of the invention, or the cell of the invention, hereinafter “the pharmaceutical composition of the invention”.

The pharmaceutical composition may additionally comprise a pharmaceutically acceptable carrier, diluent, excipient, or adjuvant. The pharmaceutical composition may optionally comprise one or more further pharmaceutically active polypeptides and/or compounds. Such a formulation may, for example, be in a form suitable for intravenous infusion.

The terms “first polypeptide of the invention”, “second polypeptide of the invention”, “third polypeptide of the invention”, “nucleic acid of the invention”, “expression cassette of the invention”, “vector of the invention”, or “cell of the invention” have been described in detail in the context of previous aspects of the invention and their definitions and particular features apply equally to this aspect of the invention.

As used herein, “pharmaceutically acceptable carrier” means any and all solvents, dispersion media, coatings, antibacterial and antifungal agents, isotonic and absorption delaying agents, and the like that are physiologically compatible. Some examples of pharmaceutically acceptable carriers are water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Additional examples of pharmaceutically acceptable substances are wetting agents or minor amounts of auxiliary substances such as wetting or emulsifying agents, preservatives or buffers, which enhance the shelf life or effectiveness of the first, second or third polypeptide of the invention.

The pharmaceutical compositions may be in a variety of forms, for example, liquid, semi-solid and solid dosage forms, such as liquid solutions (e.g. injectable and infusible solutions), dispersions or suspensions, tablets, pills, powders, and liposomes. The preferred form depends on the intended mode of administration and therapeutic application.

Administration

The administration of the first, second or third polypeptide of the invention, the nucleic acid of the invention, the expression cassette of the invention, the vector of the invention, or the cell of the invention can be accomplished using any of a variety of routes that make the active ingredient bioavailable. For example, the agent can be administered via an oral, mucosal, buccal, intranasal, inhalable, intravenous, subcutaneous, transcutaneous, intramuscular, intraperitoneal, parenteral or topical route. Oral administration may be by inhalation, by nebulisation or nasally. The first, second or third polypeptide of the invention may be administered locally, for example by catheter or stent, or systemically.

The pharmaceutical compositions comprising the first, second or third polypeptide of the invention, the nucleic acid of the invention, the expression cassette of the invention, the vector of the invention, or the cell of the invention may be administered to the subject in a variety of pharmaceutically acceptable dosing forms, which will be familiar to those skilled in the art.

For example, the first, second or third polypeptide of the invention, the nucleic acid of the invention, the expression cassette of the invention, the vector of the invention, or the cell of the invention may be administered via the nasal route using a nasal insufflator device. Examples of these are already employed for commercial powder systems intended for nasal application (e.g. Fisons Lomudal System). Details of other devices are well-known in the art.

Other delivery routes for the first, second or third polypeptide of the invention, the nucleic acid of the invention, the expression cassette of the invention, the vector of the invention, or the cell of the invention include via the pulmonary route using a powder inhaler or metered dose inhaler, via the buccal route formulated into a tablet or a buccal patch, and via the oral route in the form of a tablet, a capsule or a pellet (which compositions may administer agent via the stomach, the small intestine or the colon), all of which may be formulated in accordance with techniques which are well known to those skilled in the art.

Typically, a physician will determine the actual dosage which will be most suitable for an individual subject and it will vary with the age, weight and response of the particular patient.

10. Method of Treatment

The specificity for the coronavirus S protein of the first, second and third polypeptides of the invention makes these molecules capable of neutralising coronavirus virions and prevent cell infection. This can be exploited for therapeutic purposes.

Thus, in another aspect, the present invention provides a first polypeptide of the invention, a second polypeptide of the invention or a third polypeptide of the invention for use in medicine.

The invention provides a method for neutralising a coronavirus by administering a first, second or third polypeptide of the invention to a patient in need thereof. The coronavirus may be SARS-CoV-2 or SARS-CoV. The SARS-CoV-2 strain may be selected from wild-type, variant D614G, variant A222V, variant S477N, variant B.1.1.7, variant B.1.351, and variant B.1.1.28.

In another aspect, the present invention provides a method of treating a coronavirus infection or a condition or disorder resulting from this infection in a subject, hereinafter “the method of treatment of the invention”, which comprises the step of administering a first polypeptide of the invention, a second polypeptide of the invention or a third polypeptide of the invention to a subject in need thereof. The administration step may be in the form of a pharmaceutical composition as described above.

This aspect of the invention may be alternatively formulated as a first polypeptide of the invention, a second polypeptide of the invention or a third polypeptide of the invention for use in the treatment of a coronavirus infection or a condition or disorder resulting from this infection.

This aspect of the invention may be alternatively formulated as the use of a first polypeptide of the invention, a second polypeptide of the invention or a third polypeptide of the invention in the manufacture of a medicament for treating a coronavirus infection or a condition or disorder resulting from this infection.

The terms “first polypeptide of the invention”, “second polypeptide of the invention” and “third polypeptide of the invention” have been described in detail in the context of previous aspects of the invention and their definitions and particular features apply equally to this aspect of the invention.

A method for treating a coronavirus infection or a condition or disorder resulting from this infection relates to the therapeutic use of the first, second or third polypeptide of the invention, which may be administered to a subject who has been infected with a coronavirus, or is suspected to have been infected with a coronavirus, or has tested positive for a coronavirus in order to lessen, reduce or improve at least one symptom associated with the disease and/or to slow down, reduce or block the progression of the disease.

The method for preventing a coronavirus infection or a condition or disorder resulting from this infection relates to the prophylactic use of the first, second or third polypeptide of the invention. Herein such polypeptide may be administered to a subject who has not yet contracted the coronavirus infection or condition or disorder resulting from this infection and/or who is not showing any symptoms of the coronavirus infection or condition or disorder resulting from this infection to prevent or impair the coronavirus from infecting the cells of the subject or to reduce or prevent development of at least one symptom associated with the coronavirus infection or condition or disorder resulting from this infection. The subject may have a predisposition for or be thought to be at risk of contracting a coronavirus infection or a condition or disorder resulting from this infection.

In another aspect, the present invention provides a method for treating a subject having COVID-19 of unknown SARS-CoV-2 strain, comprising a step of administering a first polypeptide of the invention, a second polypeptide of the invention or a third polypeptide of the invention, or a pharmaceutical composition of the invention to the subject. This aspect may be alternatively formulated as a first polypeptide of the invention, a second polypeptide of the invention, a third polypeptide of the invention, or a pharmaceutical composition of the invention for use in the treatment of COVID-19 of unknown SARS-CoV-2 strain. This aspect may be alternatively formulated as the use of a first polypeptide of the invention, a second polypeptide of the invention, a third polypeptide of the invention, or a pharmaceutical composition of the invention in the manufacture of a medicament for treating COVID-19 of unknown SARS-CoV-2 strain.

In another aspect, the present invention provides a method for treating a subject previously immunised with a vaccine based on S protein depicted under Uniprot accession number P0DTC2, comprising a step of administering a first polypeptide of the invention, a second polypeptide of the invention or a third polypeptide of the invention, or a pharmaceutical composition of the invention to the subject. This aspect may be alternatively formulated as a first polypeptide of the invention, a second polypeptide of the invention, a third polypeptide of the invention, or a pharmaceutical composition of the invention for use in the treatment of a subject previously immunised with a vaccine based on S protein depicted under Uniprot accession number P0DTC2. This aspect may be alternatively formulated as the use of a first polypeptide of the invention, a second polypeptide of the invention, a third polypeptide of the invention, or a pharmaceutical composition of the invention in the manufacture of a medicament for treating a subject previously immunised with a vaccine based on S protein depicted under Uniprot accession number P0DTC2.

In another aspect, the present invention provides a method for treating a subject previously treated with antibodies specific to S protein depicted under Uniprot accession number P0DTC2, comprising a step of administering a first polypeptide of the invention, a second polypeptide of the invention or a third polypeptide of the invention, or a pharmaceutical composition of the invention to the subject. This aspect may be alternatively formulated as a first polypeptide of the invention, a second polypeptide of the invention, a third polypeptide of the invention, or a pharmaceutical composition of the invention for use in the treatment of a subject previously treated with antibodies specific to S protein depicted under Uniprot accession number P0DTC2. This aspect may be alternatively formulated as the use of a first polypeptide of the invention, a second polypeptide of the invention, a third polypeptide of the invention, or a pharmaceutical composition of the invention in the manufacture of a medicament for treating a subject previously treated with antibodies specific to S protein depicted under Uniprot accession number P0DTC2.

In another aspect, the present invention provides a method for treating a subject previously infected with a first SARS-CoV-2 strain who is currently infected with a second SARS-CoV-2 strain, wherein the first and second SARS-CoV-2 strains are different, comprising a step of administering a first polypeptide of the invention, a second polypeptide of the invention or a third polypeptide of the invention, or a pharmaceutical composition of the invention to the subject. This aspect may be alternatively formulated as a first polypeptide of the invention, a second polypeptide of the invention, a third polypeptide of the invention, or a pharmaceutical composition of the invention for use in the treatment of a subject previously infected with a first SARS-CoV-2 strain who is currently infected with a second SARS-CoV-2 strain, wherein the first and second SARS-CoV-2 strains are different. This aspect may be alternatively formulated as the use of a first polypeptide of the invention, a second polypeptide of the invention, a third polypeptide of the invention, or a pharmaceutical composition of the invention in the manufacture of a medicament for treating a subject previously infected with a first SARS-CoV-2 strain who is currently infected with a second SARS-CoV-2 strain, wherein the first and second SARS-CoV-2 strains are different.

The first and second SARS-CoV-2 strain may be selected from wild-type, variant D614G, variant A222V, variant S477N, variant B.1.1.7, variant B.1.351, and variant B.1.1.28. It will be appreciated that this aspect is not limited to the SARS-CoV-2 strains described above since the present invention is useful in the treatment of any other SARS-CoV-2 variants existent at the time of filing or of any future variants that may emerge.

In another aspect, the present invention provides a method for treating a coronavirus infection of one SARS-CoV-2 strain selected from wild-type, variant D614G, variant A222V, variant S477N, variant B.1.1.7, variant B.1.351, and variant B.1.1.28, comprising a step of administering a first polypeptide of the invention, a second polypeptide of the invention or a third polypeptide of the invention, or a pharmaceutical composition of the invention to the subject. This aspect may be alternatively formulated as a first polypeptide of the invention, a second polypeptide of the invention, a third polypeptide of the invention, or a pharmaceutical composition of the invention for use in the treatment of a coronavirus infection of one SARS-CoV-2 strain selected from wild-type, variant D614G, variant A222V, variant S477N, variant B.1.1.7, variant B.1.351, and variant B.1.1.28. This aspect may be alternatively formulated as the use of a first polypeptide of the invention, a second polypeptide of the invention, a third polypeptide of the invention, or a pharmaceutical composition of the invention in the manufacture of a medicament for treating a coronavirus infection of one SARS-CoV-2 strain selected from wild-type, variant D614G, variant A222V, variant S477N, variant B.1.1.7, variant B.1.351, and variant B.1.1.28.

It will be appreciated that this aspect is not limited to the SARS-CoV-2 strains described above since the present invention is useful in the treatment of any other SARS-CoV-2 variants existent at the time of filing or of any future variants that may emerge.

The terms “first polypeptide of the invention”, “second polypeptide of the invention” and “third polypeptide of the invention” have been described in detail in the context of previous aspects of the invention and their definitions and particular features apply equally to this aspect of the invention.

These therapeutic applications will comprise the administration of a therapeutically effective amount of the first, second or third polypeptide of the invention. The administration step may be in the form of a pharmaceutical composition as described above.

The treatment of a coronavirus disease in a subject may comprise the step of administrating the first, second or third polypeptide of the invention to the subject, to cause complete or partial neutralisation of the coronaviruses.

In another aspect, the present invention provides a method of neutralising a coronavirus infection, comprising a step of contacting a first polypeptide of the invention, a second polypeptide of the invention or a third polypeptide of the invention with a cell infected with said coronavirus. The first, second or third polypeptides of the invention may be in the form of a pharmaceutical composition as described above.

The terms “first polypeptide of the invention”, “second polypeptide of the invention” and “third polypeptide of the invention” have been described in detail in the context of previous aspects of the invention and their definitions and particular features apply equally to this aspect of the invention.

The term “subject” or “individual”, as used in the context of the present invention, refers to members of mammalian species. The subject may be a human patient of any gender, age or race.

Alternatively, the subject may be a non-human mammal infected with coronavirus. The first, second or third polypeptide of the invention may be administered to a non-human mammal infected with coronavirus for veterinary purposes or as an animal model of human disease. Such animal models may be useful for evaluating the therapeutic efficacy of the polypeptides of this invention. Non-limiting examples of non-human mammal that may be subject to treatment according to the invention include a cat or any other feline, a dog or any other canid, a mouse, a hamster, a rat or any other rodent, a pig, a primate, and a bat.

The term “therapeutically effective amount”, as used herein, refers to the amount of the first, second or third polypeptide of the invention which is required to achieve an appreciable prevention, neutralisation, cure, delay, reduction of the severity of, or amelioration of one or more symptoms of a coronavirus disease.

The term “a coronavirus infection or a condition or disorder resulting from this infection”, as used herein, refers to an infection, condition or disorder caused by a coronavirus. The coronaviruses can cause varieties of diseases in humans and other animals, including respiratory, enteric, renal, and neurological diseases. Particularly important are the diseases caused by SARS-CoV and SARS-CoV-2 coronaviruses because of the severe acute respiratory syndrome that they cause.

The coronavirus condition or disorder may be coronavirus disease 2019 (COVID-19). The disease was first identified in December 2019 in Wuhan, the capital of China's Hubei province, and has since spread globally, resulting in the ongoing 2019-20 coronavirus pandemic. Common symptoms include fever, cough and shortness of breath. Other symptoms may include fatigue, muscle pain, diarrhoea, nausea, sore throat, loss of smell and abdominal pain. While the majority of cases result in mild symptoms, some progress to viral pneumonia and multi-organ failure.

Reports have shown that COVID-19 manifests as a clotting disorder, which may cause pulmonary embolism and hypoxia. Pulmonary vasculature affected by pulmonary embolism is not fully restored and can cause permanent fibrosis of the lining of blood vessels. Pulmonary fibrosis may also be the result of prolonged mechanical ventilation; even prolonged use of high concentration oxygen can lead to lung injury and result in fibrosis. Permanent fibrosis may lead to chronic thromboembolic pulmonary hypertension (CTEPH). Additionally, the clotting disorder causes end organ damage, primarily kidney. Kidney injury does not fully recover and may lead to chronic kidney disease (CKD) in post-COVID19 patients.

Direct infection of SARS-CoV-2 of ACE2-expressing cells has a number of consequences. Infection of the heart muscle cells leads to myocarditis. Patients who have no or minimal pulmonary symptoms but presented with fatigue may experience myocarditis as the primary disease. Myocardial injury may also explain the increase incidence of cardiac arrest in COVID-19 patients. Because ACE2 receptors play a key role in the renin-angiotensin system, which is a primary regulatory mechanism for blood pressure, viral infection of ACE2-expressing cells may lead to malfunction of the system and increased blood pressure.

Severe COVID-19 presents with a cytokine storm or cytokine release syndrome (CRS), which is an immediate and intense response of the immune system to viral infection. However, there are indications that the immune response may not just be temporary. One example is the case reports of Kawasaki disease symptoms in children infected with SARS-CoV-2. Kawasaki disease is an autoimmune disease in which blood vessels throughout the body become inflamed. It is considered a “post-viral” autoimmune disease. Several reports have described COVID-19 patients suffering from Guillain-Barre syndrome Guillain-Barre syndrome is a neurological disorder where the immune system responds to an infection and ends up mistakenly attacking nerve cells, resulting in muscle weakness and eventually paralysis. Thus, severe COVID-19 may also cause an incidence of other more prevalent autoimmune diseases in recovered patients.

The loss of the sense of smell is a direct result of the virus infecting the olfactory neurons. It has been suggested that this may enable the virus to spread from the respiratory tract to the brain. Cells in the human brain express the ACE2 protein on their surface. ACE2 is also found on endothelial cells that line blood vessels. Infection of endothelial cells may allow the virus to pass from the respiratory tract to the blood and then across the blood-brain barrier into the brain. Once in the brain, replication of the virus may cause neurological disorders. Larger studies from China and France have also investigated the prevalence of neurological disorders in COVID-19 patients. These studies have shown that 36% of patients have neurological symptoms. Many of these symptoms were mild and include headache or dizziness that could be caused by a robust immune response. Other more specific and severe symptoms were also seen and include loss of smell or taste, muscle weakness, stroke, seizure and hallucinations. Case studies have described severe COVID-19 encephalitis and stroke in healthy young people with otherwise mild COVID-19 symptoms. These symptoms are seen more often in severe cases, with estimates ranging from 46% to 84% of severe cases showing neurological symptoms. Changes in consciousness, such as disorientation, inattention and movement disorders, were also seen in severe cases and found to persist after recovery. Therefore, brain inflammation in severe COVID-19 might also indirectly cause neurological damage, such as through brain swelling, which is associated with neurodegenerative diseases.

The virus is mainly spread during close contact and by small droplets produced when those infected cough, sneeze or talk. These small droplets may also be produced during breathing, but rapidly fall to the ground or surfaces and are not generally spread through the air over large distances. People may also become infected by touching a contaminated surface and then their face. The virus can survive on surfaces for up to 72 hours. It is most contagious during the first three days after onset of symptoms, although spread may be possible before symptoms appear and in later stages of the disease. The time from exposure to onset of symptoms is typically around five days, but may range from two to 14 days. The standard method of diagnosis is by real-time reverse transcription polymerase chain reaction (RT-PCR) or by lateral flow test (commonly known as rapid antigen test) from a nasopharyngeal swab. The infection can also be diagnosed from a combination of symptoms, risk factors and a chest CT scan showing features of pneumonia.

Currently, there is no vaccine or specific antiviral treatment for COVID-19. Management involves treatment of symptoms, supportive care, isolation and experimental measures.

If any of the above-mentioned effects of COVID-19 is related to the immune response, and not only to the viral infection itself, then there is a risk that it may also be triggered by a vaccine, since a vaccine could produce the same immune responses. Therefore, the method of treatment of the present invention poses a significant advantage over vaccine and other antibody-mediated therapeutic approaches.

The method of treating a coronavirus infection or a condition or disorder resulting from this infection in a subject according to the invention, or the method of neutralising a coronavirus infection according to the invention may comprise a step of administering the first, the second or the third polypeptide of the invention to the subject. The skilled person will be able to determine by conventional methods the amount of the polypeptide of the invention that are able to exert a therapeutic effect on the patient.

The first, the second or the third polypeptide of the invention may be administered once, but it may be administered multiple times. The first, the second or the third polypeptide of the invention may be administered from three times daily to once every six months or longer. The administering may be on a schedule such as three times daily, twice daily, once daily, once every two days, once every three days, once weekly, once every two weeks, once every month, once every two months, once every three months and once every six months. The first, the second or the third polypeptide of the invention may also be administered continuously via a minipump. The first, the second or the third polypeptide of the invention may be administered via an oral, mucosal, buccal, intranasal, inhalable, intravenous, subcutaneous, intramuscular, parenteral, or topical route. The first, the second or the third polypeptide of the invention may be administered locally or systemically.

The first, the second or the third polypeptide of the invention may be administered once, at least twice or for at least the period of time until the condition is treated, palliated or cured. The first, the second or the third polypeptide of the invention will generally be administered as part of a composition as described supra. The dosage of polypeptide of the invention will generally be in the range of 0.1-100 mg/kg, more preferably 0.5-50 mg/kg, more preferably 1-20 mg/kg, and even more preferably 1-10 mg/kg. The serum concentration of the polypeptide of the invention may be measured by any method known in the art.

The invention will now be further described by way of Examples, which are meant to serve to assist one of ordinary skill in the art in carrying out the invention and are not intended in any way to limit the scope of the invention.

### EXAMPLES

### Example 1: Generation of Fusion Proteins Based on hACE2

Full length hACE2 (aa18-740, Q9BYF1) or truncated hACE2 (aa18-605, Q9BYF1) was cloned in a protein expression vector using a murine IgKappa leader sequence. hACE2 was fused to a human IgG1 or a human IgM Fc domain, comprising the hinge region and constant domains CH2, CH3, and CH4 for IgM. The plasmid vector was transiently transfected onto suspension Freestyle HEK293 using polyethylenimine (PEI), and onto ExpiCHO using Expifectamine transfection reagent. Transfected cells were cultured for 5 days in a shaker incubator at 37° C., 8% CO₂ to allow for protein secretion. Culture supernatant was filtered using 0.22 μm filter units to remove large contaminants (cells and cellular debris).

Fusion proteins were purified using an AKTA™ pure system (GE Healthcare) using a HiTrap MabSelect PrismA 1 ml column (for IgG1 Fc fusion proteins) or a HiTrap IgM 1 m column (for IgM fusion proteins) (both columns from GE Healthcare). Briefly, columns were equilibrated with 5 column volumes of PBS pH 7.4. Supernatant was applied to the column at a flow rate of 1 mL/min. Following application of supernatant, the column was washed with 20 column volumes of PBS. Sample was then eluted from the column with 3 ml of IgG elution buffer (Pierce—21004) at 1 mL/min and directly loaded onto 2 HiTrap 5 ml desalting columns, previously equilibrated in PBS, and collected on a 96-well plate using a fraction collector unit. Proteins were characterised via SDS-PAGE under reducing and non-reducing conditions to assess molecular weight and purity.

### Example 2: Characterisation of Fusion Proteins Based on hACE2

ELISA Assay

ACE2-based fusion proteins were characterised by ELISA to assess binding capacity to viral spike protein. Briefly, Nunc MaxiSorp flat-bottom 96-well plates were coated with 1 μg/ml of recombinant S1 spike protein domain from SARS-CoV and SARS-CoV-2. As control, HepB peptides were coated at 1 μg/ml. Plates were blocked with a solution of 2% BSA in PBS for 1 h at RT. Antibodies were incubated at a range of concentrations, diluted in 0.5% BSA, and allowed to bind for 1 h at RT. Non-specific interactions and un-bound antibodies were washed away by 4 PBS 0.05% Tween20 buffer washes. Bound antibodies were detected via anti-human IgG (H+L) HRP conjugated secondary antibody (for IgG) (Jackson Immunotools) or anti-human IgM HRP conjugated secondary antibody (for IgM) (Abcam), diluted in PBS 0.5% BSA and allow to interact for 1 h at RT. un-bound antibodies were washed away by 4 PBS 0.05% Tween20 buffer washes. Plates were incubated with substrate reagent (1-Step Ultra TMB, Thermo Scientific) and blocked with 1M H₂SO₄. Signal was acquired using a Varioskan plate reader at 450 nm.

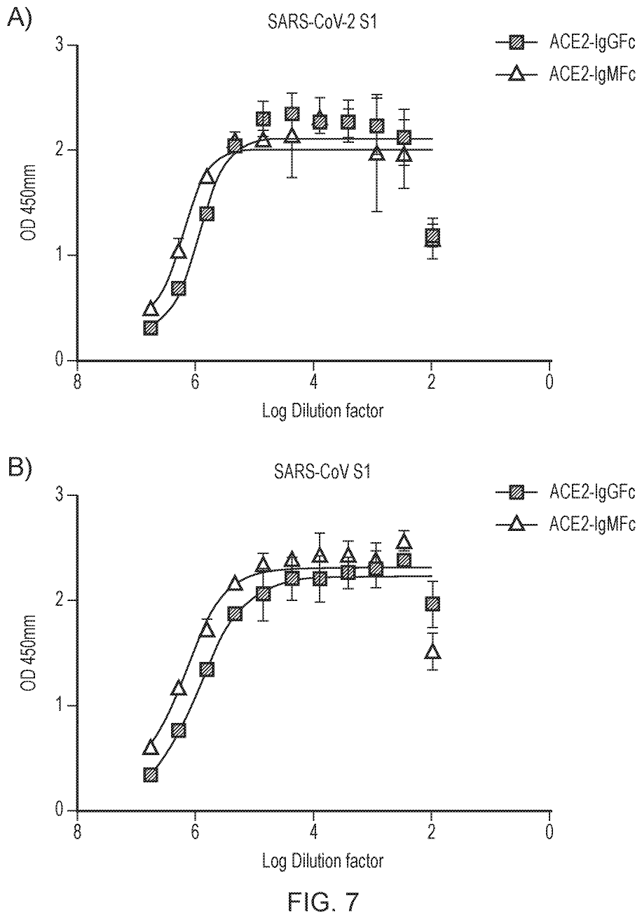

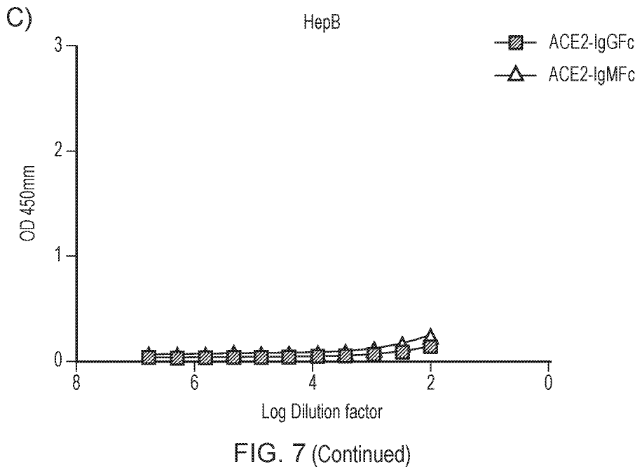

Results showed that hACE2-IgG Fc and hACE2-IgM Fc molecules bind specifically to the S1 subunit of the S protein of SARS-CoV-2 (FIG. 7A) and SARS-CoV (FIG. 7B) in a dose-dependent manner.

SPR Assay

Recombinant hACE2-Fc fusion proteins were immobilised on individual flow cells on a Series S Protein A sensor chip (GE Healthcare) to a density of 70 RU using a Biacore T200 instrument. HBS-P+ buffer was used as running buffer in all experimental conditions. Recombinant purified SARS-CoV-2 S1 protein (Acro biosystems) at known concentrations was used as the ‘analyte’ and injected over the respective flow cells with 150 s contact time and 500 s dissociation at 30 μl/min of flow rate with a constant temperature of 25° C. In each experiment, flow cell 1 was unmodified and used for reference subtraction. A ‘0 concentration’ sensorgram of buffer alone was used as a double reference subtraction to factor for drift. Data were fit to a 1:1 Langmuir binding model. Since a capture system was used, a local Rmax parameter was used for the data fitting in each case.

Fluorescence-Based Receptor Blocking Assay

Recombinant hACE2-Fc fusion proteins are serially diluted from the μM to the pM range and the decreased concentrations fractions are incubated with biotinylated recombinant soluble Spike protein at a constant concentration for 30 min at 37° C. Mixtures are then added to hACE2 expressing cells and incubated for 30 min at 37° C. Cells are then washed with PBS to remove any unbound proteins and stained with Streptavidin conjugated to a fluorophore for 30 min at room temperature. After another PBS wash, cells are analysed for Spike binding to ACE2 in the presence of recombinant therapeutics by flow cytometry.

Fluorescence-Based Spike Protein Targeting

Recombinant hACE2-Fc fusion proteins are serially diluted from the μM to the pM range and the decreased concentrations fractions are added to SARS-CoV-2 Spike protein expressing cells and incubated for 30 min at 37° C. Cells are then washed with PBS to remove any unbound proteins and stained with a secondary anti-Fc antibody conjugated to a fluorophore for 30 min at room temperature. After another PBS wash, cells are analysed by flow cytometry.

Virus Neutralisation Assay

Recombinant fusion proteins are serially diluted to cover a broad range of concentrations. Each dilution is then mixed 1:1 with lentiviral vectors pseudotyped with either control VSV-G glycoprotein or SARS-CoV-2 spike protein to a final volume of 200 μL and incubated at 37° C. for 1 h. The lentiviral vectors also encode eGFP. Mixtures of fusion proteins and virus are then cultured onto hACE2 expressing cell line for 48-72 h. Viral titers are then quantified by eGFP expression in target cells and infectivity of all dilution is determined as a percentage of viral titers in the absence of the recombinant protein.

### Example 3: Generation of Fusion Proteins Based on CD147

Full length human CD147 (aa 22-320, P35613), Domain 1 (D1) of CD147 (aa 22-219, P35613) or Domain 2 (D2) of CD147 (aa 219-320, P35613) were cloned in a protein expression vector using a murine IgKappa leader sequence. The different CD147-based sequences were fused to a human IgG1 or a human IgM Fc domain, comprising the hinge region and constant domains CH2, CH3, and CH4 for IgM. For CD147-hACE2-Fc fusion proteins, CD147 (full length, D1 or D2 domain) was cloned at the C-terminus of hACE2 full length (aa 18-740, Q9BYF1) or truncated (aa 18-605, Q9BYF1) using a flexible Gly-Ser linker. For hACE2-Fc-CD147 fusion proteins, CD147 (full length, D1 or D2 domain) was cloned at the C-terminus of IgG1 Fc using a flexible Gly-Ser linker.

The plasmid vector was transiently transfected onto suspension Freestyle HEK293 using polyethylenimine (PEI) and onto ExpiCHO using Expifectamine transfection reagent. Transfected cells were cultured for 5 days in a shaker incubator at 37° C., 8% CO₂ to allow for protein secretion. Culture supernatant was filtered using 0.22 μm filter units to remove large contaminants (cells and cellular debris).

Fusion proteins were purified using an AKTA™ pure system (GE Healthcare) using a HiTrap MabSelect PrismA 1 ml column (for IgG1 Fc fusion proteins) or a HiTrap IgM 1 ml column (for IgM fusion proteins) (both columns from GE Healthcare). Briefly, columns were equilibrated with 5 column volumes of PBS pH 7.4. Supernatant was applied to the column at a flow rate of 1 ml/min. Following application of supernatant, the column was washed with 20 column volumes of PBS. Sample was then eluted from the column with 3 ml of IgG elution buffer (Pierce—21004) at 1 ml/min and directly loaded onto 2 HiTrap 5 ml desalting columns, previously equilibrated in PBS, and collected on a 96-well plate using a fraction collector unit. Proteins were characterised via SDS-PAGE under reducing and non-reducing conditions to assess molecular weight and purity.

### Example 4: Characterisation of Fusion Proteins Based on CD147

ELISA Assay

The binding capacity of CD147-based fusion proteins to the spike protein of coronavirus was characterised by ELISA. Briefly, Nunc MaxiSorp flat-bottom 96-well plates were coated with 1 μg/ml of recombinant S1 spike protein domain from SARS-CoV or SARS-CoV-2. As control, HepB peptides were coated at 1 μg/ml. Plates were blocked with a solution of 2% BSA in PBS for 1 h at room temperature (RT). Antibodies were incubated at a range of concentrations, diluted in 0.5% BSA, and allowed to bind for 1 h at RT. Non-specific interactions and un-bound antibodies were washed away by 4 PBS 0.05% Tween20 buffer washes. Bound antibodies were detected via anti-human IgG (H+L) HRP conjugated secondary antibody (for IgG) (Jackson Immunotools) or anti-human IgM HRP conjugated secondary antibody (for IgM) (Abcam), diluted in PBS 0.5% BSA and allow to interact for 1 h at RT. un-bound antibodies were washed away by 4×PBS 0.05% Tween20 buffer washes. Plates were incubated with substrate reagent (1-Step Ultra TMB, Thermo Scientific) and blocked with 1M H₂SO₄. Signal was acquired using a Varioskan plate reader at 450 nm.

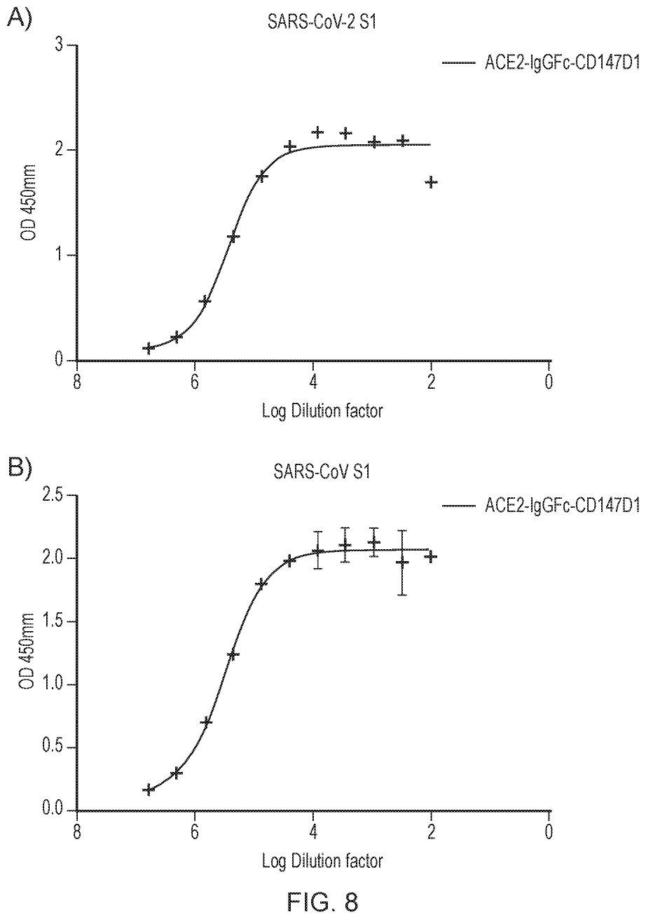

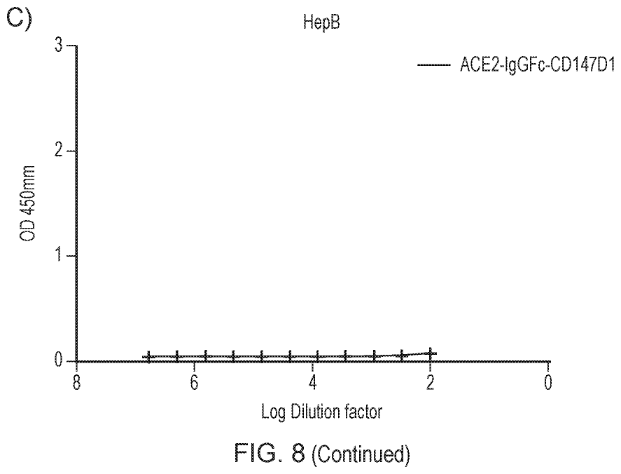

Results showed that CD147-based fusion proteins bind specifically to the S1 subunit of the S protein of SARS-CoV-2 (FIG. 8A) and SARS-CoV (FIG. 8B) in a dose-dependent manner.

SPR Assay

Recombinant CD147-Fc fusion proteins carrying a spike protein binding domain are immobilised on individual flow cells on a Series S CM5 sensor chip (GE Healthcare) previously functionalised with anti-human capture kit or protein A using a Biacore SPR instrument. HBS-P+ buffer is used as running buffer is all experimental conditions. Recombinant purified SARS-CoV-2 spike protein at known concentrations is used as the ‘analyte’ and injected over the respective flow cells with 150 s contact time and 600 s dissociation at 30 μl/min of flow rate with a constant temperature of 25° C. In each experiment, flow cell 1 is unmodified and used for reference subtraction. A ‘O concentration’ sensorgram of buffer alone is used as a double reference subtraction to factor for drift. Data are fit to a 1:1 Langmuir binding model using local Rmax.

Fluorescence-Based Receptor Blocking Assay

Recombinant CD147-based fusion proteins are serially diluted from the μM to the pM range and the decreased concentrations fractions are incubated with biotinylated recombinant soluble Spike protein from coronavirus at a constant concentration for 30 min at 37° C. Mixtures are then added to hACE2 expressing cells and incubated for 30 min at 37° C. Cells are then washed with PBS to remove any unbound proteins and stained with Streptavidin conjugated to a fluorophore for 30 min at room temperature. After another PBS wash, cells are analysed for Spike binding to hACE2 in the presence of recombinant CD147-based fusion proteins by flow cytometry.

Fluorescence-Based Spike Protein Targeting

Recombinant CD147-Fc fusion proteins carrying a spike protein binding domain are serially diluted from the μM to the pM range and the decreased concentrations fractions are added 15 to SARS-CoV-2 Spike protein expressing cells and incubated for 30 min at 37° C. Cells are then washed with PBS to remove any unbound proteins and stained with a secondary anti-Fc antibody conjugated to a fluorophore for 30 min at room temperature. After another PBS wash, cells are analysed by flow cytometry.

Virus Neutralisation Assay

Recombinant CD147-Fc fusion proteins are serially diluted to cover a broad range of concentrations. Each dilution is then mixed 1:1 with lentiviral vectors pseudotyped with either control VSV-G glycoprotein or SARS-CoV-2 spike protein to a final volume of 200 μL and incubated at 37° C. for 1 h. The lentiviral vectors also encode eGFP. Mixtures of fusion proteins and virus are then cultured onto ACE2 expressing cell line for 48-72 hrs. Viral titers are then quantified by eGFP expression in target cells and infectivity of all dilution is determined as a percentage of viral titers in the absence of the recombinant protein.

### Example 5: Generation of Fusion Proteins Based on Anti-SARS Antibodies

A number of neutralising antibodies for SARS-CoV 2003 have been described in the literature. Since the homology between the spike protein of SARS-CoV 2003 and SARS-CoV-2 is high, cross-reactivity can reasonably be expected. Antibodies from previously described SARS-CoV 2003 neutralising binders have the following VH and VL sequences:
a VH sequence of SEQ ID NO: 10 and a VL sequence of SEQ ID NO: 11 (clone 80R);
a VH sequence of SEQ ID NO: 12 and a VL sequence of SEQ ID NO: 13 (clone S230.15);
a VH sequence of SEQ ID NO: 14 and a VL sequence of SEQ ID NO: 15 (clone M396);
a VH sequence of SEQ ID NO: 16 and a VL sequence of SEQ ID NO: 17 (clone F26G19);
a VH sequence of SEQ ID NO: 18 and a VL sequence of SEQ ID NO: 19 (clone F26G8);
a VH sequence of SEQ ID NO: 20 and a VL sequence of SEQ ID NO: 21 (clone F2G8.2);
a VH sequence of SEQ ID NO: 22 and a VL sequence of SEQ ID NO: 23 (clone F26G18);
a VH sequence of SEQ ID NO: 24 and a VL sequence of SEQ ID NO: 25 (clone 92N);
a VH sequence of SEQ ID NO: 26 and a VL sequence of SEQ ID NO: 27 (clone 91M);
a VH sequence of SEQ ID NO: 28 and a VL sequence of SEQ ID NO: 29 (clone 27D);
a VH sequence of SEQ ID NO: 30 and a VL sequence of SEQ ID NO: 31 (clone 26H);
a VH sequence of SEQ ID NO: 32 and a VL sequence of SEQ ID NO: 33 (clone 12E);
a VH sequence of SEQ ID NO: 34 and a VL sequence of SEQ ID NO: 35 (clone 8C);
a VH sequence of SEQ ID NO: 36 and a VL sequence of SEQ ID NO: 37 (clone CR3009);
a VH sequence of SEQ ID NO: 38 and a VL sequence of SEQ ID NO: 39 (clone CR3006);
a VH sequence of SEQ ID NO: 40 and a VL sequence of SEQ ID NO: 41 (clone CR3018);
a VH sequence of SEQ ID NO: 42 and a VL sequence of SEQ ID NO: 43 (clone CR3013);
a VH sequence of SEQ ID NO: 44 and a VL sequence of SEQ ID NO: 45 (clone CR3014);
a VH sequence of SEQ ID NO: 48 and a VL sequence of SEQ ID NO: 49 (clone AS3-3);
a VH sequence of SEQ ID NO: 50 and a VL sequence of SEQ ID NO: 51 (clone CR3022); and
a VH sequence of SEQ ID NO: 52 and a VL sequence of SEQ ID NO: 53 (clone B1).

ScFv of anti-SARS antibodies (ABD) were fused to the N-terminus of a human IgG1 Fc domain. Additionally, scFv of anti-SARS antibodies (ABD) were cloned in fusion to hACE2 full length (aa18-740, Q9BYF1) or truncated (aa18-605, Q9BYF1), carrying a human IgG1 Fc domain at the C-terminus and a murine Igkappa leader sequence at the N-terminus. For the ABD-ACE2-Fc orientation, the antibody was fused to the N-terminus of ACE2 via a flexible Gly-Ser linker. For the ACE2-ABD-Fc orientation, the antibody was linked to the C-terminus of ACE2 via a flexible Gly-Ser linker and to the Fc via the hinge region. The plasmid vector was transiently transfected onto suspension ExpiCHO using Expifectamine transfection reagent. Transfected cells were cultured for 5 days in a shaker incubator at 37° C., 8% CO₂ to allow for protein secretion. Culture supernatant was filtered using 0.22 μm filter units to remove large contaminants (cells and cellular debris). F

Fusion proteins were purified using an AKTA™ pure system (GE Healthcare) using a HiTrap MabSelect PrismA 1 ml column (for IgG1 Fc fusion proteins) or a HiTrap IgM 1 ml column (for IgM fusion proteins) (both columns from GE Healthcare). Briefly, column was equilibrated with 5 column volumes of PBS pH 7.4. Supernatant was applied to the column at a flow rate of 1 mL/min. Following application of supernatant, the column was washed with 20 column volumes of PBS. Sample was then eluted from the column with 3 ml of IgG elution buffer (Pierce—21004) at 1 mL/min and directly loaded onto 2 HiTrap 5 ml desalting columns, previously equilibrated in PBS, and collected on a 96-well plate using a fraction collector unit. Proteins were characterised via SDS-PAGE under reducing and non-reducing conditions to assess molecular weight and purity.

### Example 6: Characterisation of Fusion Proteins Based on Anti-SARS Antibodies

ELISA Assay of Anti-SARS scFv-hACE2-Fc Fusion Proteins

Spike protein binding capacity of anti-SARS-based fusion proteins are characterised by ELISA. Briefly, Nunc MaxiSorp flat-bottom 96-well plates are coated with 1 μg/ml of recombinant S1 spike protein domain from SARS-CoV and SARS-CoV-2. As control, HepB peptides are coated at 1 μg/ml. Plates are blocked with a solution of 2% BSA in PBS for 1 h at room temperature. Test proteins are incubated at a range of concentrations, diluted in 0.5% BSA, and allowed to bind for 1 h at RT. Non-specific interactions and un-bound antibodies are washed away with 4 PBS 0.05% Tween20 buffer washes. Bound antibodies are detected via anti-mouse IgG HRP conjugated secondary antibody (Jackson Immunotools) or anti-human IgG (H+L) HRP conjugated secondary antibody (Jackson Immunotools), diluted in PBS 0.5% BSA and allowed to interact for 1 h at RT. Un-bound antibodies are washed away by 4 PBS 0.05% Tween20 buffer washes. Plates are incubated with substrate reagent (1-Step Ultra TMB, Thermo Scientific) and blocked with 1M H₂SO₄. Signal is acquired using a Varioskan plate reader at 450 nm.

ELISA Assay of Anti-SARS scFv-Fc Fusion Proteins

Antibodies in scFv-Fc format were expressed by transient transfection in ExpiCHO cells and tested as non-purified supernatants in ELISA against SARS-CoV-2 Spike protein subunit S1 or full spike protein

Nunc Maxisorp clear 96-well plates were coated with 1 μg/ml (in PBS) of recombinant protein (SARS-CoV-2 S1 or full spike protein, ACRO biosystems) overnight at 4° C. Plates were blocked with PBS 2% BSA for 1 h at RT. Test proteins were incubated at a specified range of concentrations with serial dilutions for 1 h at RT in PBS 0.5% BSA. Bound proteins were detected with anti-mouse or anti-human HRP conjugated antibodies (Jackson Immunotools) at 1:5000 and 1:3000 dilution in PBS 0.5% BSA, respectively. Incubation was allowed for 1 h at RT. All washes were performed in PBS 0.05% Tween20. Detection reagent 1-step TMB Ultra (thermo scientific) and reaction blocked with 1M H₂SO₄. Plates were acquired on a Varioskan Lux instrument at a wavelength of 450 nm.

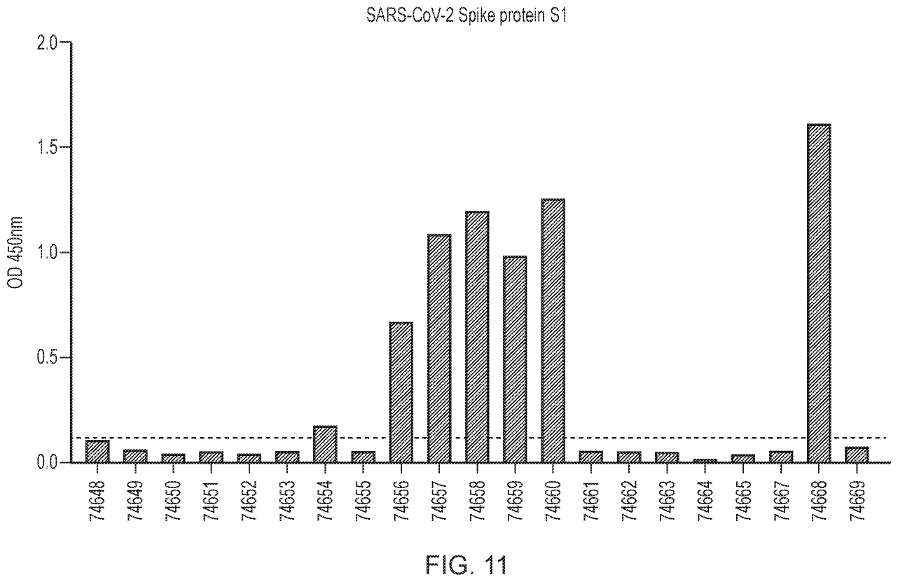

Results revealed that scFv-Fc based on clones 91M (75656), 27D (74657), 26H (74658), 12E (74659), 8C (74660) and CR3022 (74668) showed cross-reactivity for SARS-CoV-2 S1 protein (FIG. 11). Clone B1 (74669) was identified as a S2 binder.

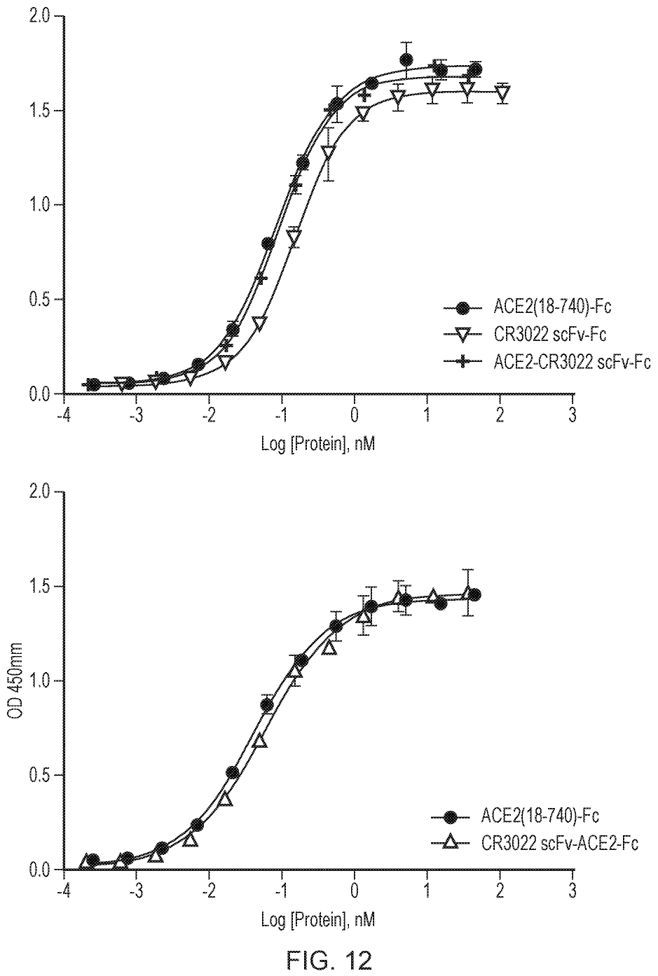

Clone CR3022, was initially recognised as a potential SARS-CoV-2 neutralising antibody. Indeed, strong binding was also observed against recombinant SARS-CoV-2 S1 protein in our hands (FIG. 11). CR3022 has been demonstrated to bind to a distinct epitope on the S1 subunit of SARS-CoV-2 spike protein, in close proximity to the ACE2 binding region, unlikely to elicit steric hindrance. Two fusion products were developed in order to take advantage of a potential second contact point to SARS-CoV-2 spike protein, ACE2-CR3022 scFv-Fc and CR3022 scFv-ACE2-Fc. Binding to the spike protein was evaluated for these constructs and ACE2 (18-740)-Fc and 3022 scFv-Fc by ELISA. Results showed that binding to the spike protein was comparable for all protein constructs (FIG. 12).

SPR Assay

Recombinant fusion proteins are immobilised on individual flow cells on a Series S CM5 sensor chip (GE Healthcare) previously functionalised with anti-human, anti-mouse capture kit or protein A, using a Biacore SPR instrument. HBS-P+ buffer is used as running buffer is all experimental conditions. Recombinant purified SARS-CoV-2 spike protein at known concentrations is used as the ‘analyte’ and injected over the respective flow cells with 150 s contact time and 600 s dissociation at 30 μl/min of flow rate with a constant temperature of 25° C. In each experiment, flow cell 1 is unmodified and used for reference subtraction. A ‘0 concentration’ sensorgram of buffer alone is used as a double reference subtraction to factor for drift. Data are fit to a 1:1 Langmuir binding model using local Rmax.

Fluorescence-Based Receptor Blocking Assay

Recombinant fusion proteins are serially diluted from the μM to the pM range and the decreased concentrations fractions are incubated with biotinylated recombinant soluble Spike protein at a constant concentration for 30 min at 37° C. Mixtures are then added to hACE2 expressing cells and incubated for 30 min at 37° C. Cells are then washed with PBS to remove any unbound proteins and stained with Streptavidin conjugated to a fluorophore for 30 min at room temperature. After another PBS wash, cells are analysed for Spike binding to ACE2 in the presence of recombinant therapeutics by flow cytometry.

Fluorescence-Based Spike Protein Targeting

Recombinant fusion proteins are serially diluted from the μM to the pM range and the decreased concentrations fractions are added to SARS-CoV-2 Spike protein expressing cells and incubated for 30 min at 37° C. Cells are then washed with PBS to remove any unbound proteins and stained with a secondary anti-Fc antibody conjugated to a fluorophore for 30 min at room temperature. After another PBS wash, cells are analysed by flow cytometry.

Virus Neutralisation Assay

Recombinant proteins are serially diluted to cover a broad range of concentrations. Each dilution is then mixed 1:1 with lentiviral vectors pseudotyped with either control VSV-G glycoprotein or SARS-CoV-2 spike protein to a final volume of 200 μL and incubated at 37° C. for 1 h. The lentiviral vectors also encode eGFP. Mixtures of fusion proteins and virus are then cultured onto hACE2 expressing cell line for 48-72 h. Viral titers are then quantified by eGFP expression in target cells and infectivity of all dilution is determined as a percentage of viral titers in the absence of the recombinant protein.

### Example 7: Generation of Binders Specific for the S Protein of SARS-CoV-2 by Immunising Rats

The nucleic acid sequence of SARS-CoV-2 Spike protein is cloned in the vector pVAC2. 3×Wistar rats are immunized with plasmid DNA encoding the spike protein, adsorbed to gold nanoparticles. A Gene-Gun™ (Biorad) system is used to deliver the coated gold nanoparticles intramuscularly. Rats are boosted 3 times over the course of 28 days. Test bleeds from the rats are screened for titres of anti-SARS-CoV-2 antibodies by ELISA and flow cytometry.

Rats with SARS-CoV-2 positive sera are selected for a final immunisation boost before the spleens are harvested for B cell isolation and hybridoma production. Hybridoma fusions of 10×96-well plates with lymphocytes from the selected rats are performed. Hybridoma supernatants are screened for reactive anti-SARS-CoV-2 antibodies by ELISA against recombinant purified protein. ELISA positive hybridoma supernatants are tested by flow cytometry spike protein expressing cell lines.

Hybridomas expressing the strongest anti-SARS-CoV-2 response by flow cytometry are identified, expanded, and stocks cloned to generate monoclonal antibody secreting hybridomas. Hybridoma clones are obtained by limiting dilution.

Total RNA is isolated from monoclonal hybridoma cells using illustra RNAspin Mini kit (GE Healthcare, product number 25050071) according to the manufacturer's instructions. The total RNA is analysed by agarose gel electrophoresis and the concentration assessed using a NanoDrop2000C. Total RNA is reverse-transcribed into cDNA using Oligo(dT)20 and SuperScript™ II Reverse Transcriptase (ThermoFisher Scientific, product number 18064022) in the presence of template-switch oligo according to manufacturer's instructions. The antibody fragments of VH and VL are amplified using the 5′RACE PCR method. DNA fragments are cloned blunt-ended into vectors using CloneJET PCR Cloning Kit (ThermoFisher Scientific, product number K1231) according to manufacturer's instructions. Five colonies for each of the heavy and light chains are sequenced and a consensus sequence was obtained.

### Example 8: Generation of Binders Specific for the S Protein of SARS-CoV-2 by Immunising Llama

One hundred micrograms of spike protein S1-His (Acro biosystems, S1N-C52H3) are injected intraperitoneally in llamas. A blood sample of about 200 ml is taken from 2×immunised llamas. An enriched lymphocyte population is obtained via Ficoll discontinuous gradient centrifugation. From these cells, total RNA is isolated by acid guanidium thiocyanate extraction. After first strand cDNA synthesis DNA fragments encoding HC-V fragments and part of the long or short hinge region are amplified by PCR. The amplified pool of dAb antibody sequences is digested using the restriction enzymes PstI and NotI, and ligated into the phagemid vector PRL114.

Single domain antibodies are expressed on phage after infection with M13K07. The phage library is panned for the presence of binders respectively on solid-phase Spike protein in wells of a microtitre plates or in solution with 100 nM biotinylated spike protein in combination with streptavidin-coated magnetic beads.

### Example 9: Generation of Binders Specific for the S Protein of SARS-CoV-2 by Phage Display

Binders specific for the S protein of SARS-CoV-2 were screened by phage display using a naïve llama library. Recombinant SARS-CoV-2 spike protein with a His tag (Acro S1N-C52H3) was immobilised on a Nunc immunotube at 1 μg/ml overnight at 4C before blocking with a 2% milk PBS solution. Phage were blocked in 2% milk PBS with 1 μg/ml of a His tagged protein included (2 ml) for 1 h before addition to the S1-His coated immunotube. After 1 h at room temp the tube wash washed 10 times using PBS 0.05% tween (4 ml/wash). Elution of specific phage was performed by addition of re-warmed trypsin (2 ml) to the tube and incubation at 37° C. for 10-15 min. Eluted phage were amplified by reinfection into log phase TG1 cells (5 ml) and plating out on Amp/Gluc agar plates. Titrations were performed to establish phage numbers and enrichment.

Further panning selection rounds were performed as above except alterations to panning antigen and elution method were made. Namely, site specifically biotinylated (avi-tagged) Spike protein 1 (sp1) was used in pan 2 and capture was performed on a streptavidin coated plate (Pierce Cat #15500) (300 ul/well). In a further panning method panning was performed in solution phase whereby pre-blocked phage were incubated with S1-avitag in solution (1 ug/ml) with addition of ACE2-Fc at equal concentration to enable high affinity antibody selection. S1-avitag as well as bound phage were then captured on streptavidin coated plates before trypsin elution as described above.

Screening

Individual bacterial colonies were picked and cultured at 37° C. in 2TY Gluc/Amp media until OD600 nm of 0.5 before induction of antibody expression by addition of IPTG (1 mM) and overnight culture at 30° C. The cells were pelleted by centrifugation and supernatant used directly for screening ELISAs and flow cytometry.

ELISA

All incubations were room temp 1 h with agitation whilst washing was 3 times with 0.05% PBS.tween. S1-avitag was plated on a streptavidin coated 96 well plate at 0.5 μg/ml (50 μl) and incubated at room temp for 1 h, a further 2 plates were coated with His tag control protein or blocking reagent (2% milk), washed and blocked with 2% milk for 1 h and washed again before supernatants were added. Detection antibody was anti-C Myc-HRP conjugate (Abcam clone 9E10 Cat #62928), and the plate was developed with TMB substrate before being read at 450 nm.

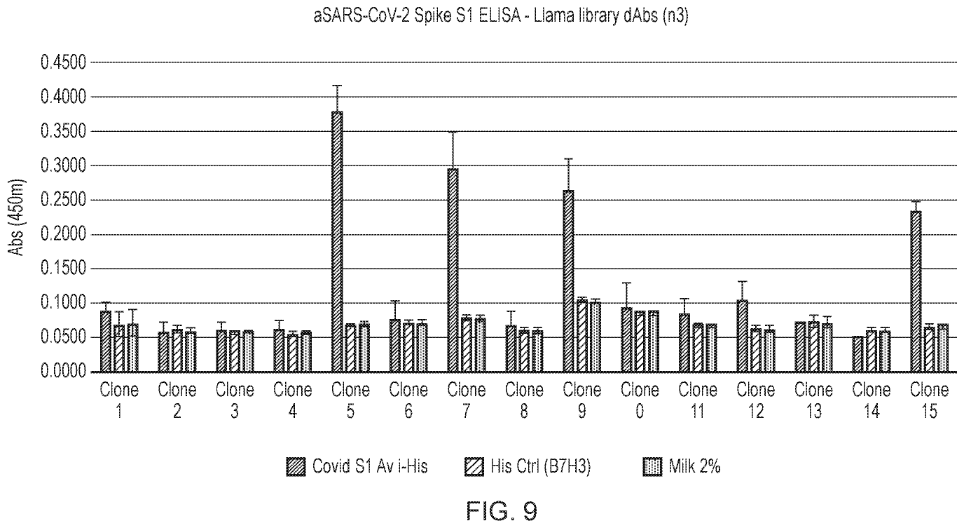

Results showed that some clones presented specific binding to S1 protein of SARS-CoV-2 (FIG. 9).

Sequencing

All sequences were obtained after PCR amplification of dAb coding region from phage expressing TG1 cells using primers M13Rvs: caggaaacagctatgac (SEQ ID NO: 94) and M13 Fwd: gtcgtctttccagacgttagt (SEQ ID NO: 95). Primer annealing temperature was 48° C. M13Rvs primer was used as sequencing primer at Source Bioscience (Cambridge, UK).

### Example 10: Characterisation of Binders Specific for the S Protein of SARS-CoV-2

ELISA Assay

The capacity of anti-SARS antibodies obtained in Example 9 to bind to the S protein of SARS-CoV-2 Spike protein was further characterised by ELISA. Briefly, Nunc MaxiSorp flat-bottom 96-well plates were coated with 1 μg/ml of recombinant S1 spike protein domain from SARS-CoV and SARS-CoV-2. As control, HepB peptides were coated at 1 μg/ml. Plates were blocked with a solution of 2% BSA in PBS for 1 h at RT. Antibodies were incubated at a range of concentrations, diluted in 0.5% BSA, and allow to bind for 1 h at RT. Non-specific interactions and un-bound antibodies were washed away by 4 PBS 0.05% Tween20 buffer washes. Bound antibodies were detected via anti-mouse IgG HRP conjugated secondary antibody (Jackson Immunotools) or anti-human IgG (H+L) HRP conjugated secondary antibody (Jackson Immunotools), diluted in PBS 0.5% BSA and allow to interact for 1 h at RT. un-bound antibodies were washed away by 4 PBS 0.05% Tween20 buffer washes. Plates were incubated with substrate reagent (1-Step Ultra TMB, Thermo Scientific) and blocked with 1M H₂SO₄. Signal was acquired using a Varioskan plate reader at 450 nm.

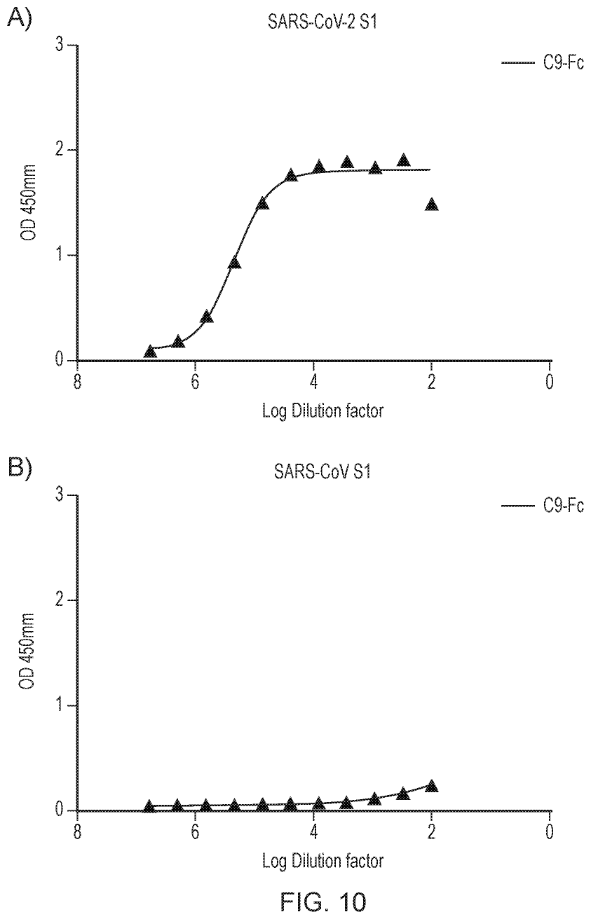

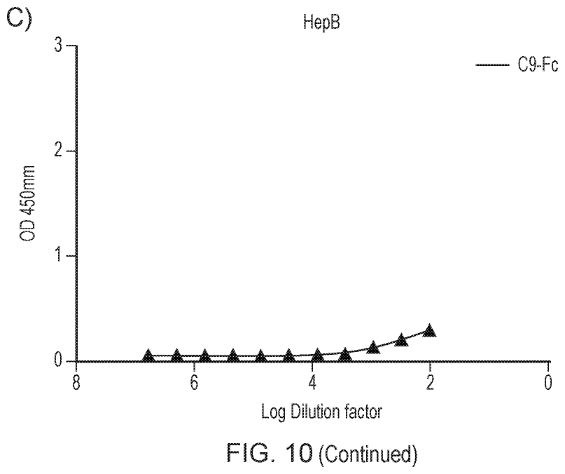

Results obtained using clone C9 fused to Fc showed that it binds specifically to the S1 subunit of the S protein of SARS-CoV-2 (FIG. 10A) in a dose-dependent manner. Importantly, clone 9 did not bind to SARS-CoV (FIG. 10B).

SPR Assay

Anti-SARS-CoV-2 antibodies are immobilised on individual flow cells on a Series S CM5 sensor chip (GE Healthcare) previously functionalised with anti-mouse capture kit or protein A using a Biacore SPR instrument. HBS-P+ buffer is used as running buffer is all experimental conditions. Recombinant purified SARS-CoV-2 spike protein at known concentrations is used as the ‘analyte’ and injected over the respective flow cells with 150 s contact time and 600 s dissociation at 30 μl/minute of flow rate with a constant temperature of 25° C. In each experiment, flow cell 1 is unmodified and used for reference subtraction. A ‘0 concentration’ sensorgram of buffer alone is used as a double reference subtraction to factor for drift. Data are fit to a 1:1 Langmuir binding model using local Rmax.

Fluorescence-Based Receptor Blocking Assay

Recombinant antibodies are serially diluted from the μM to the pM range and the decreased concentrations fractions are incubated with biotinylated recombinant soluble Spike protein at a constant concentration for 30 min at 37° C. Mixtures are then added to ACE2 expressing cells and incubated for 30 min at 37° C. Cells are then washed with PBS to remove any unbound proteins and stained with Streptavidin conjugated to a fluorophore for 30 min at room temperature. After another PBS wash, cells are analysed for Spike binding to ACE2 in the presence of recombinant therapeutics by flow cytometry.

Fluorescence-Based Spike Protein Targeting

Recombinant antibodies are serially diluted from the μM to the pM range and the decreased concentrations fractions are added to SARS-CoV-2 Spike protein expressing cells and incubated for 30 min at 37° C. Cells are then washed with PBS to remove any unbound proteins and stained with a secondary anti-Fc antibody conjugated to a fluorophore for 30 min at room temperature. After another PBS wash, cells are analysed by flow cytometry.

Virus Neutralisation Assay

Recombinant proteins are serially diluted to cover a broad range of concentrations. Each dilution is then mixed 1:1 with lentiviral vectors pseudotyped with either control VSV-G glycoprotein or SARS-CoV-2 spike protein to a final volume of 200 μL and incubated at 37° C. for 1 h. The lentiviral vectors also encode eGFP. Mixtures of fusion proteins and virus are then cultured onto ACE2 expressing cell line for 48-72 hrs. Viral titers are then quantified by eGFP expression in target cells and infectivity of all dilution is determined as a percentage of viral titers in the absence of the recombinant protein.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

### Example 11: Generation of Fusion Proteins Based on Inactive hACE2

Human ACE2 (18-740) or truncated human ACE2 (18-605) were mutated to incorporate the H374N and H378N mutations (HH:NN or inactive ACE2) to inhibit catalytic activity. These mutations are predicted to remove interaction with Zn⁺² ions mediated by the two original His residues, with a spatially conservative mutation. Then, they were fused to a human IgG1 hinge-CH2-CH3 domain and were cloned in a protein expression vector using a murine IgKappa leader sequence. hACE2 was fused to a human IgG1, comprising the hinge region and constant domains CH2 and CH3. The plasmid vector was transiently transfected onto suspension Freestyle HEK293 using polyethylenimine (PEI), and onto ExpiCHO using Expifectamine transfection reagent. Transfected cells were cultured for 5 days in a shaker incubator at 37° C., 8% CO₂ to allow for protein secretion. Culture supernatant was filtered using 0.22 μm filter units to remove large contaminants (cells and cellular debris).

Fusion proteins were purified using an AKTA™ pure system (GE Healthcare) using a HiTrap MabSelect PrismA 1 ml column (for IgG1 Fc fusion proteins). Briefly, the column was equilibrated with 5 column volumes of PBS pH 7.4. Supernatant was applied to the column at a flow rate of 1 mL/min. Following application of supernatant, the column was washed with 20 column volumes of PBS. Sample was then eluted from the column with 3 ml of IgG elution buffer (Pierce—21004) at 1 mL/min and directly loaded onto 2 HiTrap 5 ml desalting columns, previously equilibrated in PBS, and collected on a 96-well plate using a fraction collector unit. Proteins were characterised via SDS-PAGE under reducing and non-reducing conditions to assess molecular weight and purity.

### Example 12: Characterisation of Fusion Proteins Based on Inactive hACE2 (HH:NN)

ACE2 Enzymatic Activity

ACE2 activity was measured by using Mca-APK(Dnp) as substrate in 96-well black microtiter plates. The samples were added into wells containing reaction buffer (50 mM 4-morpholineethanesulfonic acid, pH=6.5, 300 mM NaCl, 10 μM ZnCl2 and 0.01% Triton X-100,) at a concentration of 0.1 μg/ml in the presence of 20 μM of Ma-APK(Dnp) or control peptide BML-P127 (Enzo Life Sciences) in a final volume of 100 μl/well. Activity was compared to the active ACE2-Fc (Acro biosystems). The reaction was performed in triplicate at room temperature for 1 h. Activity was measured as fluorescence intensity at 320 nm excitation and 393 nm emission wavelength at 1-minute intervals using a Varioskan LUX instrument (Thermo Scientific).

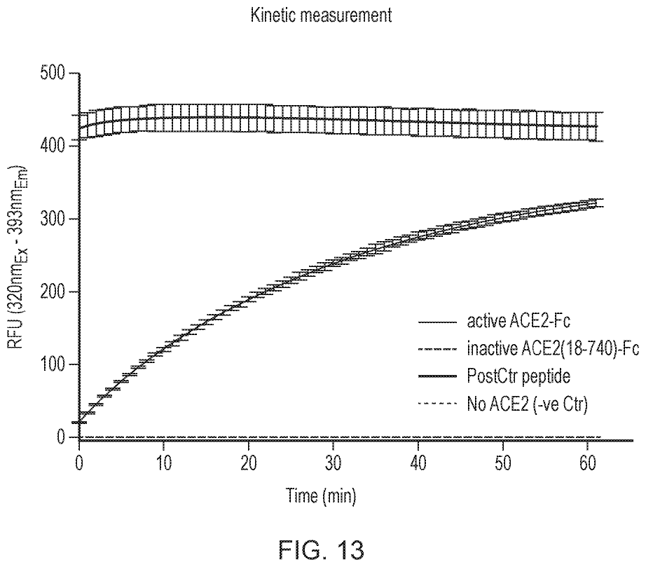

Results indicate that the active ACE2-Fc was able to efficiently process and activate fluorescence on the substrate, while the inactive ACE2 (HH:NN) (18-740)-Fc construct developed in this project was unable to convert the substrate and showed a completely abrogated enzymatic activity (FIG. 13).

SPR Assay

Recombinant ACE2-Fc constructs were captured on flow cell 2 of a Series S Protein A sensor chip (GE Healthcare) to a density of 500 RU using a Biacore 8K instrument. HBS-EP+ buffer was used as running buffer in all experimental conditions. Recombinant purified Angiotensin II (Sigma) at known concentrations (concentration range from 1 μM to 15.625 nM) was used as the ‘analyte’ and injected over the respective flow cells with 150 s contact time and 500 s dissociation at 30 l/minute of flow rate with a constant temperature of 25° C. In each experiment, flow cell 1 was unmodified and used for reference subtraction. A ‘0 concentration’ sensogram of buffer alone was used as a double reference subtraction to factor for drift. Data were fit to a 1:1 Langmuir binding model. Since a capture system was used, a local Rmax parameter was used for the data fitting in each case.

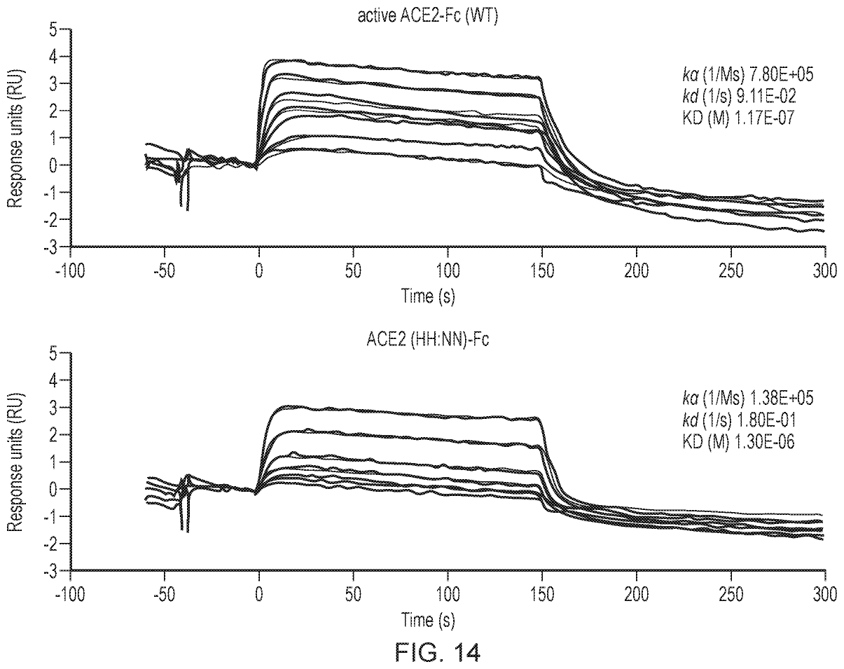

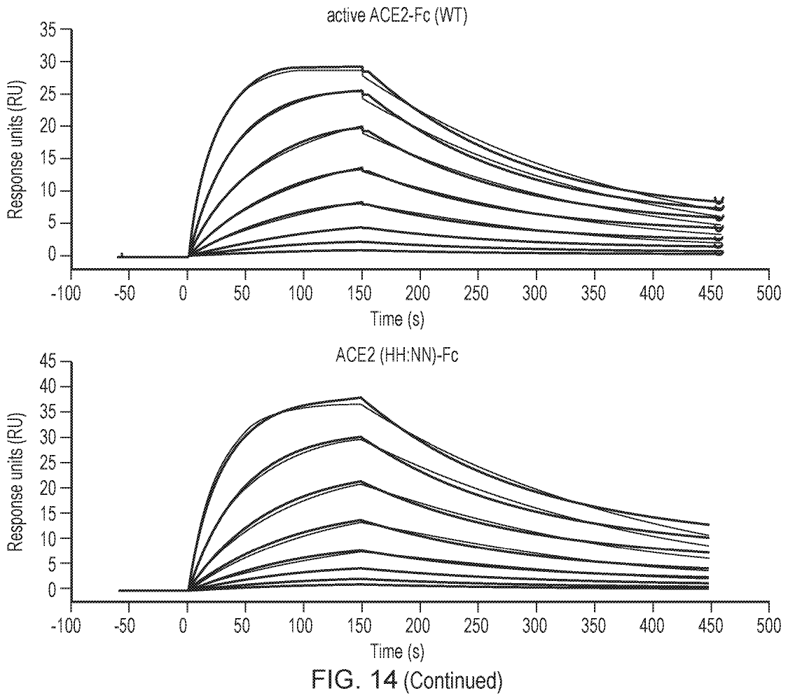

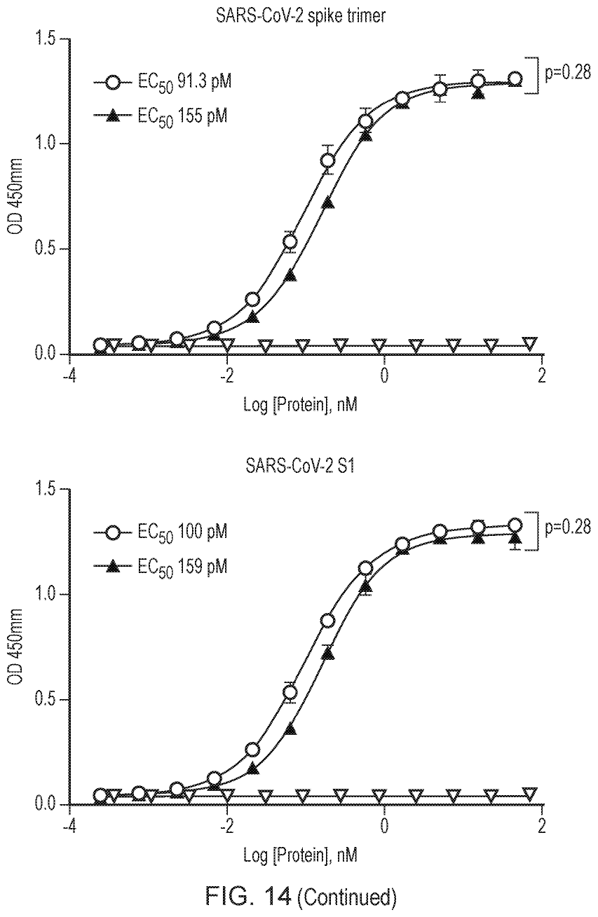

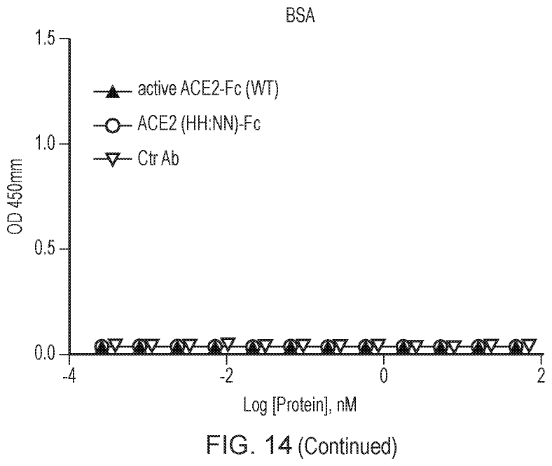

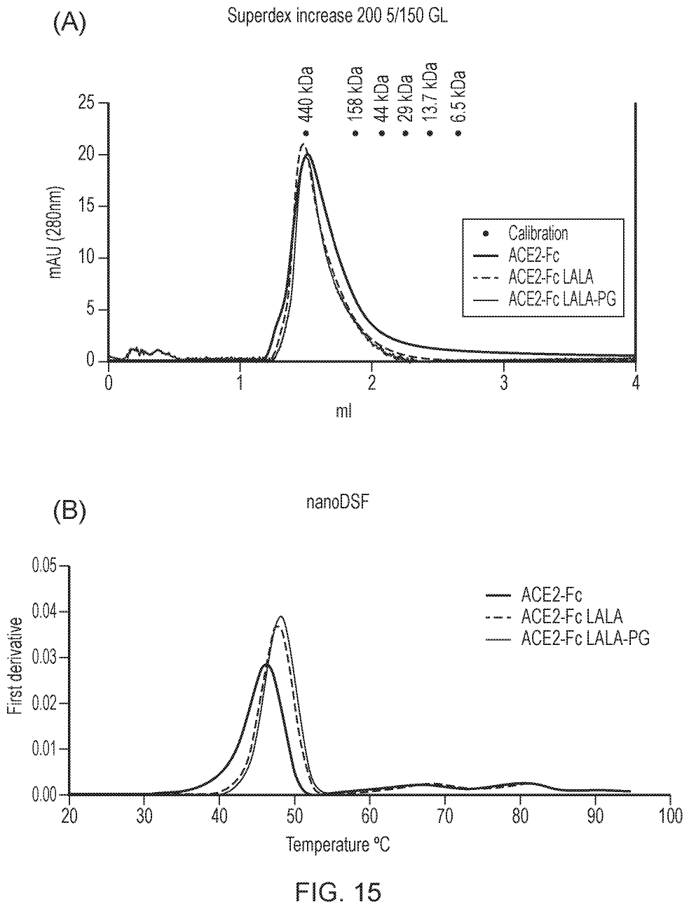

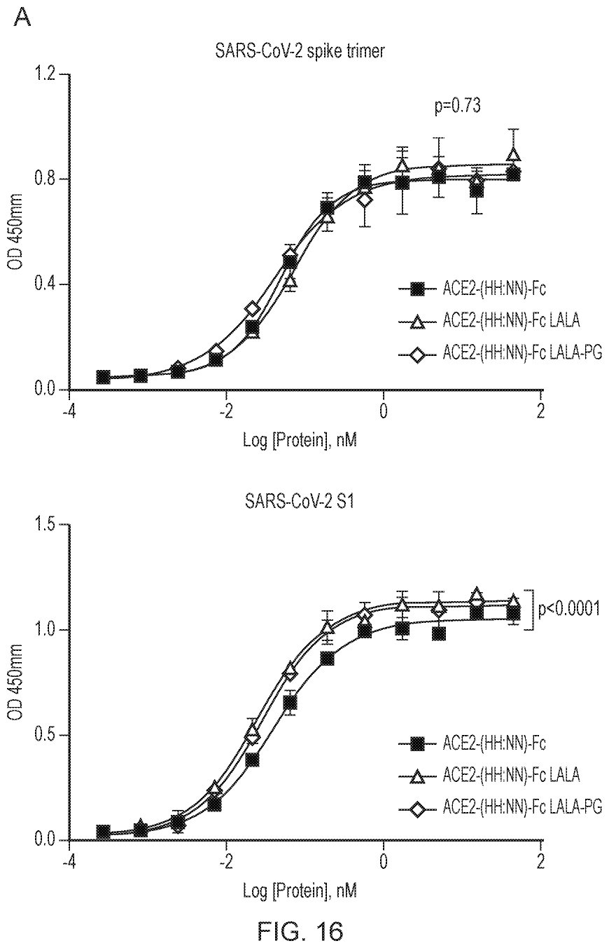

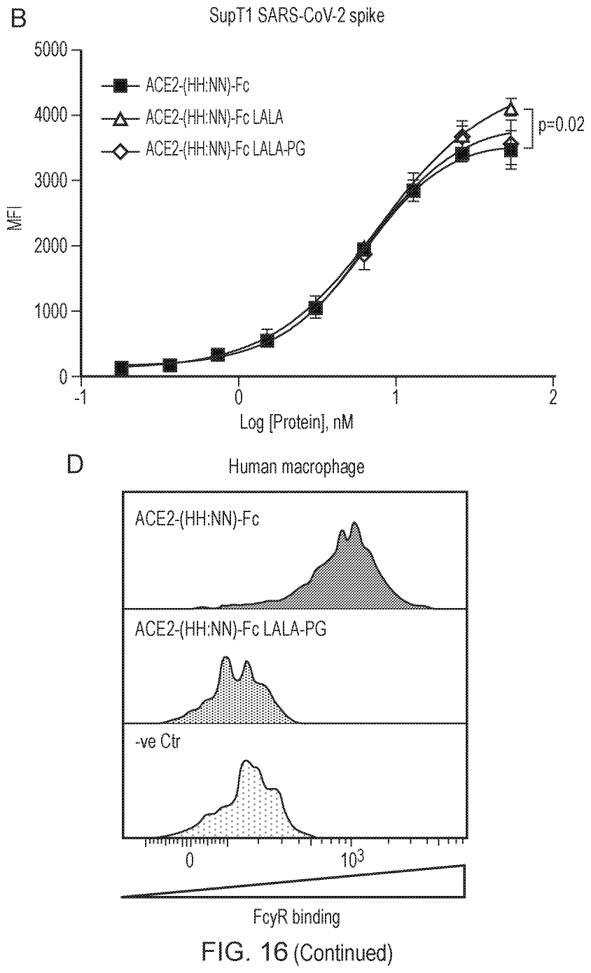

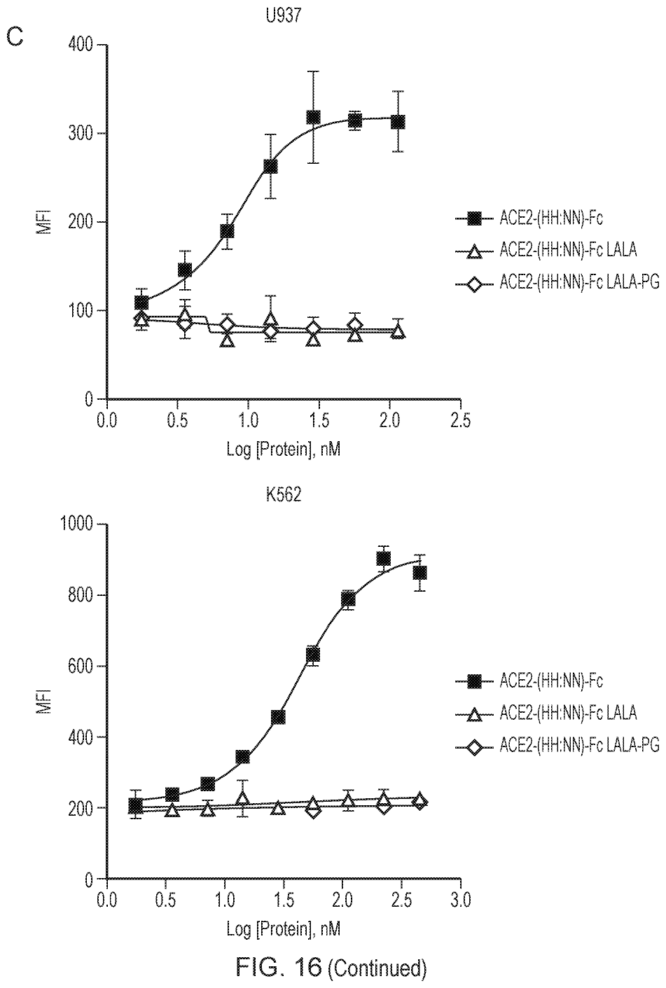

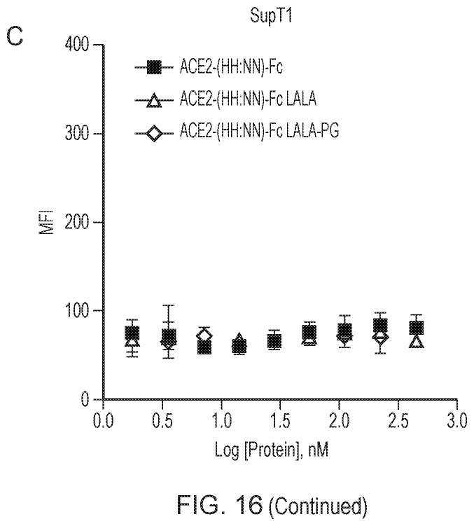

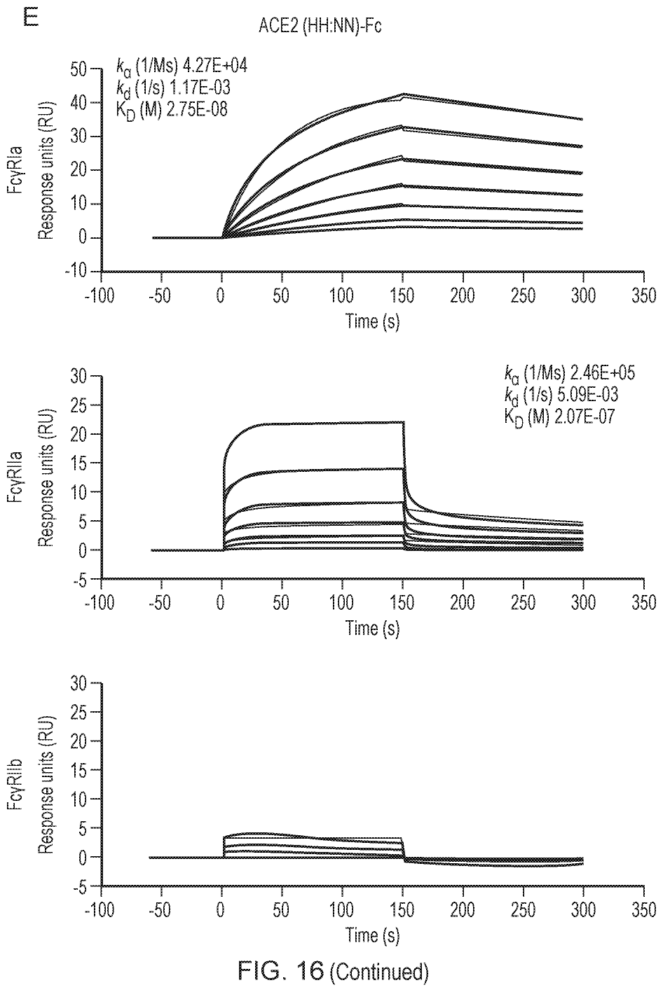

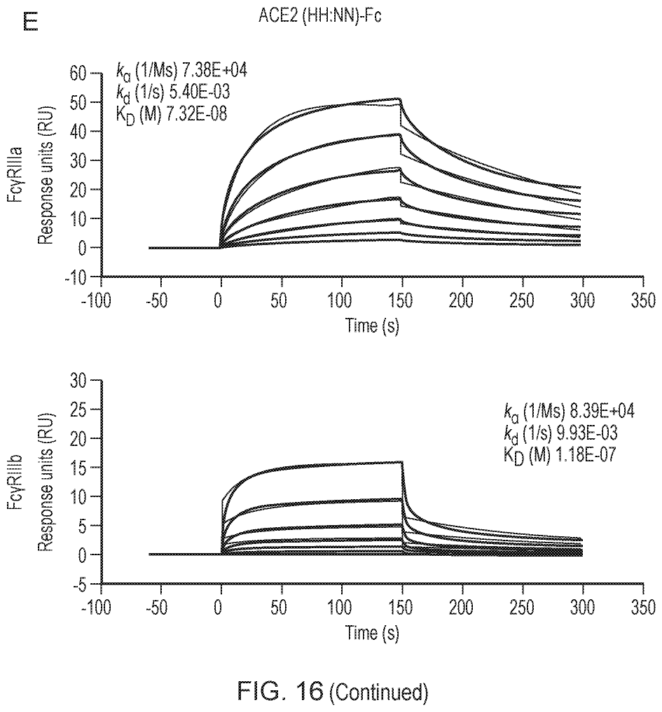

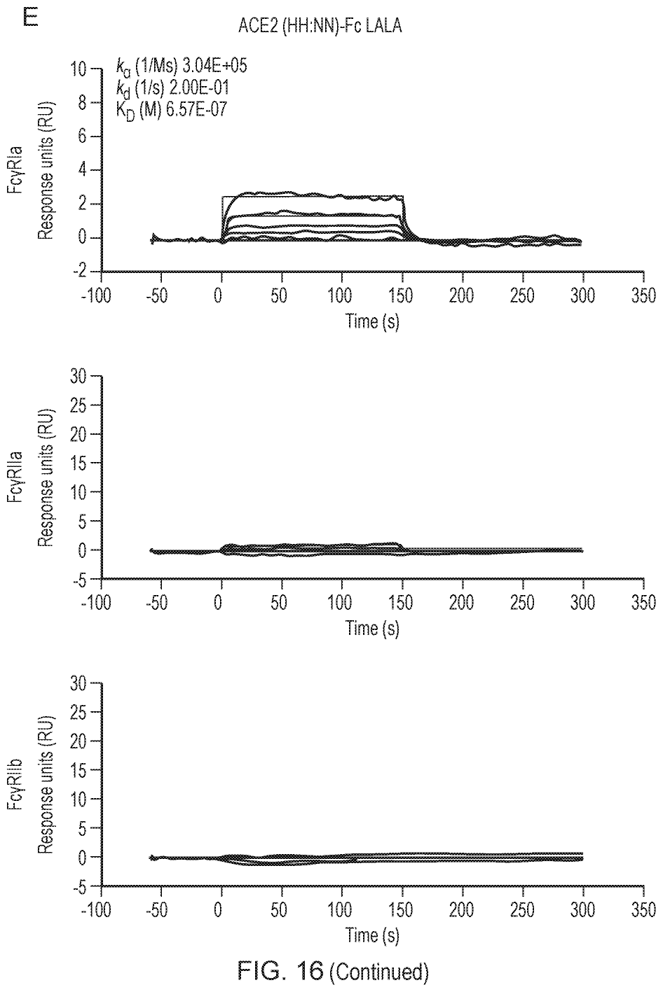

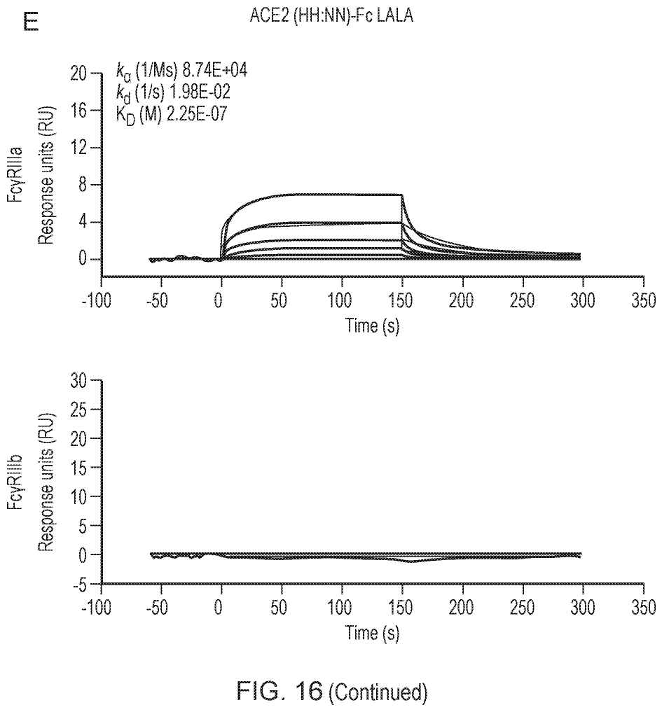

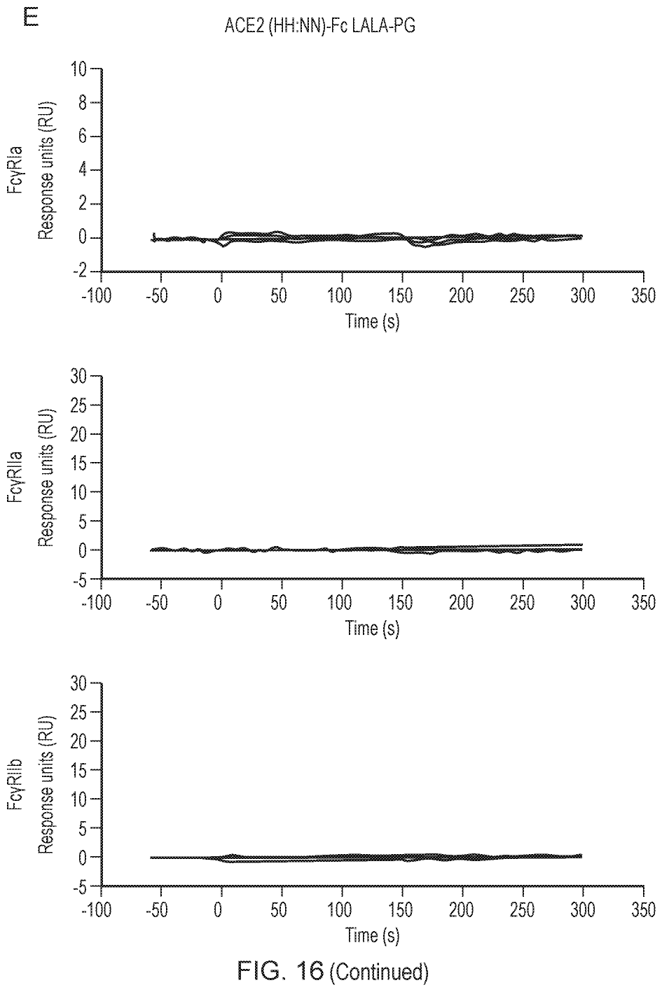

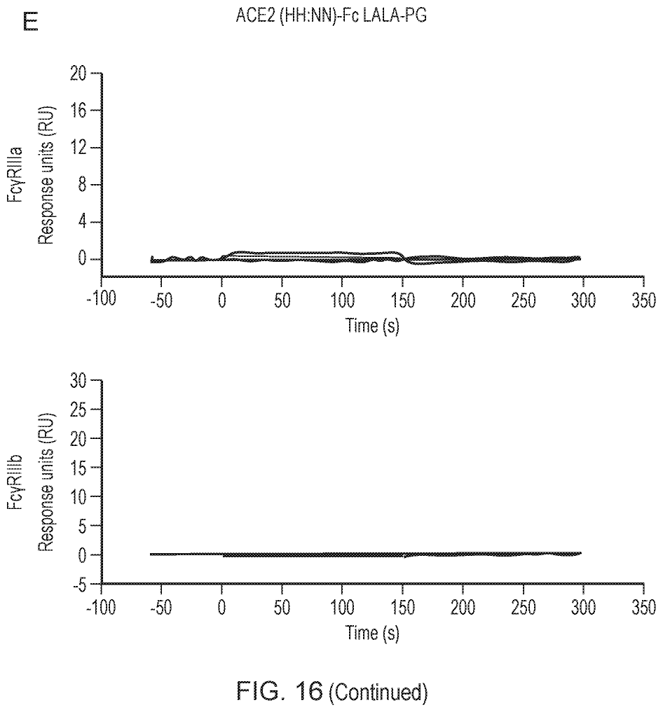

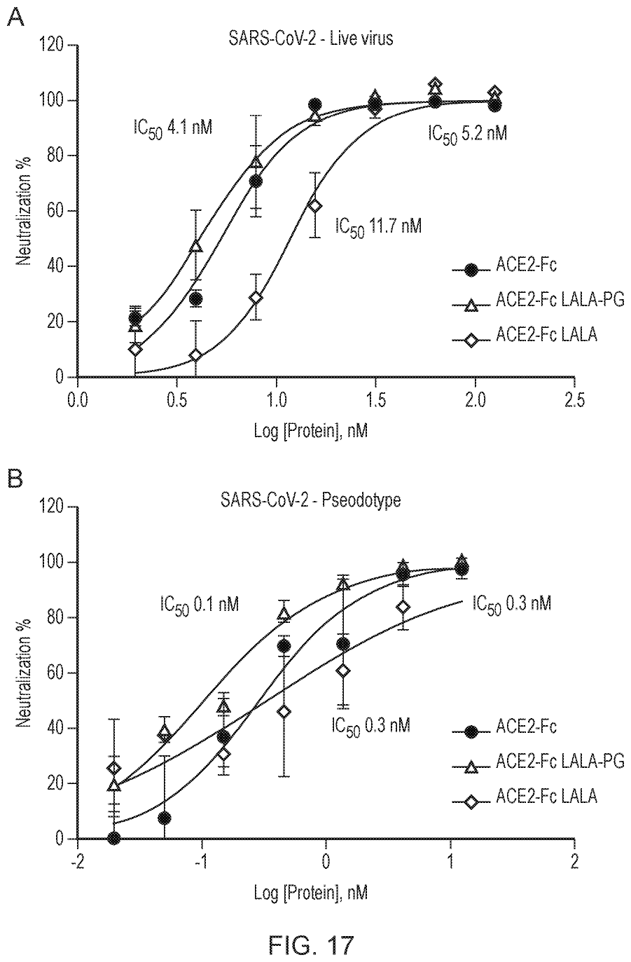

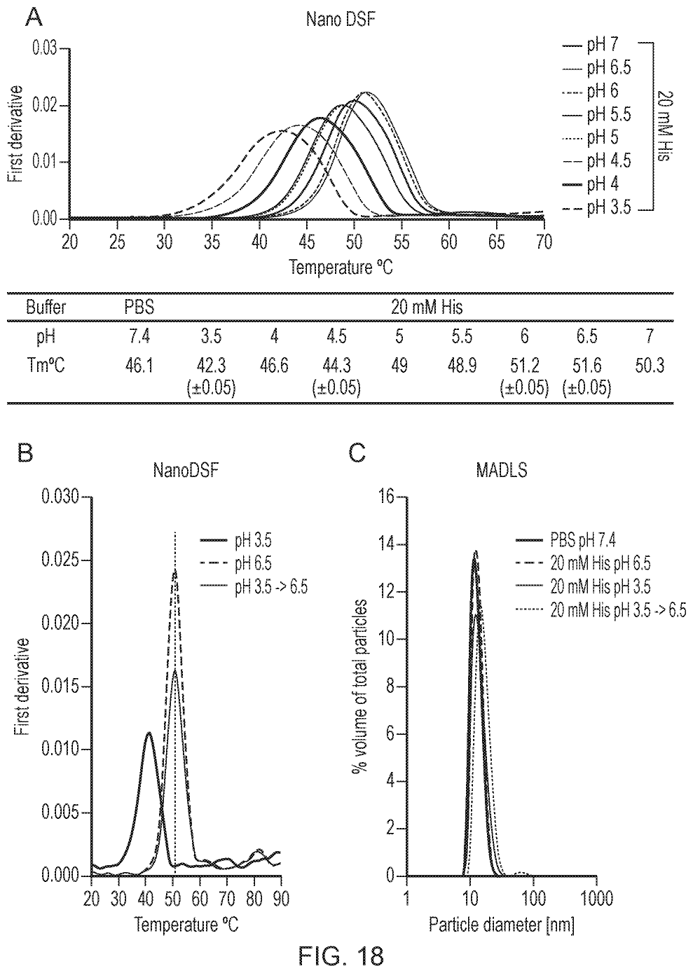

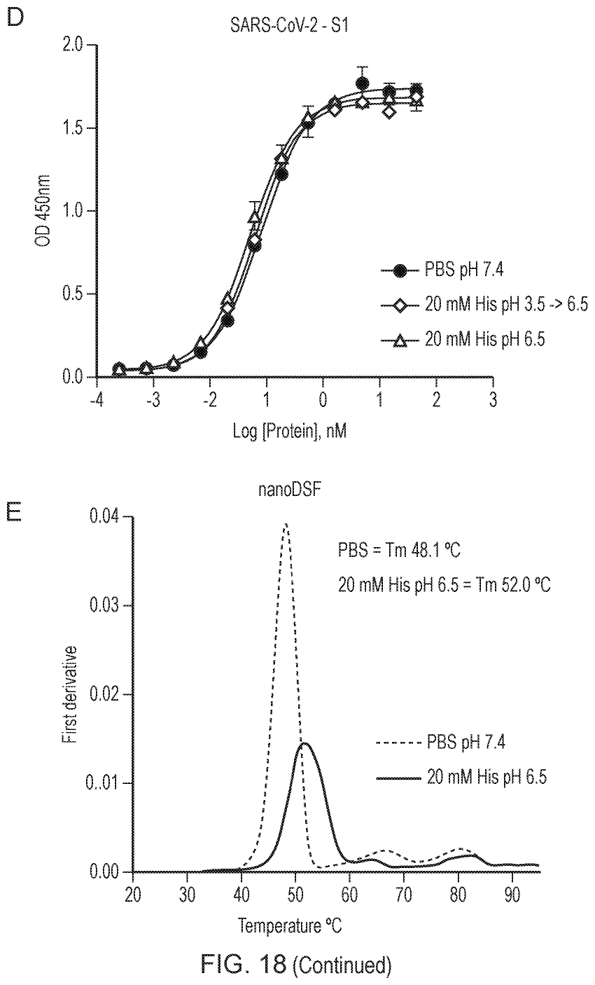

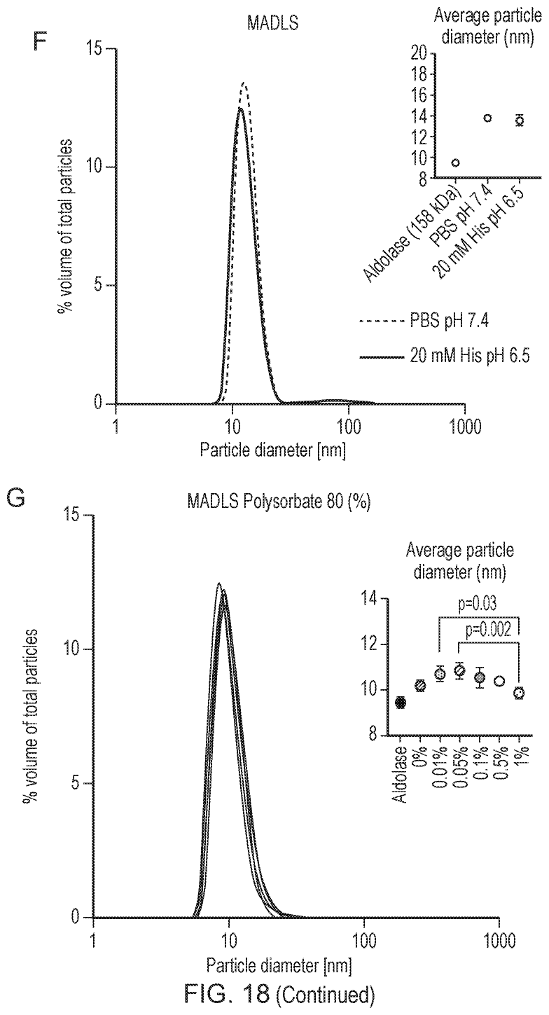

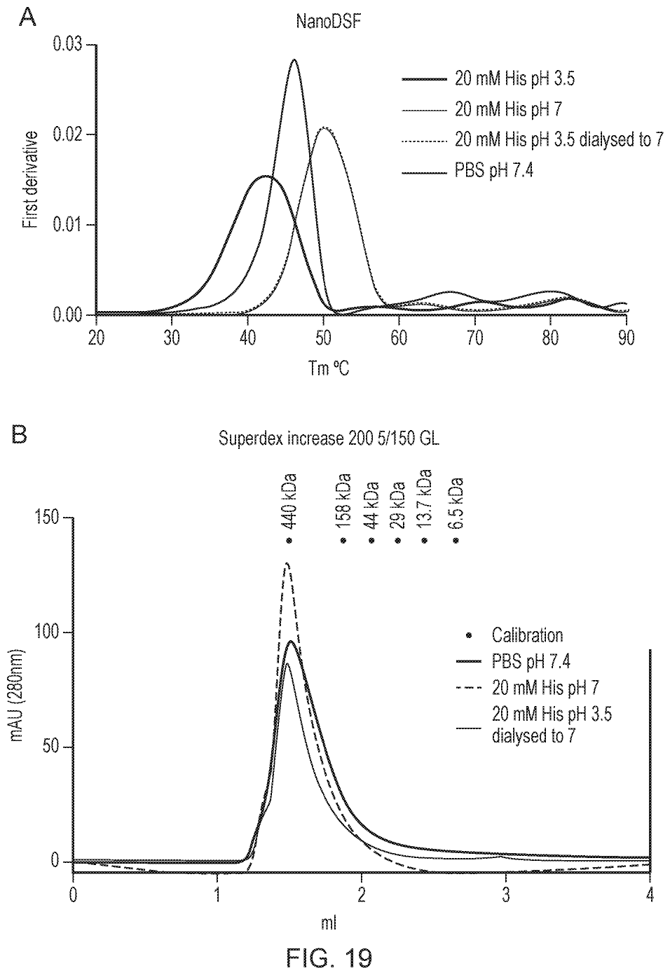

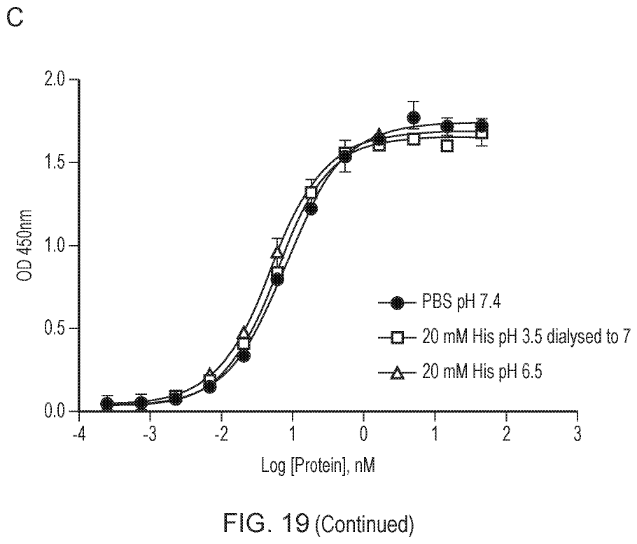

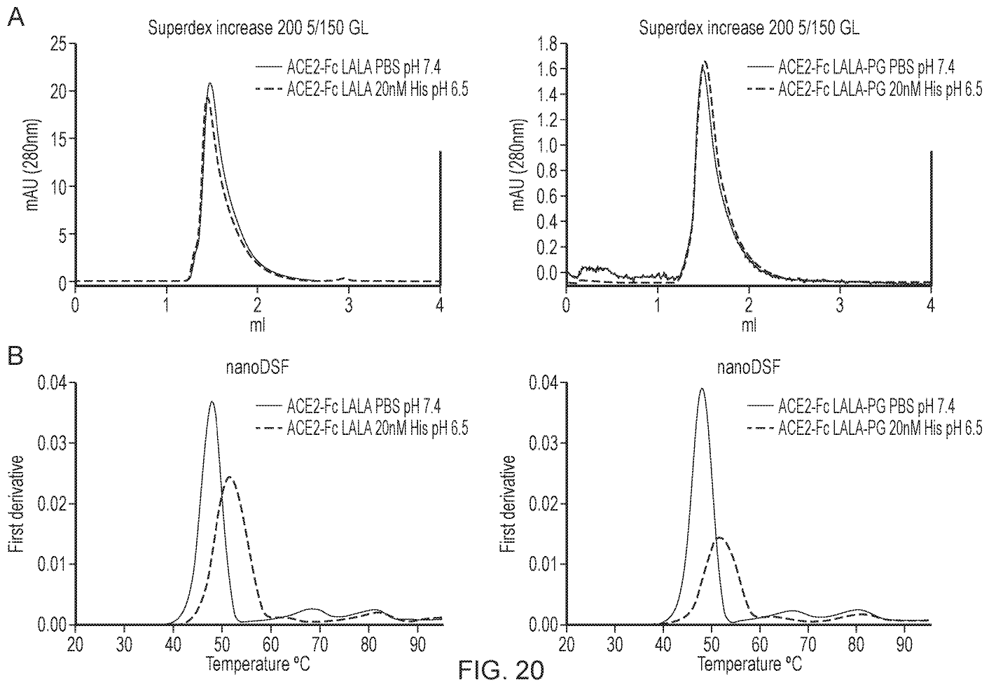

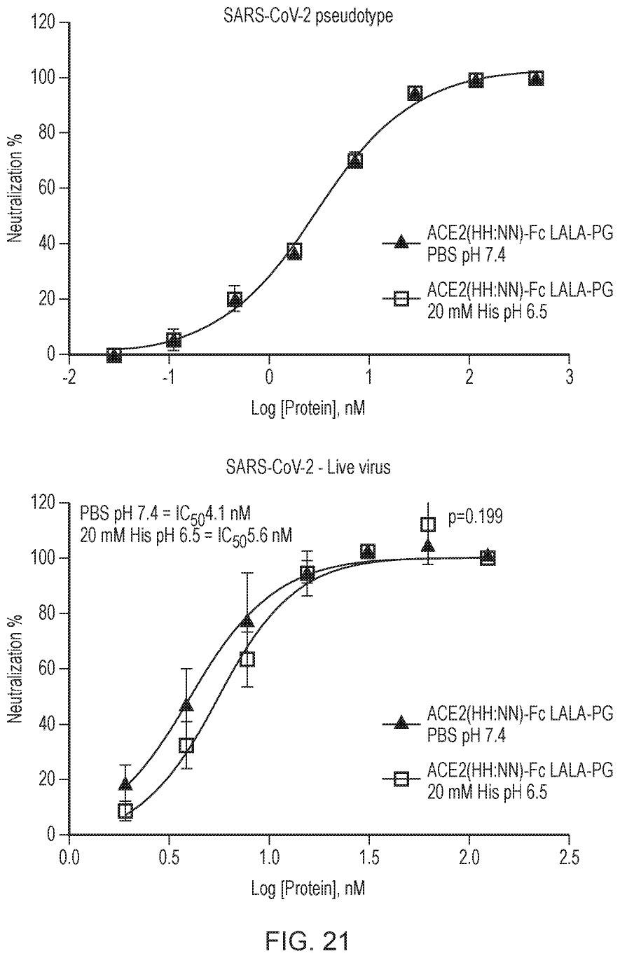

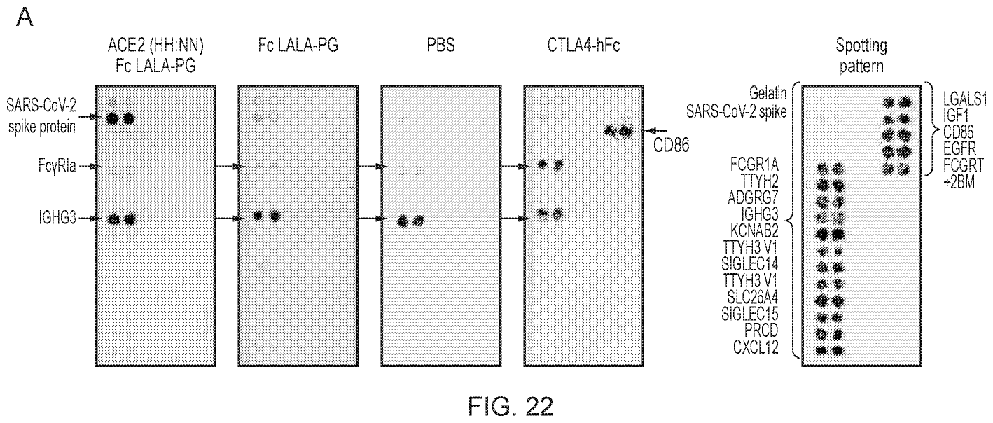

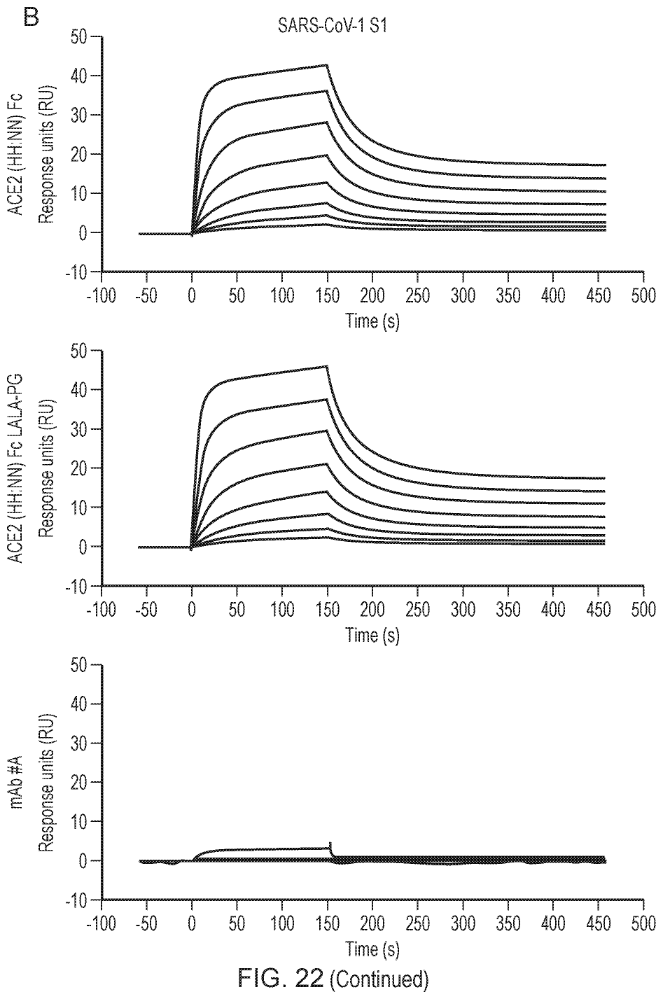

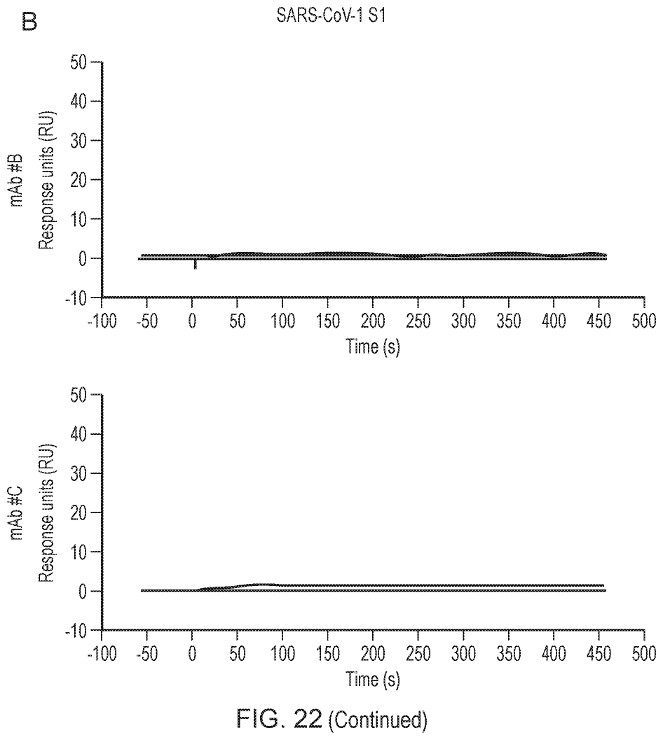

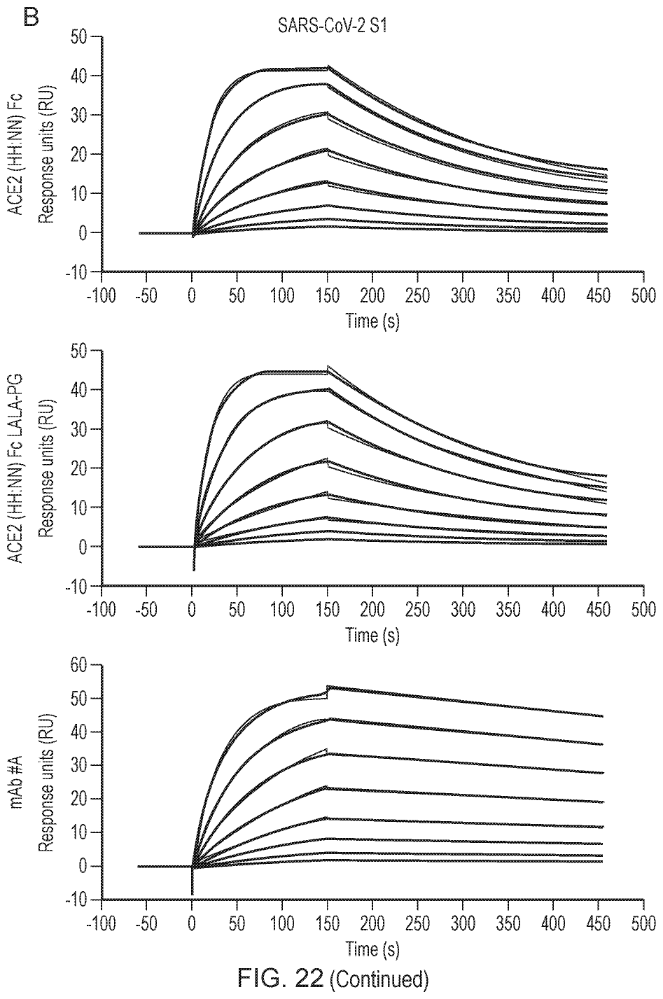

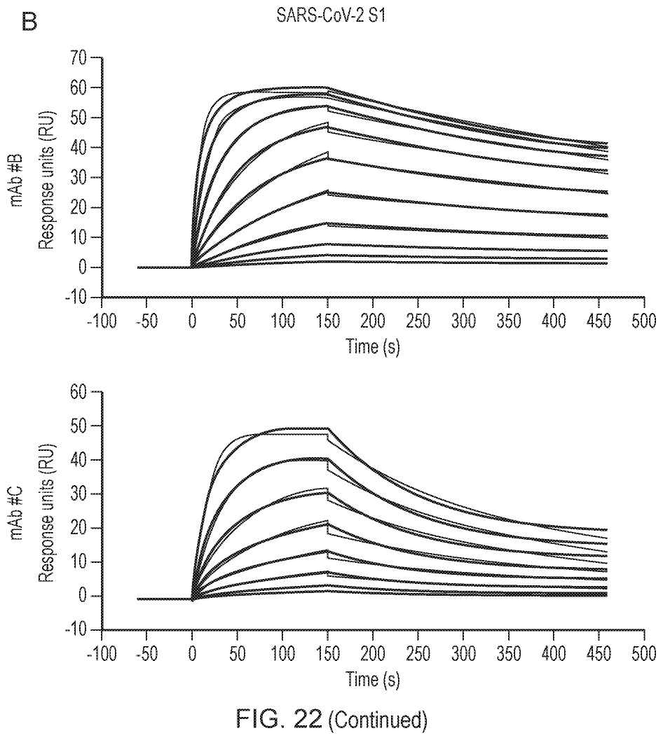

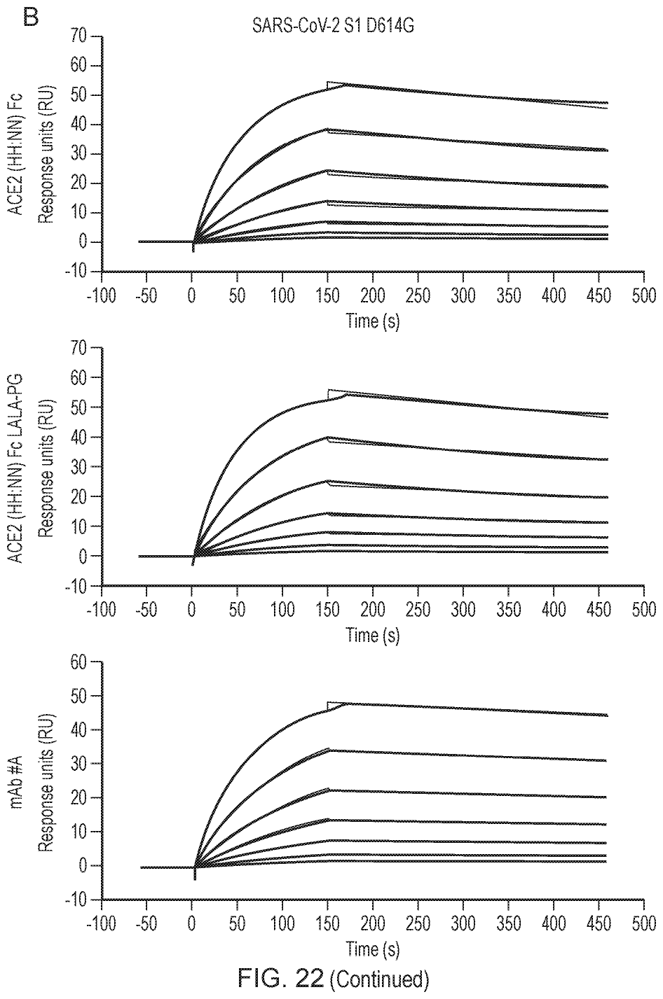

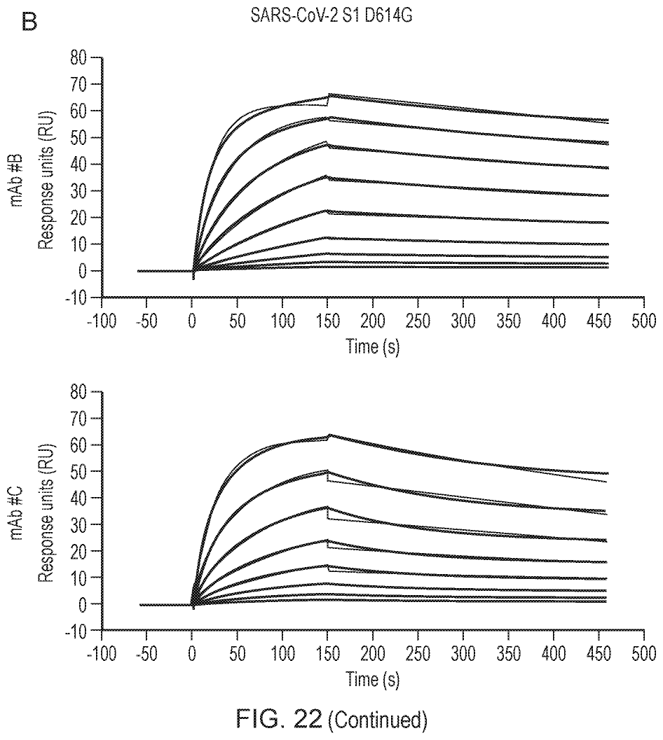

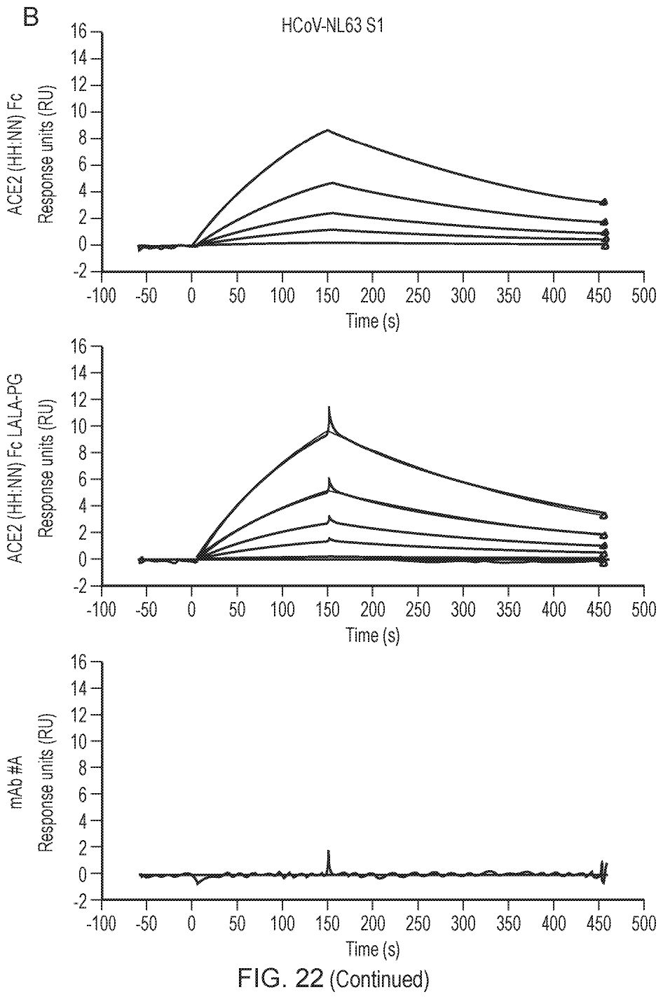

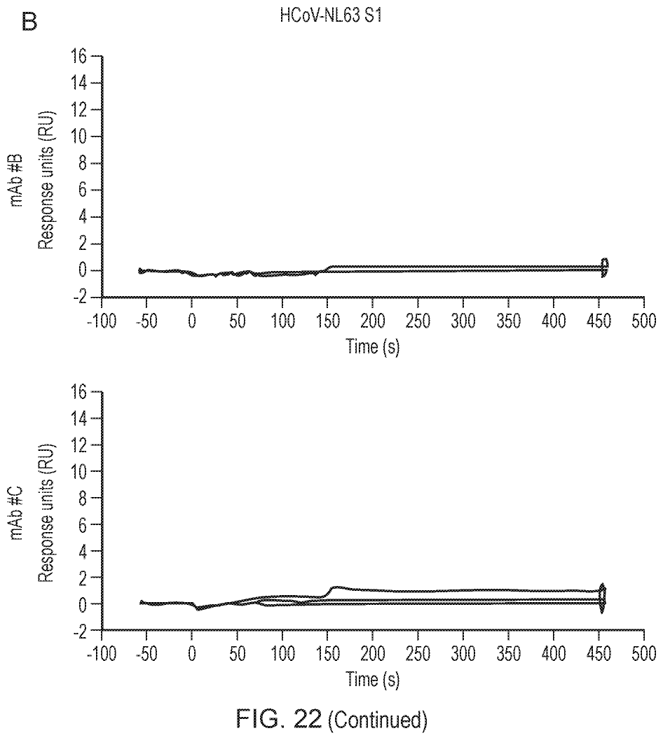

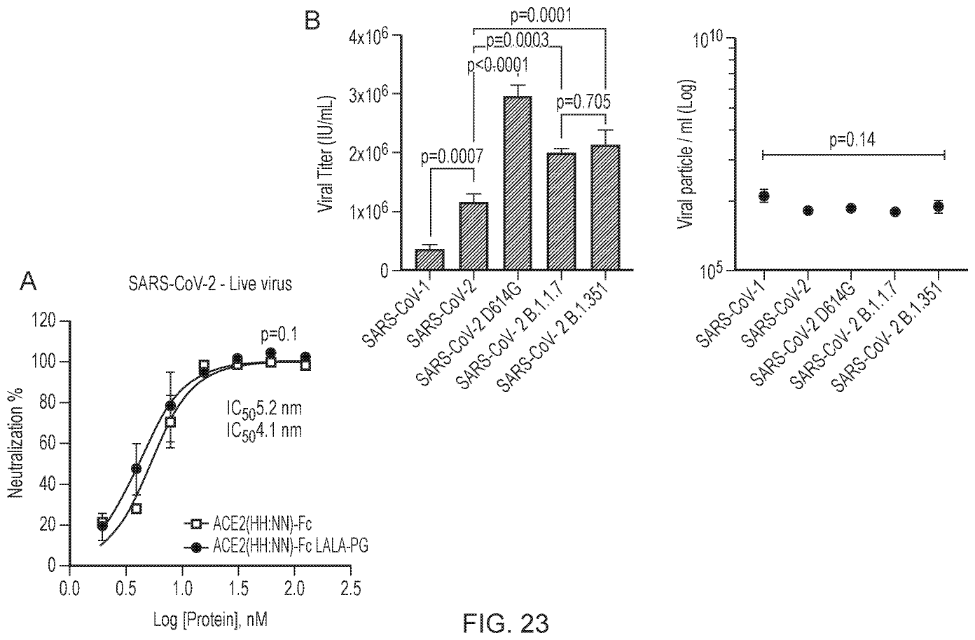

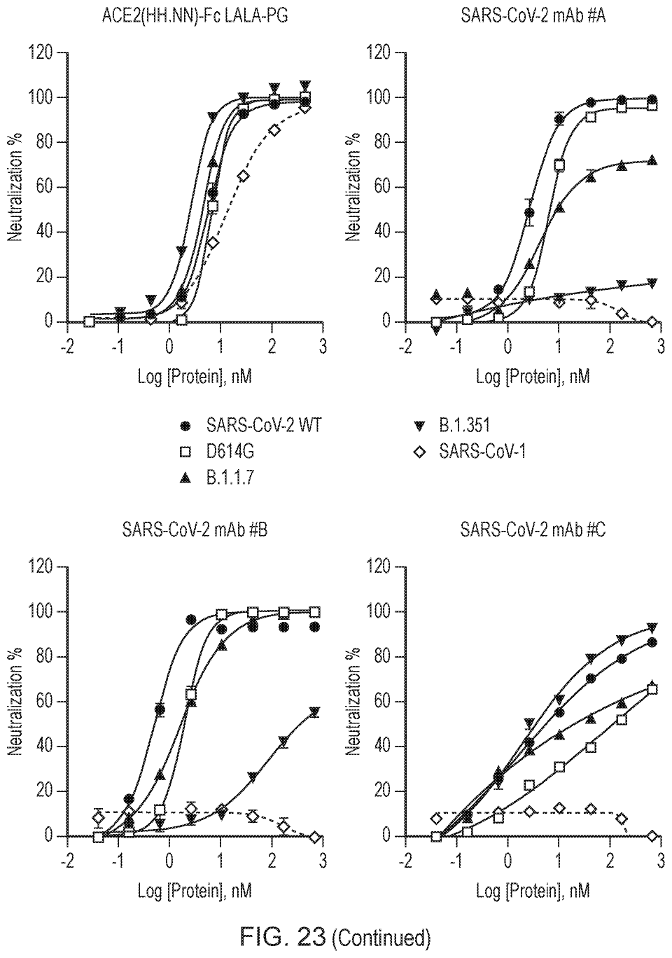

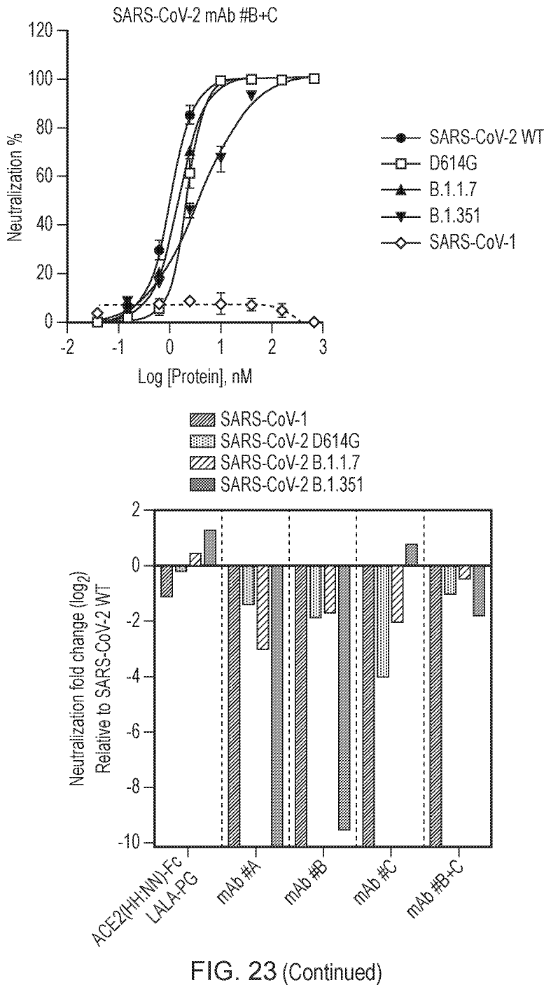

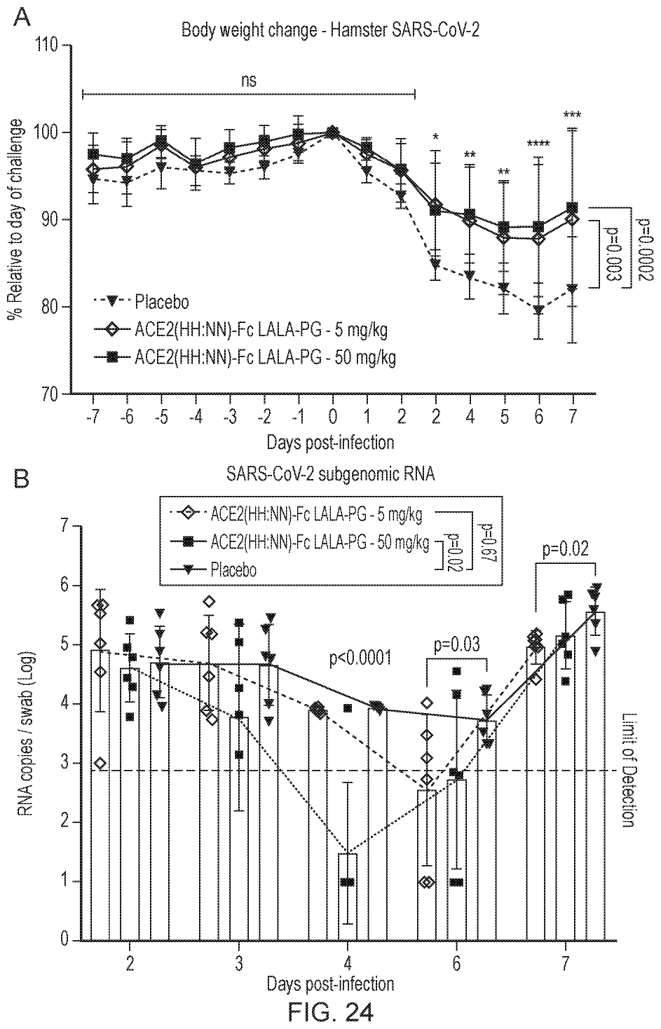

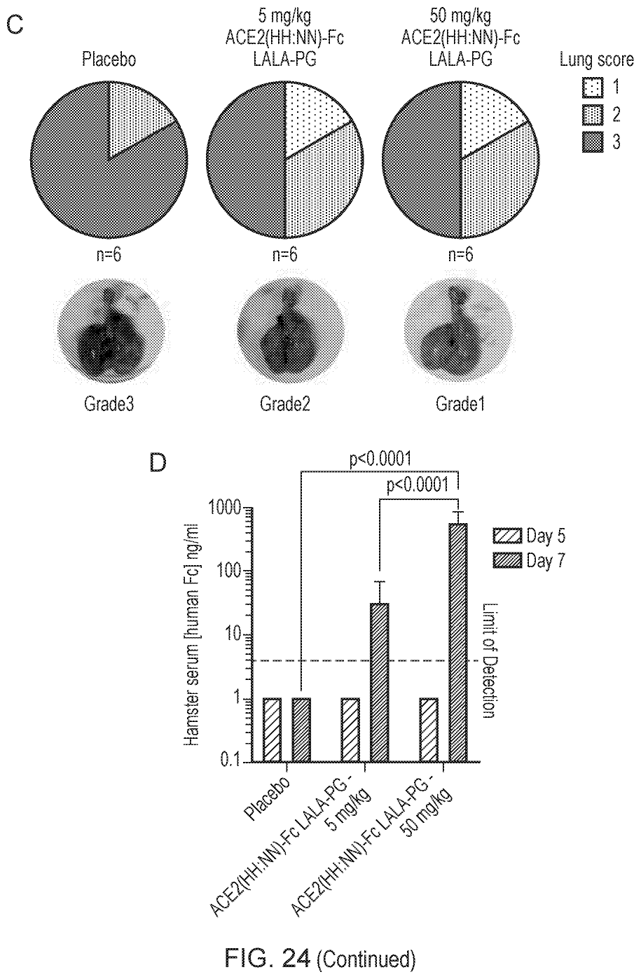

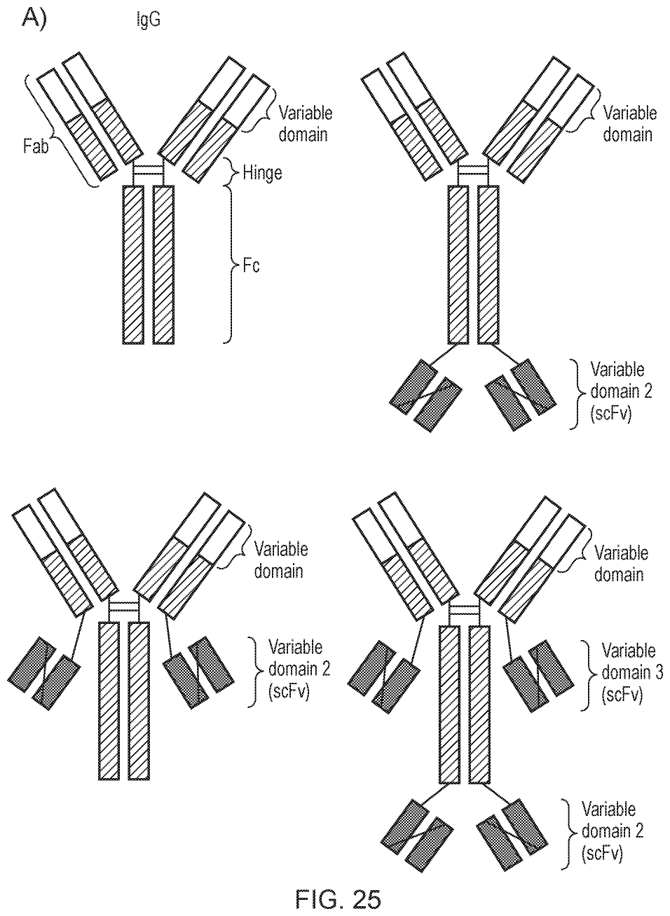

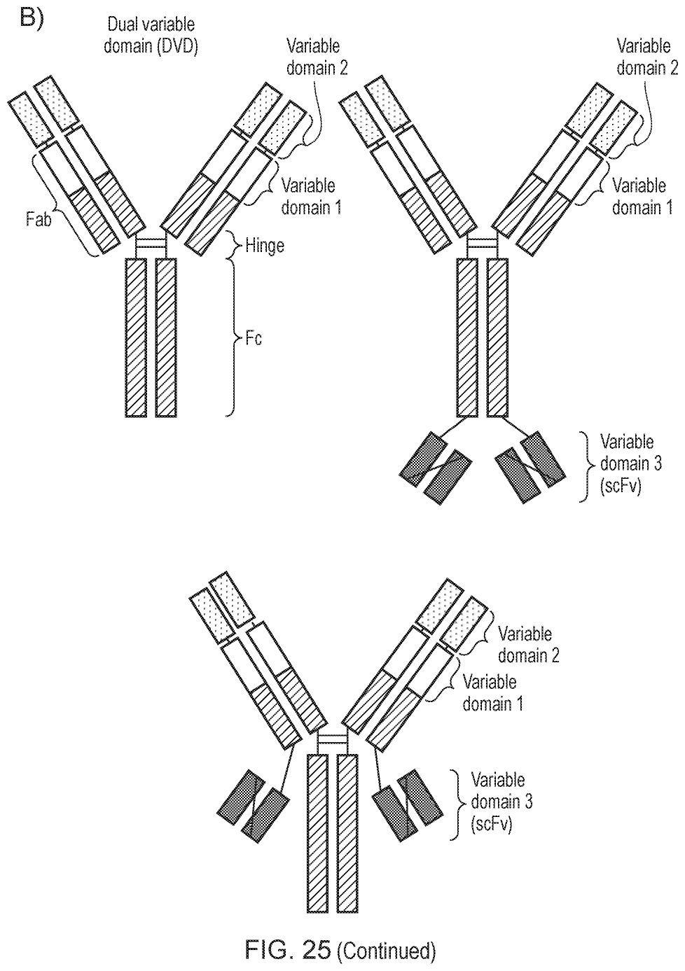

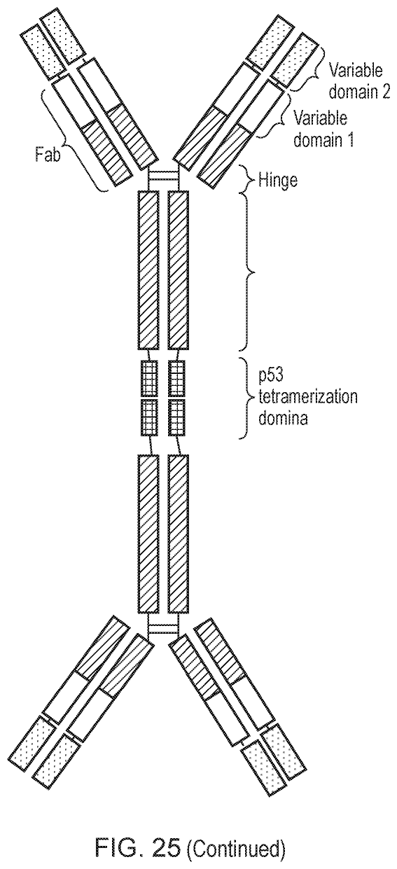

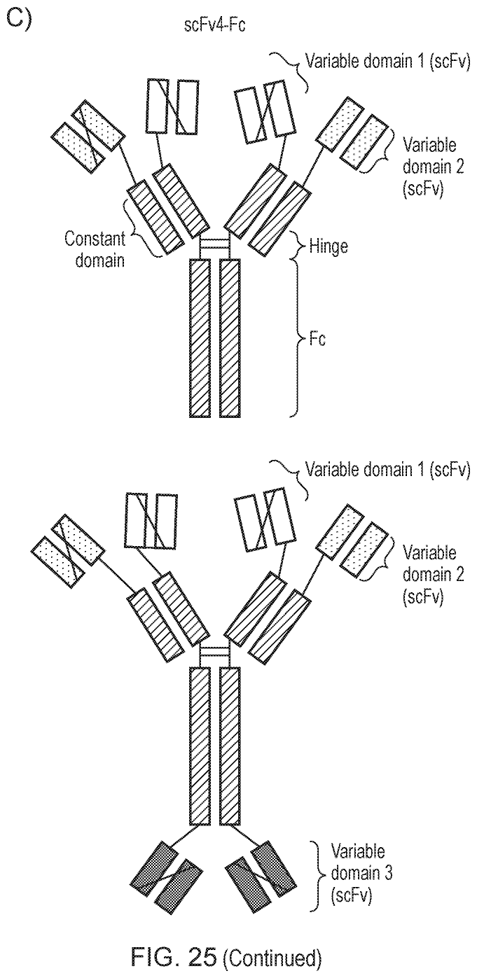

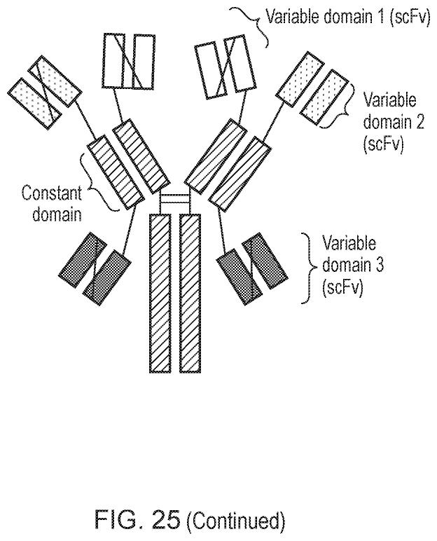

Active ACE2-Fc (ACRO biosystems) was compared to the inactive ACE2(18-740; HH:NN)-Fc molecule. A 1:1 binding kinetic model shows an 11-fold reduction in affinity for their natural substrate Ang II (Table 1), primarily due to a slower on-rate (ka) (FIG. 14A).

TABLE 1
Binding kinetics between active ACE2-Fc and
inactive ACE2 (HH:NN)-Fc for angiotensin
2, fitted to a Langmuir 1:1 binding model.

| Sample | Ka (1/Ms) | Kd (1/s) | KD (M) |
|---|---|---|---|
| Inactive ACE2 (18-740)-Fc | 1.38 × 10⁵ | 1.80 × 10⁻¹ | 1.30 × 10⁻⁶ |
| Active ACE2 (18-740)-Fc | 7.80 × 10⁵ | 9.11 × 10⁻² | 1.17 × 10⁻⁷ |

SPR measurements of kinetic interaction for the S1 domain of SARS-CoV-2 showed comparable kinetic profiles between active WT and HH:NN ACE2 (FIG. 14B and Table 3), further suggesting the preservation of an unaltered Spike binding domain.

ELISA on Spike Protein

Nunc Maxisorp clear 96-well plates were coated with 1 μg/ml (in PBS) of the relevant recombinant protein (SARS-CoV-2 spike trimer, SARS-CoV-2 S1, or BSA as control) overnight at 4° C. Plates were blocked with PBS 2% BSA for 1 h at RT. Active ACE2-Fc (ACRO biosystems) was compared to the inactive ACE2(18-740; HH:NN)-Fc molecule. were incubated at 10 μg/ml with 3-fold serial dilutions for 1 h at RT in PBS 0.5% BSA. Bound proteins were detected with anti-human HRP conjugated antibodies (Jackson Immunotools) at 1:3000 dilution in PBS 0.5% BSA, respectively. Incubation was allowed for 1 h at RT. All washes were performed in PBS 0.05% Tween20. Detection reagent 1-step TMB Ultra (thermo scientific) and reaction blocked with 1M H₂SO₄. Plates were acquired on a Varioskan Lux instrument at a wavelength of 450 nm.

The mutations introduced in the catalytic site of ACE2 did not impact the overall protein structure, and both WT and mutated ACE2 showed comparable binding capacity for recombinant SARS-CoV-2 full S trimer and S1 domain by ELISA (FIG. 14C).

Taken together, these results demonstrate an impaired capacity for the inactive ACE2(HH:NN)-Fc construct described here, to interact and process the endogenous substrate.

### Example 13: Optimisation of Fc Domain

To abrogate Fc effector functions, thus overcoming the risk of activating the host immune system and exacerbating the hyperinflammatory response often associated with severe COVID-19 development, the Fc domain was engineered to remove FcγR interactions. The human IgG1 Fc domain was mutated by introducing the LALA mutations of the CH2 domain alone or in combination with P329G (LALA-PG). Inactive ACE2 (HH:NN)-Fc LALA and ACE2-Fc LALA-PG, based on ACE2 aa18-740, were expressed by transient transfection in ExpiCHO cell line, as described above, and purified by Protein A affinity chromatography. The recombinant proteins showed a purity greater than 95% by SDS-PAGE.

Size Exclusion Chromatography

Twenty microliters of purified proteins were loaded onto a Superdex 200 increase 5/150 GL size exclusion chromatography column using Akta™ Pure system at a flow rate of 0.3 mL/min in PBS to determine protein purity and aggregation status. Eluted protein was detected at OD 280 nm.

The recombinant ACE2-Fc LALA and ACE2-Fc LALA-PG showed lower aggregation propensity compared to ACE2(18-740)-Fc (FIG. 15A and Table 2).

Differential Scanning Fluorimetry (DSF)

Thermal stability was determined by differential scanning fluorimetry nano(DSF) on a Prometheus NT.48 instrument (Nanotemper) using first derivative of 350/330 nm ratio to determine the melting temperature (Tm) value. Samples resuspended in PBS pH 7.4 were loaded on a glass capillary and temperature scanned from 20 to 95° C. with 1° C./min intervals.

The recombinant inactive ACE2-Fc LALA and ACE2-Fc LALA-PG showed a higher thermal stability (Tm) compared to ACE2(18-740)-Fc (FIG. 15B and Table 2).

TABLE 2
Yield and biophysical properties of different ACE2-Fc constructs.

| | Host | Yield | Aggregates | Apparent MW | Tm |
|---|---|---|---|---|---|
| Clone | cell | (g/L) | by SEC (%) | by SEC (kDa) | (° C.) |
| ACE2-Fc | CHO | 0.2 | 2.82 | 418 | 46.1 |
| ACE2-Fc | CHO | 0.12 | 2.34 | 488 | 47.7 |
| LALA | | | | | |
| ACE2-Fc | CHO | 0.19 | 2.48 | 454 | 48.1 |
| LALA-PG | | | | | |

Overall, these results revealed that the ACE2-Fc constructs based on an optimised Fc region have good biophysical properties, including lower aggregation propensity and a higher thermal stability (Tm) compared to ACE2(18-740)-Fc (FIG. 15 and Table 2). The LALA-PG clone showed the comparable expression yield (+25%) and higher Tm (+2° C.) compared to parental construct, and higher expression yield (+58%) and Tm (+0.4° C.) compared to the LALA construct (Table 2).

Binding to Spike Protein

Nunc Maxisorp clear 96-well plates were coated with 1 μg/ml (in PBS) of the relevant recombinant protein (SARS-CoV-2 spike trimer, SARS-CoV-2 S1, or BSA as control) overnight at 4° C. Plates were blocked with PBS 2% BSA for 1 h at RT. Active ACE2-Fc (ACRO biosystems) was compared to the inactive ACE2(18-740; HH:NN)-Fc molecule. were incubated at 10 μg/ml with 3-fold serial dilutions for 1 h at RT in PBS 0.5% BSA. Bound proteins were detected with anti-human HRP conjugated antibodies (Jackson Immunotools) at 1:3000 dilution in PBS 0.5% BSA, respectively. Incubation was allowed for 1 h at RT. All washes were performed in PBS 0.05% Tween20. Detection reagent 1-step TMB Ultra (thermo scientific) and reaction blocked with 1M H₂SO₄. Plates were acquired on a Varioskan Lux instrument at a wavelength of 450 nm.

Mutations on the Fc domain did not affect the binding capacity of ACE2 for SARS-CoV-2 S-protein and all three versions showed highly comparable dose/response curves to recombinant SARS-CoV-2 full S trimer or S1 domain by ELISA (FIG. 16A).

Similarly, all three variants were able to bind SupT1 cell lines expressing SARS-CoV-2 full S trimer as a transmembrane protein (FIG. 16B), further confirming binding capacity for the glycoprotein in a more physiological environment.

Binding to Fc Gamma Receptors (FcγR)

Fc interaction for human FcγRI, FcγRII and FcγRIII was tested on K562, U937 and SupT1 human cell lines. K562 are reported to express RNA for FcγRIIa and IIIa/b, while U937 express FcγRIa/b, IIa/b and IIIb (source www.proteinatlas.org, v20.0). A reverse flow cytometry detection assay, using biotinylated SARS-CoV-2 S1 as secondary reagent, demonstrated that the ACE2 construct with WT Fc efficiently bound both K562 and U937 in a dose-dependent manner (FIG. 16C). No binding was detected with either LALA or LALA-PG Fc mutations. SupT1 cells are not reported to express FcγR on the membrane and, consequently, failed to show binding events with the tested molecules.

Equally, human M1 polarised monocyte-derived macrophages (MDM) from healthy donors, showed strong interaction with the ACE2 carrying WT Fc, while no detectable engagement was obtained with the LALA-PG Fc mutation (FIG. 16D).

SPR Assay

Recombinant ACE2-Fc constructs were captured on flow cell 2, 3 and 4 of a Series S Protein A sensor chip (GE Healthcare) to a density of 70 RU using a Biacore T200 instrument. HBS-EP+ buffer was used as running buffer in all experimental conditions. Recombinant purified SARS-CoV-2 S1 protein (Acro biosystems) at known concentrations (concentration range from 250 nM to 3.9 nM) was used as the ‘analyte’ and injected over the respective flow cells with 150 s contact time and 500 s dissociation at 30 l/minute of flow rate with a constant temperature of 25° C. In each experiment, flow cell 1 was unmodified and used for reference subtraction. A ‘0 concentration’ sensogram of buffer alone was used as a double reference subtraction to factor for drift. Data were fit to a 1:1 Langmuir binding model. Since a capture system was used, a local Rmax parameter was used for the data fitting in each case.

InactiveACE2(HH:NN)-Fc showed strong interaction with FcγRIa and IIIa (27.5 nM and 73.2 nM, respectively) and reduced binding affinity for FcγRIIa and IIIb (207 nM and 118 nM, respectively). The LALA mutation still maintained residual binding to the FcγRIa and IIIa (657 nM and 225 nM, respectively) but no detectable binding to the remainder of the receptors. The LALA-PG mutation, however, showed a complete abrogation of FcγR binding, suggesting a more silent immunomodulatory profile (FIG. 16E).

Virus Neutralisation Assay

The ACE2-Fc wt, with LALA or LALA-PG mutations were tested in a live virus neutralisation assay. The assay is based on incubation of SARS-CoV-2 live virus or pseudotyped virus on VeroE6 cells and measuring for cell expression of viral spike protein and nucleocapsid at 48 h. Live viral neutralisation experiments were performed in a BSL3 laboratory. Briefly, VeroE6 cells were seeded at 2×10⁶ cells/ml in a 96-well flat bottom plate and incubated at 37° C. 5% CO₂ overnight. Test constructs (at specific concentrations) were incubated at 37° C. for 1 h in the presence of 100 TCID₅₀/well of virus (300 TC1D₅₀ for pseudotyped lentivirus) in MEM culture medium. Protein/virus mixture was then incubated with cells for 48 h. For live virus, cells were fixed in 2% PFA and stained with anti-SARS-CoV-2 N protein antibody (1:500 dilution) for 1 h and detected with HRP conjugated secondary antibody. Wells were incubated with TMB substrate solution and reaction neutralised with 2N H₂SO₄. Signal acquired at a wavelength of 450 nm using a microplate reader. For pseudotyped virus, infectivity was measured via luciferase reporter assay.

A range of concentrations of each test agent was used to identify half maximal inhibitory concentration (IC₅₀) values for live virus (FIG. 17A). Similarly, the neutralisation efficiency was tested on a lentiviral pseudotyped virus expressing SARS-CoV-2 spike protein (FIG. 17B). In both cases, the ACE2-Fc LALA-PG showed the best neutralisation efficiency at IC₅₀ of 4.1 nM for live virus and 0.1 nM for pseudotyped virus. These results were surprising because all three constructs share the same ACE2 domain and variations in the Fe domain were not expected to have an effect in the interaction between ACE2 and the spike protein of SARS-CoV-2.

### Example 14: Viral S Glycoprotein Binding Specificity and Neutralisation Profile

Binding specificity and cross-reactivity of the ACE2(HH:NN)-Fc LALA-PG construct was assessed using a cell-based protein microarray assay, screening 5477 full length plasma membrane and cell surface-tethered human secreted proteins, plus 371 human heterodimers and the SARS-CoV-2 S-protein. The test construct showed strong specific binding to the target protein SARS-CoV-2 S, while no other interaction was detected across the comprehensive panel of human protein (FIG. 22A). An Fc LALA-PG only construct with the ACE2 domain omitted did not display any interaction with SARS-CoV-2 S-protein or any other target tested. The control fusion protein CTLA4-hFc instead, showed strong interaction for its predicted target CD86, and the FcγRIa, due to the presence of a WT IgG1 Fc domain. A secondary anti-human Fc antibody interaction with human IgG3 was detected across all conditions tested (FIG. 22A).

Binding kinetics were generated for the S1 spike domain of SARS-CoV-1, SARS-CoV-2, SARS-CoV-2 D614G variant and HCoV-NL63, and compared to the leading anti-SARS-CoV-2 antibodies mAb #A, mAb #B and mAb #C. Remarkably, the ACE2-Fc fusion constructs mediated specific interaction towards all spike proteins tested, while the monoclonal antibodies showed specificity only for the SARS-CoV-2 related S-proteins (FIG. 22B). The ACE2(HH:NN)-Fc and ACE2(HH:NN)-Fc LALA-PG showed comparable affinities for the tested S1 domains, confirming no effect of the Fc mutations on ACE2 activity (FIG. 22B and Table 3). Interestingly, the ACE2-Fc constructs showed enhanced affinity for the SARS-CoV-2 S1 domain carrying the predominant D614G mutation, with a 4.5-fold increase in affinity, mainly driven by a slower off-rate. The monoclonal antibodies showed a 2-fold and 1.7-fold increase for the mAb #A, mAb #2 and mAb #C, respectively (FIG. 22B and Table 3).

TABLE 3
Kinetic affinities

| | | 1:1 binding kₐ | | | Two-state |
|---|---|---|---|---|---|
| Clone | Spike S1 domain | (1/Ms) | kd (1/s) | K_{D} (M) | K_{D} (M) |
| ACE2-Fc active | SARS-CoV-1 | 4.17e+5 | 1.44e−2 | 3.45e−8 | 1.98e−8 |
| | SARS-CoV-2 | 1.56e+5 | 4.44e−3 | 2.84e−8 | |
| | SARS-CoV-2 D614G | 1.39e+5 | 8.49e−4 | 6.11e−9 | |
| | HCoV-NL63 | 2.97e+3 | 3.55e−3 | 1.20e−6 | |
| ACE2 (HH:NN) Fc | SARS-CoV-1 | 4.93e+5 | 1.31e−2 | 2.65e−8 | 9.31e−9 |
| | SARS-CoV-2 | 2.66e+5 | 4.29e−3 | 1.61e−8 | |
| | SARS-CoV-2 D614G | 1.62e+5 | 5.79e−4 | 3.58e−9 | |
| | HCoV-NL63 | 4.00e+3 | 3.34e−3 | 8.35e−7 | |
| ACE2 (HH:NN)-Fc | SARS-CoV-1 | 5.05e+5 | 1.39e−2 | 2.75e−8 | 9.69e−9 |
| (LALA-PG) | SARS-CoV-2 | 2.92e+5 | 4.28e−3 | 1.47e−8 | |
| | SARS-CoV-2 D614G | 1.79e+5 | 6.00e−4 | 3.35e−9 | |
| | HCoV-NL63 | 3.72e+3 | 3.49e−3 | 9.38e−7 | |
| mAb #A | SARS-CoV-1 | N/A | N/A | N/A | |
| | SARS-CoV-2 | 1.29e+5 | 6.11e−4 | 4.73e−9 | |
| | SARS-CoV-2 D614G | 1.45e+5 | 2.79e−4 | 1.93e−9 | |
| | HCoV-NL63 | N/A | N/A | N/A | |
| mAb #B | SARS-CoV-1 | N/A | N/A | N/A | |
| | SARS-CoV-2 | 4.46e+5 | 1.23e−3 | 2.75e−9 | |
| | SARS-CoV-2 D614G | 3.78e+5 | 5.89e−4 | 1.56e−9 | |
| | HCoV-NL63 | N/A | N/A | N/A | |
| mAb #C | SARS-CoV-1 | N/A | N/A | N/A | |
| | SARS-CoV-2 | 4.45e+5 | 5.65e−3 | 1.27e−8 | |
| | SARS-CoV-2 D614G | 1.45e+5 | 1.04e−3 | 7.18e−9 | |
| | HCoV-NL63 | N/A | N/A | N/A | |

Both ACE2(HH:NN)-Fc and ACE2(HH:NN)-Fc LALA-PG showed comparable neutralisation efficiency for live SARS-CoV-2 virus in vitro, with IC₅₀ values of 5.2 and 4.1 nM, respectively, providing evidence of potent therapeutic activity (FIG. 23A). To investigate the degree of neutralisation efficiency against the SARS-CoV-2 variants of interest, replication deficient lentiviral vectors pseudotyped with the glycoproteins of SARS-CoV-2 WT (derived from the Wuhan Hu-1 strain), D614G mutation, UK B.1.1.7 and South Africa B.1.351 variants were engineered. Although viral vectors pseudotyped with protein S of SARS-CoV-1 and the four SARS-CoV-2 variants showed equivalent physical particle concentrations, as measured by p24 ELISA, they exhibited wildly different infectivity capacity (FIG. 23B). SARS-CoV-1 resulted in the lowest viral titre with a reduction of 3.2-fold in infectious units (IU)/ml compared to WT SARS-CoV-2. The SARS-CoV-2 D614G variant was instead the most efficient with 2.6-fold higher viral titre compared to WT. B.1.1.7 and B.1.351 showed 1.8 and 1.9-fold higher viral titres, compared to WT SARS-CoV-2, respectively (FIG. 23B).

The ACE2(HH:NN)-Fc LALA-PG was able to efficiently neutralise SARS-CoV-2, with tight dose-response curves among the SARS-CoV-2 variants, and SARS-CoV-1 (FIG. 23C). Interestingly, the neutralisation capacity was slightly improved for the B.1.1.7 and B.1.351 variants compared to WT SARS-CoV-2. The monoclonal antibody mAb #A showed a marked reduction in neutralisation capacity for the D614G and B.1.1.7 variants, 3 and 8-fold respectively, significantly impacting on the antibody efficacy; with an almost complete abrogation of neutralisation against the B.1.351 variant (FIG. 23C). The antibody cocktail mAb #B+C was more resilient in its response to the SARS-CoV-2 variants but was characterised by a 4-fold reduction in neutralisation for the B.1.351 variant. When the two antibodies constituting the cocktail were analysed individually, the mAb #B showed a 3-fold decrease in neutralisation capacity for the D614G and B.1.1.7 variants, with a staggering 3-Log reduction for the B.1.351 variant; while the mAb #C showed a 4-fold neutralisation reduction for the B.1.1.7 variant and a Log shift for the D614G variant (FIG. 23C).

### Example 15: In Vivo SARS-CoV-2 Neutralisation

It has been previously reported that Syrian hamsters (Mesocricetus auratus) are a relevant small animal model for SARS-CoV-2 infection reporting symptoms such as reduced body weight and pathological lesions on the lung. Syrian hamsters were challenged intranasally with 10^{4.5} TCID₅₀ viral inoculum and then dosed 24 h later via intra-peritoneal (i.p.) injections of ACE2(HH:NN)-Fc LALA-PG at either 5 mg/kg or 50 mg/kg. PBS injections were used for the placebo control group. Body weight and clinical observations were performed at regular intervals, blood samples collected for PK analysis, and throat swabs taken for analysis of viral load by PCR.

The hamster groups treated with either high or low ACE2(HH:NN)-Fc LALA-PG doses showed a significant protection against body weight loss, with a maximum average weight loss of 11% compared to 21% for the placebo group, relative to the day of viral challenge (FIG. 24A). Throat swabs revealed a substantial reduction in viral RNA copies between day 4 and day 6 post-viral challenge, compared to the placebo; several animals showed undetectable levels of RNA between day 3 and day 6 for the high ACE2(HH:NN)-Fc LALA-PG dose and an overall viral load significantly lower than placebo control group (FIG. 24B). Macro-analysis on lung necropsies (day 7) also showed an overall reduction in lung damage for the ACE2(HH:NN)-Fc LALA-PG treated groups, characterised by fewer lesions and blood clotting (FIG. 24C). Finally, i.p. administered ACE2(HH:NN)-Fc LALA-PG was still detectable in the hamster sera at day 7, with levels almost 20-fold higher for the high dose compared to low dose treatment (FIG. 24D).

### Example 16: Down-Stream Processing and Formulation Optimisation

To define a suitable formulation considering manufacturing scale-up for clinical application, the well-established antibody formulation buffer, 20 mM His, was used to solubilise the ACE2(HH:NN)-Fc at a range of pH conditions from 3.5 to 7. Thermal stability profile was determined using differential scanning fluorimetry (DSF) to identify the first transition event (Tm ° C.). The ACE2(HH:NN)-Fc in PBS at pH 7.4 showed a first unfolding event at 46.1° C., attributed to the unfolding of the ACE2 domain (FIG. 18A). When tested in 20 mM His buffer, the first unfolding event occurred at a Tm between 42.3 and 51.6° C., with the lowest Tm associated with pH 3.5 and the most stable Tm obtained at pH 6.5 (FIG. 18A).

A crucial phase during manufacturing of monoclonal antibodies lies in the viral inactivation step, often carried out at low pH, which can affect the stability and aggregation state of the proteins in solution. To investigate this, the ACE2(HH:NN)-Fc was incubated in 20 mM His pH 3.5 for 90 minutes before dialysis in 20 mM His pH 6.5. Thermal stability comparison of ACE2(HH:NN)-Fc at pH 3.5, 6.5 and 3.5 dialysed to 6.5 showed how the initial instability due to pH 3.5 could efficiently be restored to that of the ACE2(HH:NN)-Fc in 20 mM His pH 6.5 solution following dialysis (FIG. 18B). The ACE2(HH:NN)-Fc in 20 mM His pH 6.5 presented also two additional unfolding events at 61.9° C. and 81.3° C. belonging to the CH2 and CH3 domains, respectively. The distribution of particles within the solution showed a predominantly monodispersed profile for the ACE2(HH:NN)-Fc in PBS at pH 7.4 with an average diameter of 13.5 nm, in agreement with a molecule of predicted MW of 219 kDa. Similarly, the ACE2(HH:NN)-Fc in 20 mM His pH 6.5 showed a comparable monodispersed profile with a particle average of 13.3 nm. The suspension in a low pH buffer of 3.5 did not significantly enhance aggregation of ACE2(HH:NN)-Fc but depicted a slight increase in average particle diameter to 16.8 nm. Upon dialysis to 20 mM His pH 6.5, the apparent particle size was reduced to 15.0 nm, without increasing aggregates (FIG. 18C). Furthermore, the change of buffer from PBS pH 7.4 to 20 mM His pH 6.5 and, crucially, the viral inactivation step in 20 mM His pH 3.5 with subsequent dialysis to pH 6.5, did not affect the capacity of the ACE2(HH:NN)-Fc to bind the SARS-CoV-2 S1 protein, further validating the proposed process (FIG. 18D).

The ACE2(HH:NN)-Fc LALA-PG also showed an increased thermal stability when in 20 mM His pH 6.5 buffer, with Tm moving from 48.1° C. to 52.0° C. for the unfolding of the ACE2 domain (FIG. 18E). Interestingly, the unfolding of the Fc LALA-PG domain also occurred at temperatures similar to the unmodified Fc, with Tm of 66.8° C. and 80.6° C. for the CH2 and CH3 domains, respectively, in PBS pH 7.4 and 64.3° C. and 81.8° C. for CH2 and CH3, respectively, in 20 mM His pH 6.5 (FIG. 18E). The ACE2(HH:NN)-Fc LALA-PG was also characterised by a monodispersed particle profile with an average diameter size of 13.8 nm and 13.6 nm for the PBS pH 7.4 and 20 mM His pH 6.5 formulations, respectively (FIG. 18F). The construct at a concentration of 20 mg/ml in 20 mM His pH 6.5 still exhibited a monodispersed distribution with an average particle size 10.2 nm. Increasing concentrations of polysorbate 80 from 0.01 to 1% showed a slight particle size reduction to 9.9 nm with the highest concentration (FIG. 18G).

Biophysical Characterisation of Low pH Exposed ACE2-Fc Constructs

During monoclonal antibody production, a viral inactivation step at low pH (3-3.5) is generally required as a safety measure. As the 20 mM His pH 3.5 showed a low unfolding transition (Tm 42.3° C., FIG. 18), probably due to partial unfolding due to strong inter-molecular repulsion, the protein was subjected to a transition from PBS pH 7.4 to 20 mM His pH 3.5 for 90 min and then rescued in 20 mM His pH 7 and tested for thermal stability, aggregation by SEC and binding capacity by ELISA, as described previously.

Results showed that the inactive ACE2 (HH:NN)-Fc fully maintained Spike protein binding capacity comparable biophysical characteristics even after prolonged exposure to low pH (FIG. 19 and Table 4).

TABLE 4
Biophysical characterisation of low pH exposed inactive ACE2-Fc.

| | Aggregates | Apparent MW | Tm | ELISA EC₅₀ |
|---|---|---|---|---|
| Buffer | by SEC (%) | by SEC (kDa) | (° C.) | (nM) |
| PBS | 2.82 | 418 | 46.1 | 0.08 |
| 20 mM His pH 3.5 | | | 42.3 | |
| 20 mM His pH 3.5 | 6.62 | 532 | 50.3 | 0.06 |
| dialysed to pH 7 | | | | |
| 20 mM His pH 6.5 | | | 51.6 | 0.05 |
| 20 mM His pH 7 | 5.56 | 526 | 50.3 | |

Similarly, when the inactive ACE2-Fc constructs carrying the LALA or LALA-PG mutations were resuspended in 20 mM His buffer at pH 6.5, the constructs showed a 3.9° C. increase in Tm compared to PBS, while maintaining similar aggregation propensity (FIG. 20 and Table 5).

TABLE 5
Formulation optimisation for inactive
(HH:NN) ACE2-Fc LALA and LALA-PG.

| | | Aggregates | Apparent MW | Tm |
|---|---|---|---|---|
| Clone | Buffer | by SEC (%) | by SEC (kDa) | (° C.) |
| ACE2-Fc | PBS pH 7.4 | 2.46 | 544 | 47.7 |
| LALA | | | | |
| ACE2-Fc | 20 mM His pH 6.5 | 3.03 | 592 | 51.6 |
| LALA | | | | |
| ACE2-Fc | PBS pH 7.4 | 3.13 | 512 | 48.1 |
| LALA-PG | | | | |
| ACE2-Fc | 20 mM His pH 6.5 | 3.88 | 474 | 52 |
| LALA-PG | | | | |

Virus Neutralisation Assay

Virus neutralisation on live SARS-CoV-2 virus and lentiviral pseudotyped virus using inactive ACE2-Fc LALA or inactive ACE2-Fc LALA-PG constructs formulated in PBS or 20 mM His pH 6.5 was carried out as described in Example 13.

The formulation in 20 mM His pH 6.5 of ACE2(HH:NN)-Fc LALA-PG did not alter the SARS-CoV-2 neutralisation capacity of the construct. Efficient neutralisation, comparable to the PBS pH 7.4 formulation, was determined against a lentiviral vector pseudotyped with SARS-CoV-2 S glycoprotein with IC50 of 2.9 nM and 3.0 nM; and against the live SARS-CoV-2 virus with an IC50 of 4.1 nM and 5.6 nM for the PBS pH7.4 and 20 mM His pH 6.5 formulation, respectively (FIG. 21).

These results demonstrate that the neutralisation capacity of inactive ACE2-Fc LALA or ACE2-Fc LALA-PG constructs against live SARS-CoV-2 virus or pseudotyped lentivirus was maintained within range of the PBS formulated constructs.

### Example 17: Generation of Tetravalent Fusion Proteins Based on Inactive hACE2

The ectodomain of human ACE2 (18-740) is mutated to incorporate the H374N and H378N mutations (HH:NN or inactive ACE2) to inhibit catalytic activity. Then, the inactive hACE2 is fused to a human IgG1 hinge-CH2-CH3 domain and to a human IgG1 constant kappa domain (CL kappa), and cloned in a protein expression vector using a murine IgKappa leader sequence. A diagram of this molecule is shown in FIG. 2, fourth panel. The plasmid vector is transiently transfected onto suspension Freestyle HEK293 using polyethylenimine (PEI), and onto ExpiCHO using Expifectamine transfection reagent. Transfected cells are cultured for 5 days in a shaker incubator at 37° C., 8% CO₂ to allow for protein secretion. Culture supernatant is filtered using 0.22 μm filter units to remove large contaminants (cells and cellular debris).

Fusion proteins are purified using an AKTA™ pure system (GE Healthcare) using a HiTrap MabSelect PrismA 1 ml column (for IgG1 Fc fusion proteins). Briefly, the column is equilibrated with 5 column volumes of PBS pH 7.4. Supernatant is applied to the column at a flow rate of 1 mL/min. Following application of supernatant, the column is washed with 20 column volumes of PBS. Sample is then eluted from the column with 3 ml of IgG elution buffer (Pierce—21004) at 1 mL/min and directly loaded onto 2 HiTrap 5 ml desalting columns, previously equilibrated in PBS, and collected on a 96-well plate using a fraction collector unit. Proteins are characterised via SDS-PAGE under reducing and non-reducing conditions to assess molecular weight and purity.

### Example 18: Characterisation of Tetravalent Fusion Proteins Based on Inactive hACE2

SPR Assay

To determine 1:1 binding kinetics for SARS-CoV-2 S1 WT, D614G, B.1.1.7 and B.1.351 kinetics, tetrameric ACE2-Fc construct is captured to a density between 50 and 100 RU, on a Series S Protein A sensor chip (GE Healthcare—29127555) using a Biacore instrument (GE Healthcare). HBS-P+ buffer is used as running buffer in all experimental conditions. Recombinant purified spike proteins at known concentrations are used as the ‘analyte’ and injected over the respective flow cells with 150 s contact time and up to 500 s dissociation. Kinetics are performed at 25° C. with a flow rate of 30 μl/ml. Flow cell 1 is unmodified and used for reference subtraction. A ‘0 concentration’ sensogram of buffer alone is used as a double reference subtraction to factor for drift. Data are fit to a 1:1 Langmuir binding model using Biacore insight evaluation software (GE Healthcare). Since a capture system is used, a local Rmax parameter was used for the data fitting in each case.

To determine avidity effects of the poly-valent ACE2 fusion construct, spike S1 domains from SARS-CoV-2 WT, D614G, B.1.1.7 and B.1.351 are immobilised to a low, mid and high density on a anti-His Series S CM5 chip (GE Healthcare) using a Biacore T200 instrument (GE Healthcare). Tetrameric ACE2-Fc at known concentrations is used as the ‘analyte’ and injected over the respective flow cells with 150 s contact time and up to 500 s dissociation. Kinetics are performed at 25° C. with a flow rate of 30 μl/ml. Flow cell 1 is unmodified and used for reference subtraction. A ‘0 concentration’ sensogram of buffer alone is used as a double reference subtraction to factor for drift. Data are fit to a 1:1 Langmuir binding model using Biacore insight evaluation software (GE Healthcare). Since a capture system is used, a local Rmax parameter was used for the data fitting in each case.

SARS-CoV-2 Lentiviral Pseudotyped Viral Vector Neutralisation Assay

Tetrameric ACE2-Fc is serially diluted in PBS to 7 decreasing concentrations ranging from 100 mg/mL to 6.1 ng/mL (4-fold serial dilution). Each dilution is mixed 1:1 with lentiviral vectors pseudotyped with SARS-CoV S glycoproteins normalised to 1.0×10⁵ physical particle of vectors pseudotyped with WT Wuhan Hu-1 glycoprotein, to a final volume of 200 μL and incubated at 37° C. for 1 h. Antibody-virus mixtures are cultured with 3×10⁴ HEK-293T cells previously genetically engineered to express human ACE2 and TMPRSS2, in the presence of 8 μg/mL of polybrene, in 48-well plates with a final volume of 0.5 mL per well. Plates are spin-inoculated at 1000 g for 10 mins and incubated for 72 h. Viral titers are then quantified by eGFP expression in target cells using BD LSRFORTESSA X-20 cell analyser and infectivity of all fractions is determined as a percentage of viral titers in the PBS only control.

This application claims the benefit of United Kingdom application No. 2005333.6 filed on 9 Apr. 2020, United Kingdom application No. 2013372.4 filed on 26 Aug. 2020, and United Kingdom application No. 2103001.0 filed on 3 Mar. 2021. These applications are incorporated herein by reference in their entirety.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1.-21. (canceled)

22. A protein comprising:
i) a first polypeptide comprising the sequence shown as SEQ ID NO: 132, and
ii) a second polypeptide comprising the sequence shown as SEQ ID NO: 135.

23. A nucleic acid encoding the first and second polypeptides of claim 22.

24. A vector comprising the nucleic acid according to claim 23.

25. A cell comprising the nucleic acid according to claim 23.

26. A cell comprising the vector according to claim 24.

27. A method for making a protein according to claim 22, comprising culturing a cell comprising a nucleic acid encoding said first and said second polypeptides and purifying the protein from the supernatant.

28. A pharmaceutical composition comprising:
i) a protein according to claim 22, or
ii) a nucleic acid encoding said first and said second polypeptides, or a vector comprising said nucleic acid,
and a pharmaceutically acceptable carrier, diluent, excipient or adjuvant.

29. A method for treating a coronavirus infection or a condition or disorder resulting from this infection in a subject in need thereof, wherein the method comprises a step of administering to the subject:
i) a protein according to claim 22, or
ii) a pharmaceutical composition comprising a protein according to claim 22, or a nucleic acid or vector encoding said first and said second polypeptides, and a pharmaceutically acceptable carrier, diluent, excipient or adjuvant.

30. The method according to claim 29, wherein the coronavirus is SARS-CoV-2.

31. A method for treating a subject having COVID-19 of unknown SARS-CoV-2 strain, wherein the method comprises a step of administering to the subject:
i) a protein according to claim 22, or
ii) a pharmaceutical composition comprising a protein according to claim 22, or a nucleic acid or vector encoding said first and said second polypeptides, and a pharmaceutically acceptable carrier, diluent, excipient or adjuvant.

32. A method for treating a subject previously immunised with a vaccine based on S protein depicted under Uniprot accession number P0DTC2, wherein the method comprises a step of administering to the subject:
i) a protein according to claim 22, or
ii) a pharmaceutical composition comprising a protein according to claim 22, or a nucleic acid or vector encoding said first and said second polypeptides, and a pharmaceutically acceptable carrier, diluent, excipient or adjuvant.

33. A method for treating a subject previously treated with antibodies specific to S protein depicted under Uniprot accession number P0DTC2, wherein the method comprises a step of administering to the subject:
i) a protein according to claim 22, or
ii) a pharmaceutical composition comprising a protein according to claim 22, or a nucleic acid or vector encoding said first and said second polypeptides, and a pharmaceutically acceptable carrier, diluent, excipient or adjuvant.

34. A method for treating a subject previously infected with a first SARS-CoV-2 strain who is currently infected with a second SARS-CoV-2 strain, wherein the method comprises a step of administering to the subject:
i) a protein according to claim 22, or
ii) a pharmaceutical composition comprising a protein according to claim 22, or a nucleic acid or vector encoding said first and said second polypeptides, and a pharmaceutically acceptable carrier, diluent, excipient or adjuvant.

35. A method for treating a coronavirus infection of one SARS-CoV-2 strain selected from wild-type, variant D614G, variant A222V, variant S477N, variant B.1.1.7, variant B.1.351, and variant B.1.1.28, wherein the method comprises a step of administering comprising a step of administering to the subject:
i) a protein according to claim 22, or
ii) a pharmaceutical composition comprising a protein according to claim 22, or a nucleic acid or vector encoding said first and said second polypeptides, and a pharmaceutically acceptable carrier, diluent, excipient or adjuvant.

36. A method of neutralising a coronavirus infection, comprising a step of contacting a cell infected with said coronavirus with:
i) a protein according to claim 22, or
ii) a pharmaceutical composition comprising a protein according to claim 22, or a nucleic acid or vector encoding said first and said second polypeptides, and a pharmaceutically acceptable carrier, diluent, excipient or adjuvant.

37. The method according to claim 36, wherein the coronavirus is SARS-CoV-2.


SEQUENCE LISTING (140 sequences)

SEQ ID NO: 1 - protein, 723 aa, Artificial Sequence
  processed ACE2 ectodomain
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 2 - protein, 740 aa, Artificial Sequence
  full ACE2 ectodomain
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 3 - protein, 588 aa, Artificial Sequence
  truncated ACE2, aa 18-605
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVG

SEQ ID NO: 4 - protein, 59 aa, Artificial Sequence
  collagen XVIII trimerizing structural element (XVIIITSE)
SGVRLWATRQAMLGQVHEVPEGWLIFVAEQEELYVRVQNGFRKVQLEARTPLPRGTDNE

SEQ ID NO: 5 - protein, 64 aa, Artificial Sequence
  collagen XV trimerizing structural element (XVTSE)
VTAFSNMDDMLQKAHLVIEGTFIYLRDSTEFFIRVRDGWKKLQLGELIPIPADSPPPPALSSNP

SEQ ID NO: 6 - protein, 15 aa, Artificial Sequence
  linker
GGGGSGGGGSGGGGS

SEQ ID NO: 7 - protein, 299 aa, Artificial Sequence
  processed extracellular domain of CD147
AAGFVQAPLSQQRWVGGSVELHCEAVGSPVPEIQWWFEGQGPNDTCSQLWDGARLDRVHIHATYHQHAASTISIDTLVEEDTGTYECRASNDPDRNHLTRAPRVKWVRAQAVVLVLEPGTVFTTVEDLGSKILLTCSLNDSATEVTGHRWLKGGVVLKEDALPGQKTEFKVDSDDQWGEYSCVFLPEPMGTANIQLHGPPRVKAVKSSEHINEGETAMLVCKSESVPPVTDWAWYKITDSEDKALMNGSESRFFVSSSQGRSELHIENLNMEADPGQYRCNGTSSKGSDQAIITLRVRS

SEQ ID NO: 8 - protein, 198 aa, Artificial Sequence
  domain 1 of CD147
AAGFVQAPLSQQRWVGGSVELHCEAVGSPVPEIQWWFEGQGPNDTCSQLWDGARLDRVHIHATYHQHAASTISIDTLVEEDTGTYECRASNDPDRNHLTRAPRVKWVRAQAVVLVLEPGTVFTTVEDLGSKILLTCSLNDSATEVTGHRWLKGGVVLKEDALPGQKTEFKVDSDDQWGEYSCVFLPEPMGTANIQLHG

SEQ ID NO: 9 - protein, 102 aa, Artificial Sequence
  domain 2 of CD147
GPPRVKAVKSSEHINEGETAMLVCKSESVPPVTDWAWYKITDSEDKALMNGSESRFFVSSSQGRSELHIENLNMEADPGQYRCNGTSSKGSDQAIITLRVRS

SEQ ID NO: 10 - protein, 117 aa, Artificial Sequence
  heavy chain variable domain (VH) clone 80R
EVQLVQSGGGVVQPGKSLRLSCAASGFAFSSYAMHWVRQAPGKGLEWVAVISYDGSNKYYADSVKGRFTISRDNSKNTLYLQMNSLRAEDTAVYYCARDRSYYLDYWGQGTLVTVSS

SEQ ID NO: 11 - protein, 107 aa, Artificial Sequence
  light chain variable domain (VL) clone 80R
ETTLTQSPATLSLSPGERATLSCRASQSVRSNLAWYQQKPGQAPRPLIYDASTRATGIPDRFSGSGSGTDFTLTISRLEPEDFAVYYCQQRSNWPPTFGQGTKVEVK

SEQ ID NO: 12 - protein, 127 aa, Artificial Sequence
  VH domain clone S230.15
QAQLVESGGALVQPGRSLRLSCAASGFTFRNYAMHWVRQAPATGLQWLAVITSDGRNKFYADSVKGRFTISREDSKNTLYLQMDSLRGEDTAVYYCVTQRDNSRDYFPHYFHDMDVWGQGTTVAVSS

SEQ ID NO: 13 - protein, 112 aa, Artificial Sequence
  VL domain clone S230.15
DVVLTQSPLSLPVTLGQPASISCRSSQSLVYSDGDTYLNWFQQRPGQSPRRLIYQVSNRDSGVPDRFSGSGSGTDFTLKISRVEAEDVGVYYCMQGSHWPPTFGQGTKVEIK

SEQ ID NO: 14 - protein, 118 aa, Artificial Sequence
  VH domain clone M396
QVQLQQSGAEVKKPGSSVKVSCKASGGTFSSYTISWVRQAPGQGLEWMGGITPILGIANYAQKFQGRVTITTDESTSTAYMELSSLRSEDTAVYYCARDTVMGGMDVWGQGTTVTVSS

SEQ ID NO: 15 - protein, 107 aa, Artificial Sequence
  VL domain clone M396
SYELTQPPSVSVAPGKTARITCGGNNIGSKSVHWYQQKPGQAPVLVVYDDSDRPSGIPERFSGSNSGNTATLTISRVEAGDEADYYCQVWDSSSDYVFGTGTKVTVL

SEQ ID NO: 16 - protein, 121 aa, Artificial Sequence
  VH domain clone F26G19
EVQLEESGTVLARPGASVKMSCKASGYTFTTYRMHWIKQRPGQGLEWIGAIYPGNSDTTYNQKFKDKAKLTAVTSTSSAYMELSSLTNEDSAVYFCTREGIPQLLRTLDYWGQGTSVTVSS

SEQ ID NO: 17 - protein, 107 aa, Artificial Sequence
  VL domain clone F26G19
DILMTQSPSSLSASLGERVSLTCRASQEISGYLSWLQEKPDGTIKRLIYAASTLDSGVPKRFSGSRSGSDYSLTISSLESEDFADYYCLQYVSYPWTFGGGTKLEIK

SEQ ID NO: 18 - protein, 112 aa, Artificial Sequence
  VH domain clone F26G8
QVQLLESGAELVKPGASVKVSCKASGYTFTSYWIHWVKQRPGQGLEWIGEINPSNGRTNYNGNFESKATLTVDKSSNTAYMHLSSLTYEDSAVYHCTRLDYWGQGTTLTVSS

SEQ ID NO: 19 - protein, 107 aa, Artificial Sequence
  VL domain clone F26G8
ELVMTQSPASLSVITGKKVTIRCISNTDIDDDLNWSQLKAGEPPKLLISEGNIFSPGVPSRFSSSGNGTDFVFTIENTLSEDVANNYCFQSDNMPFTFGSGTKLEIK

SEQ ID NO: 20 - protein, 112 aa, Artificial Sequence
  VH domain clone F26G8.2
QVQLQQPGAELVKPGASVKVSCKASGYTFTNYWIHWVKQRPGQGLEWIGEINPGNGRTNYNGNFMNKATLTVDKSSNTAYMQLSSLTSEDSAVYHCARLDYWGQGTTLTVSS

SEQ ID NO: 21 - protein, 107 aa, Artificial Sequence
  VL domain clone F26G8.2
ELVMTQSPASLSVATGKKVTIRCISSTDIDDDMNWYQQKAGKPPKLLISEGNIFSPGVPSRFSSSGNGTDFVFTVENTLSEDVADNYCLQSDNMPFTFGSGTKLGIK

SEQ ID NO: 22 - protein, 123 aa, Artificial Sequence
  VH domain clone F26G18
QLVQLEESGTVLPRPGASVKMSCKASGYTFTSYWMHWVKQRPGQGLEWIGAIYPGNSDTNYNQKFKGRATLTAVTSTSTASMELSSLTNEDSAVYYCTRDGYGSLYYYAMDFWGQGTSVTVSS

SEQ ID NO: 23 - protein, 107 aa, Artificial Sequence
  VL domain clone F26G18
ELVMTQSPSSLSASLGDRVTISCRASQDISNYLNWYQQKPDGTVKLLIYYTSRLHAGVPSRFSGSGSGTDYSLTISNLEQEDIATYFCQQGYTLPYTFGGGTKLEIK

SEQ ID NO: 24 - protein, 123 aa, Artificial Sequence
  VH domain clone 92N
QVQLVQSGAEVKKPGASVKVSCKASGYTFTTYGISWVRQAPGQGLEWMGWISAYNGNTNYAQKLQGRVTMTTDTSTSTAYMELRSLRSDDTAVYYCARDDLSDYGEWLGPDYWGQGTLVTVSS

SEQ ID NO: 25 - protein, 110 aa, Artificial Sequence
  VL domain clone 92N
QPGLTQPPSASGTPGQRVTISCSGSRSNIGGNTVNWYQHVPGTAPKLLIYSNNQRPSGVPDRFSGSKSGTSASLAISGLRSEDEAEYYCATWEDSLSGYVFGPGTKVTVL

SEQ ID NO: 26 - protein, 124 aa, Artificial Sequence
  VH domain clone 91M
QVQLVQSGGGLVQPGGSLRLSCAASGFTFSSSWMSWVRQAPGKGLEWVAYIKQDGSEKYYVDSVKGRFTISRDNAKNSLYLQMDSLRAEDTAVYYCARDQGWGWDGTEYYSDYWGQGTLVTVSS

SEQ ID NO: 27 - protein, 110 aa, Artificial Sequence
  VL domain clone 91M
QSALTQPASVSGSPGQSITISCTGTSSDVGGYNYVSWYQQHPGKAPKLMIYDVSKRPSGVPDRFSGSKSGNTASLTISGLQAEDEADYFCSSYTITDIVVFGGGTKLTVL

SEQ ID NO: 28 - protein, 116 aa, Artificial Sequence
  VH domain clone 27D
QVQLVQSGGGLIQPGGSLRLSCAASGFTVSSNYMSWVRQAPGKGLEWVSVIYSGGSTYYADSVKGRFTISRDNSKNTLYLQMNSLRAEDTAVYYCARNFGEDFDYWGQGTLVTVSS

SEQ ID NO: 29 - protein, 110 aa, Artificial Sequence
  VL domain clone 27D
QSALTQPASVSGSPGQSITISCTGTSSDLGGHNFVSWYQQHPGKAPKLMIYDVFNRPSGVSSRFSGSKSGNTASLTISGLQAEDEADYFCSSYTITNIVVFGRGTKLTVL

SEQ ID NO: 30 - protein, 122 aa, Artificial Sequence
  VH domain clone 26H
QVQLQESGGGLVKPGGSLRLSCAASGFTFSDYYMSWIRQAPGKGLEWVSYISSSGSTIYYADSVKGRFTISRDNAKNSLYLQMNSLRAEDTAVYYCARERWLQIGEDAFDIWGQGTTVTVSS

SEQ ID NO: 31 - protein, 112 aa, Artificial Sequence
  VL domain clone 26H
QTVVTQEPSFSVSPGGTITLTCDLNSGLVSSSHYPSWYQQTPGQAPRTLIYNTNIRSSGVPDRFSGAILGNKAALTITGAQAEDESDYYCVLYMGSGISVFGGGTKLTVLGQ

SEQ ID NO: 32 - protein, 127 aa, Artificial Sequence
  VH domain clone 12E
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARVGYYGDYAWGYYYYGMDVWGQGTLVTVSS

SEQ ID NO: 33 - protein, 111 aa, Artificial Sequence
  VL domain clone 12E
SYELTQPPSASGTPGQRVTISCSGSSSSIGSETVNWYQQLPGTAPKLLIYSNNQRPSGVPDRFSGSKSGTSASLAISGLQSEDEADYYCTAWDDTLNGRVIFGGGTKLTVL

SEQ ID NO: 34 - protein, 117 aa, Artificial Sequence
  VH domain clone 8C
EVQLVQSGGGLVKPGGSLRLSCAASGFTFSDYYMSWIRQAPGKGLEWVSYISSSGSTIYYADSVKGRFTISRDNAKNSLYLQMNSLRAEDTGVYYCARDWGYSFGDWGQGTLVTVSS

SEQ ID NO: 35 - protein, 107 aa, Artificial Sequence
  VL domain clone 8C
DIQMTQSPSSLSASVGDRVTITCRATQSISTHLNWYQQRGGKAPKLLIYGASTLESGVPSRFSGSGSGTEFTLTISSLQPEDFATYYCLQHRSYPWTFGQGTNVEIK

SEQ ID NO: 36 - protein, 121 aa, Artificial Sequence
  VH domain clone CR3009
EVQLVESGGGVVQPGRSLRLSCAASGFTFSDYPMNWVRQAPGKGLEWVSSISGSGGSTYYADSVKGRFTISRDNSKNTLYLQMNSLRAEDTAVYYCAKGLFMVTTYAFDYWGQGTLVTVLE

SEQ ID NO: 37 - protein, 107 aa, Artificial Sequence
  VL domain clone CR3009
DIELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIK

SEQ ID NO: 38 - protein, 120 aa, Artificial Sequence
  VH domain clone CR3006
EVQLVESGGGLVQPGGSLRLSCAASGFTFSGYPMHWVRQAPGKGLEWVAVISYDGSNKYYADSVKGRFTISRDNSKNTLYLQMNSLRAEDTAVYYCAKDGSPRTPSFDYWGQGTLVTVLE

SEQ ID NO: 39 - protein, 107 aa, Artificial Sequence
  VL domain clone CR3006
DIQMTQSPHSLSASVGDRVTITCRASQGISNYLAWYQQKPGKVPKLLIYAASTLQSGVPSRFSGSGSGTDFTLTISSLQPEDVGVYYCQQRFRTPVTFGQGTKLEIK

SEQ ID NO: 40 - protein, 118 aa, Artificial Sequence
  VH domain clone CR3018
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKFNPFTSFDYWGQGTLVTVSS

SEQ ID NO: 41 - protein, 107 aa, Artificial Sequence; identical to SEQ ID NO: 37
  VL domain clone CR3018

SEQ ID NO: 42 - protein, 120 aa, Artificial Sequence
  VH domain clone CR3013
EVQLVESGGGLVQPGGSLRLSCAASGFTFSDHYMDWVRQAPGKGLEWVGRTRNKANSYTTEYAASVKGRFTISRDDSKNSLYLQMNSLKTEDTAVYYCAKGLTPLYFDYWGQGTLVTVSS

SEQ ID NO: 43 - protein, 107 aa, Artificial Sequence; identical to SEQ ID NO: 37
  VL domain clone CR3013

SEQ ID NO: 44 - protein, 120 aa, Artificial Sequence
  VH domain clone CR3014
EVQLVESGGGLVQPGGSLRLSCAASGFTFSDHYMDWVRQAPGKGLEWVGRTRNKANSYTTEYAASVKGRFTISRDDSKNSLYLQMNSLKTEDTAVYYCARGISPFYFDYWGQGTLVTVSS

SEQ ID NO: 45 - protein, 107 aa, Artificial Sequence; identical to SEQ ID NO: 37
  VL domain clone CR3014

SEQ ID NO: 46 - protein, 115 aa, Artificial Sequence
  VH domain clone CR3015
EVQLVESGGGVVRPGGSLRLSCAASGFTFDDYGMSWVRQAPGKGLEWVSGINWNGGSTGYADSVKGRFTISRDNAKNSLYLQMNSLRAEDTAVYYCARGLSLRPWGQGTLVTVSR

SEQ ID NO: 47 - protein, 107 aa, Artificial Sequence
  VL domain clone CR3015
SELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVL

SEQ ID NO: 48 - protein, 120 aa, Artificial Sequence
  VH domain clone AS3-3
QVQLVQSGAEVKKPGESLKISCKGSGYSFANHWIAWVRQMPGKGLEWMGIFNPDNSDTTYSPSFQGQVTFSADKSISIAYLHWSSLKASDTAIYYCARLALSDGWLHDFWGQGTLVTVSS

SEQ ID NO: 49 - protein, 112 aa, Artificial Sequence
  VL domain clone AS3-3
EIVMTQSPLSLPVTLGQPASISCKSSQSLVDTNGNTYLIWFHQRPGQSPRRLIYQVSKRDSGVPDRFSGSGSGTDFTLKISRVEAEDVGVYFCMQGTYRPPTLGQGTKVEIK

SEQ ID NO: 50 - protein, 119 aa, Artificial Sequence
  VH domain clone CR3022
QMQLVQSGTEVKKPGESLKISCKGSGYGFITYWIGWVRQMPGKGLEWMGIIYPGDSETRYSPSFQGQVTISADKSINTAYLQWSSLKASDTAIYYCAGGSGISTPMDVWGQGTTVTVSS

SEQ ID NO: 51 - protein, 113 aa, Artificial Sequence
  VL domain clone CR3022
DIQLTQSPDSLAVSLGERATINCKSSQSVLYSSINKNYLAWYQQKPGQPPKLLIYWASTRESGVPDRFSGSGSGTDFTLTISSLQAEDVAVYYCQQYYSTPYTFGQGTKVEIK

SEQ ID NO: 52 - protein, 122 aa, Artificial Sequence
  VH domain clone B1
QVQLLESGAEVKKPGASAKVSCKASGCTFTSYGISWVRQAPGQGLEWMGWISAYNGNTNYAQKLQGRVTMTTDTSTSTAYMELRSLRSDDTAVYYCARGVAVAGTWDWFDPWGQGTLVTVSS

SEQ ID NO: 53 - protein, 108 aa, Artificial Sequence
  VL domain clone B1
DIVLTQTPGTLSLSPGERATLSCRASQSVRTYLAWYQQKPGQVPRLLIYGASSRATGIPDRFSGSGSGTDFTLTISRLEPEDFAVYYCQQYGSSLWTFGQGTRLEIKR

SEQ ID NO: 54 - protein, 11 aa, Artificial Sequence
  albumin-binding-peptide core sequence
DICLPRWGCLW

SEQ ID NO: 55 - protein, 21 aa, Artificial Sequence
  signal peptide
MGTSLLCWMALCLLGADHADG

SEQ ID NO: 56 - protein, 21 aa, Artificial Sequence
  signal peptide derived from IgG1
MSLPVTALLLPLALLLHAARP

SEQ ID NO: 57 - protein, 20 aa, Artificial Sequence
  signal peptide derived from CD8
MAVPTQVLGLLLLWLTDARC

SEQ ID NO: 58 - protein, 127 aa, Artificial Sequence
  dAb clone 5
QVQLQESGGGLVQAGGSLRLSCVDSGRTFSGGTMAWFRQAPGKAREFVAADRGFTSTYYADAVKGRFTISRDNAENAAENAVYLQMNSLKPEDTAVYYCAARPTVAFSTDEIFYKFWGQGTQVTVSS

SEQ ID NO: 59 - protein, 125 aa, Artificial Sequence
  dAb clone 7
QVQLQESGGGLVEAGGSLRLSCVASGFTFEDYNIGWFRQAPGKEREGVSYIRANGGTTYYTDSVKGRFTISADNAKNTVYLQMSNLKSEDTAVYYCAADGSSWPIDLPVYEYDYWGQGTQVTVSS

SEQ ID NO: 60 - protein, 125 aa, Artificial Sequence
  dAb clone 9
QVQLQESGGGLVQAGDSLRLSCAASGRTSDTMTIGWFRQAPGKEREFVASINWSGSTTYYADSVNERFTISRDDAKNTVYLQMNSLKPEDTAVYYCAAKRGVWTIKSAAADFASWGQGTQVTVSS

SEQ ID NO: 61 - protein, 232 aa, Artificial Sequence
  hinge-IgG1 Fc region
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 228 aa, Artificial Sequence
  IgG2 Fc
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 63 - protein, 27 aa, Artificial Sequence
  foldon T4 domain
GYIPEAPRDGQAYVRKDGEWVLLSTFL

SEQ ID NO: 64 - protein, 30 aa, Artificial Sequence
  Human TenC domain (N-terminus of Tenascin C)
ACGCAAAPDVKELLSRLEELENLVSSLREQ

SEQ ID NO: 65 - protein, 30 aa, Artificial Sequence
  Chicken TenC domain
ACGCAAAPDVKDLLSRLEELEGLVSSLREQ

SEQ ID NO: 66 - protein, 55 aa, Artificial Sequence
  cartilage oligomeric matrix protein (COMP) homopentamer
AGSDLGPQMLRELQETNAALQDVRELLRQQVREITFLKNTVMECDACGSGKKDPK

SEQ ID NO: 67 - protein, 10 aa, Artificial Sequence
  linker
GGGGSGGGGS

SEQ ID NO: 68 - protein, 20 aa, Artificial Sequence
  linker
GGGGSGGGGSGGGGSGGGGS

SEQ ID NO: 69 - protein, 25 aa, Artificial Sequence
  linker
GGGGSGGGGSGGGGSGGGGSGGGGS

SEQ ID NO: 70 - protein, 16 aa, Artificial Sequence
  linker
SGGGGSGGGGSGGGGS

SEQ ID NO: 71 - protein, 15 aa, Artificial Sequence
  linker
GGGGSGGGGSGGGGS

SEQ ID NO: 72 - protein, 15 aa, Artificial Sequence
  linker
GGGGSGGGGSGGGAS

SEQ ID NO: 73 - protein, 1273 aa, Severe acute respiratory syndrome coronavirus (SARS-CoV-2)
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 74 - protein, 668 aa, SARS-CoV-2
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSP

SEQ ID NO: 75 - protein, 592 aa, SARS-CoV-2
RRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 76 - protein, 955 aa, Artificial Sequence
  hACE2_18-740_HuIgG1Fc polypeptide
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 1057 aa, Artificial Sequence
  hACE2_18-740_HuIgMFc polypeptide
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSSGGGSIAELPPKVSVFVPPRDGFFGNPRSKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKST

SEQ ID NO: 78 - protein, 922 aa, Artificial Sequence
  ACE2_18-605_HuIgMFc polypeptide
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGSGGGSIAELPPKVSVFVPPRDGFFGNPRSKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKST

SEQ ID NO: 79 - protein, 820 aa, Artificial Sequence
  ACE2_18-605_HuIgG1Fc polypeptide
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 1270 aa, Artificial Sequence
  hACE2_18-740_HuIgG1Fc_CD147_22-320 polypeptide
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSGGGGSGGGGSGGGGSAAGFVQAPLSQQRWVGGSVELHCEAVGSPVPEIQWWFEGQGPNDTCSQLWDGARLDRVHIHATYHQHAASTISIDTLVEEDTGTYECRASNDPDRNHLTRAPRVKWVRAQAVVLVLEPGTVFTTVEDLGSKILLTCSLNDSATEVTGHRWLKGGVVLKEDALPGQKTEFKVDSDDQWGEYSCVFLPEPMGTANIQLHGPPRVKAVKSSEHINEGETAMLVCKSESVPPVTDWAWYKITDSEDKALMNGSESRFFVSSSQGRSELHIENLNMEADPGQYRCNGTSSKGSDQAIITLRVRS

SEQ ID NO: 81 - protein, 1169 aa, Artificial Sequence
  hACE2_18-740_HuIgG1Fc_CD147_22-219 polypeptide
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKSGGGGSGGGGSGGGGSAAGFVQAPLSQQRWVGGSVELHCEAVGSPVPEIQWWFEGQGPNDTCSQLWDGARLDRVHIHATYHQHAASTISIDTLVEEDTGTYECRASNDPDRNHLTRAPRVKWVRAQAVVLVLEPGTVFTTVEDLGSKILLTCSLNDSATEVTGHRWLKGGVVLKEDALPGQKTEFKVDSDDQWGEYSCVFLPEPMGTANIQLHG

SEQ ID NO: 82 - protein, 1073 aa, Artificial Sequence
  hACE2_18-740_HuIgG1Fc_CD147_219-320 polypeptide
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSGGGGSGGGGSGGGGSGPPRVKAVKSSEHINEGETAMLVCKSESVPPVTDWAWYKITDSEDKALMNGSESRFFVSSSQGRSELHIENLNMEADPGQYRCNGTSSKGSDQAIITLRVRS

SEQ ID NO: 83 - protein, 1270 aa, Artificial Sequence
  CD147_22-320_16gs_hACE2_18-740_HuIgG1Fc polypeptide
AAGFVQAPLSQQRWVGGSVELHCEAVGSPVPEIQWWFEGQGPNDTCSQLWDGARLDRVHIHATYHQHAASTISIDTLVEEDTGTYECRASNDPDRNHLTRAPRVKWVRAQAVVLVLEPGTVFTTVEDLGSKILLTCSLNDSATEVTGHRWLKGGVVLKEDALPGQKTEFKVDSDDQWGEYSCVFLPEPMGTANIQLHGPPRVKAVKSSEHINEGETAMLVCKSESVPPVTDWAWYKITDSEDKALMNGSESRFFVSSSQGRSELHIENLNMEADPGQYRCNGTSSKGSDQAIITLRVRSSGGGGSGGGGSGGGGSQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - protein, 1084 aa, Artificial Sequence
  hACE2_18-605-L-SARS_aCoV_CR3022_ScFv_ HL-HuIgG1Fc polypeptide
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGGGGGSGGGGSGGGASQMQLVQSGTEVKKPGESLKISCKGSGYGFITYWIGWVRQMPGKGLEWMGIIYPGDSETRYSPSFQGQVTISADKSINTAYLQWSSLKASDTAIYYCAGGSGISTPMDVWGQGTTVTVSSGGGGSGGGGSGGGGSDIQLTQSPDSLAVSLGERATINCKSSQSVLYSSINKNYLAWYQQKPGQPPKLLIYWASTRESGVPDRFSGSGSGTDFTLTISSLQAEDVAVYYCQQYYSTPYTFGQGTKVEIKGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 1219 aa, Artificial Sequence
  hACE2_18-740-L-SARS_aCoV_CR3022_ScFv_HL- HuIgG1Fc polypeptide
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSGGGGSGGGGSGGGASQMQLVQSGTEVKKPGESLKISCKGSGYGFITYWIGWVRQMPGKGLEWMGIIYPGDSETRYSPSFQGQVTISADKSINTAYLQWSSLKASDTAIYYCAGGSGISTPMDVWGQGTTVTVSSGGGGSGGGGSGGGGSDIQLTQSPDSLAVSLGERATINCKSSQSVLYSSINKNYLAWYQQKPGQPPKLLIYWASTRESGVPDRFSGSGSGTDFTLTISSLQAEDVAVYYCQQYYSTPYTFGQGTKVEIKGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - protein, 1082 aa, Artificial Sequence
  hACE2_18-605-L-SARS_aCoV-S2_B1_ScFv_HL- HuIgG1Fc polypeptide
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGGGGGSGGGGSGGGASQVQLLESGAEVKKPGASAKVSCKASGCTFTSYGISWVRQAPGQGLEWMGWISAYNGNTNYAQKLQGRVTMTTDTSTSTAYMELRSLRSDDTAVYYCARGVAVAGTWDWFDPWGQGTLVTVSSGGGGSGGGGSGGGGSDIVLTQTPGTLSLSPGERATLSCRASQSVRTYLAWYQQKPGQVPRLLIYGASSRATGIPDRFSGSGSGTDFTLTISRLEPEDFAVYYCQQYGSSLWTFGQGTRLEIKRGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - protein, 1217 aa, Artificial Sequence
  hACE2_18-740-L-SARS_aCoV-S2_B1_ScFv_HL- HuIgG1Fc polypeptide
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSGGGGSGGGGSGGGASQVQLLESGAEVKKPGASAKVSCKASGCTFTSYGISWVRQAPGQGLEWMGWISAYNGNTNYAQKLQGRVTMTTDTSTSTAYMELRSLRSDDTAVYYCARGVAVAGTWDWFDPWGQGTLVTVSSGGGGSGGGGSGGGGSDIVLTQTPGTLSLSPGERATLSCRASQSVRTYLAWYQQKPGQVPRLLIYGASSRATGIPDRFSGSGSGTDFTLTISRLEPEDFAVYYCQQYGSSLWTFGQGTRLEIKRGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - protein, 633 aa, Artificial Sequence
  CD147_22-320_HuIgMFc polypeptide
AAGFVQAPLSQQRWVGGSVELHCEAVGSPVPEIQWWFEGQGPNDTCSQLWDGARLDRVHIHATYHQHAASTISIDTLVEEDTGTYECRASNDPDRNHLTRAPRVKWVRAQAVVLVLEPGTVFTTVEDLGSKILLTCSLNDSATEVTGHRWLKGGVVLKEDALPGQKTEFKVDSDDQWGEYSCVFLPEPMGTANIQLHGPPRVKAVKSSEHINEGETAMLVCKSESVPPVTDWAWYKITDSEDKALMNGSESRFFVSSSQGRSELHIENLNMEADPGQYRCNGTSSKGSDQAIITLRVRSSGGGSIAELPPKVSVFVPPRDGFFGNPRSKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKST

SEQ ID NO: 89 - protein, 430 aa, Artificial Sequence
  CD147-D1_22-219_HuIgG1Fc polypeptide
AAGFVQAPLSQQRWVGGSVELHCEAVGSPVPEIQWWFEGQGPNDTCSQLWDGARLDRVHIHATYHQHAASTISIDTLVEEDTGTYECRASNDPDRNHLTRAPRVKWVRAQAVVLVLEPGTVFTTVEDLGSKILLTCSLNDSATEVTGHRWLKGGVVLKEDALPGQKTEFKVDSDDQWGEYSCVFLPEPMGTANIQLHGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - protein, 334 aa, Artificial Sequence
  CD147-D2_219-320_HuIgG1Fc polypeptide
GPPRVKAVKSSEHINEGETAMLVCKSESVPPVTDWAWYKITDSEDKALMNGSESRFFVSSSQGRSELHIENLNMEADPGQYRCNGTSSKGSDQAIITLRVRSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - protein, 531 aa, Artificial Sequence
  CD147_22-320_HuIgG1Fc polypeptide
AAGFVQAPLSQQRWVGGSVELHCEAVGSPVPEIQWWFEGQGPNDTCSQLWDGARLDRVHIHATYHQHAASTISIDTLVEEDTGTYECRASNDPDRNHLTRAPRVKWVRAQAVVLVLEPGTVFTTVEDLGSKILLTCSLNDSATEVTGHRWLKGGVVLKEDALPGQKTEFKVDSDDQWGEYSCVFLPEPMGTANIQLHGPPRVKAVKSSEHINEGETAMLVCKSESVPPVTDWAWYKITDSEDKALMNGSESRFFVSSSQGRSELHIENLNMEADPGQYRCNGTSSKGSDQAIITLRVRSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 92 - protein, 21 aa, Artificial Sequence
  signal peptide derived from mouse Ig Kappa
METDTLLLWVLLLWVPGSTGD

SEQ ID NO: 93 - protein, 19 aa, Artificial Sequence
  signal peptide derived from human IgG2 heavy chain
MGWSCIILFLVATATGVHS

SEQ ID NO: 94 - DNA, 17 bp, Artificial Sequence
  primer M13Rvs
caggaaacagctatgac

SEQ ID NO: 95 - DNA, 21 bp, Artificial Sequence
  primer M13 Fwd
gtcgtctttccagacgttagt

SEQ ID NO: 96 - protein, 1084 aa, Artificial Sequence
  SARS_aCoV_CR3022_ScFv_HL-L _ACE2_18-605_ shuttle_HuIgG1Fc polypeptide
QMQLVQSGTEVKKPGESLKISCKGSGYGFITYWIGWVRQMPGKGLEWMGIIYPGDSETRYSPSFQGQVTISADKSINTAYLQWSSLKASDTAIYYCAGGSGISTPMDVWGQGTTVTVSSGGGGSGGGGSGGGGSDIQLTQSPDSLAVSLGERATINCKSSQSVLYSSINKNYLAWYQQKPGQPPKLLIYWASTRESGVPDRFSGSGSGTDFTLTISSLQAEDVAVYYCQQYYSTPYTFGQGTKVEIKGSGGGGSGGGGSGGGGSQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - protein, 1219 aa, Artificial Sequence
  SARS_aCoV_CR3022_ScFv_HL-L_ACE2_18-740_ shuttle_HuIgG1Fc polypeptide
QMQLVQSGTEVKKPGESLKISCKGSGYGFITYWIGWVRQMPGKGLEWMGIIYPGDSETRYSPSFQGQVTISADKSINTAYLQWSSLKASDTAIYYCAGGSGISTPMDVWGQGTTVTVSSGGGGSGGGGSGGGGSDIQLTQSPDSLAVSLGERATINCKSSQSVLYSSINKNYLAWYQQKPGQPPKLLIYWASTRESGVPDRFSGSGSGTDFTLTISSLQAEDVAVYYCQQYYSTPYTFGQGTKVEIKGSGGGGSGGGGSGGGGSQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 98 - protein, 1082 aa, Artificial Sequence
  SARS_aCoV-S2_B1_ScFv_HL-L _ACE2_18-605_ shuttle_HuIgG1Fc polypeptide
QVQLLESGAEVKKPGASAKVSCKASGCTFTSYGISWVRQAPGQGLEWMGWISAYNGNTNYAQKLQGRVTMTTDTSTSTAYMELRSLRSDDTAVYYCARGVAVAGTWDWFDPWGQGTLVTVSSGGGGSGGGGSGGGGSDIVLTQTPGTLSLSPGERATLSCRASQSVRTYLAWYQQKPGQVPRLLIYGASSRATGIPDRFSGSGSGTDFTLTISRLEPEDFAVYYCQQYGSSLWTFGQGTRLEIKRGSGGGGSGGGGSGGGGSQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - protein, 1217 aa, Artificial Sequence
  SARS_aCoV-S2_B1_ScFv_HL-L _ACE2_18-740_ shuttle_HuIgG1Fc polypeptide
QVQLLESGAEVKKPGASAKVSCKASGCTFTSYGISWVRQAPGQGLEWMGWISAYNGNTNYAQKLQGRVTMTTDTSTSTAYMELRSLRSDDTAVYYCARGVAVAGTWDWFDPWGQGTLVTVSSGGGGSGGGGSGGGGSDIVLTQTPGTLSLSPGERATLSCRASQSVRTYLAWYQQKPGQVPRLLIYGASSRATGIPDRFSGSGSGTDFTLTISRLEPEDFAVYYCQQYGSSLWTFGQGTRLEIKRGSGGGGSGGGGSGGGGSQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 100 - protein, 579 aa, Artificial Sequence
  SARS_aCoV_CR3022_scFv4-Ig_HL_HC_mIgG2aFc polypeptide
QMQLVQSGTEVKKPGESLKISCKGSGYGFITYWIGWVRQMPGKGLEWMGIIYPGDSETRYSPSFQGQVTISADKSINTAYLQWSSLKASDTAIYYCAGGSGISTPMDVWGQGTTVTVSSGGGGSGGGGSGGGGSDIQLTQSPDSLAVSLGERATINCKSSQSVLYSSINKNYLAWYQQKPGQPPKLLIYWASTRESGVPDRFSGSGSGTDFTLTISSLQAEDVAVYYCQQYYSTPYTFGQGTKVEIKSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 101 - protein, 353 aa, Artificial Sequence
  SARS_aCoV_CR3022_scFv4-Ig_HL_LC_mKappa polypeptide
QMQLVQSGTEVKKPGESLKISCKGSGYGFITYWIGWVRQMPGKGLEWMGIIYPGDSETRYSPSFQGQVTISADKSINTAYLQWSSLKASDTAIYYCAGGSGISTPMDVWGQGTTVTVSSGGGGSGGGGSGGGGSDIQLTQSPDSLAVSLGERATINCKSSQSVLYSSINKNYLAWYQQKPGQPPKLLIYWASTRESGVPDRFSGSGSGTDFTLTISSLQAEDVAVYYCQQYYSTPYTFGQGTKVEIKADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 102 - protein, 577 aa, Artificial Sequence
  SARS_aCoV-S2_B1_scFv4-Ig_HL_HC_mIgG2aFc polypeptide
QVQLLESGAEVKKPGASAKVSCKASGCTFTSYGISWVRQAPGQGLEWMGWISAYNGNTNYAQKLQGRVTMTTDTSTSTAYMELRSLRSDDTAVYYCARGVAVAGTWDWFDPWGQGTLVTVSSGGGGSGGGGSGGGGSDIVLTQTPGTLSLSPGERATLSCRASQSVRTYLAWYQQKPGQVPRLLIYGASSRATGIPDRFSGSGSGTDFTLTISRLEPEDFAVYYCQQYGSSLWTFGQGTRLEIKRSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 103 - protein, 351 aa, Artificial Sequence
  SARS_aCoV-S2_B1_scFv4-Ig_HL_LC_mKappa polypeptide
QVQLLESGAEVKKPGASAKVSCKASGCTFTSYGISWVRQAPGQGLEWMGWISAYNGNTNYAQKLQGRVTMTTDTSTSTAYMELRSLRSDDTAVYYCARGVAVAGTWDWFDPWGQGTLVTVSSGGGGSGGGGSGGGGSDIVLTQTPGTLSLSPGERATLSCRASQSVRTYLAWYQQKPGQVPRLLIYGASSRATGIPDRFSGSGSGTDFTLTISRLEPEDFAVYYCQQYGSSLWTFGQGTRLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 104 - protein, 723 aa, Artificial Sequence
  processed ACE2 ectodomain variant (HH:NN)
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 105 - protein, 588 aa, Artificial Sequence
  truncated ACE2 variant, aa 18-605
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVG

SEQ ID NO: 106 - protein, 57 aa, Artificial Sequence
  kinesin motor protein: parallel homodimer
MHAALSTEVVHLRQRTEELLRCNEQQAAELETCKEQLFQSNMERKELHNTVMDLRGN

SEQ ID NO: 107 - protein, 50 aa, Artificial Sequence [sequence omitted]

SEQ ID NO: 108 - protein, 61 aa, Artificial Sequence
  Archaeal box C/D sRNP core protein: anti-parallel heterodimer
RYVVALVKALEEIDESINMLNEKLEDIRAVKESEITEKFEKKIRELRELRRDVEREIEEVM

SEQ ID NO: 109 - protein, 31 aa, Artificial Sequence
  mannose-binding protein A: parallel homotrimer
AIEVKLANMEAEINTLKSKLELTNKLHAFSM

SEQ ID NO: 110 - protein, 29 aa, Artificial Sequence
  coiled-coil serine-rich protein 1: parallel homotrimer
EWEALEKKLAALESKLQALEKKLEALEHG

SEQ ID NO: 111 - protein, 51 aa, Artificial Sequence
  polypeptide release factor 2: anti-parallel heterotrimer, Chain B
VVDTLDQMKQGLEDVSGLLELAVEADDEETFNEAVAELDALEEKLAQLEFR

SEQ ID NO: 112 - protein, 51 aa, Artificial Sequence
  SNAP-25 and SNARE: parallel heterotetramer, Chain A
IETRHSEIIKLENSIRELHDMFMDMAMLVESQGEMIDRIEYNVEHAVDYVE

SEQ ID NO: 113 - protein, 67 aa, Artificial Sequence
  SNAP-25 and SNARE: parallel heterotetramer, Chain B
ALSEIETRHSEIIKLENSIRELHDMFMDMAMLVESQGEMIDRIEYNVEHAVDYVERAVSDTKKAVKY

SEQ ID NO: 114 - protein, 69 aa, Artificial Sequence
  SNAP-25 and SNARE: parallel heterotetramer, Chain C
ELEEMQRRADQLADESLESTRRMLQLVEESKDAGIRTLVMLDEQGEQLERIEEGMDQINKDMKEAEKNL

SEQ ID NO: 115 - protein, 51 aa, Artificial Sequence; identical to SEQ ID NO: 112
  SNAP-25 and SNARE: parallel heterotetramer, Chain D

SEQ ID NO: 116 - protein, 20 aa, Artificial Sequence
  Lac repressor: parallel homotetramer
SPRALADSLMQLARQVSRLE

SEQ ID NO: 117 - protein, 136 aa, Artificial Sequence
  Apolipoprotein E: anti-parallel heterotetramer
SGQRWELALGRFWDYLRWVQTLSEQVQEELLSSQVTQELRALMDETMKELKAYKSELEEQLTARLSKELQAAQARLGADMEDVCGRLVQYRGEVQAMLGQSTEELRVRLASHLRKLRKRLLRDADDLQKRLAVYQA

SEQ ID NO: 118 - protein, 121 aa, Artificial Sequence
  truncated Fc region (Hinge-CH3)
EPKSCDKTHTCPPCGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 119 - protein, 131 aa, Artificial Sequence
  truncated Fc region (Hinge-CH3)
EPKSCDKTHTCPPCGGGSSGGGSGGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 120 - protein, 232 aa, Artificial Sequence
  HuIgGFc with LALA mutations
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 121 - protein, 232 aa, Artificial Sequence
  HuIgGFc with LALA and PG mutations
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 122 - protein, 531 aa, Artificial Sequence
  hCD147-Fc polypeptide
AAGFVQAPLSQQRWVGGSVELHCEAVGSPVPEIQWWFEGQGPNDTCSQLWDGARLDRVHIHATYHQHAASTISIDTLVEEDTGTYECRASNDPDRNHLTRAPRVKWVRAQAVVLVLEPGTVFTTVEDLGSKILLTCSLNDSATEVTGHRWLKGGVVLKEDALPGQKTEFKVDSDDQWGEYSCVFLPEPMGTANIQLHGPPRVKAVKSSEHINEGETAMLVCKSESVPPVTDWAWYKITDSEDKALMNGSESRFFVSSSQGRSELHIENLNMEADPGQYRCNGTSSKGSDQAIITLRVRSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 123 - protein, 430 aa, Artificial Sequence; identical to SEQ ID NO: 89
  hCD147 D1-Fc polypeptide

SEQ ID NO: 124 - protein, 334 aa, Artificial Sequence; identical to SEQ ID NO: 90
  CD147 D2-Fc polypeptide

SEQ ID NO: 125 - protein, 955 aa, Artificial Sequence
  Inactive ACE2-Fc (LALA-PG) (ACE2(HH:NN)-Fc LALA-PG) polypeptide
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 126 - protein, 12 aa, Artificial Sequence
  IgG1 hinge
EPKSCDKTHTCP

SEQ ID NO: 127 - protein, 98 aa, Artificial Sequence
  human IgG1 CH1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 128 - protein, 232 aa, Artificial Sequence
  HuIgG1Fc with LALA, PG and YTE mutations
EPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 129 - protein, 955 aa, Artificial Sequence
  hACE2_18-740_HuIgG1Fc (LALA-PG, YTE) polypeptide
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 130 - protein, 955 aa, Artificial Sequence; identical to SEQ ID NO: 129
  inactive ACE2-Fc (LALA-PG, YTE) (ACE2(HH:NN)- Fc LALA-PG, YTE) polypeptide

SEQ ID NO: 131 - protein, 105 aa, Artificial Sequence [sequence omitted]

SEQ ID NO: 132 - protein, 1055 aa, Artificial Sequence
  inactive ACE2 (HH:NN)-Heavy chain (CH1-hinge- CH2-CH3)
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 133 - protein, 1055 aa, Artificial Sequence
  inactive ACE2 (HH:NN)-Heavy chain (IgG1 LALA- PG)
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 134 - protein, 1055 aa, Artificial Sequence
  inactive ACE2 (HH:NN)-Heavy chain (IgG1 LALA- PG, YTE)
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 135 - protein, 832 aa, Artificial Sequence
  inactive ACE2 (HH:NN)-Light chain (constant kappa)
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHNEMGNIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 136 - protein, 42 aa, Artificial Sequence
  p53 oligomerisation domain
KKKPLDGEYFTLQIRGRERFEMFRELNEALELKDAQAGKEPG

SEQ ID NO: 137 - protein, 805 aa, Homo sapiens [sequence omitted]

SEQ ID NO: 138 - protein, 24 aa, Artificial Sequence
  polypeptide release factor 2: anti-parallel heterotrimer, Chain A
INPVNNRIQDLTERSDVLRGYLDY

SEQ ID NO: 139 - protein, 591 aa, Homo sapiens
RGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 140 - protein, 20 aa, Artificial Sequence
  cleavage site, 2A-like sequence
RAEGRGSLLTCGDVEENPGP